SEQUENCE LISTING (130 sequences)

SEQ ID NO: 1 - DNA, 696 bp, Synechococcus elongatus
  PCC7942 Synpcc7942_1593 (YP_400610) nucleotide
atgccgcagcttgaagccagccttgaactggactttcaaagcgagtcctacaaagacgcttacagccgcatcaacgcgatcgtgattgaaggcgaacaagaggcgttcgacaactacaatcgccttgctgagatgctgcccgaccagcgggatgagcttcacaagctagccaagatggaacagcgccacatgaaaggctttatggcctgtggcaaaaatctctccgtcactcctgacatgggttttgcccagaaatttttcgagcgcttgcacgagaacttcaaagcggcggctgcggaaggcaaggtcgtcacctgcctactgattcaatcgctaatcatcgagtgctttgcgatcgcggcttacaacatctacatcccagtggcggatgcttttgcccgcaaaatcacggagggggtcgtgcgcgacgaatacctgcaccgcaacttcggtgaagagtggctgaaggcgaattttgatgcttccaaagccgaactggaagaagccaatcgtcagaacctgcccttggtttggctaatgctcaacgaagtggccgatgatgctcgcgaactcgggatggagcgtgagtcgctcgtcgaggactttatgattgcctacggtgaagctctggaaaacatcggcttcacaacgcgcgaaatcatgcgtatgtccgcctatggccttgcggccgtttga

SEQ ID NO: 2 - protein, 231 aa, Synechococcus elongatus
  PCC7942 Synpcc7942_1593 (YP_400610) amino acid
MPQLEASLELDFQSESYKDAYSRINAIVIEGEQEAFDNYNRLAEMLPDQRDELHKLAKMEQRHMKGFMACGKNLSVTPDMGFAQKFFERLHENFKAAAAEGKVVTCLLIQSLIIECFAIAAYNIYIPVADAFARKITEGVVRDEYLHRNFGEEWLKANFDASKAELEEANRQNLPLVWLMLNEVADDARELGMERESLVEDFMIAYGEALENIGFTTREIMRMSAYGLAAV

SEQ ID NO: 3 - DNA, 696 bp, Synechocystis sp.
  PCC6803 sll0208 (NP_442147) nucleotide
atgcccgagcttgctgtccgcaccgaatttgactattccagcgaaatttacaaagacgcctatagccgcatcaacgccattgtcattgaaggcgaacaggaagcctacagcaactacctccagatggcggaactcttgccggaagacaaagaagagttgacccgcttggccaaaatggaaaaccgccataaaaaaggtttccaagcctgtggcaacaacctccaagtgaaccctgatatgccctatgcccaggaatttttcgccggtctccatggcaatttccagcacgcttttagcgaagggaaagttgttacctgtttattgatccaggctttgattatcgaagcttttgcgatcgccgcctataacatatatatccctgtggcggacgactttgctcggaaaatcactgagggcgtagtcaaggacgaatacacccacctcaactacggggaagaatggctaaaggccaactttgccaccgctaaggaagaactggagcaggccaacaaagaaaacctacccttagtgtggaaaatgctcaaccaagtgcagggggacgccaaggtattgggcatggaaaaagaagccctagtggaagattttatgatcagctacggcgaagccctcagtaacatcggcttcagcaccagggaaattatgcgtatgtcttcctacggtttggccggagtctag

SEQ ID NO: 4 - protein, 231 aa, Synechocystis sp.
  PCC6803 sll0208 (NP_442147) amino acid
MPELAVRTEFDYSSEIYKDAYSRINAIVIEGEQEAYSNYLQMAELLPEDKEELTRLAKMENRHKKGFQACGNNLQVNPDMPYAQEFFAGLHGNFQHAFSEGKVVTCLLIQALIIEAFAIAAYNIYIPVADDFARKITEGVVKDEYTHLNYGEEWLKANFATAKEELEQANKENLPLVWKMLNQVQGDAKVLGMEKEALVEDFMISYGEALSNIGFSTREIMRMSSYGLAGV

SEQ ID NO: 5 - DNA, 699 bp, Nostoc punctiforme
  PCC 73102 Npun02004178 (ZP_00108838) nucleotide
atgcagcagcttacagaccaatctaaagaattagatttcaagagcgaaacatacaaagatgcttatagccggattaatgcgatcgtgattgaaggggaacaagaagcccatgaaaattacatcacactagcccaactgctgccagaatctcatgatgaattgattcgcctatccaagatggaaagccgccataagaaaggatttgaagcttgtgggcgcaatttagctgttaccccagatttgcaatttgccaaagagtttttctccggcctacaccaaaattttcaaacagctgccgcagaagggaaagtggttacttgtctgttgattcagtctttaattattgaatgttttgcgatcgcagcatataacatttacatccccgttgccgacgatttcgcccgtaaaattactgaaggagtagttaaagaagaatacagccacctcaattttggagaagtttggttgaaagaacactttgcagaatccaaagctgaacttgaacttgcaaatcgccagaacctacccatcgtctggaaaatgctcaaccaagtagaaggtgatgcccacacaatggcaatggaaaaagatgctttggtagaagacttcatgattcagtatggtgaagcattgagtaacattggtttttcgactcgcgatattatgcgcttgtcagcctacggactcataggtgcttaa

SEQ ID NO: 6 - protein, 232 aa, Nostoc punctiforme
  PCC 73102 Npun02004178 (ZP_00108838) amino acid
MQQLTDQSKELDFKSETYKDAYSRINAIVIEGEQEAHENYITLAQLLPESHDELIRLSKMESRHKKGFEACGRNLAVTPDLQFAKEFFSGLHQNFQTAAAEGKVVTCLLIQSLIIECFAIAAYNIYIPVADDFARKITEGVVKEEYSHLNFGEVWLKEHFAESKAELELANRQNLPIVWKMLNQVEGDAHTMAMEKDALVEDFMIQYGEALSNIGFSTRDIMRLSAYGLIGA

SEQ ID NO: 7 - DNA, 696 bp, Nostoc sp.
  PCC 7120 alr5283 (NP_489323) nucleotide
atgcagcaggttgcagccgatttagaaattgatttcaagagcgaaaaatataaagatgcctatagtcgcataaatgcgatcgtgattgaaggggaacaagaagcatacgagaattacattcaactatcccaactgctgccagacgataaagaagacctaattcgcctctcgaaaatggaaagccgtcacaaaaaaggatttgaagcttgtggacggaacctacaagtatcaccagatatggagtttgccaaagaattctttgctggactacacggtaacttccaaaaagcggcggctgaaggtaaaatcgttacctgtctattgattcagtccctgattattgaatgttttgcgatcgccgcatacaatatctacattcccgttgctgacgattttgctcgtaaaatcactgagggtgtagtcaaagatgaatacagccacctcaacttcggcgaagtttggttacagaaaaattttgcccaatccaaagcagaattagaagaagctaatcgtcataatcttcccatagtttggaaaatgctcaatcaagtcgcggatgatgccgcagtcttagctatggaaaaagaagccctagtcgaagattttatgattcagtacggcgaagcgttaagtaatattggcttcacaaccagagatattatgcggatgtcagcctacggacttacagcagcttaa

SEQ ID NO: 8 - protein, 231 aa, Nostoc sp.
  PCC 7120 alr5283 (NP_489323) amino acid
MQQVAADLEIDFKSEKYKDAYSRINAIVIEGEQEAYENYIQLSQLLPDDKEDLIRLSKMESRHKKGFEACGRNLQVSPDMEFAKEFFAGLHGNFQKAAAEGKIVTCLLIQSLIIECFAIAAYNIYIPVADDFARKITEGVVKDEYSHLNFGEVWLQKNFAQSKAELEEANRHNLPIVWKMLNQVADDAAVLAMEKEALVEDFMIQYGEALSNIGFTTRDIMRMSAYGLTAA

SEQ ID NO: 9 - DNA, 696 bp, Acaryochloris marina
  MBIC11017 AM1_4041 (YP_001518340) nucleotide
atgccccaaactcaggctatttcagaaattgacttctatagtgacacctacaaagatgcttacagtcgtattgacggcattgtgatcgaaggtgagcaagaagcgcatgaaaactatattcgtcttggcgaaatgctgcctgagcaccaagacgactttatccgcctgtccaagatggaagcccgtcataagaaagggtttgaagcctgcggtcgcaacttaaaagtaacctgcgatctagactttgcccggcgtttcttttccgacttacacaagaattttcaagatgctgcagctgaggataaagtgccaacttgcttagtgattcagtccttgatcattgagtgttttgcgatcgcagcttacaacatctatatccccgtcgctgatgactttgcccgtaagattacagagtctgtggttaaggatgagtatcaacacctcaattatggtgaagagtggcttaaagctcacttcgatgatgtgaaagcagaaatccaagaagctaatcgcaaaaacctccccatcgtttggagaatgctgaacgaagtggacaaggatgcggccgttttaggaatggaaaaagaagccctggttgaagacttcatgatccagtatggtgaagcccttagcaatattggtttctctacaggcgaaattatgcggatgtctgcctatggtcttgtggctgcgtaa

SEQ ID NO: 10 - protein, 231 aa, Acaryochloris marina
  MBIC11017 AM1_4041 (YP_001518340) amino acid
MPQTQAISEIDFYSDTYKDAYSRIDGIVIEGEQEAHENYIRLGEMLPEHQDDFIRLSKMEARHKKGFEACGRNLKVTCDLDFARRFFSDLHKNFQDAAAEDKVPTCLVIQSLIIECFAIAAYNIYIPVADDFARKITESVVKDEYQHLNYGEEWLKAHFDDVKAEIQEANRKNLPIVWRMLNEVDKDAAVLGMEKEALVEDFMIQYGEALSNIGFSTGEIMRMSAYGLVAA

SEQ ID NO: 11 - DNA, 696 bp, Thermosynechococcus elongatus
  BP-1 tll1313 (NP_682103) nucleotide
atgacaacggctaccgctacacctgttttggactaccatagcgatcgctacaaggatgcctacagccgcattaacgccattgtcattgaaggtgaacaggaagctcacgataactatatcgatttagccaagctgctgccacaacaccaagaggaactcacccgccttgccaagatggaagctcgccacaaaaaggggtttgaggcctgtggtcgcaacctgagcgtaacgccagatatggaatttgccaaagccttctttgaaaaactgcgcgctaactttcagagggctctggcggagggaaaaactgcgacttgtcttctgattcaagctttgatcatcgaatcctttgcgatcgcggcctacaacatctacatcccaatggcggatcctttcgcccgtaaaattactgagagtgttgttaaggacgaatacagccacctcaactttggcgaaatctggctcaaggaacactttgaaagcgtcaaaggagagctcgaagaagccaatcgcgccaatttacccttggtctggaaaatgctcaaccaagtggaagcagatgccaaagtgctcggcatggaaaaagatgcccttgtggaagacttcatgattcagtacagtggtgccctagaaaatatcggctttaccacccgcgaaattatgaagatgtcagtttatggcctcactggggcataa

SEQ ID NO: 12 - protein, 231 aa, Thermosynechococcus elongatus
  BP-1 tll1313 (NP_682103) amino acid
MTTATATPVLDYHSDRYKDAYSRINAIVIEGEQEAHDNYIDLAKLLPQHQEELTRLAKMEARHKKGFEACGRNLSVTPDMEFAKAFFEKLRANFQRALAEGKTATCLLIQALIIESFAIAAYNIYIPMADPFARKITESVVKDEYSHLNFGEIWLKEHFESVKGELEEANRANLPLVWKMLNQVEADAKVLGMEKDALVEDFMIQYSGALENIGFTTREIMKMSVYGLTGA

SEQ ID NO: 13 - DNA, 732 bp, Synechococcus sp.
  JA-3-3A CYA_0415 (YP_473897) nucleotide
atggccccagcgaacgtcctgcccaacacccccccgtcccccactgatgggggcggcactgccctagactacagcagcccaaggtatcggcaggcctactcccgcatcaacggtattgttatcgaaggcgaacaagaagcccacgacaactacctcaagctggccgaaatgctgccggaagctgcagaggagctgcgcaagctggccaagatggaattgcgccacatgaaaggcttccaggcctgcggcaaaaacctgcaggtggaacccgatgtggagtttgcccgcgcctttttcgcgcccttgcgggacaatttccaaagcgccgcagcggcaggggatctggtctcctgttttgtcattcagtctttgatcatcgagtgctttgccattgccgcctacaacatctacatcccggttgccgatgactttgcccgcaagatcaccgagggggtagttaaggacgagtatctgcacctcaattttggggagcgctggctgggcgagcactttgccgaggttaaagcccagatcgaagcagccaacgcccaaaatctgcctctagttcggcagatgctgcagcaggtagaggcggatgtggaagccatttacatggatcgcgaggccattgtagaagacttcatgatcgcctacggcgaggccctggccagcatcggcttcaacacccgcgaggtaatgcgcctctcggcccagggtctgcgggccgcctga

SEQ ID NO: 14 - protein, 243 aa, Synechococcus sp.
  JA-3-3A CYA_0415 (YP_473897) amino acid
MAPANVLPNTPPSPTDGGGTALDYSSPRYRQAYSRINGIVIEGEQEAHDNYLKLAEMLPEAAEELRKLAKMELRHMKGFQACGKNLQVEPDVEFARAFFAPLRDNFQSAAAAGDLVSCFVIQSLIIECFAIAAYNIYIPVADDFARKITEGVVKDEYLHLNFGERWLGEHFAEVKAQIEAANAQNLPLVRQMLQQVEADVEAIYMDREAIVEDFMIAYGEALASIGFNTREVMRLSAQGLRAA

SEQ ID NO: 15 - DNA, 708 bp, Gloeobacter violaceus
  PCC 7421 gll3146 (NP_926092) nucleotide
gtgaaccgaaccgcaccgtccagcgccgcgcttgattaccgctccgacacctaccgcgatgcgtactcccgcatcaatgccatcgtccttgaaggcgagcgggaagcccacgccaactaccttaccctcgctgagatgctgccggaccatgccgaggcgctcaaaaaactggccgcgatggaaaatcgccacttcaaaggcttccagtcctgcgcccgcaacctcgaagtcacgccggacgacccgtttgcaagggcctacttcgaacagctcgacggcaactttcagcaggcggcggcagaaggtgaccttaccacctgcatggtcatccaggcactgatcatcgagtgcttcgcaattgcggcctacaacgtctacattccggtggccgacgcgtttgcccgcaaggtgaccgagggcgtcgtcaaggacgagtacacccacctcaactttgggcagcagtggctcaaagagcgcttcgtgaccgtgcgcgagggcatcgagcgcgccaacgcccagaatctgcccatcgtctggcggatgctcaacgccgtcgaagcggacaccgaagtgctgcagatggataaagaagcgatcgtcgaagactttatgatcgcctacggtgaagccttgggcgacatcggtttttcgatgcgcgacgtgatgaagatgtccgcccgcggccttgcctctgccccccgccagtga

SEQ ID NO: 16 - protein, 235 aa, Gloeobacter violaceus
  PCC 7421 gll3146 (NP_926092) amino acid
MNRTAPSSAALDYRSDTYRDAYSRINAIVLEGEREAHANYLTLAEMLPDHAEALKKLAAMENRHFKGFQSCARNLEVTPDDPFARAYFEQLDGNFQQAAAEGDLTTCMVIQALIIECFAIAAYNVYIPVADAFARKVTEGVVKDEYTHLNFGQQWLKERFVTVREGIERANAQNLPIVWRMLNAVEADTEVLQMDKEAIVEDFMIAYGEALGDIGFSMRDVMKMSARGLASAPRQ

SEQ ID NO: 17 - DNA, 732 bp, Prochlorococcus marinus
  MIT9313 PM1231 (NP_895059) nucleotide
atgcctacgcttgagatgcctgtggcagctgttcttgacagcactgttggatcttcagaagccctgccagacttcacttcagatagatataaggatgcatacagcagaatcaacgcaatagtcattgagggcgaacaggaagcccatgacaattacatcgcgattggcacgctgcttcccgatcatgtcgaagagctcaagcggcttgccaagatggagatgaggcacaagaagggctttacagcttgcggcaagaaccttggcgttgaggctgacatggacttcgcaagggagttttttgctcctttgcgtgacaacttccagacagctttagggcaggggaaaacacctacatgcttgctgatccaggcgctcttgattgaagcctttgctatttcggcttatcacacctatatccctgtttctgacccctttgctcgcaagattactgaaggtgtcgtgaaggacgagtacacacacctcaattatggcgaggcttggctcaaggccaatctggagagttgccgtgaggagttgcttgaggccaatcgcgagaacctgcctctgattcgccggatgcttgatcaggtagcaggtgatgctgccgtgctgcagatggataaggaagatctgattgaggatttcttaatcgcctaccaggaatctctcactgagattggctttaacactcgtgaaattacccgtatggcagcggcagctcttgtgagctga

SEQ ID NO: 18 - protein, 243 aa, Prochlorococcus marinus
  MIT9313 PM1231 (NP_895059) amino acid
MPTLEMPVAAVLDSTVGSSEALPDFTSDRYKDAYSRINAIVIEGEQEAHDNYIAIGTLLPDHVEELKRLAKMEMRHKKGFTACGKNLGVEADMDFAREFFAPLRDNFQTALGQGKTPTCLLIQALLIEAFAISAYHTYIPVSDPFARKITEGVVKDEYTHLNYGEAWLKANLESCREELLEANRENLPLIRRMLDQVAGDAAVLQMDKEDLIEDFLIAYQESLTEIGFNTREITRMAAAALVS

SEQ ID NO: 19 - DNA, 717 bp, Prochlorococcus marinus
  subsp. pastoris str. CCMP1986 PMM0532 (NP_892650) nucleotide
atgcaaacactcgaatctaataaaaaaactaatctagaaaattctattgatttacccgattttactactgattcttacaaagacgcttatagcaggataaatgcaatagttattgaaggtgaacaagaggctcatgataattacatttccttagcaacattaattcctaacgaattagaagagttaactaaattagcgaaaatggagcttaagcacaaaagaggctttactgcatgtggaagaaatctaggtgttcaagctgacatgatttttgctaaagaattcttttccaaattacatggtaattttcaggttgcgttatctaatggcaagacaactacatgcctattaatacaggcaattttaattgaagcttttgctatatccgcgtatcacgtttacataagagttgctgatcctttcgcgaaaaaaattacccaaggtgttgttaaagatgaatatcttcatttaaattatggacaagaatggctaaaagaaaatttagcgacttgtaaagatgagctaatggaagcaaataaggttaaccttccattaatcaagaagatgttagatcaagtctcggaagatgcttcagtactagctatggatagggaagaattaatggaagaattcatgattgcctatcaggacactctccttgaaataggtttagataatagagaaattgcaagaatggcaatggctgctatagtttaa

SEQ ID NO: 20 - protein, 238 aa, Prochlorococcus marinus
  subsp. pastoris str. CCMP1986 PMM0532 (NP_892650) amino acid
MQTLESNKKTNLENSIDLPDFTTDSYKDAYSRINAIVIEGEQEAHDNYISLATLIPNELEELTKLAKMELKHKRGFTACGRNLGVQADMIFAKEFFSKLHGNFQVALSNGKTTTCLLIQAILIEAFAISAYHVYIRVADPFAKKITQGVVKDEYLHLNYGQEWLKENLATCKDELMEANKVNLPLIKKMLDQVSEDASVLAMDREELMEEFMIAYQDTLLEIGLDNREIARMAMAAIV

SEQ ID NO: 21 - DNA, 726 bp, Prochlorococcus marinus
  str. NATL2A PMN2A_1863 (YP_293054) nucleotide
atgcaagcttttgcatccaacaatttaaccgtagaaaaagaagagctaagttctaactctcttccagatttcacctcagaatcttacaaagatgcttacagcagaatcaatgcagttgtaattgaaggggagcaagaagcttattctaattttcttgatctcgctaaattgattcctgaacatgcagatgagcttgtgaggctagggaagatggagaaaaagcatatgaatggtttttgtgcttgcgggagaaatcttgctgtaaagcctgatatgccttttgcaaagacctttttctcaaaactccataataattttttagaggctttcaaagtaggagatacgactacctgtctcctaattcaatgcatcttgattgaatcttttgcaatatccgcatatcacgtttatatacgtgttgctgatccattcgccaaaagaatcacagagggtgttgtccaagatgaatacttgcatttgaactatggtcaagaatggcttaaggccaatctagagacagttaagaaagatcttatgagggctaataaggaaaacttgcctcttataaagtccatgctcgatgaagtttcaaacgacgccgaagtccttcatatggataaagaagagttaatggaggaatttatgattgcttatcaagattcccttcttgaaataggtcttgataatagagaaattgcaagaatggctcttgcagcggtgatataa

SEQ ID NO: 22 - protein, 241 aa, Prochlorococcus marinus
  str. NATL2A PMN2A_1863 (YP_293054) amino acid
MQAFASNNLTVEKEELSSNSLPDFTSESYKDAYSRINAVVIEGEQEAYSNFLDLAKLIPEHADELVRLGKMEKKHMNGFCACGRNLAVKPDMPFAKTFFSKLHNNFLEAFKVGDTTTCLLIQCILIESFAISAYHVYIRVADPFAKRITEGVVQDEYLHLNYGQEWLKANLETVKKDLMRANKENLPLIKSMLDEVSNDAEVLHMDKEELMEEFMIAYQDSLLEIGLDNREIARMALAAVI

SEQ ID NO: 23 - DNA, 732 bp, Synechococcus sp.
  RS9917 RS9917_09941 (ZP_01079772) nucleotide
atgccgacccttgagacgtctgaggtcgccgttcttgaagactcgatggcttcaggctcccggctgcctgatttcaccagcgaggcttacaaggacgcctacagccgcatcaatgcgatcgtgatcgagggtgagcaggaagcgcacgacaactacatcgccctcggcacgctgatccccgagcagaaggatgagctggcccgtctcgcccgcatggagatgaagcacatgaaggggttcacctcctgtggccgcaatctcggcgtggaggcagaccttccctttgctaaggaattcttcgcccccctgcacgggaacttccaggcagctctccaggagggcaaggtggtgacctgcctgttgattcaggcgctgctgattgaagcgttcgccatttccgcctatcacatctacatcccggtggcggatcccttcgctcgcaagatcactgaaggtgtggtgaaggatgagtacacccacctcaattacggccaggaatggctgaaggccaattttgaggccagcaaggatgagctgatggaggccaacaaggccaatctgcctctgatccgctcgatgctggagcaggtggcagccgacgccgccgtgctgcagatggaaaaggaagatctgatcgaagatttcctgatcgcttaccaggaggccctctgcgagatcggtttcagctcccgtgacattgctcgcatggccgccgctgccctcgcggtctga

SEQ ID NO: 24 - protein, 243 aa, Synechococcus sp.
  RS9917 RS9917_09941 (ZP_01079772) amino acid
MPTLETSEVAVLEDSMASGSRLPDFTSEAYKDAYSRINAIVIEGEQEAHDNYIALGTLIPEQKDELARLARMEMKHMKGFTSCGRNLGVEADLPFAKEFFAPLHGNFQAALQEGKVVTCLLIQALLIEAFAISAYHIYIPVADPFARKITEGVVKDEYTHLNYGQEWLKANFEASKDELMEANKANLPLIRSMLEQVAADAAVLQMEKEDLIEDFLIAYQEALCEIGFSSRDIARMAAAALAV

SEQ ID NO: 25 - DNA, 681 bp, Synechococcus sp.
  RS9917 RS9917_12945 (ZP_01080370) nucleotide
atgacccagctcgactttgccagtgcggcctaccgcgaggcctacagccggatcaacggcgttgtgattgtgggcgaaggtctcgccaatcgccatttccagatgttggcgcggcgcattcccgctgatcgcgacgagctgcagcggctcggacgcatggagggagaccatgccagcgcctttgtgggctgtggtcgcaacctcggtgtggtggccgatctgcccctggcccggcgcctgtttcagcccctccatgatctgttcaaacgccacgaccacgacggcaatcgggccgaatgcctggtgatccaggggttgatcgtggaatgtttcgccgtggcggcttaccgccactacctgccggtggccgatgcctacgcccggccgatcaccgcagcggtgatgaacgatgaatcggaacacctcgactacgctgagacctggctgcagcgccatttcgatcaggtgaaggcccgggtcagcgcggtggtggtggaggcgttgccgctcaccctggcgatgttgcaatcgcttgctgcagacatgcgacagatcggcatggatccggtggagaccctggccagcttcagtgaactgtttcgggaagcgttggaatcggtggggtttgaggctgtggaggccaggcgactgctgatgcgagcggccgcccggatggtctga

SEQ ID NO: 26 - protein, 226 aa, Synechococcus sp.
  RS9917 RS9917_12945 (ZP_01080370) amino acid
MTQLDFASAAYREAYSRINGVVIVGEGLANRHFQMLARRIPADRDELQRLGRMEGDHASAFVGCGRNLGVVADLPLARRLFQPLHDLFKRHDHDGNRAECLVIQGLIVECFAVAAYRHYLPVADAYARPITAAVMNDESEHLDYAETWLQRHFDQVKARVSAVVVEALPLTLAMLQSLAADMRQIGMDPVETLASFSELFREALESVGFEAVEARRLLMRAAARMV

SEQ ID NO: 27 - DNA, 696 bp, Cyanothece sp.
  ATCC51142 cce_0778 (YP_001802195) nucleotide
atgcaagagcttgctttacgctcagagcttgattttaacagcgaaacctataaagatgcttacagtcgcatcaatgctattgtcattgaaggggaacaagaagcctatcaaaattatcttgatatggcgcaacttctcccagaagacgaggctgagttaattcgtctctccaagatggaaaaccgtcacaaaaaaggctttcaagcctgtggcaagaatttgaatgtgaccccagatatggactacgctcaacaattttttgctgaacttcatggcaacttccaaaaggcaaaagccgaaggcaaaattgtcacttgcttattaattcaatctttgatcatcgaagcctttgcgatcgccgcttataatatttatattcctgtggcagatccctttgctcgtaaaatcaccgaaggggtagttaaggatgaatatacccacctcaattttggggaagtctggttaaaagagcattttgaagcctctaaagcagaattagaagacgcaaataaagaaaatttaccccttgtttggcaaatgctcaaccaagttgaaaaagatgccgaagtgttagggatggagaaagaagccttagtggaagatttcatgattagttatggagaagctttaagtaatattggtttctctacccgtgagatcatgaaaatgtctgcttacgggctacgggctgcttaa

SEQ ID NO: 28 - protein, 231 aa, Cyanothece sp.
  ATCC51142 cce_0778 (YP_001802195) amino acid
MQELALRSELDFNSETYKDAYSRINAIVIEGEQEAYQNYLDMAQLLPEDEAELIRLSKMENRHKKGFQACGKNLNVTPDMDYAQQFFAELHGNFQKAKAEGKIVTCLLIQSLIIEAFAIAAYNIYIPVADPFARKITEGVVKDEYTHLNFGEVWLKEHFEASKAELEDANKENLPLVWQMLNQVEKDAEVLGMEKEALVEDFMISYGEALSNIGFSTREIMKMSAYGLRAA

SEQ ID NO: 29 - DNA, 696 bp, Cyanothece sp.
  PCC7245 Cyan7425_0398 (YP_002481151) nucleotide
atgcctcaagtgcagtccccatcggctatagacttctacagtgagacctaccaggatgcttacagccgcattgatgcgatcgtgatcgagggagaacaggaagcccacgacaattacctgaagctgacggaactgctgccggattgtcaagaagatctggtccggctggccaaaatggaagcccgtcacaaaaaagggtttgaagcttgtggccgcaatctcaaggtcacacccgatatggagtttgctcaacagttctttgctgacctgcacaacaatttccagaaagctgctgcggccaacaaaattgccacctgtctggtgatccaggccctgattattgagtgctttgccatcgccgcttataacatctatattcctgtcgctgatgactttgcccgcaaaattaccgaaaacgtggtcaaagacgaatacacccacctcaactttggtgaagagtggctcaaagctaactttgatagccagcgggaagaagtggaagcggccaaccgggaaaacctgccgatcgtctggcggatgctcaatcaggtagagactgatgctcacgttttaggtatggaaaaagaggctttagtggaaagcttcatgatccaatatggtgaagccctggaaaatattggtttctcgacccgtgagatcatgcgcatgtccgtttacggcctctctgcggcataa

SEQ ID NO: 30 - protein, 231 aa, Cyanothece sp.
  PCC7245  Cyan7425_0398 (YP_002481151) amino acid
MPQVQSPSAIDFYSETYQDAYSRIDAIVIEGEQEAHDNYLKLTELLPDCQEDLVRLAKMEARHKKGFEACGRNLKVTPDMEFAQQFFADLHNNFQKAAAANKIATCLVIQALIIECFAIAAYNIYIPVADDFARKITENVVKDEYTHLNFGEEWLKANFDSQREEVEAANRENLPIVWRMLNQVETDAHVLGMEKEALVESFMIQYGEALENIGFSTREIMRMSVYGLSAA

SEQ ID NO: 31 - DNA, 702 bp, Cyanothece sp.
  PCC7245 Cyan7425_2986 (YP_002483683) nucleotide
atgtctgattgcgccacgaacccagccctcgactattacagtgaaacctaccgcaatgcttaccggcgggtgaacggtattgtgattgaaggcgagaagcaagcctacgacaactttatccgcttagctgagctgctcccagagtatcaagcggaattaacccgtctggctaaaatggaagcccgccaccagaagagctttgttgcctgtggccaaaatctcaaggttagcccggacttagactttgcggcacagttttttgctgaactgcatcaaatttttgcatctgcagcaaatgcgggccaggtggctacctgtctggttgtgcaagccctgatcattgaatgctttgcgatcgccgcctacaatacctatttgccagtagcggatgaatttgcccgtaaagtcaccgcatccgttgttcaggacgagtacagccacctaaactttggtgaagtctggctgcagaatgcgtttgagcagtgtaaagacgaaattatcacagctaaccgtcttgctctgccgctgatctggaaaatgctcaaccaggtgacaggcgaattgcgcattctgggcatggacaaagcttctctggtagaagactttagcactcgctatggagaggccctgggccagattggtttcaaactatctgaaattctctccctgtccgttcagggtttacaggcggttacgccttag

SEQ ID NO: 32 - protein, 233 aa, Cyanothece sp.
  PCC7245 Cyan7425_2986 (YP_002483683) amino acid
MSDCATNPALDYYSETYRNAYRRVNGIVIEGEKQAYDNFIRLAELLPEYQAELTRLAKMEARHQKSFVACGQNLKVSPDLDFAAQFFAELHQIFASAANAGQVATCLVVQALIIECFAIAAYNTYLPVADEFARKVTASVVQDEYSHLNFGEVWLQNAFEQCKDEIITANRLALPLIWKMLNQVTGELRILGMDKASLVEDFSTRYGEALGQIGFKLSEILSLSVQGLQAVTP

SEQ ID NO: 33 - DNA, 696 bp, Anabaena variabilis
  ATCC29413 YP_323043 (Ava_2533) nucleotide
atgcagcaggttgcagccgatttagaaatcgatttcaagagcgaaaaatataaagatgcctatagtcgcataaatgcgatcgtgattgaaggggaacaagaagcatatgagaattacattcaactatcccaactgctgccagacgataaagaagacctaattcgcctctcgaaaatggaaagtcgccacaaaaaaggatttgaagcttgtggacggaacctgcaagtatccccagacatagagttcgctaaagaattctttgccgggctacacggtaatttccaaaaagcggcagctgaaggtaaagttgtcacttgcctattgattcaatccctgattattgaatgttttgcgatcgccgcatacaatatctacatccccgtggctgacgatttcgcccgtaaaatcactgagggtgtagttaaagatgaatacagtcacctcaacttcggcgaagtttggttacagaaaaatttcgctcaatcaaaagcagaactagaagaagctaatcgtcataatcttcccatagtctggaaaatgctcaatcaagttgccgatgatgcggcagtcttagctatggaaaaagaagccctagtggaagattttatgattcagtacggcgaagcactaagtaatattggcttcacaaccagagatattatgcggatgtcagcctacggactcacagcagcttaa

SEQ ID NO: 34 - protein, 231 aa, Anabaena variabilis
  ATCC29413 YP_323043 (Ava_2533) amino acid
MQQVAADLEIDFKSEKYKDAYSRINAIVIEGEQEAYENYIQLSQLLPDDKEDLIRLSKMESRHKKGFEACGRNLQVSPDIEFAKEFFAGLHGNFQKAAAEGKVVTCLLIQSLIIECFAIAAYNIYIPVADDFARKITEGVVKDEYSHLNFGEVWLQKNFAQSKAELEEANRHNLPIVWKMLNQVADDAAVLAMEKEALVEDFMIQYGEALSNIGFTTRDIMRMSAYGLTAA

SEQ ID NO: 35 - DNA, 765 bp, Synechococcus elongatus
  PCC6301 YP_170760 (syc0050_d) nucleotide
gtgcgtaccccctgggatccaccaaatcccacattctccctctcatccgtgtcaggagaccgcagactcatgccgcagcttgaagccagccttgaactggactttcaaagcgagtcctacaaagacgcttacagccgcatcaacgcgatcgtgattgaaggcgaacaagaggcgttcgacaactacaatcgccttgctgagatgctgcccgaccagcgggatgagcttcacaagctagccaagatggaacagcgccacatgaaaggctttatggcctgtggcaaaaatctctccgtcactcctgacatgggttttgcccagaaatttttcgagcgcttgcacgagaacttcaaagcggcggctgcggaaggcaaggtcgtcacctgcctactgattcaatcgctaatcatcgagtgctttgcgatcgcggcttacaacatctacatcccagtggcggatgcttttgcccgcaaaatcacggagggggtcgtgcgcgacgaatacctgcaccgcaacttcggtgaagagtggctgaaggcgaattttgatgcttccaaagccgaactggaagaagccaatcgtcagaacctgcccttggtttggctaatgctcaacgaagtggccgatgatgctcgcgaactcgggatggagcgtgagtcgctcgtcgaggactttatgattgcctacggtgaagctctggaaaacatcggcttcacaacgcgcgaaatcatgcgtatgtccgcctatggccttgcggccgtttga

SEQ ID NO: 36 - protein, 254 aa, Synechococcus elongatus
  PCC6301 YP_170760 (syc0050_d) amino acid
MRTPWDPPNPTFSLSSVSGDRRLMPQLEASLELDFQSESYKDAYSRINAIVIEGEQEAFDNYNRLAEMLPDQRDELHKLAKMEQRHMKGFMACGKNLSVTPDMGFAQKFFERLHENFKAAAAEGKVVTCLLIQSLIIECFAIAAYNIYIPVADAFARKITEGVVRDEYLHRNFGEEWLKANFDASKAELEEANRQNLPLVWLMLNEVADDARELGMERESLVEDFMIAYGEALENIGFTTREIMRMSAYGLAAV

SEQ ID NO: 37 - protein, 19 aa, Artificial Sequence
  Description of Artificial Sequence Synthetic motif 1 peptide
YXXAYXRXXXXVXXGEXXA

SEQ ID NO: 38 - protein, 15 aa, Artificial Sequence
  Description of Artificial Sequence Synthetic motif 2 peptide
LXXMEXXHXXXFXXC

SEQ ID NO: 39 - protein, 17 aa, Artificial Sequence
  Description of Artificial Sequence Synthetic motif 3 peptide
CXXXQXXXXEXFAXXAY

SEQ ID NO: 40 - protein, 19 aa, Artificial Sequence
  Description of Artificial Sequence Synthetic motif 4 peptide
TXXVXXXEXXHXXXXXXWL

SEQ ID NO: 41 - protein, 23 aa, Artificial Sequence
  Description of Artificial Sequence Synthetic motif 5 peptide
YXXAYXRXXXXVXXGEXXAXXXX

SEQ ID NO: 42 - protein, 21 aa, Artificial Sequence
  Description of Artificial Sequence Synthetic motif 6 peptide
LXXMEXXHXXXFXXCXXNLXX

SEQ ID NO: 43 - protein, 21 aa, Artificial Sequence
  Description of Artificial Sequence Synthetic motif 7 peptide
CXXXQXXXXEXFAXXAYXXYX

SEQ ID NO: 44 - protein, 26 aa, Artificial Sequence
  Description of Artificial Sequence Synthetic motif 8 peptide
DXXAXXXTXXVXXXEXXHXXXXXXWL

SEQ ID NO: 45 - DNA, 699 bp, Artificial Sequence
  Description of Artificial Sequence Synthetic PCC 73102 Npun02004178 (ZP_00108838) polynucleotide
atgcagcaactgacggatcagagcaaagaactggacttcaaaagcgaaacctacaaggacgcgtattctcgtatcaacgctatcgttatcgagggtgaacaagaagcgcacgagaattacattaccctggcgcagctgctgcctgaatcccacgatgaactgattcgtctgagcaaaatggagtcgcgtcacaaaaagggttttgaggcctgcggtcgtaacctggcggtcactccggacctgcagttcgctaaggagttcttcagcggcctgcatcaaaactttcagacggcagcggcggaaggtaaggttgtcacctgcctgctgattcaaagcctgatcattgagtgtttcgctatcgcagcctataacatttacatcccggtggcggacgattttgcacgcaagatcactgagggtgtggttaaagaagaatacagccacctgaacttcggtgaggtctggttgaaggagcactttgcggaaagcaaggcggagctggaattggcaaatcgtcaaaacctgccgatcgtgtggaaaatgctgaatcaagtggagggtgatgcacacacgatggctatggaaaaagacgctctggtggaggacttcatgatccagtacggcgaggcgctgagcaacattggctttagcacccgtgacattatgcgcctgagcgcgtatggcctgatcggtgcgtaa

SEQ ID NO: 46 - DNA, 696 bp, Artificial Sequence
  Description of Artificial Sequence Synthetic MBIC11017 AM1_4041 (YP_001518340) polynucleotide
atgccgcaaacgcaagctattagcgaaattgatttctattctgacacctataaggacgcttactctcgtatcgatggtatcgtgatcgagggtgagcaagaggcgcatgagaactacattcgtctgggtgaaatgttgcctgagcatcaagacgactttatccgtttgagcaagatggaggcccgtcacaagaagggctttgaggcttgtggtcgtaacttgaaggtgacttgcgatctggacttcgcgcgtcgcttcttctcggacctgcacaagaacttccaagatgctgcggccgaggataaagttccgacctgcttggttattcagtccctgatcatcgaatgcttcgcgattgcagcgtataacatttacatcccggttgccgatgatttcgctcgtaagattaccgagagcgtcgtcaaggacgaataccagcatctgaactatggcgaggagtggctgaaggcccatttcgacgacgtgaaggccgagatccaggaagcaaatcgcaagaatctgccgatcgtttggcgtatgctgaacgaggttgacaaggacgcagcagtgctgggcatggagaaggaagcgttggttgaagacttcatgattcaatacggtgaggccctgtccaacattggcttttctaccggcgagatcatgcgtatgtctgcgtacggtctggtggcagcctaa

SEQ ID NO: 47 - DNA, 696 bp, Artificial Sequence
  Description of Artificial Sequence Synthetic BP-1 tll1313 (NP_682103) polynucleotide
atgaccaccgcgaccgcaacgccggtgctggactatcacagcgaccgctacaaggacgcatacagccgcatcaacgcgattgtcatcgaaggtgaacaagaggcccacgacaattacattgatctggctaaactgctgcctcaacaccaagaagagctgacccgtctggcgaagatggaggcccgccacaagaagggttttgaagcgtgcggtcgcaatctgtccgttaccccggatatggagttcgcgaaagcgttctttgagaagctgcgcgcgaactttcagcgtgccctggcggagggtaagaccgcaacctgtctgctgatccaggcgttgatcattgaatccttcgcaattgccgcgtacaacatttacatccctatggccgatccgtttgcgcgcaagattaccgaaagcgtcgtcaaggatgaatactctcacttgaactttggcgaaatctggttgaaggaacatttcgagagcgtcaagggcgagttggaggaagctaaccgtgcgaatctgccgctggtttggaagatgttgaatcaggtcgaggcagacgcaaaggtcctgggcatggagaaggatgctctggtggaagactttatgatccagtactccggtgcgctggagaacatcggctttaccacccgtgaaatcatgaaaatgtctgtgtatggcctgaccggcgcgtaa

SEQ ID NO: 48 - DNA, 732 bp, Artificial Sequence
  Description of Artificial Sequence Synthetic JA-3-3A CYA_0415 (YP_473897) polynucleotide
atggcgcctgcaaacgtgctgccaaatacgccgccgagcccgaccgatggtggtggtacggccctggactacagctctccgcgttaccgtcaggcgtacagccgtatcaatggcattgttatcgaaggcgagcaggaagcgcacgataactacctgaagttggcggagatgctgcctgaggctgccgaggaactgcgtaagctggcaaagatggaattgcgtcacatgaagggctttcaggcttgcggcaagaacttgcaggtggagcctgacgtcgagtttgcccgcgctttcttcgcgccgctgcgcgacaacttccaatccgcagcagcggccggtgatctggtttcctgtttcgtcatccaaagcctgatcatcgagtgttttgcgatcgctgcgtataacatttacatcccggttgcagacgacttcgcccgtaagatcacggagggcgtggttaaggacgagtatctgcatctgaatttcggcgagcgttggttgggtgaacacttcgcagaggttaaagcacagatcgaggcagccaatgcccagaacctgccgctggtgcgccaaatgctgcagcaagttgaggcggacgtcgaggcaatctatatggaccgtgaggcgatcgttgaggatttcatgattgcttatggcgaagcgctggcaagcattggcttcaacacgcgcgaagtgatgcgtctgagcgcacagggcttgcgtgcagcataa

SEQ ID NO: 49 - DNA, 732 bp, Artificial Sequence
  Description of Artificial Sequence Synthetic MIT9313 PM123 (NP_895059) polynucleotide
atgccgacgttggagatgccggtcgctgcggtcctggacagcacggtcggtagctctgaggcgctgccggactttaccagcgaccgctacaaagacgcttattcgcgtatcaacgcgattgtgatcgagggtgaacaagaagcccacgacaactacatcgcaattggcaccctgttgccggaccatgtggaagaactgaaacgtctggcgaaaatggaaatgcgtcacaagaaaggtttcaccgcgtgcggtaagaacttgggtgtggaagccgatatggacttcgcccgtgagttctttgccccgttgcgcgacaactttcaaaccgcgctgggtcaaggcaagacccctacgtgtctgttgatccaagcgctgctgattgaagcgttcgcgatctcggcctaccacacttacattccggttagcgatccgttcgcacgtaagatcactgaaggtgtcgttaaggacgaatacacccatctgaactacggtgaggcatggctgaaggcgaatctggagagctgccgcgaggaactgctggaagcgaaccgtgagaatctgccgctgatccgccgcatgctggatcaggtcgcgggcgacgcggcagtcctgcagatggataaggaagacctgatcgaagacttcctgattgcttaccaagagagcttgactgagatcggctttaacacgcgtgaaatcacccgtatggccgcagcggcgctggtcagctaa

SEQ ID NO: 50 - DNA, 717 bp, Artificial Sequence
  Description of Artificial Sequence Synthetic PMM0532 (NP_892650) polynucleotide
atgcaaaccctggagagcaacaagaaaaccaacctggaaaacagcattgacctgccagatttcacgacggacagctacaaggatgcgtattcccgtatcaatgctatcgtcattgaaggtgaacaggaagcccatgacaactatatcagcctggccaccctgatcccgaatgaactggaggaattgaccaaactggccaagatggagctgaaacacaaacgtggctttacggcatgcggtcgcaatctgggtgttcaggccgatatgatctttgcgaaagagtttttctctaagctgcacggcaacttccaagttgcgctgagcaacggtaagacgaccacctgcttgctgatccaggccatcttgattgaagccttcgcgatttccgcgtaccacgtgtacattcgtgtcgcggacccgtttgcgaaaaagattactcaaggtgtggtgaaggatgagtacctgcacctgaactatggtcaggaatggttgaaggagaatctggcaacctgtaaggacgaactgatggaagcaaacaaagttaatctgccgctgattaagaaaatgctggatcaggtgagcgaggatgcctctgtgttggctatggatcgtgaggagctgatggaggagttcatgatcgcgtatcaggacaccctgttggaaatcggtctggacaatcgtgaaattgcgcgtatggcaatggctgcgattgtgtaa

SEQ ID NO: 51 - DNA, 726 bp, Artificial Sequence
  Description of Artificial Sequence Synthetic PMN2A_1863 (YP_293054) polynucleotide
atgcaggccttcgcaagcaataacctgacggtcgaaaaggaagaactgagctccaatagcctgccggatttcaccagcgagagctataaggatgcatactctcgtatcaatgccgtggttatcgaaggtgaacaagaggcttattctaactttctggacctggccaagctgatcccggagcacgccgacgagctggtgcgcttgggtaagatggaaaagaaacacatgaacggcttctgcgcgtgtggtcgtaacttggcagttaaaccagacatgccgttcgcgaagacgttctttagcaagctgcacaacaatttcctggaggcgtttaaggtgggcgatacgacgacctgtttgttgatccaatgcatcttgatcgagtcctttgccatcagcgcgtaccacgtgtacattcgcgtggcagatccgtttgccaagcgtatcacggaaggtgttgttcaagacgagtacctgcatttgaattacggtcaagagtggctgaaagcgaacctggagactgtgaagaaagacctgatgcgcgcgaacaaagagaatctgccattgattaagtctatgctggacgaagtctccaacgacgctgaagtgctgcacatggataaagaagagctgatggaagagtttatgattgcatatcaggacagcctgctggaaattggcctggacaaccgcgagatcgcacgcatggcgctggcagcggttatttaa

SEQ ID NO: 52 - DNA, 732 bp, Artificial Sequence
  Description of Artificial Sequence Synthetic RS9917 RS9917_09941 (ZP_01079772) polynucleotide
atgccgaccctggaaactagcgaggtggcagttctggaagactcgatggccagcggtagccgcctgccggactttaccagcgaggcctataaggacgcgtatagccgtatcaatgcgatcgtgattgaaggcgagcaagaagcgcatgacaactacattgcactgggcacgctgatcccagaacagaaggacgagctggctcgcctggctcgtatggaaatgaaacacatgaagggctttaccagctgtggtcgtaacctgggtgtggaagcggatctgccgttcgcgaaggagttcttcgcaccgctgcatggtaactttcaggcggcgctgcaggaaggtaaggtggtgacctgtctgctgattcaggcactgctgattgaggcgttcgccattagcgcttatcacatttacattccggttgctgacccgtttgcacgcaagattaccgaaggtgttgtgaaagacgagtatacccatctgaactacggtcaagagtggttgaaggcgaatttcgaagcctccaaagacgaactgatggaagccaacaaggcgaatctgccgctgatccgttctatgctggaacaagtcgctgctgatgcggccgtgctgcaaatggagaaagaggacctgattgaagacttcctgatcgcatatcaagaagctctgtgtgagattggcttctcgtcccgtgatatcgcccgcatggcggcagccgcactggcggtttaa

SEQ ID NO: 53 - DNA, 681 bp, Artificial Sequence
  Description of Artificial Sequence Synthetic RS9917 RS9917_12945 (ZP_01080370) polynucleotide
atgacccaattggactttgcatctgcggcataccgtgaggcatacagccgtatcaatggtgtcgttattgttggcgagggcctggcgaatcgtcacttccaaatgctggcgcgtcgcattccggcagaccgtgacgaattgcaacgtttgggccgcatggagggtgaccacgcaagcgcctttgttggttgcggtcgcaatctgggtgtggtcgctgatctgccgctggcacgccgcctgttccagccgctgcatgatctgttcaagcgtcacgaccacgacggtaaccgtgctgaatgcctggtgatccagggtctgattgttgagtgctttgcggttgccgcgtatcgtcattacctgccggtggcagacgcgtatgcccgtccgatcaccgctgcggttatgaatgacgagagcgaacacctggactacgcagaaacctggctgcagcgccacttcgaccaagttaaagcccgcgtgagcgctgtggttgtggaggcgctgccgctgacgctggcgatgttgcaaagcctggctgcagatatgcgccaaatcggcatggacccggtggaaacgctggcgagcttcagcgagctgtttcgtgaagcgctggaaagcgttggttttgaagcggtcgaagcgcgccgtttgctgatgcgtgctgcagctcgtatggtttaa

SEQ ID NO: 54 - protein, 28 aa, Artificial Sequence
  Description of Artificial Sequence Synthetic motif 9 peptide
GAXGDIGSXXXXWXXXXXXXXXXXXXAR

SEQ ID NO: 55 - protein, 34 aa, Artificial Sequence
  Description of Artificial Sequence Synthetic motif 10 peptide
ATVAXXGATGDIGSAVXRWLXXKXXXXXLXLXAR

SEQ ID NO: 56 - protein, 11 aa, Artificial Sequence
  Description of Artificial Sequence Synthetic motif 11 peptide
XLXXXRFTTGN

SEQ ID NO: 57 - protein, 14 aa, Artificial Sequence
  Description of Artificial Sequence Synthetic motif 12 peptide
MFGLIGHXXXXXXA

SEQ ID NO: 58 - protein, 19 aa, Artificial Sequence
  Description of Artificial Sequence Synthetic motif 13 peptide
LXXWXXAPPXXXXXXXXXS

SEQ ID NO: 59 - protein, 21 aa, Artificial Sequence
  Description of Artificial Sequence Synthetic motif 14 peptide
SXXGXXIXGXYXXSXFXPEML

SEQ ID NO: 60 - protein, 27 aa, Artificial Sequence
  Description of Artificial Sequence Synthetic motif 15 peptide
KXAXRKXXXAMXXXQXXXXXIXXLGGF

SEQ ID NO: 61 - protein, 14 aa, Artificial Sequence
  Description of Artificial Sequence Synthetic motif 16 peptide
AXXXXXXXXWVASX

SEQ ID NO: 62 - protein, 12 aa, Artificial Sequence
  Description of Artificial Sequence Synthetic motif 17 peptide
PXXXXDGGYPKN

SEQ ID NO: 63 - protein, 25 aa, Artificial Sequence
  Description of Artificial Sequence Synthetic motif 18 peptide
NFSWGRNXIXXXXXXXIGXXSXXHG

SEQ ID NO: 64 - protein, 9 aa, Artificial Sequence
  Description of Artificial Sequence Synthetic motif 19 peptide
FTTGNTHTA

SEQ ID NO: 65 - DNA, 1026 bp, Synechococcus elongatus
  PCC7942 Synpcc7942_1594 (YP_400611) nucleotide
atgttcggtcttatcggtcatctcaccagtttggagcaggcccgcgacgtttctcgcaggatgggctacgacgaatacgccgatcaaggattggagttttggagtagcgctcctcctcaaatcgttgatgaaatcacagtcaccagtgccacaggcaaggtgattcacggtcgctacatcgaatcgtgtttcttgccggaaatgctggcggcgcgccgcttcaaaacagccacgcgcaaagttctcaatgccatgtcccatgcccaaaaacacggcatcgacatctcggccttggggggctttacctcgattattttcgagaatttcgatttggccagtttgcggcaagtgcgcgacactaccttggagtttgaacggttcaccaccggcaatactcacacggcctacgtaatctgtagacaggtggaagccgctgctaaaacgctgggcatcgacattacccaagcgacagtagcggttgtcggcgcgactggcgatatcggtagcgctgtctgccgctggctcgacctcaaactgggtgtcggtgatttgatcctgacggcgcgcaatcaggagcgtttggataacctgcaggctgaactcggccggggcaagattctgcccttggaagccgctctgccggaagctgactttatcgtgtgggtcgccagtatgcctcagggcgtagtgatcgacccagcaaccctgaagcaaccctgcgtcctaatcgacgggggctaccccaaaaacttgggcagcaaagtccaaggtgagggcatctatgtcctcaatggcggggtagttgaacattgcttcgacatcgactggcagatcatgtccgctgcagagatggcgcggcccgagcgccagatgtttgcctgctttgccgaggcgatgctcttggaatttgaaggctggcatactaacttctcctggggccgcaaccaaatcacgatcgagaagatggaagcgatcggtgaggcatcggtgcgccacggcttccaacccttggcattggcaatttga

SEQ ID NO: 66 - protein, 341 aa, Synechococcus elongatus
  PCC7942 Synpcc7942_1594 (YP_400611) amino acid
MFGLIGHLTSLEQARDVSRRMGYDEYADQGLEFWSSAPPQIVDEITVTSATGKVIHGRYIESCFLPEMLAARRFKTATRKVLNAMSHAQKHGIDISALGGFTSIIFENFDLASLRQVRDTTLEFERFTTGNTHTAYVICRQVEAAAKTLGIDITQATVAVVGATGDIGSAVCRWLDLKLGVGDLILTARNQERLDNLQAELGRGKILPLEAALPEADFIVWVASMPQGVVIDPATLKQPCVLIDGGYPKNLGSKVQGEGIYVLNGGVVEHCFDIDWQIMSAAEMARPERQMFACFAEAMLLEFEGWHTNFSWGRNQITIEKMEAIGEASVRHGFQPLALAI

SEQ ID NO: 67 - DNA, 1023 bp, Synechocystis sp.
  PCC6803 sll0209 (NP_442146) nucleotide
atgtttggtcttattggtcatctcacgagtttagaacacgcccaagcggttgctgaagatttaggctatcctgagtacgccaaccaaggcctggatttttggtgttcggctcctccccaagtggttgataattttcaggtgaaaagtgtgacggggcaggtgattgaaggcaaatatgtggagtcttgctttttgccggaaatgttaacccaacggcggatcaaagcggccattcgtaaaatcctcaatgctatggccctggcccaaaaggtgggcttggatattacggccctgggaggcttttcttcaatcgtatttgaagaatttaacctcaagcaaaataatcaagtccgcaatgtggaactagattttcagcggttcaccactggtaatacccacaccgcttatgtgatctgccgtcaggtcgagtctggagctaaacagttgggtattgatctaagtcaggcaacggtagcggtttgtggcgccacgggagatattggtagcgccgtatgtcgttggttagatagcaaacatcaagttaaggaattattgctaattgcccgtaaccgccaaagattggaaaatctccaagaggaattgggtcggggcaaaattatggatttggaaacagccctgccccaggcagatattattgtttgggtggctagtatgcccaagggggtagaaattgcgggggaaatgctgaaaaagccctgtttgattgtggatgggggctatcccaagaatttagacaccagggtgaaagcggatggggtgcatattctcaagggggggattgtagaacattcccttgatattacctgggaaattatgaagattgtggagatggatattccctcccggcaaatgttcgcctgttttgcggaggccattttgctagagtttgagggctggcgcactaatttttcctggggccgcaaccaaatttccgttaataaaatggaggcgattggtgaagcttctgtcaagcatggcttttgccctttagtagctctttag

SEQ ID NO: 68 - protein, 340 aa, Synechocystis sp.
  PCC6803 sll0209 (NP_442146) amino acid
MFGLIGHLTSLEHAQAVAEDLGYPEYANQGLDFWCSAPPQVVDNFQVKSVTGQVIEGKYVESCFLPEMLTQRRIKAAIRKILNAMALAQKVGLDITALGGFSSIVFEEFNLKQNNQVRNVELDFQRFTTGNTHTAYVICRQVESGAKQLGIDLSQATVAVCGATGDIGSAVCRWLDSKHQVKELLLIARNRQRLENLQEELGRGKIMDLETALPQADIIVWVASMPKGVEIAGEMLKKPCLIVDGGYPKNLDTRVKADGVHILKGGIVEHSLDITWEIMKIVEMDIPSRQMFACFAEAILLEFEGWRTNFSWGRNQISVNKMEAIGEASVKHGFCPLVAL

SEQ ID NO: 69 - DNA, 1023 bp, Cyanothece sp.
  ATCC51142 cce_1430 (YP_001802846) nucleotide
atgtttggtttaattggtcatcttacaagtttagaacacgcccactccgttgctgatgcctttggctatggcccatacgccactcagggacttgatttgtggtgttctgctccaccccaattcgtcgagcattttcatgttactagcatcacaggacaaaccatcgaaggaaagtatatagaatccgctttcttaccagaaatgctgataaagcgacggattaaagcagcaattcgcaaaatactgaatgcgatggcctttgctcagaaaaataaccttaacatcacagcattagggggcttttcttcgattatttttgaagaatttaatctcaaagagaatagacaagttcgtaatgtctctttagagtttgatcgcttcaccaccggaaacacccatactgcttatatcatttgtcgtcaagttgaacaggcatccgctaaactagggattgacttatcccaagcaacggttgctatttgcggggcaaccggagatattggcagtgcagtgtgtcgttggttagatagaaaaaccgatacccaggaactattcttaattgctcgcaataaagaacgattacaacgactgcaagatgagttgggacggggtaaaattatgggattggaggaggctttacccgaagcagatattatcgtttgggtggcgagtatgcccaaaggagtggaaattaatgccgaaactctcaaaaaaccctgtttaattatcgatggtggttatcctaagaatttagacacaaaaattaaacatcctgatgtccatatcctgaaagggggaattgtagaacattctctagatattgactggaagattatggaaactgtcaatatggatgttccttctcgtcaaatgtttgcttgttttgccgaagccattttattagagtttgaacaatggcacactaatttttcttggggacgcaatcaaattacagtgactaaaatggaacaaataggagaagcttctgtcaaacatgggttacaaccgttgttgagttggtaa

SEQ ID NO: 70 - protein, 340 aa, Cyanothece sp.
  ATCC51142 cce_1430 (YP_001802846) amino acid
MFGLIGHLTSLEHAHSVADAFGYGPYATQGLDLWCSAPPQFVEHFHVTSITGQTIEGKYIESAFLPEMLIKRRIKAAIRKILNAMAFAQKNNLNITALGGFSSIIFEEFNLKENRQVRNVSLEFDRFTTGNTHTAYIICRQVEQASAKLGIDLSQATVAICGATGDIGSAVCRWLDRKTDTQELFLIARNKERLQRLQDELGRGKIMGLEEALPEADIIVWVASMPKGVEINAETLKKPCLIIDGGYPKNLDTKIKHPDVHILKGGIVEHSLDIDWKIMETVNMDVPSRQMFACFAEAILLEFEQWHTNFSWGRNQITVTKMEQIGEASVKHGLQPLLSW

SEQ ID NO: 71 - DNA, 1041 bp, Prochlorococcus marinus
  subsp. pastoris  str. CCMP1986 PMM0533 (NP_892651) nucleotide
atgtttgggcttataggtcattcaactagttttgaagatgcaaaaagaaaggcttcattattgggctttgatcatattgcggatggtgatttagatgtttggtgcacagctccacctcaactagttgaaaatgtagaggttaaaagtgctataggtatatcaattgaaggttcttatattgattcatgtttcgttcctgaaatgctttcaagatttaaaacggcaagaagaaaagtattaaatgcaatggaattagctcaaaaaaaaggtattaatattaccgctttgggggggttcacttctatcatctttgaaaattttaatctccttcaacataagcagattagaaacacttcactagagtgggaaaggtttacaactggtaatactcatactgcgtgggttatttgcaggcaattagagatgaatgctcctaaaataggtattgatcttaaaagcgcaacagttgctgtagttggtgctactggagatataggcagtgctgtttgtcgatggttaatcaataaaacaggtattggggaacttcttttggtagctaggcaaaaggaacccttggattctttgcaaaaggaattagatggtggaactatcaaaaatctagatgaagcattgcctgaagcagatattgttgtatgggtagcaagtatgccaaagacaatggaaatcgatgctaataatcttaaacaaccatgtttaatgattgatggaggttatccaaagaatctagatgaaaaatttcaaggaaataatatacatgttgtaaaaggaggtatagtaagattcttcaatgatataggttggaatatgatggaactagctgaaatgcaaaatccccagagagaaatgtttgcatgctttgcagaagcaatgattttagaatttgaaaaatgtcatacaaactttagctggggaagaaataatatatctctcgagaaaatggagtttattggagctgcttctgtaaagcatggcttctctgcaattggcctagataagcatccaaaagtactagcagtttga

SEQ ID NO: 72 - protein, 346 aa, Prochlorococcus marinus
  subsp. pastoris str. CCMP1986 PMM0533 (NP_892651) amino acid
MFGLIGHSTSFEDAKRKASLLGFDHIADGDLDVWCTAPPQLVENVEVKSAIGISIEGSYIDSCFVPEMLSRFKTARRKVLNAMELAQKKGINITALGGFTSIIFENFNLLQHKQIRNTSLEWERFTTGNTHTAWVICRQLEMNAPKIGIDLKSATVAVVGATGDIGSAVCRWLINKTGIGELLLVARQKEPLDSLQKELDGGTIKNLDEALPEADIVVWVASMPKTMEIDANNLKQPCLMIDGGYPKNLDEKFQGNNIHVVKGGIVRFFNDIGWNMMELAEMQNPQREMFACFAEAMILEFEKCHTNFSWGRNNISLEKMEFIGAASVKHGFSAIGLDKHPKVLAV

SEQ ID NO: 73 - DNA, 1053 bp, Gloeobacter violaceus
  PCC7421 NP_96091 (gll3145) nucleotide
atgtttggcctgatcggacacttgaccaatctttcccatgcccagcgggtcgcccgcgacctgggctacgacgagtatgcaagccacgacctcgaattctggtgcatggcccctccccaggcggtcgatgaaatcacgatcaccagcgtcaccggtcaggtgatccacggtcagtacgtcgaatcgtgctttctgccggagatgctcgcccagggccgcttcaagaccgccatgcgcaagatcctcaatgccatggccctggtccagaagcgcggcatcgacattacggccctgggaggcttctcgtcgatcatcttcgagaatttcagcctcgataaattgctcaacgtccgcgacatcaccctcgacatccagcgcttcaccaccggcaacacccacacggcctacatcctttgtcagcaggtcgagcagggtgcggtacgctacggcatcgatccggccaaagcgaccgtggcggtagtcggggccaccggcgacatcggtagcgccgtctgccgatggctcaccgaccgcgccggcatccacgaactcttgctggtggcccgcgacgccgaaaggctcgaccggctgcagcaggaactcggcaccggtcggatcctgccggtcgaagaagcacttcccaaagccgacatcgtcgtctgggtcgcctcgatgaaccagggcatggccatcgaccccgccggcctgcgcaccccctgcctgctcatcgacggcggctaccccaagaacatggccggcaccctgcagcgcccgggcatccatatcctcgacggcggcatggtcgagcactcgctcgacatcgactggcagatcatgtcgtttctaaatgtgcccaaccccgcccgccagttcttcgcctgcttcgccgagtcgatgctgctggaattcgaagggcttcacttcaatttttcctggggccgcaaccacatcaccgtcgagaagatggcccagatcggctcgctgtctaaaaaacatggctttcgtcccctgcttgaacccagtcagcgcagcggcgaactcgtacacggataa

SEQ ID NO: 74 - protein, 350 aa, Gloeobacter violaceus
  PCC7421 NP_96091 (gll3145) amino acid
MFGLIGHLTNLSHAQRVARDLGYDEYASHDLEFWCMAPPQAVDEITITSVTGQVIHGQYVESCFLPEMLAQGRFKTAMRKILNAMALVQKRGIDITALGGFSSIIFENFSLDKLLNVRDITLDIQRFTTGNTHTAYILCQQVEQGAVRYGIDPAKATVAVVGATGDIGSAVCRWLTDRAGIHELLLVARDAERLDRLQQELGTGRILPVEEALPKADIVVWVASMNQGMAIDPAGLRTPCLLIDGGYPKNMAGTLQRPGIHILDGGMVEHSLDIDWQIMSFLNVPNPARQFFACFAESMLLEFEGLHFNFSWGRNHITVEKMAQIGSLSKKHGFRPLLEPSQRSGELVHG

SEQ ID NO: 75 - DNA, 1020 bp, Nostoc punctiforme
  PCC73102 ZP_00108837 (Npun02004176) nucleotide
atgtttggtctaattggacatctgactagtttagaacacgctcaagccgtagcccaagaattgggatacccagaatatgccgatcaagggctagacttttggtgcagcgccccgccgcaaattgtcgatagtattattgtcaccagtgttactgggcaacaaattgaaggacgatatgtagaatcttgctttttgccggaaatgctagctagtcgccgcatcaaagccgcaacacggaaaatcctcaacgctatggcccatgcacagaagcacggcattaacatcacagctttaggcggattttcctcgattatttttgaaaactttaagttagagcagtttagccaagtccgaaatatcaagctagagtttgaacgcttcaccacaggaaacacgcatactgcctacattatttgtaagcaggtggaagaagcatccaaacaactgggaattaatctatcaaacgcgactgttgcggtatgtggagcaactggggatattggtagtgccgttacacgctggctagatgcgagaacagatgtccaagaactcctgctaatcgcccgcgatcaagaacgtctcaaagagttgcaaggcgaactggggcgggggaaaatcatgggtttgacagaagcactaccccaagccgatgttgtagtttgggttgctagtatgcccagaggcgtggaaattgaccccaccactttgaaacaaccctgtttgttgattgatggtggctatcctaaaaacttagcaacaaaaattcaatatcctggcgtacacgtgttaaatggtgggattgtagagcattccctggatattgactggaaaattatgaaaatagtcaatatggacgtgccagcccgtcagttgtttgcctgttttgccgaatcaatgctactggaatttgagaagttatacacgaacttttcgtggggacggaatcagattaccgtagataaaatggagcagattggccgggtgtcagtaaaacatggatttagaccgttgttggtttag

SEQ ID NO: 76 - protein, 339 aa, Nostoc punctiforme
  PCC73102 ZP_00108837 (Npun02004176) amino acid
MFGLIGHLTSLEHAQAVAQELGYPEYADQGLDFWCSAPPQIVDSIIVTSVTGQQIEGRYVESCFLPEMLASRRIKAATRKILNAMAHAQKHGINITALGGFSSIIFENFKLEQFSQVRNIKLEFERFTTGNTHTAYIICKQVEEASKQLGINLSNATVAVCGATGDIGSAVTRWLDARTDVQELLLIARDQERLKELQGELGRGKIMGLTEALPQADVVVWVASMPRGVEIDPTTLKQPCLLIDGGYPKNLATKIQYPGVHVLNGGIVEHSLDIDWKIMKIVNMDVPARQLFACFAESMLLEFEKLYTNFSWGRNQITVDKMEQIGRVSVKHGFRPLLV

SEQ ID NO: 77 - DNA, 1020 bp, Anabaena variabilis
  ATCC29413 YP_323044 (Ava_2534) nucleotide
atgtttggtctaattggacatctgacaagtttagaacacgctcaagcggtagctcaagaactgggatacccagaatacgccgaccaagggctagatttttggtgcagcgctccaccgcaaatagttgaccacattaaagttactagcattactggtgaaataattgaagggaggtatgtagaatcttgctttttaccagaaatgctagccagccgtaggattaaagccgcaacccgcaaagtcctcaatgctatggctcatgctcaaaaacatggcattgacatcaccgctttgggtggtttctcctccattatttttgaaaacttcaaattggaacagtttagccaagttcgtaatgtcacactagagtttgaacgcttcactacaggcaacactcacacagcttatatcatttgtcggcaggtagaacaagcatcacaacaactcggcattgaactctcccaagcaacagtagctatatgtggggctactggtgacattggtagtgcagttactcgctggctggatgccaaaacagacgtaaaagaattactgttaatcgcccgtaatcaagaacgtctccaagagttgcaaagcgagttgggacgcggtaaaatcatgagcctagatgaagcattgcctcaagctgatattgtagtttgggtagctagtatgcctaaaggcgtggaaattaatcctcaagttttgaaacaaccctgtttattgattgatggtggttatccgaaaaacttgggtacaaaagttcagtatcctggtgtttatgtactgaacggaggtatcgtcgaacattccctagatattgactggaaaatcatgaaaatagtcaatatggatgtacctgcacgccaattatttgcttgttttgcggaatctatgctcttggaatttgagaagttgtacacgaacttttcttgggggcgcaatcagattaccgtagacaaaatggagcagattggtcaagcatcagtgaaacatgggtttagaccactgctggtttag

SEQ ID NO: 78 - protein, 339 aa, Anabaena variabilis
  ATCC29413 YP_323044 (Ava_2534) amino acid
MFGLIGHLTSLEHAQAVAQELGYPEYADQGLDFWCSAPPQIVDHIKVTSITGEIIEGRYVESCFLPEMLASRRIKAATRKVLNAMAHAQKHGIDITALGGFSSIIFENFKLEQFSQVRNVTLEFERFTTGNTHTAYIICRQVEQASQQLGIELSQATVAICGATGDIGSAVTRWLDAKTDVKELLLIARNQERLQELQSELGRGKIMSLDEALPQADIVVWVASMPKGVEINPQVLKQPCLLIDGGYPKNLGTKVQYPGVYVLNGGIVEHSLDIDWKIMKIVNMDVPARQLFACFAESMLLEFEKLYTNFSWGRNQITVDKMEQIGQASVKHGFRPLLV

SEQ ID NO: 79 - DNA, 1026 bp, Synechococcus elongatus; identical to SEQ ID NO: 65
  PCC6301 YP_170761 (syc0051_d) nucleotide

SEQ ID NO: 80 - protein, 340 aa, Synechococcus elongatus
  PCC6301 YP_170761 (syc0051_d) amino acid
MFGLIGHLTSLEQARDVSRRMGYDEYADQGLEFWSSAPPQIVDEITVTSATGKVIHGRYIESCFLPEMLAARRFKTATRKVLNAMSHAQKHGIDISALGGFTSIIFENFDLASLRQVRDTTLEFERFTTGNTHTAYVICRQVEAAAKTLGIDITQATVAVVGATGDIGSAVCRWLDLKLGVGDLILTARNQERLDNLQAELGRGKILPLEAALPEADFIVWVASMPQGVVIDPATLKQPCVLIDGGYPKNLGSKVQGEGIYVLNGGVVEHCFDIDWQIMSAAEMARPERQMFACFAEAMLLEFEGWHTNFSWGRNQITIEKMEAIGEASVRHGFQPLALA

SEQ ID NO: 81 - DNA, 1020 bp, Nostoc sp.
  PCC 7120 alr5284 (NP_489324) nucleotide
atgtttggtctaattggacatctgacaagtttagaacacgctcaagcggtagctcaagaactgggatacccagaatacgccgaccaagggctagatttttggtgtagcgctccaccgcaaatagttgaccacattaaagttactagtattactggtgaaataattgaagggaggtatgtagaatcttgctttttaccggagatgctagccagtcgtcggattaaagccgcaacccgcaaagtcctcaatgctatggctcatgctcaaaagaatggcattgatatcacagctttgggtggtttctcctccattatttttgaaaactttaaattggagcagtttagccaagttcgtaatgtgacactagagtttgaacgcttcactacaggcaacactcacacagcatatattatttgtcggcaggtagaacaagcatcacaacaactcggcattgaactctcccaagcaacagtagctatatgtggggctactggtgatattggtagtgcagttactcgctggctggatgctaaaacagacgtgaaagaattgctgttaatcgcccgtaatcaagaacgtctccaagagttgcaaagcgagctgggacgcggtaaaatcatgagccttgatgaagcactgccccaagctgatatcgtagtttgggtagccagtatgcctaaaggtgtggaaattaatcctcaagttttgaagcaaccctgtttgctgattgatgggggttatccgaaaaacttgggtacaaaagttcagtatcctggtgtttatgtactgaacggcggtatcgtcgaacattcgctggatattgactggaaaatcatgaaaatagtcaatatggatgtacctgcacgccaattatttgcttgttttgcggaatctatgctcttggaatttgagaagttgtacacgaacttttcttgggggcgcaatcagattaccgtagacaaaatggagcagattggtcaagcatcagtgaaacatgggtttagaccactgctggtttag

SEQ ID NO: 82 - protein, 339 aa, Nostoc sp.
  PCC 7120 alr5284 (NP_489324) amino acid
MFGLIGHLTSLEHAQAVAQELGYPEYADQGLDFWCSAPPQIVDHIKVTSITGEIIEGRYVESCFLPEMLASRRIKAATRKVLNAMAHAQKNGIDITALGGFSSIIFENFKLEQFSQVRNVTLEFERFTTGNTHTAYIICRQVEQASQQLGIELSQATVAICGATGDIGSAVTRWLDAKTDVKELLLIARNQERLQELQSELGRGKIMSLDEALPQADIVVWVASMPKGVEINPQVLKQPCLLIDGGYPKNLGTKVQYPGVYVLNGGIVEHSLDIDWKIMKIVNMDVPARQLFACFAESMLLEFEKLYTNFSWGRNQITVDKMEQIGQASVKHGFRPLLV

SEQ ID NO: 83 - DNA, 1026 bp, Artificial Sequence
  Description of Artificial Sequence Synthetic PCC7942 Synpcc7942_1594 (YP_400611) polynucleotide
atgtttggtctgattggtcacctgaccagcttggaacaagcgcgtgacgtcagccgccgtatgggttatgatgaatacgctgatcaaggcctggagttttggagcagcgcgccaccgcagatcgtcgatgagatcaccgtgacctccgcaaccggtaaggtcatccacggccgctacattgagtcctgcttcctgcctgagatgctggcagctcgccgtttcaaaacggccactcgtaaggttctgaatgcgatgtcccatgcgcaaaagcatggcattgacattagcgccttgggcggttttacgtcgattatcttcgagaacttcgatctggcctctttgcgccaggtgcgtgacacgaccttggagtttgagcgttttaccacgggtaatacgcacaccgcttacgttatctgtcgccaagtcgaagcagcagccaaaaccctgggtattgatatcacccaggccaccgtcgccgtggtgggtgctaccggtgatattggttccgcggtttgccgttggctggatctgaaactgggtgttggcgatctgatcctgacggcgcgtaatcaggagcgtctggacaacctgcaagccgagttgggtcgcggtaagatcctgccgttggaggcagcgttgccggaggcagacttcatcgtctgggttgcgtctatgccgcagggtgttgttatcgacccggcgaccttgaaacagccgtgcgtgctgattgatggcggctatccgaaaaacctgggcagcaaggtccaaggcgagggtatctatgtcctgaatggcggtgtggttgagcattgcttcgacattgactggcagatcatgagcgcagcagaaatggcgcgtccggagcgccaaatgtttgcctgttttgcagaagccatgctgctggagttcgaaggctggcatacgaatttcagctggggtcgtaatcagattaccattgaaaagatggaagcgattggtgaagcaagcgtgcgtcatggttttcagccactggcgctggctatttaa

SEQ ID NO: 84 - DNA, 1041 bp, Artificial Sequence
  Description of Artificial Sequence Synthetic PMM0533 (NP_892651) polynucleotide
atgtttggtctgattggccacagcacgagctttgaggacgcaaagcgtaaggcgagcctgctgggctttgatcatattgctgatggcgacctggacgtctggtgcacggcacctccgcaactggttgagaatgtcgaggtgaaatcggcgattggcatttccatcgaaggctcctacatcgacagctgtttcgtgccggagatgttgagccgtttcaaaaccgcacgtcgcaaagttctgaatgcaatggagctggcacaaaagaagggcatcaacatcacggcgctgggtggtttcaccagcattatctttgagaacttcaatctgttgcagcataaacagatccgtaataccagcctggagtgggaacgctttaccacgggtaacacccacaccgcgtgggtgatctgccgccagctggagatgaatgcgccgaaaatcggtattgacctgaaaagcgcgacggtggcagttgttggcgcaactggcgacattggttcggccgtttgtcgctggctgattaacaagaccggtatcggtgaattgttgctggtcgctcgccagaaggagcctctggacagcctgcaaaaagagctggacggtggtacgatcaagaacctggatgaagcgctgccagaagcggacatcgtcgtctgggtcgcatctatgccgaaaactatggaaatcgatgccaacaatctgaaacaaccgtgcctgatgatcgatggcggctacccgaagaacttggatgagaagtttcaaggcaataacatccacgttgtgaagggtggtattgtccgtttcttcaatgatatcggttggaacatgatggaactggctgaaatgcagaacccgcaacgtgagatgttcgcttgttttgcggaggccatgattctggagttcgagaaatgccataccaatttcagctggggtcgcaacaacattagcctggagaaaatggagttcatcggcgctgcgagcgttaagcacggcttcagcgcgattggtttggataaacatccgaaggtcctggcagtttaa

SEQ ID NO: 85 - DNA, 3522 bp, Mycobacterium smegmatis
  strain MC2 155 orf MSMEG_5739 (YP_889972) nucleotide
atgaccagcgatgttcacgacgccacagacggcgtcaccgaaaccgcactcgacgacgagcagtcgacccgccgcatcgccgagctgtacgccaccgatcccgagttcgccgccgccgcaccgttgcccgccgtggtcgacgcggcgcacaaacccgggctgcggctggcagagatcctgcagaccctgttcaccggctacggtgaccgcccggcgctgggataccgcgcccgtgaactggccaccgacgagggcgggcgcaccgtgacgcgtctgctgccgcggttcgacaccctcacctacgcccaggtgtggtcgcgcgtgcaagcggtcgccgcggccctgcgccacaacttcgcgcagccgatctaccccggcgacgccgtcgcgacgatcggtttcgcgagtcccgattacctgacgctggatctcgtatgcgcctacctgggcctcgtgagtgttccgctgcagcacaacgcaccggtcagccggctcgccccgatcctggccgaggtcgaaccgcggatcctcaccgtgagcgccgaatacctcgacctcgcagtcgaatccgtgcgggacgtcaactcggtgtcgcagctcgtggtgttcgaccatcaccccgaggtcgacgaccaccgcgacgcactggcccgcgcgcgtgaacaactcgccggcaagggcatcgccgtcaccaccctggacgcgatcgccgacgagggcgccgggctgccggccgaaccgatctacaccgccgaccatgatcagcgcctcgcgatgatcctgtacacctcgggttccaccggcgcacccaagggtgcgatgtacaccgaggcgatggtggcgcggctgtggaccatgtcgttcatcacgggtgaccccacgccggtcatcaacgtcaacttcatgccgctcaaccacctgggcgggcgcatccccatttccaccgccgtgcagaacggtggaaccagttacttcgtaccggaatccgacatgtccacgctgttcgaggatctcgcgctggtgcgcccgaccgaactcggcctggttccgcgcgtcgccgacatgctctaccagcaccacctcgccaccgtcgaccgcctggtcacgcagggcgccgacgaactgaccgccgagaagcaggccggtgccgaactgcgtgagcaggtgctcggcggacgcgtgatcaccggattcgtcagcaccgcaccgctggccgcggagatgagggcgttcctcgacatcaccctgggcgcacacatcgtcgacggctacgggctcaccgagaccggcgccgtgacacgcgacggtgtgatcgtgcggccaccggtgatcgactacaagctgatcgacgttcccgaactcggctacttcagcaccgacaagccctacccgcgtggcgaactgctggtcaggtcgcaaacgctgactcccgggtactacaagcgccccgaggtcaccgcgagcgtcttcgaccgggacggctactaccacaccggcgacgtcatggccgagaccgcacccgaccacctggtgtacgtggaccgtcgcaacaacgtcctcaaactcgcgcagggcgagttcgtggcggtcgccaacctggaggcggtgttctccggcgcggcgctggtgcgccagatcttcgtgtacggcaacagcgagcgcagtttccttctggccgtggtggtcccgacgccggaggcgctcgagcagtacgatccggccgcgctcaaggccgcgctggccgactcgctgcagcgcaccgcacgcgacgccgaactgcaatcctacgaggtgccggccgatttcatcgtcgagaccgagccgttcagcgccgccaacgggctgctgtcgggtgtcggaaaactgctgcggcccaacctcaaagaccgctacgggcagcgcctggagcagatgtacgccgatatcgcggccacgcaggccaaccagttgcgcgaactgcggcgcgcggccgccacacaaccggtgatcgacaccctcacccaggccgctgccacgatcctcggcaccgggagcgaggtggcatccgacgcccacttcaccgacctgggcggggattccctgtcggcgctgacactttcgaacctgctgagcgatttcttcggtttcgaagttcccgtcggcaccatcgtgaacccggccaccaacctcgcccaactcgcccagcacatcgaggcgcagcgcaccgcgggtgaccgcaggccgagtttcaccaccgtgcacggcgcggacgccaccgagatccgggcgagtgagctgaccctggacaagttcatcgacgccgaaacgctccgggccgcaccgggtctgcccaaggtcaccaccgagccacggacggtgttgctctcgggcgccaacggctggctgggccggttcctcacgttgcagtggctggaacgcctggcacctgtcggcggcaccctcatcacgatcgtgcggggccgcgacgacgccgcggcccgcgcacggctgacccaggcctacgacaccgatcccgagttgtcccgccgcttcgccgagctggccgaccgccacctgcgggtggtcgccggtgacatcggcgacccgaatctgggcctcacacccgagatctggcaccggctcgccgccgaggtcgacctggtggtgcatccggcagcgctggtcaaccacgtgctcccctaccggcagctgttcggccccaacgtcgtgggcacggccgaggtgatcaagctggccctcaccgaacggatcaagcccgtcacgtacctgtccaccgtgtcggtggccatggggatccccgacttcgaggaggacggcgacatccggaccgtgagcccggtgcgcccgctcgacggcggatacgccaacggctacggcaacagcaagtgggccggcgaggtgctgctgcgggaggcccacgatctgtgcgggctgcccgtggcgacgttccgctcggacatgatcctggcgcatccgcgctaccgcggtcaggtcaacgtgccagacatgttcacgcgactcctgttgagcctcttgatcaccggcgtcgcgccgcggtcgttctacatcggagacggtgagcgcccgcgggcgcactaccccggcctgacggtcgatttcgtggccgaggcggtcacgacgctcggcgcgcagcagcgcgagggatacgtgtcctacgacgtgatgaacccgcacgacgacgggatctccctggatgtgttcgtggactggctgatccgggcgggccatccgatcgaccgggtcgacgactacgacgactgggtgcgtcggttcgagaccgcgttgaccgcgcttcccgagaagcgccgcgcacagaccgtactgccgctgctgcacgcgttccgcgctccgcaggcaccgttgcgcggcgcacccgaacccacggaggtgttccacgccgcggtgcgcaccgcgaaggtgggcccgggagacatcccgcacctcgacgaggcgctgatcgacaagtacatacgcgatctgcgtgagttcggtctgatctga

SEQ ID NO: 86 - protein, 1173 aa, Mycobacterium smegmatis
  strain MC2 155 orf MSMEG_5739 (YP_889972) amino acid
MTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 87 - DNA, 921 bp, Nostoc punctiforme
  PCC73102 Npun02003626 (ZP_00109192) nucleotide
atgactcaagcgaaagccaaaaaagaccacggtgacgttcctgttaacacttaccgtcccaatgctccatttattggcaaggtaatatctaatgaaccattagtcaaagaaggtggtattggtattgttcaacaccttaaatttgacctatctggtggggatttgaagtatatagaaggtcaaagtattggcattattccgccaggtttagacaagaacggcaagcctgaaaaactcagactatattccatcgcctcaactcgtcatggtgatgatgtagatgataagacagtatcactgtgcgtccgccagttggagtacaagcacccagaaactggcgaaacagtctacggtgtttgctctacgcacctgtgtttcctcaagccaggggaagaggtaaaaattacagggcctgtgggtaaggaaatgttgttacccaatgaccctgatgctaatgttatcatgatggctactggaacaggtattgcgccgatgcgggcttacttgtggcgtcagtttaaagatgcggaaagagcggctaacccagaataccaatttaaaggattctcttggctaatatttggcgtacctacaactccaaaccttttatataaggaagaactggaagagattcaacaaaaatatcctgagaacttccgcctaactgctgccatcagccgcgaacagaaaaatccccaaggcggtagaatgtatattcaagaccgcgtagcagaacatgctgatgaattgtggcagttgattaaaaatgaaaaaacccacacttacatttgcggtttgcgcggtatggaagaaggtattgatgcagccttaactgctgctgctgctaaggaaggcgtaacctggagtgattaccagaagcaactcaagaaagccggtcgctggcacgtagaaacttactaa

SEQ ID NO: 88 - protein, 437 aa, Nostoc punctiforme
  PCC73102 Npun02003626 (ZP_00109192) amino acid
MYNQGAVEGAANIELGSRIFVYEVVGLRQGEETDQTNYPIRKSGSVFIRVPYNRMNQEMRRITRLGGTIVSIQPITALEPVNGKASFGNATSVVSELAKSGETANSEGNGKATPVNAHSAEEQNKDKKGNTMTQAKAKKDHGDVPVNTYRPNAPFIGKVISNEPLVKEGGIGIVQHLKFDLSGGDLKYIEGQSIGIIPPGLDKNGKPEKLRLYSIASTRHGDDVDDKTVSLCVRQLEYKHPETGETVYGVCSTHLCFLKPGEEVKITGPVGKEMLLPNDPDANVIMMATGTGIAPMRAYLWRQFKDAERAANPEYQFKGFSWLIFGVPTTPNLLYKEELEEIQQKYPENFRLTAAISREQKNPQGGRMYIQDRVAEHADELWQLIKNEKTHTYICGLRGMEEGIDAALTAAAAKEGVTWSDYQKQLKKAGRWHVETY

SEQ ID NO: 89 - DNA, 300 bp, Nostoc punctiforme
  PCC73102 Npun02001001 (ZP_00111633) nucleotide
atgccaacttataaagtgacactaattaacgaggctgaagggctgaacacaacccttgatgttgaggacgatacctatattctagacgcagctgaagaagctggtattgacctgccctactcttgccgcgctggtgcttgctctacttgtgcaggtaaactcgtatcaggtaccgtcgatcaaggcgatcaatcattcttagatgacgatcaaatagaagctggatatgtactgacctgtgttgcttacccaacttctaatgtcacgatcgaaactcacaaagaagaagaactctattaa

SEQ ID NO: 90 - protein, 99 aa, Nostoc punctiforme
  PCC73102 Npun02001001 (ZP_00111633) amino acid
MPTYKVTLINEAEGLNTTLDVEDDTYILDAAEEAGIDLPYSCRAGACSTCAGKLVSGTVDQGDQSFLDDDQIEAGYVLTCVAYPTSNVTIETHKEEELY

SEQ ID NO: 91 - DNA, 369 bp, Nostoc punctiforme
  PCC73102 Npun02003530 (ZP_00109422) nucleotide
atgtcccgtacatacacaattaaagttcgcgatcgcgccactggcaaaacacacaccctaaaagtgccagaagaccgttatatcctgcacactgccgaaaaacaaggtgtggaactaccgttttcctgtcgcaacggagcttgcaccgcttgtgctgtgagggtattgtcaggagaaatttatcaaccagaggcgatcggattgtcaccagatttacgtcagcaaggttatgccctgttgtgtgtgagttatccccgttctgacttggaagtagagacacaagacgaagatgaagtctacgaactccagtttgggcgctattttgctaaggggaaagttaaagcgggtttaccgttagatgaggaataa

SEQ ID NO: 92 - protein, 122 aa, Nostoc punctiforme
  PCC73102 Npun02003530 (ZP_00109422) amino acid
MSRTYTIKVRDRATGKTHTLKVPEDRYILHTAEKQGVELPFSCRNGACTACAVRVLSGEIYQPEAIGLSPDLRQQGYALLCVSYPRSDLEVETQDEDEVYELQFGRYFAKGKVKAGLPLDEE

SEQ ID NO: 93 - DNA, 321 bp, Nostoc punctiforme
  PCC73102 Npun02003123 (ZP_00109501) nucleotide
atgcccaaaacttacaccgtagaaatcgatcatcaaggcaaaattcataccttgcaagttcctgaaaatgaaacgatcttatcagttgccgatgctgctggtttggaactgccgagttcttgtaatgcaggtgtttgcacaacttgcgccggtcaaataagccagggaactgtggatcaaactgatggcatgggcgttagtccagatttacaaaagcaaggttacgtattgctttgtgttgcgaaacccctttctgatttgaaacttgaaacagaaaaggaagacatagtttatcagttacaatttggcaaagacaaataa

SEQ ID NO: 94 - protein, 106 aa, Nostoc punctiforme
  PCC73102 Npun02003123 (ZP_00109501) amino acid
MPKTYTVEIDHQGKIHTLQVPENETILSVADAAGLELPSSCNAGVCTTCAGQISQGTVDQTDGMGVSPDLQKQGYVLLCVAKPLSDLKLETEKEDIVYQLQFGKDK

SEQ ID NO: 95 - DNA, 1020 bp, Nostoc punctiforme; identical to SEQ ID NO: 75
  PCC73102 ferrodoxin Npun_R1710 (NC_010628.1, petF) gi|186680550c2097273-2096254 Nostoc punctiforme PCC 73102, complete genome

SEQ ID NO: 96 - DNA, 1314 bp, Nostoc punctiforme
  Ferrodoxin oxidoreductase Npun02003623 petH gi|186680550c3410418-3409105 PCC 73102, complete genome
atgtacaatcaaggtgctgttgagggtgctgccaacatagaattaggtagccgcatcttcgtttatgaagtagtgggtttgcgtcagggggaagaaaccgatcaaactaactacccaattcggaaaagtggcagtgtgttcatcagagtgccttacaaccgcatgaatcaagaaatgcgacgtatcactcgtctaggcggcacaattgttagcatccaacctataactgctctagaaccagttaatggtaaagcctcatttgggaatgctacaagcgttgtcagcgaattagctaaatctggggaaactgctaacagtgaagggaatggtaaagccacacctgtaaatgctcatagtgctgaagaacagaacaaggacaagaaaggcaacaccatgactcaagcgaaagccaaaaaagaccacggtgacgttcctgttaacacttaccgtcccaatgctccatttattggcaaggtaatatctaatgaaccattagtcaaagaaggtggtattggtattgttcaacaccttaaatttgacctatctggtggggatttgaagtatatagaaggtcaaagtattggcattattccgccaggtttagacaagaacggcaagcctgaaaaactcagactatattccatcgcctcaactcgtcatggtgatgatgtagatgataagacagtatcactgtgcgtccgccagttggagtacaagcacccagaaactggcgaaacagtctacggtgtttgctctacgcacctgtgtttcctcaagccaggggaagaggtaaaaattacagggcctgtgggtaaggaaatgttgttacccaatgaccctgatgctaatgttatcatgatggctactggaacaggtattgcgccgatgcgggcttacttgtggcgtcagtttaaagatgcggaaagagcggctaacccagaataccaatttaaaggattctcttggctaatatttggcgtacctacaactccaaaccttttatataaggaagaactggaagagattcaacaaaaatatcctgagaacttccgcctaactgctgccatcagccgcgaacagaaaaatccccaaggcggtagaatgtatattcaagaccgcgtagcagaacatgctgatgaattgtggcagttgattaaaaatgaaaaaacccacacttacatttgcggtttgcgcggtatggaagaaggtattgatgcagccttaactgctgctgctgctaaggaaggcgtaacctggagtgattaccagaagcaactcaagaaagccggtcgctggcacgtagaaacttactaa

SEQ ID NO: 97 - DNA, 70 bp, Artificial Sequence
  Description of Artificial Sequence Synthetic primer Del-fadE-F
aaaaacagcaacaatgtgagctttgttgtaattatattgtaaacatattgattccggggatccgtcgacc

SEQ ID NO: 98 - DNA, 68 bp, Artificial Sequence
  Description of Artificial Sequence Synthetic primer Del-fadE-R
aaacggagcctttcggctccgttattcatttacgcggcttcaactttcctgtaggctggagctgcttc

SEQ ID NO: 99 - DNA, 23 bp, Artificial Sequence
  Description of Artificial Sequence Synthetic primer fadE-L2
cgggcaggtgctatgaccaggac

SEQ ID NO: 100 - DNA, 23 bp, Artificial Sequence
  Description of Artificial Sequence Synthetic primer fadE-R1
cgcggcgttgaccggcagcctgg

SEQ ID NO: 101 - DNA, 70 bp, Artificial Sequence
  Description of Artificial Sequence Synthetic primer Del-fhuA-F
atcattctcgtttacgttatcattcactttacatcagagatataccaatgattccggggatccgtcgacc

SEQ ID NO: 102 - DNA, 69 bp, Artificial Sequence
  Description of Artificial Sequence Synthetic primer Del-fhuA-R
gcacggaaatccgtgccccaaaagagaaattagaaacggaaggttgcggttgtaggctggagctgcttc

SEQ ID NO: 103 - DNA, 21 bp, Artificial Sequence
  Description of Artificial Sequence Synthetic primer fhuA-verF
caacagcaacctgctcagcaa

SEQ ID NO: 104 - DNA, 21 bp, Artificial Sequence
  Description of Artificial Sequence Synthetic primer fhuA-verR
aagctggagcagcaaagcgtt

SEQ ID NO: 105 - DNA, 3533 bp, Artificial Sequence
  Description of Artificial Sequence Synthetic vector OP-183
cggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatctggtttgacagcttatcatcgactgcacggtgcaccaatgcttctggcgtcaggcagccatccccgggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataacatatgccaacttataaagtgacactaattaacgaggctgaagggctgaacacaacccttgatgttgaggacgatacctatattctagacgcagctgaagaagctggtattgacctgccctactcttgccgcgctggtgcttgctctacttgtgcaggtaaactcgtatcaggtaccgtcgatcaaggcgatcaatcattcttagatgacgatcaaatagaagctggatatgtactgacctgtgttgcttacccaacttctaatgtcacgatcgaaactcacaaagaagaagaactctattaataaggaggaaaacaaaatgactcaagcgaaagccaaaaaagaccacggtgacgttcctgttaacacttaccgtcccaatgctccatttattggcaaggtaatatctaatgaaccattagtcaaagaaggtggtattggtattgttcaacaccttaaatttgacctatctggtggggatttgaagtatatagaaggtcaaagtattggcattattccgccaggtttagacaagaacggcaagcctgaaaaactcagactatattccatcgcctcaactcgtcatggtgatgatgtagatgataagacagtatcactgtgcgtccgccagttggagtacaagcacccagaaactggcgaaacagtctacggtgtttgctctacgcacctgtgtttcctcaagccaggggaagaggtaaaaattacagggcctgtgggtaaggaaatgttgttacccaatgaccctgatgctaatgttatcatgatggctactggaacaggtattgcgccgatgcgggcttacttgtggcgtcagtttaaagatgcggaaagagcggctaacccagaataccaatttaaaggattctcttggctaatatttggcgtacctacaactccaaaccttttatataaggaagaactggaagagattcaacaaaaatatcctgagaacttccgcctaactgctgccatcagccgcgaacagaaaaatccccaaggcggtagaatgtatattcaagaccgcgtagcagaacatgctgatgaattgtggcagttgattaaaaatgaaaaaacccacacttacatttgcggtttgcgcggtatggaagaaggtattgatgcagccttaactgctgctgctgctaaggaaggcgtaacctggagtgattaccagaagcaactcaagaaagccggtcgctggcacgtagaaacttactaagaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgacaggaacgtcgtgctgacgcttcatcagaagggcactggtgcaacggaaattgctcatcagctcagtattgcccgctccacggtttataaaattcttgaagacgaaagggcctcgtgatacgcctatttttataggttaatgtcatgataataatggtttctta

SEQ ID NO: 106 - DNA, 40 bp, Artificial Sequence
  Description of Artificial Sequence Synthetic primer petF-forward
gcaattcatatgccaacttataaagtgacactaattaacg

SEQ ID NO: 107 - DNA, 50 bp, Artificial Sequence
  Description of Artificial Sequence Synthetic primer petF-reverse
tgagtcattttgttttcctccttattaatagagttcttcttctttgtgag

SEQ ID NO: 108 - DNA, 50 bp, Artificial Sequence
  Description of Artificial Sequence Synthetic primer petH-forward
tctattaataaggaggaaaacaaaatgactcaagcgaaagccaaaaaaga

SEQ ID NO: 109 - DNA, 35 bp, Artificial Sequence
  Description of Artificial Sequence Synthetic primer petH-reverse
agcttcgaattcttagtaagtttctacgtgccagc

SEQ ID NO: 110 - DNA, 7314 bp, Artificial Sequence
  Description of Artificial Sequence Synthetic plasmid pDS57 polynucleotide
cactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcgtcggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatgaaacgtctcggaaccctggacgcctcctggctggcggttgaatctgaagacaccccgatgcatgtgggtacgcttcagattttctcactgccggaaggcgcaccagaaaccttcctgcgtgacatggtcactcgaatgaaagaggccggcgatgtggcaccaccctggggatacaaactggcctggtctggtttcctcgggcgcgtgatcgccccggcctggaaagtcgataaggatatcgatctggattatcacgtccggcactcagccctgcctcgccccggcggggagcgcgaactgggtattctggtatcccgactgcactctaaccccctggatttttcccgccctctttgggaatgccacgttattgaaggcctggagaataaccgttttgccctttacaccaaaatgcaccactcgatgattgacggcatcagcggcgtgcgactgatgcagagggtgctcaccaccgatcccgaacgctgcaatatgccaccgccctggacggtacgcccacaccaacgccgtggtgcaaaaaccgacaaagaggccagcgtgcccgcagcggtttcccaggcaatggacgccctgaagctccaggcagacatggcccccaggctgtggcaggccggcaatcgcctggtgcattcggttcgacacccggaagacggactgaccgcgcccttcactggaccggtttcggtgctcaatcaccgggttaccgcgcagcgacgttttgccacccagcattatcaactggaccggctgaaaaacctggcccatgcttccggcggttccttgaacgacatcgtgctttacctgtgtggcaccgcattgcggcgctttctggctgagcagaacaatctgccagacaccccgctgacggctggtataccggtgaatatccggccggcagacgacgagggtacgggcacccagatcagttttatgattgcctcgctggccaccgacgaagctgatccgttgaaccgcctgcaacagatcaaaacctcgacccgacgggccaaggagcacctgcagaaacttccaaaaagtgccctgacccagtacaccatgctgctgatgtcaccctacattctgcaattgatgtcaggtctcggggggaggatgcgaccagtcttcaacgtgaccatttccaacgtgcccggcccggaaggcacgctgtattatgaaggagcccggcttgaggccatgtatccggtatcgctaatcgctcacggcggcgccctgaacatcacctgcctgagctatgccggatcgctgaatttcggttttaccggctgtcgggatacgctgccgagcatgcagaaactggcggtttataccggtgaagctctggatgagctggaatcgctgattctgccacccaagaagcgcgcccgaacccgcaagtaactcgagatctgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgacgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaat

SEQ ID NO: 111 - DNA, 564 bp, Artificial Sequence
  Description of Artificial Sequence Synthetic (1/2 kan) polynucleotide
ttagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgc

SEQ ID NO: 112 - DNA, 72 bp, Artificial Sequence
  Description of Artificial Sequence Synthetic primer 1
aaagaggtatatattaatgtatcgattaaataaggaggaataacatatgccaacttataaagtgacactaat

SEQ ID NO: 113 - DNA, 48 bp, Artificial Sequence
  Description of Artificial Sequence Synthetic primer 2
gccttcttgacgagttcttctaagatgagtttttgttcgggcccaagc

SEQ ID NO: 114 - DNA, 5903 bp, Artificial Sequence
  Description of Artificial Sequence Synthetic Plasmid OP-80 polynucleotide
cactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcgtcggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagcgccgctgagaaaaagcgaagcggcactgctctttaacaatttatcagacaatctgtgtgggcactcgaccggaattatcgattaactttattattaaaaattaaagaggtatatattaatgtatcgattaaataaggaggaataaaccatggatccgagctcgagatctgcagctggtaccatatgggaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgacgcctgatgcggtattttctccttacgcatctgtgcggtatttcacaccgcatatggtgcactctcagtacaatctgctctgatgccgcatagttaagccagccccgacacccgccaacacccgctgacgagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaat

SEQ ID NO: 115 - DNA, 28 bp, Artificial Sequence
  Description of Artificial Sequence Synthetic primer LF302
atatgacgtcggcatccgcttacagaca

SEQ ID NO: 116 - DNA, 32 bp, Artificial Sequence
  Description of Artificial Sequence Synthetic primer LF303
aattcttaagtcaggagagcgttcaccgacaa

SEQ ID NO: 117 - DNA, 1269 bp, Jeotgalicoccus sp.
  ATCC8456 orf880
atggcaacacttaagagggataagggcttagataatactttgaaagtattaaagcaaggttatctttacacaacaaatcagagaaatcgtctaaacacatcagttttccaaactaaagcactcggtggtaaaccattcgtagttgtgactggtaaggaaggcgctgaaatgttctacaacaatgatgttgttcaacgtgaaggcatgttaccaaaacgtatcgttaatacgctttttggtaaaggtgcaatccatacggtagatggtaaaaaacacgtagacagaaaagcattgttcatgagcttgatgactgaaggtaacttgaattatgtacgagaattaacgcgtacattatggcatgcgaacacacaacgtatggaaagtatggatgaggtaaatatttaccgtgaatctatcgtactacttacaaaagtaggaacacgttgggcaggcgttcaagcaccacctgaagatatcgaaagaatcgcaacagacatggacatcatgatcgattcatttagagcacttggtggtgcctttaaaggttacaaggcatcaaaagaagcacgtcgtcgtgttgaagattggttagaagaacaaattattgagactcgtaaagggaatattcatccaccagaaggtacagcactttacgaatttgcacattgggaagactacttaggtaacccaatggactcaagaacttgtgcgattgacttaatgaacacattccgcccattaatcgcaatcaacagattcgtttcattcggtttacacgcgatgaacgaaaacccaatcacacgtgaaaaaattaaatcagaacctgactatgcatataaattcgctcaagaagttcgtcgttactatccattcgttccattccttccaggtaaagcgaaagtagacatcgacttccaaggcgttacaattcctgcaggtgtaggtcttgcattagatgtttatggtacaacgcatgatgaatcactttgggacgatccaaatgaattccgcccagaaagattcgaaacttgggacggatcaccatttgaccttattccacaaggtggtggagattactggacaaatcaccgttgtgcaggtgaatggatcacagtaatcatcatggaagaaacaatgaaatactttgcagaaaaaataacttatgatgttccagaacaagatttagaagtggacttaaacagtatcccaggatacgttaagagtggctttgtaatcaaaaatgttcgcgaagttgtagacagaacataa

SEQ ID NO: 118 - protein, 422 aa, Jeotgalicoccus sp.
  ATCC8456 orf880
MATLKRDKGLDNTLKVLKQGYLYTTNQRNRLNTSVFQTKALGGKPFVVVTGKEGAEMFYNNDVVQREGMLPKRIVNTLFGKGAIHTVDGKKHVDRKALFMSLMTEGNLNYVRELTRTLWHANTQRMESMDEVNIYRESIVLLTKVGTRWAGVQAPPEDIERIATDMDIMIDSFRALGGAFKGYKASKEARRRVEDWLEEQIIETRKGNIHPPEGTALYEFAHWEDYLGNPMDSRTCAIDLMNTFRPLIAINRFVSFGLHAMNENPITREKIKSEPDYAYKFAQEVRRYYPFVPFLPGKAKVDIDFQGVTIPAGVGLALDVYGTTHDESLWDDPNEFRPERFETWDGSPFDLIPQGGGDYWTNHRCAGEWITVIIMEETMKYFAEKITYDVPEQDLEVDLNSIPGYVKSGFVIKNVREVVDRT

SEQ ID NO: 119 - DNA, 1507 bp, Jeotgalicoccus sp.
  ATCC8456 16s rRNA (partial sequence)
ggttaccttgttacgacttcaccccaattatcaatcccacctttgacggctacctccattaaggttagtccaccggcttcaggtgttaycgactttcgtggtgtgacgggcggtgtgtacaagacccgggaacgtattcaccgtagcatgctgatctacgattactagcgattccagcttcatggagtcgagttgcagactccaatccgaactgagaacagttttatgggattcgcttggcctcgcggcttcgctgccctttgtaacctgcccattgtagcacgtgtgtagcccaaatcataaggggcatgatgatttgacgtcatccccaccttcctccggtttgtcaccggcagtcaatctagagtgcccaactgaatgatggcaactaaatttaagggttgcgctcgttgcgggacttaacccaacatctcacgacacgagctgacgacaaccatgcaccacctgtctctctgcccaaaagggaaaccatatctctrtggcgatcagaggatgtcaagatttggtaaggttcttcgcgttgcttcgaattaaaccacatgctccaccgcttgtgcgggtccccgtcaattcctttgagtttcaaccttgcggtcgtactccccaggcggagtgcttaatgcgttagctgcagcactgaggggcggaaaccccccaacacttagcactcatcgtttacggcgtggactaccagggtatctaatcctgtttgatccccacgctttcgcacctcagcgtcagttacagaccagagagccgccttcgcccactggtgttcctccatatctctgcgcatttcaccgctacacatggaattccactctcctcttctgcactcaagtaaaacagtttccaatgaccctccccggttgagccgggggctttcacatcagacttattctaccgcctacgcgcgctttacgcccaataattccggataacgcttgccacctacgtattaccgcggctgctggcacgtagttagccgtggctttctggttaagtaccgtcatctctaggccagttactacctaaagtgttcttccttaacaacagagttttacgagccgaaacccttcttcactcacgcggcgttgctccgtcagacttgcgtycattgcggaagattccctactgctgcctcccgtaggagtctgggccgtgtctcagtcccagtgtggccgatcaccctctcaggtcggctatgcatcgttgccttggtgagccactacctcaccaactagctaatgcaccgcaggcccatcctttagtgacagataaatccgcctttcattaagattacttgtgtaatccaacttatccggtattagctaccgtttccggtagttatcccagtctaaagggtaggttgcccacgtgttactcacccgtccgccgctcgattgtaaggagcaagctccttacgctcgcgctcgacttgcatgtattaggcacgccgccagcgttcatcctgagccaggatcaa

SEQ ID NO: 120 - DNA, 1209 bp, Artificial Sequence
  Description of Artificial Sequence Synthetic ATCC8456 orf880, codon-optimized DNA polynucleotide
atggctactctgaaacgtgacaaaggtctggataacactctgaaagttctgaaacaaggttacctgtacactaccaaccagcgcaaccgtctgaacaccagcgtctttcaaaccaaagccctgggtggcaaaccgttcgtggttgtgaccggcaaagaaggcgcagagatgttctataacaacgatgtggtgcagcgtgagggcatgctgccgaaacgtattgtaaacaccctgttcggcaagggtgcgatccataccgtggatggcaagaaacacgtagaccgtaaagcactgttcatgtctctgatgactgagggcaacctgaactatgtacgtgaactgacccgcaccctgtggcatgcgaacacgcagcgtatggaatctatggatgaggtgaacatctaccgtgaaagcatcgttctgctgacgaaggtgggcacccgctgggcaggtgttcaggcaccgccggaggacattgagcgcatcgctaccgatatggatattatgatcgatagcttccgtgctctgggtggcgcatttatcatcgaaacccgtaaaggtaacatccacccaccggaaggtacggctctgtacgaatttgcacactgggaggattatctgggtaatccaatggactctcgtacctgcgcgatcgatctgatgaacacgtttcgcccgctgatcgctatcaaccgctttgtttctttcggtctgcacgcgatgaacgaaaacccgatcactcgtgagaagattaagtccgagccggattacgcatacaaattcgcacaggaggtccgtcgctactacccgttcgttcctttcctgccgggtaaagcaaaggtagacatcgacttccagggtgtaaccatcccagccggtgtgggcctggcactggatgtttacggtaccacccatgatgaaagcctgtgggatgatccgaacgaatttcgcccggagcgtttcgagacttgggatggttctccatttgacctgattccgcaaggtggtggtgattactggaccaatcaccgctgtgccggcgagtggatcaccgtcattatcatggaagaaacgatgaaatacttcgccgagaaaatcacttatgacgttccagaacaggacctggaagtagatctgaactccatcccgggctatgtcaaaagcggctttgttatcaaaaacgtccgtgaagtagtcgatcgcacctaa

SEQ ID NO: 121 - protein, 422 aa, Jeotgalicoccus sp.; identical to SEQ ID NO: 118
  ATCC8456 orf880, Protein

SEQ ID NO: 122 - DNA, 1308 bp, Corynebacterium efficiens
  orf_CE2459 (NP_739069) DNA
tcagcgttccacgcgcacccgcatgccggtttcggagcgggtgagcatctgtgtccaggggaaacgggtatcagccggatccgtggagagcaccacaccgggccggcataaagcctccaccatggctgtgagcgcggccatggcgatcttctcgcccgggcagcggtgaccggtgtacaccccggctccgccctggggcacgaagctggtgagcctctcatagtcctcctgggtgcccaggtcctcccgggacaggaaacgctccggttgaaacgcactcgggttctcccactcattggggtcggtgttggtgccgtagatgtcgatgatcacgcgttcaccctcatgcacggggcagccctggatttcggtgtcggtggtggcgatggccggcagcatgggcacaaacggatagacgcggcggacctcctgggcgaaggcgaaggccacgggctgtcctccctcgcggatcttctccacccactcggggtgctcgaccagggcgctgccggcgaaggaggcgaacagtgatactgccacggtgggacgggtgaggttctgtaattcgatgccggcgatggaggcgtcgacaagctccccgtccggaccgaccaaccgggacatggcctccagggcgctacccggcgccacgtgccgctccccggcgcgcgcctgcctgatgagcttcaaggcccacctgttcaaccgcgcccggttgatccagcccagggcgtgccctttgaggggatggccgaactgatagaccagctcggccatctgatgggcgcgccggctggcttccttctggctaagctcaatgcccgcccaacggtaggccgcacgcccgaaggccagcgccgcaccgtcatagaccgtcccgggttcgcgggcccagtcctgcaccacacggtccacctcacggcggacgagtgcatcgaactcggcgaccttgtcatcgtcataggcgacatcggcgagctgacgtttgcgcagacggtgctcctcgccgtccagcgaatgcaccgcaccctcaccgaacagggggatgcggatcaccgcgggcatggctccgtcacgtttcatccggtcattgtcatagaacagctccactccggctgaaccgcgcacgatggtgacgggtttgaacagcatgcgcgaccgcagcggggtgttggcatcgggtgagataccggccttgcggcgcagacgggagagaaaaaggtagccgtggcgcagcaggttgggggcctgttcgccgggggcaaaggggcaggaggatgtctgagtcatcggtgggacctcttccaa

SEQ ID NO: 123 - protein, 435 aa, Corynebacterium efficiens
  orf_CE2459 (NP_739069) Protein
MEEVPPMTQTSSCPFAPGEQAPNLLRHGYLFLSRLRRKAGISPDANTPLRSRMLFKPVTIVRGSAGVELFYDNDRMKRDGAMPAVIRIPLFGEGAVHSLDGEEHRLRKRQLADVAYDDDKVAEFDALVRREVDRVVQDWAREPGTVYDGAALAFGRAAYRWAGIELSQKEASRRAHQMAELVYQFGHPLKGHALGWINRARLNRWALKLIRQARAGERHVAPGSALEAMSRLVGPDGELVDASIAGIELQNLTRPTVAVSLFASFAGSALVEHPEWVEKIREGGQPVAFAFAQEVRRVYPFVPMLPAIATTDTEIQGCPVHEGERVIIDIYGTNTDPNEWENPSAFQPERFLSREDLGTQEDYERLTSFVPQGGAGVYTGHRCPGEKIAMAALTAMVEALCRPGVVLSTDPADTRFPWTQMLTRSETGMRVRVER

SEQ ID NO: 124 - DNA, 1287 bp, Kokuria rhizophila
  orf_KRH21570 (YP_001856010) DNA
atgacttcaccgttcggtcagacccgttccgagcagggcccgtccctactccgctccggctacctctttgcctcccgcgcacgacgccgcgcgggcctctcctccgactcggggtgccccgtccgcatgcctctgctgggcaagcagaccgtcctggtccgcggcgaggagggcgtcaagctcttctacgacacctcccgcgtgcggcgcgacggcgccatgcccggagtcgtgcaggggccgctcttcggtgcgggcgccgtgcacgggctggacggcgaggcccaccgggtgcgcaagaaccaactcgcggacatggcctacgaggacgagcgcgtggcggcctacaagcccttcgtggcggaggagctcgagaacctcgtcgcgcggtggaaggacggcgataacgtctacgacagcaccgccatcgccttcggccgcgcgtccttccggtgggccggtctgcagtggggcgtgccggagatggaccgctgggcccgccgcatgagccgcctgctggacaccttcgggcgccccgccacgcacctggtgtcccggctggaccggatcgccctggaccgccgcttcgccgcgctcatcaaggacgtgcgcgcgggcaaggtcaacgcacccgaggactccgtgctcgcgcacatggccgccctggtggacgagcacggcgagctggtggacgcgaagaccgcgggcatcgagctgcagaacctcacccgcccgaacgtggccgtggcccgcttcgccgcgttcgcggccaccgccctggtggagcaccctgagtgggtcgagcgcatccgcgccgcctccgagcagcgcggcggcaccctgctggacgtccccgaggccgtggccttcgcgcaggaggtccgccgcgtctacccgttcgtgcccatgctccccgcggaggtcacacaggacaccgagatccagggctgccccgtgcacaagggggagcgcgtggtcctggacatcctgggcaccaacacggatccgacgtcctgggaccgcgcggccacgttcgaccccgagcgcttcctgggggtcgaggacgccgaggcgatcaccacgttcatcccccagggcggcgctgaggtccgcacgggccaccgctgccccggcgagaagatcgcggtcacgtccctctccgccgccgtggtggcgctgtgccggccggaggtccagctgccgggcgaccaggacgacctcacgttctcgtggacccacatgctgacccgcccggtcaccggggtgcgggtccgcaccacccgctga

SEQ ID NO: 125 - DNA, 1287 bp, Artificial Sequence
  Description of Artificial Sequence Synthetic orf_KRH21570 (YP_001856010) codon-optimized DNA polynucleotide
atgacgagcccgttcggccagacccgtagcgaacagggcccgagcctgctgcgttcgggttacttgtttgcaagccgcgctcgccgccgtgctggcctgagcagcgatagcggttgtccagttcgcatgccgctgctgggtaagcaaacggttctggtgcgcggcgaggaaggcgtcaaactgttctatgataccagccgtgttcgtcgtgacggcgcgatgccaggcgtcgtgcagggccctctgttcggtgcaggtgcggttcacggtctggacggcgaagcgcaccgcgttcgcaagaaccaactggcggatatggcttatgaagatgaacgtgtggctgcgtacaagccgttcgttgcggaagagttggagaatctggttgcacgttggaaagatggtgacaacgtctacgacagcacggcaattgcatttggccgcgcatcttttcgttgggccggtctgcagtggggtgtgccggagatggatcgctgggcacgccgcatgagccgtctgttggataccttcggtcgtccggccacgcacctggtgagccgtttggaccgtattgctttggatcgccgctttgcagcattgattaaggacgtgcgtgccggtaaagtgaacgctccggaagacagcgtcctggcccacatggcagctctggtcgacgagcatggtgaattggtggatgctaagacggcgggtatcgaactgcagaatttgacccgtccgaatgtggcggtggctcgttttgcggcctttgcggcgacggcactggttgagcatccggagtgggtcgaacgtattcgtgcagcctccgaacagcgtggcggtaccttgctggacgttccggaggccgtggcgttcgcgcaggaagttcgtcgcgtctacccgtttgtcccgatgctgccagctgaagttacccaggacaccgagatccagggttgtccggttcacaagggtgagcgcgtggttctggatattttgggtaccaataccgatccgaccagctgggaccgtgcggcgacctttgacccggagcgctttctgggtgttgaggacgcggaagccatcaccacctttatcccgcagggcggtgcagaggtgcgtacgggccatcgctgtccgggtgagaagatcgccgtcaccagcctgagcgctgctgtcgttgcgctgtgtcgcccggaggtgcaactgccgggtgatcaggatgatctgacttttagctggacccacatgctgacgcgccctgtcacgggtgttcgcgtccgcaccacgcgctaa

SEQ ID NO: 126 - protein, 428 aa, Kokuria rhizophila
  orf_KRH21570 (YP_001856010) Protein
MTSPFGQTRSEQGPSLLRSGYLFASRARRRAGLSSDSGCPVRMPLLGKQTVLVRGEEGVKLFYDTSRVRRDGAMPGVVQGPLFGAGAVHGLDGEAHRVRKNQLADMAYEDERVAAYKPFVAEELENLVARWKDGDNVYDSTAIAFGRASFRWAGLQWGVPEMDRWARRMSRLLDTFGRPATHLVSRLDRIALDRRFAALIKDVRAGKVNAPEDSVLAHMAALVDEHGELVDAKTAGIELQNLTRPNVAVARFAAFAATALVEHPEWVERIRAASEQRGGTLLDVPEAVAFAQEVRRVYPFVPMLPAEVTQDTEIQGCPVHKGERVVLDILGTNTDPTSWDRAATFDPERFLGVEDAEAITTFIPQGGAEVRTGHRCPGEKIAVTSLSAAVVALCRPEVQLPGDQDDLTFSWTHMLTRPVTGVRVRTTR

SEQ ID NO: 127 - DNA, 1275 bp, Artificial Sequence
  Description of Artificial Sequence Synthetic orf_Mpop1292 (YP_001923998) codon-optimized DNA polynucleotide
atgccggctgccattgccacccaccgtttccgcaaagcacgcaccctgccgcgtgagccagctccagatagcacgctggcgctgctgcgcgagggttacggtttcatccgtaaccgttgtcgccgtcacgacagcgacctgttcgcagcccgtttgttgctgagcccggtcatctgcatgtctggcgcggaggcggcacgccacttttacgacggtcaccgctttactcgtcgtcatgcactgccgccgaccagcttcgctctgatccaagaccacggtagcgttatggttctggatggcgccgcacacctggcacgtaaggctatgttcctgagcctggtcggtgaagaggccctgcaacgtttggcgggcctggcggaacgtcactggcgcgaagcggtgtccggctgggcacgtaaagatacggtggttctgctggacgaggcacatcgcgtgctgaccgcagcggtctgcgaatgggtgggtttgccgctgggcccgaccgaagtggatgctcgcgcgcgtgagttcgcagcgatgattgatggcacgggtgcagtgggtccgcgcaactggcgcggtcacttgtatcgtgcacgcacggagcgttgggttcgcaaggttatcgacgagatccgctccggtcgtcgcgatgtccctccgggtgccgcacgcactatcgcggagcatcaagatgccgacggtcaacgtctggatcgtacggtcgcgggtgttgaactgatcaacgttctgcgcccgaccgttgcgaacgcacgttacattgtctttgcagctatggcgctgcacgatcaccctcatcagcgcgctgcgttggcggacggtggtgaagctgcggaacgctttaccgatgaagtgcgtcgcttctacccattcatcccgtttatcggcggtcgtgtccgtgcgccgttccattttggtggccacgactttcgcgaaggtgaatgggtgctgatggatctgtatggtaccaatcgtgacccacgtctgtggcacgagccagaacgtttcgacccggatcgttttgctcgtgaaaccatcgatccgtttaatatggtttctcatggtgcgggtagcgctcgcgatggtcaccgctgtccgggtgagggtattacccgcatcctgttgcgtacgctgagccgccaactggccgcgacgcgctacacggttccgccacaagacctgaccctggacctggcgcatgtgcctgcccgtccgcgcagcggttttgttatgcgtgctgtgcacgcgccgtaa

SEQ ID NO: 128 - protein, 424 aa, Methylobacterium populi
  orf_Mpop1292 (YP_001923998) Protein
MPAAIATHRFRKARTLPREPAPDSTLALLREGYGFIRNRCRRHDSDLFAARLLLSPVICMSGAEAARHFYDGHRFTRRHALPPTSFALIQDHGSVMVLDGAAHLARKAMFLSLVGEEALQRLAGLAERHWREAVSGWARKDTVVLLDEAHRVLTAAVCEWVGLPLGPTEVDARAREFAAMIDGTGAVGPRNWRGHLYRARTERWVRKVIDEIRSGRRDVPPGAARTIAEHQDADGQRLDRTVAGVELINVLRPTVANARYIVFAAMALHDHPHQRAALADGGEAAERFTDEVRRFYPFIPFIGGRVRAPFHFGGHDFREGEWVLMDLYGTNRDPRLWHEPERFDPDRFARETIDPFNMVSHGAGSARDGHRCPGEGITRILLRTLSRQLAATRYTVPPQDLTLDLAHVPARPRSGFVMRAVHAP

SEQ ID NO: 129 - DNA, 1254 bp, Bacillus subtilis
  CYP152A1 (NP_388092) DNA
atgaatgagcagattccacatgacaaaagtctcgataacagtctgacactgctgaaggaagggtatttatttattaaaaacagaacagagcgctacaattcagatctgtttcaggcccgtttgttgggaaaaaactttatttgcatgactggcgctgaggcggcgaaggtgttttatgatacggatcgattccagcggcagaacgctttgcctaagcgggtgcagaaatcgctgtttggtgttaatgcgattcagggaatggatggcagcgcgcatatccatcggaagatgctttttctgtcattgatgacaccgccgcatcaaaaacgtttggctgagttgatgacagaggagtggaaagcagcagtcacaagatgggagaaggcagatgaggttgtgttatttgaagaagcaaaagaaatcctgtgccgggtagcgtgctattgggcaggtgttccgttgaaggaaacggaagtcaaagagagagcggatgacttcattgacatggtcgacgcgttcggtgctgtgggaccgcggcattggaaaggaagaagagcaaggccgcgtgcggaagagtggattgaagtcatgattgaagatgctcgtgccggcttgctgaaaacgacttccggaacagcgctgcatgaaatggcttttcacacacaagaagatggaagccagctggattcccgcatggcagccattgagctgattaatgtactgcggcctattgtcgccatttcttactttctggtgttttcagctttggcgcttcatgagcatccgaagtataaggaatggctgcggtctggaaacagccgggaaagagaaatgtttgtgcaggaggtccgcagatattatccgttcggcccgtttttaggggcgcttgtcaaaaaagattttgtatggaataactgtgagtttaagaagggcacatcggtgctgcttgatttatatggaacgaaccacgaccctcgtctatgggatcatcccgatgaattccggccggaacgatttgcggagcgggaagaaaatctgtttgatatgattcctcaaggcggggggcacgccgagaaaggccaccgctgtccaggggaaggcattacaattgaagtcatgaaagcgagcctggatttcctcgtccatcagattgaatacgatgttccggaacaatcactgcattacagtctcgccagaatgccatcattgcctgaaagcggcttcgtaatgagcggaatcagacgaaaaagttaa

SEQ ID NO: 130 - protein, 417 aa, Bacillus subtilis
  CYP152A1 (NP_388092) Protein
MNEQIPHDKSLDNSLTLLKEGYLFIKNRTERYNSDLFQARLLGKNFICMTGAEAAKVFYDTDRFQRQNALPKRVQKSLFGVNAIQGMDGSAHIHRKMLFLSLMTPPHQKRLAELMTEEWKAAVTRWEKADEVVLFEEAKEILCRVACYWAGVPLKETEVKERADDFIDMVDAFGAVGPRHWKGRRARPRAEEWIEVMIEDARAGLLKTTSGTALHEMAFHTQEDGSQLDSRMAAIELINVLRPIVAISYFLVFSALALHEHPKYKEWLRSGNSREREMFVQEVRRYYPFGPFLGALVKKDFVWNNCEFKKGTSVLLDLYGTNHDPRLWDHPDEFRPERFAEREENLFDMIPQGGGHAEKGHRCPGEGITIEVMKASLDFLVHQIEYDVPEQSLHYSLARMPSLPESGFVMSGIRRKS

(19) United States
(12) Patent Application Publication
(10) Pub. No.: US 2012/0157717 A1
(43) Pub. Date: Jun. 21, 2012

(54) METHODS AND COMPOSITIONS FOR PRODUCING LINEAR ALKYL BENZENES

(75) Inventors: Mathew A. RUDE, South San Francisco, CA (US); Andreas W. SCHIRMER, South San Francisco, CA (US)

(73) Assignee: LS9, INC., South San Francisco, CA (US)

(21) Appl. No.: 13/233,489

(22) Filed: Sep. 15, 2011

Related U.S. Application Data
(60) Provisional application No. 61/383,086, filed on Sep. 15, 2010.

(51) Int. Cl. C07C 317/02; C12P 5/00; C12N 1/13; C12N 1/21; C12N 1/19; C12N 1/15; C12N 5/10; C12P 11/00

(52) U.S. Cl. 568/28; 435/419; 435/166; 435/130; 435/325; 435/366; 435/252.3; 435/254.2; 435/254.11; 435/252.33; 435/254.22; 435/254.21; 435/257.2

(57) ABSTRACT

Compositions and methods for producing hydrocarbons using recombinant cells are described herein. Also described herein are recombinant cells, recombinant cell cultures and methods for producing linear alkyl benzenes (LABs) using hydrocarbons produced by such recombinant cell cultures.

### CROSS-REFERENCE TO RELATED APPLICATIONS

This application claims priority to and benefit of U.S. Provisional Patent Application No. 61/383,086, filed Sep. 15, 2010, the entire content of which is hereby incorporated by reference.

### BACKGROUND OF THE INVENTION

Petroleum is a limited, natural resource found in the Earth in liquid, gaseous, or solid forms. In its natural form, crude petroleum extracted from the Earth has few commercial uses. It is a mixture of hydrocarbons (e.g., paraffins (or alkanes), olefins (or alkenes), alkynes, napthenes (or cylcoalkanes), aliphatic compounds, aromatic compounds, etc.) of varying length and complexity. In addition, crude petroleum contains other organic compounds (e.g., organic compounds containing nitrogen, oxygen, sulfur, etc.) and impurities (e.g., sulfur, salt, acid, metals, etc.). Hence, crude petroleum must be refined and purified before it can be used commercially.

Crude petroleum is a primary source of raw materials for producing petrochemicals. These petrochemicals can then be used to make specialty chemicals, such as plastics, resins, fibers, elastomers, pharmaceuticals, lubricants, or gels. Particular specialty chemicals which can be produced from petrochemical raw materials are: fatty acids, hydrocarbons (e.g., long chain, branched chain, saturated, unsaturated, etc.), fatty alcohols, esters, fatty aldehydes, ketones, lubricants, and the like.

Linear alkylbenzene (“LAB”) is a family of organic compounds with the formula C₆H₅CₙH₂ₙ₊₁. They are mainly produced as intermediate in the production of surfactants, for use in detergent. The alkylation of aromatic hydrocarbons such as benzene is practiced commercially using solid catalysts in large scale industrial units. The alkylation of benzene with olefins having from 8 to 28 carbons produces alkylbenzenes that have various commercial uses. One use is to sulfonate the alkylbenzenes to produced sulfonated alkylbenzenes for use as detergents. Alkylbenzenes are produced as a commodity product for detergent production, often in amounts from 50,000 to 200,000 metric tons per year per plant.

Due to the inherent challenges posed by petroleum as a source of various chemicals and fuels, there is a need for a renewable petroleum source which does not need to be explored, extracted, transported over long distances, or substantially refined like petroleum. There is also a need for a renewable petroleum source that can be produced economically without creating the type of environmental damage produced by the petroleum industry and the burning of petroleum based fuels. For similar reasons, there is also a need for a renewable source of chemicals that are typically derived from petroleum.

### SUMMARY OF THE INVENTION

The invention is based, at least in part, on the identification of cyanobacterial genes that encode hydrocarbon biosynthetic polypeptides. Accordingly, in one aspect, the invention features a method of producing a hydrocarbon, the method comprising producing in a host cell a polypeptide comprising the amino acid sequence of SEQ ID NO:2, 4, 6, 8, 10, 12, 14, 16, 18, 20, 22, 24, 26, 28, 30, 32, 34, or 36, or a variant thereof, and isolating the hydrocarbon from the host cell.

In some embodiments, the polypeptide comprises an amino acid sequence having at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO:2, 4, 6, 8, 10, 12, 14, 16, 18, 20, 22, 24, 26, 28, 30, 32, 34, or 36.

In some embodiments, the polypeptide comprises the amino acid sequence of SEQ ID NO:2, 4, 6, 8, 10, 12, 14, 16, 18, 20, 22, 24, 26, 28, 30, 32, 34, or 36 with one or more amino acid substitutions, additions, insertions, or deletions. In some embodiments, the polypeptide has decarbonylase activity. In yet other embodiments, the polypeptide comprises the amino acid sequence of SEQ ID NO:2, 4, 6, 8, 10, 12, 14, 16, 18, 20, 22, 24, 26, 28, 30, 32, 34, or 36, with one or more conservative amino acid substitutions. For example, the polypeptide comprises one or more of the following conservative amino acid substitutions: replacement of an aliphatic amino acid, such as alanine, valine, leucine, and isoleucine, with another aliphatic amino acid; replacement of a serine with a threonine; replacement of a threonine with a serine; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue. In some embodiments, the polypeptide has about 1, 2, 3, 4, 5, 6, 7, 8, 9, 10, 15, 20, 30, 40, 50, 60, 70, 80, 90, 100, or more amino acid substitutions, additions, insertions, or deletions. In some embodiments, the polypeptide has decarbonylase activity.

In other embodiments, the polypeptide comprises the amino acid sequence of: (i) SEQ ID NO:37 or SEQ ID NO:38 or SEQ ID NO:39; or (ii) SEQ ID NO:40 and any one of (a) SEQ ID NO:37, (b) SEQ ID NO:38, and (c) SEQ ID NO:39; or (iii) SEQ ID NO:41 or SEQ ID NO:42 or SEQ ID NO:43 or SEQ ID NO:44. In certain embodiments, the polypeptide has decarbonylase activity.

In another aspect, the invention features a method of producing a hydrocarbon, the method comprising expressing in a host cell a polynucleotide comprising a nucleotide sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO:1, 3, 5, 7, 9, 11, 13, 15, 17, 19, 21, 23, 25, 27, 29, 31, 33, or 35. In some embodiments, the nucleotide sequence is SEQ ID NO:1, 3, 5, 7, 9, 11, 13, 15, 17, 19, 21, 23, 25, 27, 29, 31, 33, or 35. In some embodiments, the method further comprises isolating the hydrocarbon from the host cell.

In other embodiments, the nucleotide sequence hybridizes to a complement of SEQ ID NO:1, 3, 5, 7, 9, 11, 13, 15, 17, 19, 21, 23, 25, 27, 29, 31, 33, or 35, or to a fragment thereof, for example, under low stringency, medium stringency, high stringency, or very high stringency conditions.

In other embodiments, the nucleotide sequence encodes a polypeptide comprising: (i) the amino acid sequence of SEQ ID NO:2, 4, 6, 8, 10, 12, 14, 16, 18, 20, 22, 24, 26, 28, 30, 32, 34, or 36; or (ii) the amino acid sequence of SEQ ID NO:2, 4, 6, 8, 10, 12, 14, 16, 18, 20, 22, 24, 26, 28, 30, 32, 34, or 36 with one or more amino acid substitutions, additions, insertions, or deletions. In some embodiments, the polypeptide comprises the amino acid sequence of SEQ ID NO:2, 4, 6, 8, 10, 12, 14, 16, 18, 20, 22, 24, 26, 28, 30, 32, 34, or 36 with one or more conservative amino acid substitutions. In some embodiments, the polypeptide has decarbonylase activity.

In other embodiments, the nucleotide sequence encodes a polypeptide having the same biological activity as a polypeptide comprising the amino acid sequence of SEQ ID NO:2, 4, 6, 8, 10, 12, 14, 16, 18, 20, 22, 24, 26, 28, 30, 32, 34, or 36. In some embodiments, the nucleotide sequence is SEQ ID NO:1, 3, 5, 7, 9, 11, 13, 15, 17, 19, 21, 23, 25, 27, 29, 31, 33, or 35 or a fragment thereof. In other embodiments, the nucleotide sequence hybridizes to a complement of SEQ ID NO:1, 3, 5, 7, 9, 11, 13, 15, 17, 19, 21, 23, 25, 27, 29, 31, 33, or 35 or to a fragment thereof, for example, under low stringency, medium stringency, high stringency, or very high stringency conditions. In some embodiments, the biological activity is decarbonylase activity.

In some embodiments, the method comprises transforming a host cell with a recombinant vector comprising a nucleotide sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO:1, 3, 5, 7, 9, 11, 13, 15, 17, 19, 21, 23, 25, 27, 29, 31, 33, or 35. In some embodiments, the recombinant vector further comprises a promoter operably linked to the nucleotide sequence. In some embodiments, the promoter is a developmentally-regulated, an organelle-specific, a tissue-specific, an inducible, a constitutive, or a cell-specific promoter. In particular embodiments, the recombinant vector comprises at least one sequence selected from the group consisting of (a) a regulatory sequence operatively coupled to the nucleotide sequence; (b) a selection marker operatively coupled to the nucleotide sequence; (c) a marker sequence operatively coupled to the nucleotide sequence; (d) a purification moiety operatively coupled to the nucleotide sequence; (e) a secretion sequence operatively coupled to the nucleotide sequence; and (f) a targeting sequence operatively coupled to the nucleotide sequence. In certain embodiments, the nucleotide sequence is stably incorporated into the genomic DNA of the host cell, and the expression of the nucleotide sequence is under the control of a regulated promoter region.

In certain embodiments, a recombinant host cell culture that produces a composition comprising one or more fatty acid derivatives is provided.

In some embodiments, the hydrocarbon is secreted from by the host cell.

In certain embodiments, the host cell overexpresses a substrate described herein. In some embodiments, the method further includes transforming the host cell with a nucleic acid that encodes an enzyme described herein, and the host cell overexpresses a substrate described herein. In other embodiments, the method further includes culturing the host cell in the presence of at least one substrate described herein. In some embodiments, the substrate is a fatty acid derivative, an acyl-ACP, a fatty acid, an acyl-CoA, a fatty aldehyde, a fatty alcohol, or a fatty ester.

In some embodiments, the fatty acid derivative substrate is an unsaturated fatty acid derivative substrate, a monounsaturated fatty acid derivative substrate, or a saturated fatty acid derivative substrate. In other embodiments, the fatty acid derivative substrate is a straight chain fatty acid derivative substrate, a branched chain fatty acid derivative substrate, or a fatty acid derivative substrate that includes a cyclic moiety.

In certain embodiments of the aspects described herein, the hydrocarbon produced is an alkane. In some embodiments, the alkane is a C₃-C₂₅ alkane. For example, the alkane is a C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, or C₂₅ alkane. In some embodiments, the alkane is tridecane, methyltridecane, nonadecane, methylnonadecane, heptadecane, methylheptadecane, pentadecane, or methylpentadecane.

In some embodiments, the alkane is a straight chain alkane, a branched chain alkane, or a cyclic alkane.

In certain embodiments, the method further comprises culturing the host cell in the presence of a saturated fatty acid derivative, and the hydrocarbon produced is an alkane. In certain embodiments, the saturated fatty acid derivative is a C₆-C₂₆ fatty acid derivative substrate. For example, the fatty acid derivative substrate is a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₆ fatty acid derivative substrate. In particular embodiments, the fatty acid derivative substrate is 2-methylicosanal, icosanal, octadecanal, tetradecanal, 2-methyloctadecanal, stearaldehyde, or palmitaldehyde.

In some embodiments, the method further includes isolating the alkane from the host cell or from the culture medium. In other embodiments, the method further includes cracking or refining the alkane.

In certain embodiments of the aspects described herein, the hydrocarbon produced is an alkene. In some embodiments, the alkene is a C₃-C₂₅ alkene. For example, the alkene is a C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, or C₂₅ alkene. In some embodiments, the alkene is pentadecene, heptadecene, methylpentadecene, or methylheptadecene.

In some embodiments, the alkene is a straight chain alkene, a branched chain alkene, or a cyclic alkene.

In certain embodiments, the method further comprises culturing the host cell in the presence of an unsaturated fatty acid derivative, and the hydrocarbon produced is an alkene. In certain embodiments, the unsaturated fatty acid derivative is a C₆-C₂₆ fatty acid derivative substrate. For example, the fatty acid derivative substrate is a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₆ unsaturated fatty acid derivative substrate. In particular embodiments, the fatty acid derivative substrate is octadecenal, hexadecenal, methylhexadecenal, or methyloctadecenal.

In another aspect, the invention features a genetically engineered microorganism comprising an exogenous control sequence stably incorporated into the genomic DNA of the microorganism. In one embodiment, the control sequence is integrated upstream of a polynucleotide comprising a nucleotide sequence having at least about 70% sequence identity to SEQ ID NO:1, 3, 5, 7, 9, 11, 13, 15, 17, 19, 21, 23, 25, 27, 29, 31, 33, or 35. In some embodiments, the nucleotide sequence has at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO:1, 3, 5, 7, 9, 11, 13, 15, 17, 19, 21, 23, 25, 27, 29, 31, 33, or 35. In some embodiments, the nucleotide sequence is SEQ ID NO:1, 3, 5, 7, 9, 11, 13, 15, 17, 19, 21, 23, 25, 27, 29, 31, 33, or 35.

In some embodiments, the polynucleotide is endogenous to the microorganism. In some embodiments, the microorganism expresses an increased level of a hydrocarbon relative to a wild-type microorganism. In some embodiments, the microorganism is a cyanobacterium.

In another aspect, the invention features a method of making a hydrocarbon, the method comprising culturing a genetically engineered microorganism described herein under conditions suitable for gene expression, and isolating the hydrocarbon.

In another aspect, the invention features a method of making a hydrocarbon, comprising contacting a substrate with (i) a polypeptide having at least 70% identity to the amino acid sequence of SEQ ID NO:2, 4, 6, 8, 10, 12, 14, 16, 18, 20, 22, 24, 26, 28, 30, 32, 34, or 36, or a variant thereof; (ii) a polypeptide encoded by a nucleotide sequence having at least 70% identity to SEQ ID NO:1, 3, 5, 7, 9, 11, 13, 15, 17, 19, 21, 23, 25, 27, 29, 31, 33, or 35, or a variant thereof; or (iii) a polypeptide comprising the amino acid sequence of SEQ ID NO:37, 38, or 39. In some embodiments, the polypeptide has decarbonylase activity.

In some embodiments, the polypeptide has at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO:2, 4, 6, 8, 10, 12, 14, 16, 18, 20, 22, 24, 26, 28, 30, 32, 34, or 36. In some embodiments, the polypeptide has the amino acid sequence of SEQ ID NO:2, 4, 6, 8, 10, 12, 14, 16, 18, 20, 22, 24, 26, 28, 30, 32, 34, or 36.

In some embodiments, the polypeptide is encoded by a nucleotide sequence having at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to SEQ ID NO:1, 3, 5, 7, 9, 11, 13, 15, 17, 19, 21, 23, 25, 27, 29, 31, 33, or 35. In some embodiments, the polypeptide is encoded by a nucleotide sequence having SEQ ID NO:1, 3, 5, 7, 9, 11, 13, 15, 17, 19, 21, 23, 25, 27, 29, 31, 33, or 35.

In some embodiments, the biological substrate is a fatty acid derivative, an acyl-ACP, a fatty acid, an acyl-CoA, a fatty aldehyde, a fatty alcohol, or a fatty ester.

In some embodiments, the substrate is a saturated fatty acid derivative, and the hydrocarbon is an alkane, for example, a C₃-C₂₅ alkane. For example, the alkane is a C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, or C₂₅ alkane. In some embodiments, the alkane is tridecane, methyltridecane, nonadecane, methylnonadecane, heptadecane, methylheptadecane, pentadecane, or methylpentadecane.

In some embodiments, the alkane is a straight chain alkane, a branched chain alkane, or a cyclic alkane.

In some embodiments, the saturated fatty acid derivative is 2-methylicosanal, icosanal, octadecanal, tetradecanal, 2-methyloctadecanal, stearaldehyde, or palmitaldehyde.

In other embodiments, the biological substrate is an unsaturated fatty acid derivative, and the hydrocarbon is an alkene, for example, a C₃-C₂₅ alkene. For example, the alkene is a C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, or C₂₅ alkene. In some embodiments, the alkene is pentadecene, heptadecene, methylpentadecene, or methylheptadecene. In some embodiments, the alkene is a straight chain alkene, a branched chain alkene, or a cyclic alkene.

In some embodiments, the unsaturated fatty acid derivative is octadecenal, hexadecenal, methylhexadecenal, or methyloctadecenal.

In another aspect, the invention features a hydrocarbon produced by any of the methods or microorganisms described herein. In particular embodiments, the hydrocarbon is an alkane or an alkene having a δ¹³C of about −15.4 or greater. For example, the alkane or alkene has a δ¹³C of about −15.4 to about −10.9, for example, about −13.92 to about −13.84. In other embodiments, the alkane or alkene has an f_{M}¹⁴C of at least about 1.003. For example, the alkane or alkene has an f_{M}¹⁴C of at least about 1.01 or at least about 1.5. In some embodiments, the alkane or alkene has an f_{M}¹⁴C of about 1.111 to about 1.124.

In another aspect, the invention features a biofuel that includes a hydrocarbon produced by any of the methods or microorganisms described herein. In particular embodiments, the hydrocarbon is an alkane or alkene having a δ¹³C of about −15.4 or greater. For example, the alkane or alkene has a δ¹³C of about −15.4 to about −10.9, for example, about −13.92 to about −13.84. In other embodiments, the alkane or alkene has an f_{M}¹⁴C of at least about 1.003. For example, the alkane or alkene has an f_{M}¹⁴C of at least about 1.01 or at least about 1.5. In some embodiments, the alkane or alkene has an f_{M}¹⁴C of about 1.111 to about 1.124. In some embodiments, the biofuel is diesel, gasoline, or jet fuel.

In another aspect, the invention features an isolated nucleic acid consisting of no more than about 500 nucleotides of SEQ ID NO:1, 3, 5, 7, 9, 11, 13, 15, 17, 19, 21, 23, 25, 27, 29, 31, 33, or 35. In some embodiments, the nucleic acid consists of no more than about 300 nucleotides, no more than about 350 nucleotides, no more than about 400 nucleotides, no more than about 450 nucleotides, no more than about 550 nucleotides, no more than about 600 nucleotides, or no more than about 650 nucleotides, of SEQ ID NO:1, 3, 5, 7, 9, 11, 13, 15, 17, 19, 21, 23, 25, 27, 29, 31, 33, or 35. In some embodiments, the nucleic acid encodes a polypeptide having decarbonylase activity.

In another aspect, the invention features an isolated nucleic acid consisting of no more than about 99%, no more than about 98%, no more than about 97%, no more than about 96%, no more than about 95%, no more than about 94%, no more than about 93%, no more than about 92%, no more than about 91%, no more than about 90%, no more than about 85%, or no more than about 80% of the nucleotides of SEQ ID NO:1, 3, 5, 7, 9, 11, 13, 15, 17, 19, 21, 23, 25, 27, 29, 31, 33, or 35. In some embodiments, the nucleic acid encodes a polypeptide having decarbonylase activity.

In another aspect, the invention features an isolated polypeptide consisting of no more than about 200, no more than about 175, no more than about 150, or no more than about 100 of the amino acids of SEQ ID NO:2, 4, 6, 8, 10, 12, 14, 16, 18, 20, 22, 24, 26, 28, 30, 32, 34, or 36. In some embodiments, the polypeptide has decarbonylase activity.

In another aspect, the invention features an isolated polypeptide consisting of no more than about 99%, no more than about 98%, no more than about 97%, no more than about 96%, no more than about 95%, no more than about 94%, no more than about 93%, no more than about 92%, no more than about 91%, no more than about 90%, no more than about 85%, or no more than about 80% of the amino acids of SEQ ID NO:2, 4, 6, 8, 10, 12, 14, 16, 18, 20, 22, 24, 26, 28, 30, 32, 34, or 36. In some embodiments, the polypeptide has decarbonylase activity.

In another aspect, the invention features a method of producing a linear alkyl benzene, the method comprising producing a linear alkene described herein, e.g., using any method described herein; isolating the linear alkene from the host cell; and reacting the linear alkene with benzene in the presence of a catalyst under reaction conditions sufficient for alkylation of the benzene, thereby producing a linear alkyl benzene.

In some embodiments, the method further comprises sulfonating the linear alkyl benzene to produce a linear alkyl sulfonate.

In another aspect, the invention features a linear alkyl benzene produced using any of the methods described herein.

In another aspect, the invention features a linear alkyl sulfonate produced using any of the methods described herein.

In another aspect, the invention features a surfactant composition comprising a linear alkyl sulfonate described herein.

### DEFINITIONS

As used in this specification and the appended claims, the singular forms “a,” “an” and “the” include plural referents unless the context clearly dictates otherwise. Thus, for example, reference to “a recombinant microorganism” includes two or more such recombinant microorganisms, reference to “a fatty acid derivative” includes one or more fatty acid derivative, or mixtures of fatty acids derivatives, reference to “a polynucleotide sequence” includes one or more polynucleotide sequences, reference to “an enzyme” includes one or more enzymes, reference to “a control sequence” includes one or more control sequences, and the like.

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, the preferred materials and methods are described herein.

In describing and claiming the present invention, the following terminology will be used in accordance with the definitions set out below.

Throughout the specification, a reference may be made using an abbreviated gene name or polypeptide name, but it is understood that such an abbreviated gene or polypeptide name represents the genus of genes or polypeptides. Such gene names include all genes encoding the same polypeptide and homologous polypeptides having the same physiological function. Polypeptide names include all polypeptides that have the same activity (e.g., that catalyze the same fundamental chemical reaction).

The accession numbers referenced herein are derived from the NCBI database (National Center for Biotechnology Information) maintained by the National Institute of Health, U.S.A. Unless otherwise indicated, the accession numbers are as provided in the database as of April 2009.

EC numbers are established by the Nomenclature Committee of the International Union of Biochemistry and Molecular Biology (NC-IUBMB) (available at http://www.chem.qmul.ac.uk/iubmb/enzyme/). The EC numbers referenced herein are derived from the KEGG Ligand database, maintained by the Kyoto Encyclopedia of Genes and Genomics, sponsored in part by the University of Tokyo. Unless otherwise indicated, the EC numbers are as provided in the database as of March 2008.

As used in this specification and the appended claims, the singular forms “a,” “an” and “the” include plural referents unless the context clearly dictates otherwise. Thus, for example, reference to “a recombinant microorganism” includes two or more such recombinant microorganisms, reference to “a fatty acid derivative” includes one or more fatty acid derivative, or mixtures of fatty acids derivatives, reference to “a polynucleotide sequence” includes one or more polynucleotide sequences, reference to “an enzyme” includes one or more enzymes, reference to “a control sequence” includes one or more control sequences, and the like.

Unless defined otherwise, all technical and scientific terms used herein have the same meaning as commonly understood by one of ordinary skill in the art to which the invention pertains. Although other methods and materials similar, or equivalent, to those described herein can be used in the practice of the present invention, the preferred materials and methods are described herein.

As used herein, “fatty aldehyde” means an aldehyde having the formula RCHO characterized by a carbonyl group (C═O). In some embodiments, the fatty aldehyde is any aldehyde made from a fatty acid or fatty acid derivative. In certain embodiments, the R group is at least 5, at least 6, at least 7, at least 8, at least 9, at least 10, at least 11, at least 12, at least 13, at least 14, at least 15, at least 16, at least 17, at least 18, or at least 19, carbons in length. Alternatively, or in addition, the R group is 20 or less, 19 or less, 18 or less, 17 or less, 16 or less, 15 or less, 14 or less, 13 or less, 12 or less, 11 or less, 10 or less, 9 or less, 8 or less, 7 or less, or 6 or less carbons in length. Thus, the R group can have an R group bounded by any two of the above endpoints. For example, the R group can be 6-16 carbons in length, 10-14 carbons in length, or 12-18 carbons in length. In some embodiments, the fatty aldehyde is a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₆ fatty aldehyde. In certain embodiments, the fatty aldehyde is a C₆, C₈, C₁₀, C₁₂, C₁₃, C₁₄, C₁₅, C₁₆, C₁₇, or C₁₈ fatty aldehyde.

As used herein, an “aldehyde biosynthetic gene” or an “aldehyde biosynthetic polynucleotide” is a nucleic acid that encodes an aldehyde biosynthetic polypeptide. A suitable fatty acid substrate can be converted into a fatty aldehyde substrate by, for example, a fatty aldehyde biosynthetic polypeptide such as a carboxylic acid reductase, or an acyl-ACP reductase. For example, the fatty aldehyde biosynthetic polypeptide can be selected from those described herein, or variants thereof. Alternatively, the acyl-ACP reductase can be one selected from those described herein, or a variant thereof. Then, the fatty aldehyde substrate can be converted into a fatty alcohol by, for example, a gene encoding a fatty alcohol biosynthetic polypeptide of the present invention. In some example, a gene encoding a fatty alcohol biosynthetic polypeptide described herein can be expressed in a host cell that expresses an endogenous fatty alcohol biosynthetic polypeptide capable of converting a fatty aldehyde produced by the fatty aldehyde biosynthetic polypeptide into a corresponding fatty alcohol.

As used herein, an “aldehyde biosynthetic polypeptide” is a polypeptide that is a part of the biosynthetic pathway of an aldehyde. Such polypeptides can act on a biological substrate to yield an aldehyde. In some instances, the aldehyde biosynthetic polypeptide has reductase activity.

As used herein, “fatty alcohol” means an alcohol having the formula ROH. In some embodiments, the fatty alcohol is any alcohol made from a fatty acid or fatty acid derivative. In certain embodiments, the R group is at least 5, at least 6, at least 7, at least 8, at least 9, at least 10, at least 11, at least 12, at least 13, at least 14, at least 15, at least 16, at least 17, at least 18, or at least 19, carbons in length. Alternatively, or in addition, the R group is 20 or less, 19 or less, 18 or less, 17 or less, 16 or less, 15 or less, 14 or less, 13 or less, 12 or less, 11 or less, 10 or less, 9 or less, 8 or less, 7 or less, or 6 or less carbons in length. Thus, the R group can have an R group bounded by any two of the above endpoints. For example, the R group can be 6-16 carbons in length, 10-14 carbons in length, or 12-18 carbons in length. In some embodiments, the fatty alcohol is a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or a C₂₆ fatty alcohol. In certain embodiments, the fatty alcohol is a C₆, C₈, C₁₀, C₁₂, C₁₃, C₁₄, C₁₅, C₁₆, C₁₇, or C₁₈ fatty alcohol. A microorganism engineered to produce fatty aldehyde may convert some of the fatty aldehyde to a fatty alcohol. When a microorganism that produces fatty alcohols is engineered to express a polynucleotide encoding an ester synthase, wax esters are produced. In a preferred embodiment, fatty alcohols are made from a fatty acid biosynthetic pathway. As an example, Acyl-ACP can be converted to fatty acids via the action of a thioesterase (e.g., E. coli tesA), which are converted to fatty aldehydes and fatty alcohols via the action of a carboxylic acid reductase (e.g., E. coli carB, or Mycobacterium carA or fadD9). Conversion of fatty aldehydes to fatty alcohols can be further facilitated, for example, via the action of an alcohol dehydrogenase (e.g., E. coli YqhD or Acinetobacter alrAadp1).

As used herein, the term “fatty alcohol forming peptides” means a peptide capable of catalyzing the conversion of acyl-CoA to fatty alcohol, including fatty alcohol forming acyl-CoA reductase (FAR, EC 1.1.1.*), acyl-CoA reductase (EC 1.2.1.50), or alcohol dehydrogenase (EC 1.1.1.1). Additionally, one of ordinary skill in the art will appreciate that some fatty alcohol forming peptides will catalyze other reactions as well. For example, some acyl-CoA reductase peptides will accept other substrates in addition to fatty acids. Such non-specific peptides are, therefore, also included. Nucleic acid sequences encoding fatty alcohol forming peptides are known in the art, and such peptides are publicly available. Exemplary GenBank Accession Numbers are provided in FIG. 40 of W02009/140646, expressly incorporated by reference herein.

As used herein, the term “fatty acid” means a carboxylic acid having the formula RCOOH. R represents an aliphatic group, preferably an alkyl group. R can comprise between about 4 and about 22 carbon atoms. Fatty acids can be saturated or monounsaturated. In a preferred embodiment, the fatty acid is made from a fatty acid biosynthetic pathway.

As used herein, the term “fatty acid biosynthetic pathway” means a biosynthetic pathway that produces acyl thioesters. The fatty acid biosynthetic pathway includes fatty acid synthases that can be engineered to produce acyl thioesters, and in some embodiments can be expressed with additional enzymes to produce fatty acids having desired carbon chain characteristics. It is understood by those skilled in the art that fatty acids are biosynthesized not as the “acids”, but as acyl thioesters, i.e., the acid is bound as a thioester to the 4-phosphopantethionyl prosthetic group of ACP or CoA. The fatty acyl group can them be used in the cell to build membranes, cell walls, fats, hydrolyzed to fatty acids, and may be further modified biochemically to produce fatty acid derivatives, such as aldehydes, alcohols, alkenes, alkanes, esters, and the like.

As used herein, the term “fatty acid derivatives” means products made in part by way of the fatty acid biosynthetic pathway. The term “fatty acid derivatives” may be used interchangeably herein with the term “fatty acids or derivatives thereof” and includes products made in part from acyl-ACP or acyl-ACP derivatives. Exemplary “fatty acid derivatives” include, for example, fatty acids, acyl-CoA, fatty aldehydes, short and long chain alcohols, hydrocarbons (e.g., alkanes, alkenes or olefins, such as terminal or internal olefins), fatty alcohols, esters (e.g., wax esters, fatty acid esters (e.g., methyl or ethyl esters), and ketones. As used herein, the term “target fatty acid derivatives” means fatty acid derivatives having desired aliphatic chain lengths and saturation characteristics.

As used herein, the term “fatty acid derivative enzymes” means all enzymes that may be expressed or overexpressed in the production of fatty acid derivatives. These enzymes are collectively referred to herein as fatty acid derivative enzymes. These enzymes may be part of the fatty acid biosynthetic pathway. Non-limiting examples of fatty acid derivative enzymes include fatty acid synthases, thioesterases, acyl-CoA synthases, acyl-CoA reductases, alcohol dehydrogenases, alcohol acyltransferases, fatty alcohol-forming acyl-CoA reductase, ester synthases, aldehyde biosynthetic polypeptides, and alkane biosynthetic polypeptides. Fatty acid derivative enzymes convert a substrate into a fatty acid derivative. In some examples, the substrate may be a fatty acid derivative which the fatty acid derivative enzyme converts into a different fatty acid derivative.

As used herein, “fatty acid enzyme” means any enzyme involved in fatty acid biosynthesis. Fatty acid enzymes can be expressed or overexpressed in host cells to produce fatty acids. Non-limiting examples of fatty acid enzymes include fatty acid synthases and thioesterases. As used herein, the term “alkane” means saturated hydrocarbons or compounds that consist only of carbon (C) and hydrogen (H), wherein these atoms are linked together by single bonds (i.e., they are saturated compounds).

As used herein, an “alkane biosynthetic gene” or an “alkane biosynthetic polynucleotide” is a nucleic acid that encodes an alkane biosynthetic polypeptide.

As used herein, an “alkane biosynthetic polypeptide” is a polypeptide that is a part of the biosynthetic pathway of an alkane. Such polypeptides can act on a biological substrate to yield an alkane. In some instances, the alkane biosynthetic polypeptide has decarbonylase activity.

As used herein, the terms “olefin” and “alkene” are used interchangeably and refer to hydrocarbons containing at least one carbon-to-carbon double bond (i.e., they are unsaturated compounds).

As used herein, the terms “terminal olefin,” “α-olefin”, “terminal alkene” and “1-alkene” are used interchangeably herein with reference to α-olefins or alkenes with a chemical formula CₓH₂ₓ, distinguished from other olefins with a similar molecular formula by linearity of the hydrocarbon chain and the position of the double bond at the primary or alpha position.

As used herein, an “alkene biosynthetic gene” or an “alkene biosynthetic polynucleotide” is a nucleic acid that encodes an alkene biosynthetic polypeptide.

As used herein, an “alkene biosynthetic polypeptide” is a polypeptide that is a part of the biosynthetic pathway of an alkene. Such polypeptides can act on a biological substrate to yield an alkene. In some instances, the alkene biosynthetic polypeptide has decarbonylase activity.

As used herein, the term “fatty ester” refers to any ester made from a fatty acid, for example a fatty acid ester. In some embodiments, a fatty ester contains an A side and a B side. As used herein, an “A side” of an ester refers to the carbon chain attached to the carboxylate oxygen of the ester. As used herein, a “B side” of an ester refers to the carbon chain comprising the parent carboxylate of the ester. In embodiments where the fatty ester is derived from the fatty acid biosynthetic pathway, the A side is contributed by an alcohol (e.g., ethanol or methanol), and the B side is contributed by a fatty acid.

Any alcohol can be used to form the A side of the fatty esters. For example, the alcohol can be derived from the fatty acid biosynthetic pathway. Alternatively, the alcohol can be produced through non-fatty acid biosynthetic pathways. Moreover, the alcohol can be provided exogenously. For example, the alcohol can be supplied in the fermentation broth in instances where the fatty ester is produced by an organism. Alternatively, a carboxylic acid, such as a fatty acid or acetic acid, can be supplied exogenously in instances where the fatty ester is produced by an organism that can also produce alcohol.

The carbon chains comprising the A side or B side can be of any length. In one embodiment, the A side of the ester is at least about 1, 2, 3, 4, 5, 6, 7, 8, 10, 12, 14, 16, or 18 carbons in length. When the fatty ester is a fatty acid methyl ester, the A side of the ester is 1 carbon in length. When the fatty ester is a fatty acid ethyl ester, the A side of the ester is 2 carbons in length. The B side of the ester can be at least about 4, 6, 8, 10, 12, 14, 16, 18, 20, 22, 24, or 26 carbons in length. Furthermore, the A side and/or B side can be saturated or unsaturated.

As used herein, the term “ester synthase” means a peptide capable of producing fatty esters. More specifically, an ester synthase is a peptide which converts a thioester to a fatty ester. In a preferred embodiment, the ester synthase converts a thioester (e.g., acyl-CoA) to a fatty ester.

In an alternate embodiment, an ester synthase uses a thioester and an alcohol as substrates to produce a fatty ester. Ester synthases are capable of using short and long chain thioesters as substrates. In addition, ester synthases are capable of using short and long chain alcohols as substrates.

Non-limiting examples of ester synthases are wax synthases, wax-ester synthases, acyl CoA:alcohol transacylases, acyltransferases, and fatty acyl-coenzyme A:fatty alcohol acyltransferases. Exemplary ester synthases are classified in enzyme classification number EC 2.3.1.75. Exemplary GenBank Accession Numbers are provided in FIG. 40 of W02009/140646, expressly incorporated by reference herein.

In one embodiment, the fatty ester is a wax. The wax can be derived from a long chain alcohol and a long chain fatty acid. In another embodiment, the fatty ester is a fatty acid thioester, for example Acyl-ACP. Fatty esters can be used, for example, as biofuels or surfactants.

As used herein, the term “attenuate” means to weaken, reduce or diminish. For example, a polypeptide can be attenuated by modifying the polypeptide to reduce its activity (e.g., by modifying a nucleotide sequence that encodes the polypeptide).

As used herein, the term “carbon source” refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, and gases (e.g., CO and CO₂). Exemplary carbon sources include, but are not limited to, monosaccharides, such as glucose, fructose, mannose, galactose, xylose, and arabinose; oligosaccharides, such as fructo-oligosaccharide and galacto-oligosaccharide; polysaccharides such as starch, cellulose, pectin, and xylan; disaccharides, such as sucrose, maltose, cellobiose, and turanose; cellulosic material and variants such as hemicelluloses, methyl cellulose and sodium carboxymethyl cellulose; saturated or unsaturated fatty acids, succinate, lactate, and acetate; alcohols, such as ethanol, methanol, and glycerol, or mixtures thereof. The carbon source can also be a product of photosynthesis, such as glucose. In certain preferred embodiments, the carbon source is biomass. In other preferred embodiments, the carbon source is glucose, sucrose, fructose or combinations thereof. In other preferred embodiments, the carbon source is directly or indirectly derived from a natural feed stock such as sugar cane, sweet sorghum, switchgrass, sugar beets and others.

As used herein, the term “biomass” refers to any biological material from which a carbon source is derived. In some embodiments, a biomass is processed into a carbon source, which is suitable for bioconversion. In other embodiments, the biomass does not require further processing into a carbon source. The carbon source can be converted into any combination of fatty acids or fatty acid derivatives. An exemplary source of biomass is plant matter or vegetation, such as corn, sugar cane, or switchgrass. Another exemplary source of biomass is metabolic waste products, such as animal matter (e.g., cow manure). Further exemplary sources of biomass include algae and other marine plants. Biomass also includes waste products from industry, agriculture, forestry, and households, including, but not limited to, fermentation waste, ensilage, straw, lumber, sewage, garbage, cellulosic urban waste, and food leftovers. The term “biomass” also can refer to sources of carbon, such as carbohydrates (e.g., monosaccharides, disaccharides, or polysaccharides).

A nucleotide sequence is “complementary” to another nucleotide sequence if each of the bases of the two sequences matches (i.e., is capable of forming Watson Crick base pairs). The term “complementary strand” is used herein interchangeably with the term “complement”. The complement of a nucleic acid strand can be the complement of a coding strand or the complement of a non-coding strand.

As used herein, the term “conditions sufficient to allow expression” means any conditions that allow a host cell to produce a desired product, such as a polypeptide, aldehyde, or alkane described herein. Suitable conditions include, for example, fermentation conditions. Fermentation conditions can comprise many parameters, such as temperature ranges, levels of aeration, and media composition. Each of these conditions, individually and in combination, allows the host cell to grow. Exemplary culture media include broths or gels. Generally, the medium includes a carbon source, such as glucose, fructose, cellulose, or the like, that can be metabolized by a host cell directly. In addition, enzymes can be used in the medium to facilitate the mobilization (e.g., the depolymerization of starch or cellulose to fermentable sugars) and subsequent metabolism of the carbon source.

To determine if conditions are sufficient to allow expression, a host cell can be cultured, for example, for about 4, 8, 12, 24, 36, or 48 hours. During and/or after culturing, samples can be obtained and analyzed to determine if the conditions allow expression. For example, the host cells in the sample or the medium in which the host cells were grown can be tested for the presence of a desired product. When testing for the presence of a product, assays, such as, but not limited to, TLC, HPLC, GC/FID, GC/MS, LC/MS, MS, can be used.

It is understood that the polypeptides described herein may have additional conservative or non-essential amino acid substitutions, which do not have a substantial effect on the polypeptide functions. Whether or not a particular substitution will be tolerated (i.e., will not adversely affect desired biological properties, such as decarboxylase activity) can be determined as described in Bowie et al., Science (1990) 247:1306 1310. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

As used herein, “control element” means a transcriptional control element. Control elements include promoters and enhancers. The term “promoter element,” “promoter,” or “promoter sequence” refers to a DNA sequence that functions as a switch that activates the expression of a gene. If the gene is activated, it is said to be transcribed or participating in transcription. Transcription involves the synthesis of mRNA from the gene. A promoter, therefore, serves as a transcriptional regulatory element and also provides a site for initiation of transcription of the gene into mRNA. Control elements interact specifically with cellular proteins involved in transcription (Maniatis et al., Science 236:1237, 1987).

As used herein, “fraction of modern carbon” or “f_{M}” has the same meaning as defined by National Institute of Standards and Technology (NIST) Standard Reference Materials (SRMs) 4990B and 4990C, known as oxalic acids standards HOxI and HOxII, respectively. The fundamental definition relates to 0.95 times the ¹⁴C/¹²C isotope ratio HOxI (referenced to AD 1950). This is roughly equivalent to decay-corrected pre-Industrial Revolution wood. For the current living biosphere (plant material), f_{M} is approximately 1.1.

Calculations of “homology” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence that is aligned for comparison purposes is at least about 30%, preferably at least about 40%, more preferably at least about 50%, even more preferably at least about 60%, and even more preferably at least about 70%, at least about 80%, at least about 90%, or about 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein, amino acid or nucleic acid “identity” is equivalent to amino acid or nucleic acid “homology”). The percent identity between the two sequences is a function of the number of identical positions shared by the sequences, taking into account the number of gaps and the length of each gap, which need to be introduced for optimal alignment of the two sequences.

The comparison of sequences and determination of percent homology between two sequences can be accomplished using a mathematical algorithm. In a preferred embodiment, the percent homology between two amino acid sequences is determined using the Needleman and Wunsch (1970), J. Mol. Biol. 48:444 453, algorithm that has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent homology between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about which parameters should be applied to determine if a molecule is within a homology limitation of the claims) are a Blossum 62 scoring matrix with a gap penalty of 12, a gap extend penalty of 4, and a frameshift gap penalty of 5.

As used herein, a “host cell” is a cell used to produce a product described herein (e.g., an aldehyde or alkane described herein). A host cell can be modified to express or overexpress selected genes or to have attenuated expression of selected genes. Non-limiting examples of host cells include plant, animal, human, bacteria, yeast, or filamentous fungi cells.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either method can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions unless otherwise specified.

The term “isolated” as used herein with respect to nucleic acids, such as DNA or RNA, refers to molecules separated from other DNAs or RNAs, respectively that are present in the natural source of the nucleic acid. Moreover, an “isolated nucleic acid” includes nucleic acid fragments, such as fragments that are not naturally occurring. The term “isolated” is also used herein to refer to polypeptides, which are isolated from other cellular proteins, and encompasses both purified endogenous polypeptides and recombinant polypeptides. The term “isolated” as used herein also refers to a nucleic acid or polypeptide that is substantially free of cellular material, viral material, or culture medium when produced by recombinant DNA techniques. The term “isolated” as used herein also refers to a nucleic acid or polypeptide that is substantially free of chemical precursors or other chemicals when chemically synthesized.

As used herein, the “level of expression of a gene in a cell” refers to the level of mRNA, pre-mRNA nascent transcript(s), transcript processing intermediates, mature mRNA(s), and/or degradation products encoded by the gene in the cell.

As used herein, the term “microorganism” means prokaryotic and eukaryotic microbial species from the domains Archaea, Bacteria and Eucarya, the latter including yeast and filamentous fungi, protozoa, algae, or higher Protista. The term “microbial cell”, as used herein, means a cell from a microorganism.

As used herein, the term “recombinant host cell” refers to a host whose genetic makeup has been altered relative to the corresponding wild-type host cell, for example, by deliberate introduction of new genetic elements and/or deliberate modification of genetic elements naturally present in the host cell. The offspring of such recombinant host cells also contain these new and/or modified genetic elements. In any of the aspects of the invention described herein, the host cell can be selected from the group consisting of a mammalian cell, plant cell, insect cell, fungus cell (e.g., a filamentous fungus, such as Candida sp., or a budding yeast, such as Saccharomyces sp.), algal cell, and bacterial cell. In a preferred embodiment, recombinant host cells are “recombinant microorganisms.”

As used herein, a “host cell of the same kind as the recombinant host cell” typically means a host cell of the same species that does not have the recombinant modification described for the recombinant host cell. For example, “a microorganism of the same kind as the recombinant microorganism” typically refers to a microorganism of the same species, (e.g., E. coli), and the same strain (e.g., E. coli K-12) as the recombinant microorganism, wherein the microorganism does not comprise the recombinant modification described for the recombinant microorganism.

The term “or” is used herein to mean, and is used interchangeably with, the term “and/or,” unless context clearly indicates otherwise.

As used herein, “overexpress” means to express or cause to be expressed a nucleic acid, polypeptide, or hydrocarbon in a cell at a greater concentration than is normally expressed in a corresponding wild-type cell. For example, a polypeptide can be “overexpressed” in a recombinant host cell when the polypeptide is present in a greater concentration in the recombinant host cell compared to its concentration in a non-recombinant host cell of the same species.

As used herein, “partition coefficient” or “P,” is defined as the equilibrium concentration of a compound in an organic phase divided by the concentration at equilibrium in an aqueous phase (e.g., fermentation broth). In one embodiment of a bi-phasic system described herein, the organic phase is formed by the aldehyde or alkane during the production process. However, in some examples, an organic phase can be provided, such as by providing a layer of octane, to facilitate product separation. When describing a two phase system, the partition characteristics of a compound can be described as log P. For example, a compound with a log P of 1 would partition 10:1 to the organic phase. A compound with a log P of −1 would partition 1:10 to the organic phase. By choosing an appropriate fermentation broth and organic phase, an aldehyde or alkane with a high log P value can separate into the organic phase even at very low concentrations in the fermentation vessel.

As used herein, the term “purify,” “purified,” or “purification” means the removal or isolation of a molecule from its environment by, for example, isolation or separation. “Substantially purified” molecules are at least about 60% free, preferably at least about 75% free, and more preferably at least about 90% free from other components with which they are associated. As used herein, these terms also refer to the removal of contaminants from a sample. For example, the removal of contaminants can result in an increase in the percentage of aldehydes or alkanes in a sample. For example, when aldehydes or alkanes are produced in a host cell, the aldehydes or alkanes can be purified by the removal of host cell proteins. After purification, the percentage of aldehydes or alkanes in the sample is increased.

The terms “purify,” “purified,” and “purification” do not require absolute purity. They are relative terms. Thus, for example, when aldehydes or alkanes are produced in host cells, a purified aldehyde or purified alkane is one that is substantially separated from other cellular components (e.g., nucleic acids, polypeptides, lipids, carbohydrates, or other hydrocarbons). In another example, a purified aldehyde or purified alkane preparation is one in which the aldehyde or alkane is substantially free from contaminants, such as those that might be present following fermentation. In some embodiments, an aldehyde or an alkane is purified when at least about 50% by weight of a sample is composed of the aldehyde or alkane. In other embodiments, an aldehyde or an alkane is purified when at least about 60%, 70%, 80%, 85%, 90%, 92%, 95%, 98%, or 99% or more by weight of a sample is composed of the aldehyde or alkane.

As used herein, the term “recombinant polypeptide” refers to a polypeptide that is produced by recombinant DNA techniques, wherein generally DNA encoding the expressed polypeptide or RNA is inserted into a suitable expression vector and that is in turn used to transform a host cell to produce the polypeptide or RNA.

As used herein, the term “synthase” means an enzyme which catalyzes a synthesis process. As used herein, the term synthase includes synthases, synthetases, and ligases.

As used herein, the term “transfection” means the introduction of a nucleic acid (e.g., via an expression vector) into a recipient cell by nucleic acid-mediated gene transfer.

As used herein, “transformation” refers to a process in which a cell's genotype is changed as a result of the cellular uptake of exogenous nucleic acid. This may result in the transformed cell expressing a recombinant form of an RNA or polypeptide. In the case of antisense expression from the transferred gene, the expression of a naturally-occurring form of the polypeptide is disrupted.

As used herein, a “transport protein” is a polypeptide that facilitates the movement of one or more compounds in and/or out of a cellular organelle and/or a cell.

As used herein, a “variant” of polypeptide X refers to a polypeptide having the amino acid sequence of polypeptide X in which one or more amino acid residues is altered. The variant may have conservative changes or nonconservative changes. Guidance in determining which amino acid residues may be substituted, inserted, or deleted without affecting biological activity may be found using computer programs well known in the art, for example, LASERGENE software (DNASTAR).

The term “variant,” when used in the context of a polynucleotide sequence, may encompass a polynucleotide sequence related to that of a gene or the coding sequence thereof. This definition may also include, for example, “allelic,” “splice,” “species,” or “polymorphic” variants. A splice variant may have significant identity to a reference polynucleotide, but will generally have a greater or fewer number of polynucleotides due to alternative splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variant is a variation in the polynucleotide sequence of a particular gene between individuals of a given species.

As used herein, the term “vector” refers to a nucleic acid molecule capable of transporting another nucleic acid to which it has been linked. One type of useful vector is an episome (i.e., a nucleic acid capable of extra-chromosomal replication). Useful vectors are those capable of autonomous replication and/or expression of nucleic acids to which they are linked Vectors capable of directing the expression of genes to which they are operatively linked are referred to herein as “expression vectors”. In general, expression vectors of utility in recombinant DNA techniques are often in the form of “plasmids,” which refer generally to circular double stranded DNA loops that, in their vector form, are not bound to the chromosome. In the present specification, “plasmid” and “vector” are used interchangeably, as the plasmid is the most commonly used form of vector. However, also included are such other forms of expression vectors that serve equivalent functions and that become known in the art subsequently hereto.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

### BRIEF DESCRIPTION OF THE DRAWINGS

FIG. 1A is a GC/MS trace of hydrocarbons produced by Prochlorococcus marinus CCMP1986 cells. FIG. 1B is a mass fragmentation pattern of the peak at 7.55 min of FIG. 1A.

FIG. 2A is a GC/MS trace of hydrocarbons produced by Nostoc punctiforme PCC73102 cells. FIG. 2B is a mass fragmentation pattern of the peak at 8.73 min of FIG. 2A.

FIG. 3A is a GC/MS trace of hydrocarbons produced by Gloeobaceter violaceus ATCC29082 cells. FIG. 3B is a mass fragmentation pattern of the peak at 8.72 min of FIG. 3A.

FIG. 4A is a GC/MS trace of hydrocarbons produced by Synechocystic sp. PCC6803 cells. FIG. 4B is a mass fragmentation pattern of the peak at 7.36 min of FIG. 4A.

FIG. 5A is a GC/MS trace of hydrocarbons produced by Synechocystis sp. PCC6803 wild type cells. FIG. 5B is a GC/MS trace of hydrocarbons produced by Synechocystis sp. PCC6803 cells with a deletion of the sll0208 and sll0209 genes.

FIG. 6A is a GC/MS trace of hydrocarbons produced by E. coli MG1655 wild type cells. FIG. 6B is a GC/MS trace of hydrocarbons produced by E. coli MG1655 cells expressing Synechococcus elongatus PCC7942 YP_{—}400611 (Synpcc7942_{—}1594) (SEQ ID NO:65).

FIG. 7 is a GC/MS trace of hydrocarbons produced by E. coli cells expressing Cyanothece sp. ATCC51142 cce_{—}1430 (YP_{—}001802846) (SEQ ID NO:69).

FIG. 8A is a GC/MS trace of hydrocarbons produced by E. coli cells expressing Synechococcus elongatus PCC7942 YP_{—}400611 (Synpcc7942_{—}1594) (SEQ ID NO:65) and Synechococcus elongatus PCC7942 YP_{—}400610 (Synpcc7942_{—}1593) (SEQ ID NO:1). FIG. 8B depicts mass fragmentation patterns of the peak at 6.98 min of FIG. 8A and of pentadecane. FIG. 8C depicts mass fragmentation patterns of the peak at 8.12 min of FIG. 8A and of 8-heptadecene.

FIG. 9 is a GC/MS trace of hydrocarbons produced by E. coli MG1655 cells expressing Synechococcus elongatus PCC7942 YP_{—}400611 (Synpcc7942_{—}1594) (SEQ ID NO:65) and Nostoc punctiforme PCC73102 Npun02004178 (ZP_{—}00108838) (SEQ ID NO:5).

FIG. 10 is a GC/MS trace of hydrocarbons produced by E. coli MG1655 cells expressing Synechococcus elongatus PCC7942 YP_{—}400611 (Synpcc7942_{—}1594) (SEQ ID NO:65) and Synechocystis sp. PCC6803 sll0208 (NP_{—}442147) (SEQ ID NO:3).

FIG. 11 is a GC/MS trace of hydrocarbons produced by E. coli MG1655 cells expressing Synechococcus elongatus PCC7942 YP_{—}400611 (Synpcc7942_{—}1594) (SEQ ID NO:65) and Nostoc sp. PCC7210 alr5283 (NP_{—}489323) (SEQ ID NO:7).

FIG. 12 is a GC/MS trace of hydrocarbons produced by E. coli MG1655 cells expressing Synechococcus elongatus PCC7942 YP_{—}400611 (Synpcc7942_{—}1594) (SEQ ID NO:65) and codon-optimized Acaryochloris marina MBIC11017 AM1_{—}4041 (YP_{—}001518340) (SEQ ID NO:46).

FIG. 13 is a GC/MS trace of hydrocarbons produced by E. coli MG1655 cells expressing Synechococcus elongatus PCC7942 YP_{—}400611 (Synpcc7942_{—}1594) (SEQ ID NO:65) and codon-optimized Thermosynechococcus elongatus BP-1 tll1313 (NP_{—}682103) (SEQ ID NO:47).

FIG. 14 is a GC/MS trace of hydrocarbons produced by E. coli MG1655 cells expressing Synechococcus elongatus PCC7942 YP_{—}400611 (Synpcc7942_{—}1594) (SEQ ID NO:65) and codon-optimized Synechococcus sp. JA-3-3Ab CYA_{—}0415 (YP_{—}473897) (SEQ ID NO:48).

FIG. 15 is a GC/MS trace of hydrocarbons produced by E. coli MG1655 cells expressing Synechococcus elongatus PCC7942 YP_{—}400611 (Synpcc7942_{—}1594) (SEQ ID NO:65) and Gloeobacter violaceus PCC7421 gll3146 (NP_{—}926092) (SEQ ID NO:15).

FIG. 16 is a GC/MS trace of hydrocarbons produced by E. coli MG1655 cells expressing Synechococcus elongatus PCC7942 YP_{—}400611 (Synpcc7942_{—}1594) (SEQ ID NO:65) and codon-optimized Prochlorococcus marinus MIT9313 PMT1231 (NP_{—}895059) (SEQ ID NO:49).

FIG. 17 is a GC/MS trace of hydrocarbons produced by E. coli MG1655 cells expressing Synechococcus elongatus PCC7942 YP_{—}400611 (Synpcc7942_{—}1594) (SEQ ID NO:65) and Prochlorococcus marinus CCMP1986 PMM0532 (NP_{—}892650) (SEQ ID NO:19).

FIG. 18 is a GC/MS trace of hydrocarbons produced by E. coli MG1655 cells expressing Synechococcus elongatus PCC7942 YP_{—}400611 (Synpcc7942_{—}1594) (SEQ ID NO:65) and codon-optimized Prochlorococcus marinus NATL2A PMN2A_{—}1863 (YP_{—}293054) (SEQ ID NO:51).

FIG. 19 is a GC/MS trace of hydrocarbons produced by E. coli MG1655 cells expressing Synechococcus elongatus PCC7942 YP_{—}400611 (Synpcc7942_{—}1594) (SEQ ID NO:65) and codon-optimized Synechococcus sp. RS9917 RS9917_{—}09941 (ZP_{—}01079772) (SEQ ID NO:52).

FIG. 20 is a GC/MS trace of hydrocarbons produced by E. coli MG1655 cells expressing Synechococcus elongatus PCC7942 YP_{—}400611 (Synpcc7942_{—}1594) (SEQ ID NO:65) and codon-optimized Synechococcus sp. RS9917 RS9917_{—}12945 (ZP_{—}01080370) (SEQ ID NO:53).

FIG. 21 is a GC/MS trace of hydrocarbons produced by E. coli MG1655 cells expressing Synechococcus elongatus PCC7942 YP_{—}400611 (Synpcc7942_{—}1594) (SEQ ID NO:65) and Cyanothece sp. ATCC51142 cce_{—}0778 (YP_{—}001802195) (SEQ ID NO:27).

FIG. 22 is a GC/MS trace of hydrocarbons produced by E. coli MG1655 cells expressing Synechococcus elongatus PCC7942 YP_{—}400611 (Synpcc7942_{—}1594) (SEQ ID NO:65) and Cyanothece sp. PCC7425 Cyan7425_{—}0398 (YP_{—}002481151) (SEQ ID NO:29).

FIG. 23 is a GC/MS trace of hydrocarbons produced by E. coli MG1655 cells expressing Synechococcus elongatus PCC7942 YP_{—}400611 (Synpcc7942_{—}1594) (SEQ ID NO:65) and Cyanothece sp. PCC7425 Cyan7425_{—}2986 (YP_{—}002483683) (SEQ ID NO:31).

FIG. 24A is a GC/MS trace of hydrocarbons produced by E. coli MG1655 cells expressing Prochlorococcus marinus CCMP1986 PMM0533 (NP_{—}892651) (SEQ ID NO:71). FIG. 24B is a GC/MS trace of hydrocarbons produced by E. coli MG1655 cells expressing Prochlorococcus marinus CCMP1986 PMM0533 (NP_{—}892651) (SEQ ID NO:71) and Prochlorococcus marinus CCMP1986 PMM0532 (NP_{—}892650) (SEQ ID NO:19).

FIG. 25A is a GC/MS trace of hydrocarbons produced by E. coli MG1655 ΔfadE lacZ::P_{trc} ′tesA-fadD cells. FIG. 25B is a GC/MS trace of hydrocarbons produced by E. coli MG1655 ΔfadE lacZ::P_{trc} ′tesA-fadD cells expressing Synechococcus elongatus PCC7942 YP_{—}400611 (Synpcc7942_{—}1594) (SEQ ID NO:65) and Acaryochloris marina MBIC11017 AM1_{—}4041 (YP_{—}001518340) (SEQ ID NO:9).

FIG. 26A is a GC/MS trace of hydrocarbons produced by E. coli MG1655 ΔfadE lacZ::P_{trc} ′tesA-fadD cells expressing Synechocystis sp. PCC6803 sll0209 (NP_{—}442146) (SEQ ID NO:67). FIG. 26B is a GC/MS trace of hydrocarbons produced by E. coli MG1655 ΔfadE lacZ::P_{trc} ′tesA-fadD cells expressing Synechocystis sp. PCC6803 sll0209 (NP_{—}442146) (SEQ ID NO:67) and Synechocystis sp. PCC6803 sll0208 (NP_{—}442147) (SEQ ID NO:3).

FIG. 27A is a GC/MS trace of hydrocarbons produced by E. coli MG1655 fadD lacZ::P_{trc}-′tesA cells expressing M. smegmatis strain MC2 155 MSMEG_{—}5739 (YP_{—}889972) (SEQ ID NO:85). FIG. 27B is a GC/MS trace of hydrocarbons produced by E. coli MG1655 fadD lacZ::P_{trc}-′tesA cells expressing M. smegmatis strain MC2 155 MSMEG_{—}5739 (YP_{—}889972) (SEQ ID NO:85) and Nostoc punctiforme PCC73102 Npun02004178 (ZP_{—}00108838) (SEQ ID NO:5).

FIG. 28 is a graphic representation of hydrocarbons produced by E. coli MG1655 fadD lacZ::P_{trc}-′tesA cells expressing M. smegmatis strain MC2 155 MSMEG_{—}5739 (YP_{—}889972) (SEQ ID NO:85) either alone or in combination with Nostoc sp. PCC7120 alr5283 (SEQ ID NO:7), Nostoc punctiforme PCC73102 Npun02004178 (SEQ ID NO:5), P. marinus CCMP1986 PMM0532 (SEQ ID NO:19), G. violaceus PCC7421 gll3146 (SEQ ID NO:15), Synechococcus sp. RS9917_{—}09941 (SEQ ID NO:23), Synechococcus sp. RS9917_{—}12945 (SEQ ID NO:25), or A. marina MBIC11017 AM1_{—}4041 (SEQ ID NO:9).

FIG. 29A is a representation of the three-dimensional structure of a class I ribonuclease reductase subunit β protein, RNRβ. FIG. 29B is a representation of the three-dimensional structure of Prochlorococcus marinus MIT9313 PMT1231 (NP_{—}895059) (SEQ ID NO:17). FIG. 29C is a representation of the three-dimensional structure of the active site of Prochlorococcus marinus MIT9313 PMT1231 (NP_{—}895059) (SEQ ID NO:17).

FIG. 30A is a GC/MS trace of hydrocarbons produced by E. coli MG1655 cells expressing Nostoc punctiforme PCC73102 Npun02004178 (ZP_{—}00108838) (SEQ ID NO:5). FIG. 30B is a GC/MS trace of hydrocarbons produced by E. coli MG1655 cells expressing Nostoc punctiforme PCC73102 Npun02004178 (ZP_{—}00108838) Y123F variant. FIG. 30C is a GC/MS trace of hydrocarbons produced by E. coli MG1655 cells expressing Nostoc punctiforme PCC73102 Npun02004178 (ZP_{—}00108838) Y126F variant.

FIG. 31 depicts GC/MS traces of hydrocarbons produced in vitro using Nostoc punctiforme PCC73102 Npun02004178 (ZP_{—}00108838) (SEQ ID NO:6) and octadecanal (A); Npun02004178 (ZP_{—}00108838) (SEQ ID NO:6), octadecanal, spinach ferredoxin reductase, and NADPH (B); octadecanal, spinach ferredoxin, spinach ferredoxin reductase, and NADPH(C); or Npun02004178 (ZP_{—}00108838) (SEQ ID NO:6), spinach ferredoxin, and spinach ferredoxin (D).

FIG. 32 depicts GC/MS traces of hydrocarbons produced in vitro using Nostoc punctiforme PCC73102 Npun02004178 (ZP_{—}00108838) (SEQ ID NO:6), NADPH, octadecanal, and either (A) spinach ferredoxin and spinach ferredoxin reductase; (B) N. punctiforme PCC73102 Npun02003626 (ZP_{—}00109192) (SEQ ID NO:88) and N. punctiforme PCC73102 Npun02001001 (ZP_{—}00111633) (SEQ ID NO:90); (C) Npun02003626 (ZP_{—}00109192) (SEQ ID NO:88) and N. punctiforme PCC73102 Npun02003530 (ZP_{—}00109422) (SEQ ID NO:92); or (D) Npun02003626 (ZP_{—}00109192) (SEQ ID NO:88) and N. punctiforme PCC73102 Npun02003123 (ZP_{—}00109501) (SEQ ID NO:94).

FIG. 33A is a GC/MS trace of hydrocarbons produced in vitro using octadecanoyl-CoA, Synechococcus elongatus PCC7942 YP_{—}400611 (Synpcc7942_{—}1594) (SEQ ID NO:66), NADPH, and Mg²⁺. FIG. 33B is a GC/MS trace of hydrocarbons produced in vitro using octadecanoyl-CoA, Synechococcus elongatus PCC7942 YP_{—}400611 (Synpcc7942_{—}1594) (SEQ ID NO:66), NADPH, and Mg²⁺. FIG. 33C is a GC/MS trace of hydrocarbons produced in vitro using octadecanoyl-CoA, Synechococcus elongatus PCC7942 YP_{—}400611 (Synpcc7942_{—}1594) (SEQ ID NO:66) and NADPH.

FIG. 34A is a GC/MS trace of hydrocarbons produced in vitro using octadecanoyl-CoA, labeled NADPH, Synechococcus elongatus PCC7942 YP_{—}400611 (Synpcc7942_{—}1594) (SEQ ID NO:66), and unlabeled NADPH. FIG. 34B is a GC/MS trace of hydrocarbons produced in vitro using octadecanoyl-CoA, labeled NADPH, Synechococcus elongatus PCC7942 YP_{—}400611 (Synpcc7942_{—}1594) (SEQ ID NO:66), and S-(4-²H)NADPH. FIG. 34C is a GC/MS trace of hydrocarbons produced in vitro using octadecanoyl-CoA, labeled NADPH, Synechococcus elongatus PCC7942 YP_{—}400611 (Synpcc7942_{—}1594) (SEQ ID NO:66), and R-(4-²H)NADPH.

FIG. 35 is a GC/MS trace of hydrocarbons in the cell-free supernatant produced by E. coli MG1655 ΔfadE cells in Che-9 media expressing Synechococcus elongatus PCC7942 YP_{—}400611 (Synpcc7942_{—}1594) (SEQ ID NO:65).

FIG. 36 is a GC/MS trace of hydrocarbons in the cell-free supernatant produced by E. coli MG1655 ΔfadE cells in Che-9 media expressing Synechococcus elongatus PCC7942 YP_{—}400611 (Synpcc7942_{—}1594) (SEQ ID NO:65) and Nostoc punctiforme PCC73102 Npun02004178 (ZP_{—}00108838) (SEQ ID NO:5).

FIG. 37 is a GC/MS trace of hydrocarbons produced by E. coli MG1655 cells expressing Nostoc sp. PCC7120 alr5283 (NP_{—}489323) (SEQ ID NO:7) and Nostoc sp. PCC7120 alr5284 (NP_{—}489324) (SEQ ID NO:81).

FIG. 38 is a graph of cell growth throughout a bioreactor run.

FIG. 39A is a graph of glucose consumption throughout a bioreactor run. FIG. 39B is a graph of glucose concentration in the medium throughout a bioreactor run.

FIG. 40 is a graph of canola oil concentration in the culture medium of hydrocarbon production cells.

FIG. 41A is a graph of alkane concentration produced by hydrocarbon production cells. FIG. 41B is a graph of fatty matters concentration produced by hydrocarbon production cells.

FIG. 42 is a graph of alkane yield vs. glucose feed.

### DETAILED DESCRIPTION

The invention provides compositions and methods of producing aldehydes, fatty alcohols, and hydrocarbons (such as alkanes, alkenes, and alkynes) from substrates, for example, an acyl-ACP, a fatty acid, an acyl-CoA, a fatty aldehyde, or a fatty alcohol substrate (e.g., as described in WO/2008/119082, expressly incorporated by reference herein). Such aldehydes, alkanes, and alkenes are useful as biofuels (e.g., substitutes for gasoline, diesel, jet fuel, etc.), specialty chemicals (e.g., lubricants, fuel additive, etc.), or feedstock for further chemical conversion (e.g., fuels, polymers, plastics, textiles, solvents, adhesives, etc.). The invention is based, in part, on the identification of genes that are involved in aldehyde, alkane, and alkene biosynthesis.

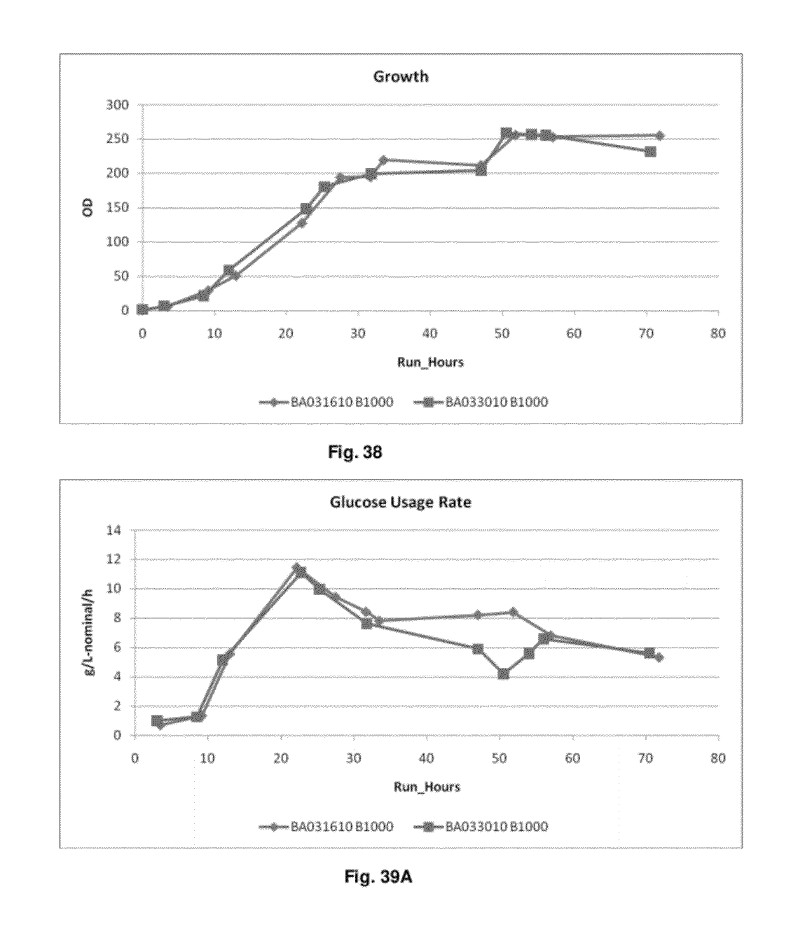

Such alkane and alkene biosynthetic genes include, for example, Synechococcus elongatus PCC7942 Synpcc7942_{—}1593 (SEQ ID NO:1), Synechocystis sp. PCC6803 sll0208 (SEQ ID NO:3), Nostoc punctiforme PCC 73102 Npun02004178 (SEQ ID NO:5), Nostoc sp. PCC 7120 alr5283 (SEQ ID NO:7), Acaryochloris marina MBIC11017 AM1_{—}4041 (SEQ ID NO:9), Thermosynechococcus elongatus BP-1 tll1313 (SEQ ID NO:11), Synechococcus sp. JA-3-3A CYA_{—}0415 (SEQ ID NO:13), Gloeobacter violaceus PCC 7421 gll3146 (SEQ ID NO:15), Prochlorococcus marinus MIT9313 PM123 (SEQ ID NO:17), Prochlorococcus marinus subsp. pastoris str. CCMP1986 PMM0532 (SEQ ID NO:19), Prochlorococcus marinus str. NATL2A PMN2A_{—}1863 (SEQ ID NO:21), Synechococcus sp. RS9917 RS9917_{—}09941 (SEQ ID NO:23), Synechococcus sp. RS9917 RS9917_{—}12945 (SEQ ID NO:25), Cyanothece sp. ATCC51142 cce_{—}0778 (SEQ ID NO:27), Cyanothece sp. PCC7245 Cyan7425DRAFT_{—}1220 (SEQ ID NO:29), Cyanothece sp. PCC7245 cce_{—}0778 (SEQ ID NO:31), Anabaena variabilis ATCC29413 YP_{—}323043 (Ava_{—}2533) (SEQ ID NO:33), and Synechococcus elongatus PCC6301 YP_{—}170760 (syc0050_d) (SEQ ID NO:35). Other alkane and alkene biosynthetic genes are listed in Table 1 and FIG. 38 of W02009/140646, expressly incorporated by reference herein.

Aldehyde biosynthetic genes include, for example, Synechococcus elongatus PCC7942 Synpcc7942_{—}1594 (SEQ ID NO:65), Synechocystis sp. PCC6803 sll0209 (SEQ ID NO:67), Cyanothece sp. ATCC51142 cce_{—}1430 (SEQ ID NO:69), Prochlorococcus marinus subsp. pastoris str. CCMP1986 PMM0533 (SEQ ID NO:71), Gloeobacter violaceus PCC7421 NP_{—}96091 (gll3145) (SEQ ID NO:73), Nostoc punctiforme PCC73102 ZP_{—}00108837 (Npun02004176) (SEQ ID NO:75), Anabaena variabilis ATCC29413 YP_{—}323044 (Ava_{—}2534) (SEQ ID NO:77), Synechococcus elongatus PCC6301 YP_{—}170761 (syc0051_d) (SEQ ID NO:79), and Nostoc sp. PCC 7120 alr5284 (SEQ ID NO:81). Other aldehyde biosynthetic genes are listed in Table 1 and FIG. 39 of W02009/140646, expressly incorporated by reference herein.

TABLE 1
Aldehyde and alkane biosynthetic gene homologs in cyanobacterial genomes

| | Alkane Biosynth. Gene | | Aldehyde Biosynth. Gene | |
|---|---|---|---|---|
| Cyanobacterium | accession number | % ID | accession number | % ID |
| Synechococcus elongatus PCC 7942 | YP_400610 | 100 | YP_400611 | 100 |
| Synechococcus elongatus PCC 6301 | YP_170760 | 100 | YP_170761 | 100 |
| Microcoleus chthonoplastes PCC 7420 | EDX75019 | 77 | EDX74978 | 70 |
| Arthrospira maxima CS-328 | EDZ94963 | 78 | EDZ94968 | 68 |
| Lyngbya sp. PCC 8106 | ZP_01619575 | 77 | ZP_01619574 | 69 |
| Nodularia spumigena CCY9414 | ZP_01628096 | 77 | ZP_01628095 | 70 |
| Trichodesmium erythraeum IMS101 | YP_721979 | 76 | YP_721978 | 69 |
| Microcystis aeruginosa NIES-843 | YP_001660323 | 75 | YP_001660322 | 68 |
| Microcystis aeruginosa PCC 7806 | CAO90780 | 74 | CAO90781 | 67 |
| Nostoc sp. PCC 7120 | NP_489323 | 74 | NP_489324 | 72 |
| Nostoc azollae 0708 | EEG05692 | 73 | EEG05693 | 70 |
| Anabaena variabilis ATCC 29413 | YP_323043 | 74 | YP_323044 | 73 |
| Crocosphaera watsonii WH 8501 | ZP_00514700 | 74 | ZP_00516920 | 67 |
| Synechocystis sp. PCC 6803 | NP_442147 | 72 | NP_442146 | 68 |
| Synechococcus sp. PCC 7335 | EDX86803 | 73 | EDX87870 | 67 |
| Cyanothece sp. ATCC 51142 | YP_001802195 | 73 | YP_001802846 | 67 |
| Cyanothece sp. CCY0110 | ZP_01728578 | 72 | ZP_01728620 | 68 |
| Nostoc punctiforme PCC 73102 | ZP_00108838 | 72 | ZP_00108837 | 71 |
| Acaryochloris marina MBIC11017 | YP_001518340 | 71 | YP_001518341 | 66 |
| Cyanothece sp. PCC 7425 | YP_002481151 | 71 | YP_002481152 | 70 |
| Cyanothece sp. PCC 8801 | ZP_02941459 | 70 | ZP_02942716 | 69 |
| Thermosynechococcus elongatus BP-1 | NP_682103 | 70 | NP_682102 | 70 |
| Synechococcus sp. JA-2-3B′a(2-13) | YP_478639 | 68 | YP_478638 | 63 |
| Synechococcus sp. RCC307 | YP_001227842 | 67 | YP_001227841 | 64 |
| Synechococcus sp. WH 7803 | YP_001224377 | 68 | YP_001224378 | 65 |
| Synechococcus sp. WH 8102 | NP_897829 | 70 | NP_897828 | 65 |
| Synechococcus sp. WH 7805 | ZP_01123214 | 68 | ZP_01123215 | 65 |
| uncultured marine type-A | ABD96376 | 70 | ABD96375 | 65 |
| Synechococcus GOM 3O12 | | | | |
| Synechococcus sp. JA-3-3Ab | YP_473897 | 68 | YP_473896 | 62 |
| uncultured marine type-A | ABD96328 | 70 | ABD96327 | 65 |
| Synechococcus GOM 3O6 | | | | |
| uncultured marine type-A | ABD96275 | 68 | ABD96274 | 65 |
| Synechococcus GOM 3M9 | | | | |
| Synechococcus sp. CC9311 | YP_731193 | 63 | YP_731192 | 63 |
| uncultured marine type-A | ABB92250 | 69 | ABB92249 | 64 |
| Synechococcus 5B2 | | | | |
| Synechococcus sp. WH 5701 | ZP_01085338 | 66 | ZP_01085337 | 67 |
| Gloeobacter violaceus PCC 7421 | NP_926092 | 63 | NP_926091 | 67 |
| Synechococcus sp. RS9916 | ZP_01472594 | 69 | ZP_01472595 | 66 |
| Synechococcus sp. RS9917 | ZP_01079772 | 68 | ZP_01079773 | 65 |
| Synechococcus sp. CC9605 | YP_381055 | 66 | YP_381056 | 66 |
| Cyanobium sp. PCC 7001 | EDY39806 | 64 | EDY38361 | 64 |
| Prochlorococcus marinus str. MIT 9303 | YP_001016795 | 63 | YP_001016797 | 66 |
| Prochlorococcus marinus str. MIT9313 | NP_895059 | 63 | NP_895058 | 65 |
| Synechococcus sp. CC9902 | YP_377637 | 66 | YP_377636 | 65 |
| Prochlorococcus marinus str. MIT 9301 | YP_001090782 | 62 | YP_001090783 | 62 |
| Synechococcus sp. BL107 | ZP_01469468 | 65 | ZP_01469469 | 65 |
| Prochlorococcus marinus str. AS9601 | YP_001008981 | 62 | YP_001008982 | 61 |
| Prochlorococcus marinus str. MIT9312 | YP_397029 | 62 | YP_397030 | 61 |
| Prochlorococcus marinus subsp. | NP_892650 | 60 | NP_892651 | 63 |
| pastoris str. CCMP1986 | | | | |
| Prochlorococcus marinus str. MIT 9211 | YP_001550420 | 61 | YP_001550421 | 63 |
| Cyanothece sp. PCC 7425 | YP_002483683 | 59 | — | |
| Prochlorococcus marinus str. NATL2A | YP_293054 | 59 | YP_293055 | 62 |
| Prochlorococcus marinus str. NATL1A | YP_001014415 | 59 | YP_001014416 | 62 |
| Prochlorococcus marinus subsp. | NP_874925 | 59 | NP_874926 | 64 |
| marinus str. CCMP1375 | | | | |
| Prochlorococcus marinus str. MIT | YP_001010912 | 57 | YP_001010913 | 63 |
| 9515_05961 | | | | |
| Prochlorococcus marinus str. MIT | YP_001483814 | 59 | YP_001483815 | 62 |
| 9215_06131 | | | | |
| Synechococcus sp. RS9917 | ZP_01080370 | 43 | — | |
| uncultured marine type-A | | | ABD96480 | 65 |
| Synechococcus GOM 5D20 | | | | |

Using the methods described herein, aldehydes, fatty alcohols, alkanes, and alkenes can be prepared using one or more aldehyde, alkane, and/or alkene biosynthetic genes or polypeptides described herein, or variants thereof, utilizing host cells or cell-free methods.

In some instances, alkanes and alkenes prepared using the methods described herein can be used to produce linear alkyl benzene and/or linear alkyl sulfonates, as described herein.

### Aldehyde, Alkane, and Alkene Biosynthetic Genes and Variants

The methods and compositions described herein include, for example, alkane or alkene biosynthetic genes having the nucleotide sequence of SEQ ID NO:1, 3, 5, 7, 9, 11, 13, 15, 17, 19, 21, 23, 25, 27, 29, 31, 33, or 35, as well as polynucleotide variants thereof. In some instances, the alkane or alkene biosynthetic gene encodes one or more of the amino acid motifs described herein. For example, the alkane or alkene biosynthetic gene can encode a polypeptide comprising SEQ ID NO:37, 38, 39, 41, 42, 43, or 44. The alkane or alkene biosynthetic gene can also include a polypeptide comprising SEQ ID NO:40 and also any one of SEQ ID NO:37, 38, or 39.

The methods and compositions described herein also include, for example, aldehyde biosynthetic genes having the nucleotide sequence of SEQ ID NO:65, 67, 69, 71, 73, 75, 77, 79, or 81, as well as polynucleotide variants thereof. In some instances, the aldehyde biosynthetic gene encodes one or more of the amino acid motifs described herein. For example, the aldehyde biosynthetic gene can encode a polypeptide comprising SEQ ID NO:54, 55, 56, 57, 58, 59, 60, 61, 62, 63, or 64.

The variants can be naturally occurring or created in vitro. In particular, such variants can be created using genetic engineering techniques, such as site directed mutagenesis, random chemical mutagenesis, Exonuclease III deletion procedures, and standard cloning techniques. Alternatively, such variants, fragments, analogs, or derivatives can be created using chemical synthesis or modification procedures.

Methods of making variants are well known in the art. These include procedures in which nucleic acid sequences obtained from natural isolates are modified to generate nucleic acids that encode polypeptides having characteristics that enhance their value in industrial or laboratory applications. In such procedures, a large number of variant sequences having one or more nucleotide differences with respect to the sequence obtained from the natural isolate are generated and characterized. Typically, these nucleotide differences result in amino acid changes with respect to the polypeptides encoded by the nucleic acids from the natural isolates.

For example, variants can be created using error prone PCR (see, e.g., Leung et al., Technique 1:11-15, 1989; and Caldwell et al., PCR Methods Applic. 2:28-33, 1992). In error prone PCR, PCR is performed under conditions where the copying fidelity of the DNA polymerase is low, such that a high rate of point mutations is obtained along the entire length of the PCR product. Briefly, in such procedures, nucleic acids to be mutagenized (e.g., an aldehyde or alkane biosynthetic polynucleotide sequence), are mixed with PCR primers, reaction buffer, MgCl₂, MnCl₂, Taq polymerase, and an appropriate concentration of dNTPs for achieving a high rate of point mutation along the entire length of the PCR product. For example, the reaction can be performed using 20 fmoles of nucleic acid to be mutagenized (e.g., an aldehyde or alkane biosynthetic polynucleotide sequence), 30 pmole of each PCR primer, a reaction buffer comprising 50 mM KCl, 10 mM Tris HCl (pH 8.3), and 0.01% gelatin, 7 mM MgCl₂, 0.5 mM MnCl₂, 5 units of Taq polymerase, 0.2 mM dGTP, 0.2 mM dATP, 1 mM dCTP, and 1 mM dTTP. PCR can be performed for 30 cycles of 94° C. for 1 min, 45° C. for 1 min, and 72° C. for 1 min. However, it will be appreciated that these parameters can be varied as appropriate. The mutagenized nucleic acids are then cloned into an appropriate vector and the activities of the polypeptides encoded by the mutagenized nucleic acids are evaluated.

Variants can also be created using oligonucleotide directed mutagenesis to generate site-specific mutations in any cloned DNA of interest. Oligonucleotide mutagenesis is described in, for example, Reidhaar-Olson et al., Science 241:53-57, 1988. Briefly, in such procedures a plurality of double stranded oligonucleotides bearing one or more mutations to be introduced into the cloned DNA are synthesized and inserted into the cloned DNA to be mutagenized (e.g., an aldehyde or alkane biosynthetic polynucleotide sequence). Clones containing the mutagenized DNA are recovered, and the activities of the polypeptides they encode are assessed.

Another method for generating variants is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, for example, U.S. Pat. No. 5,965,408.

Still another method of generating variants is sexual PCR mutagenesis. In sexual PCR mutagenesis, forced homologous recombination occurs between DNA molecules of different, but highly related, DNA sequence in vitro as a result of random fragmentation of the DNA molecule based on sequence homology. This is followed by fixation of the crossover by primer extension in a PCR reaction. Sexual PCR mutagenesis is described in, for example, Stemmer, PNAS, USA 91:10747-10751, 1994.

Variants can also be created by in vivo mutagenesis. In some embodiments, random mutations in a nucleic acid sequence are generated by propagating the sequence in a bacterial strain, such as an E. coli strain, which carries mutations in one or more of the DNA repair pathways. Such “mutator” strains have a higher random mutation rate than that of a wild-type strain. Propagating a DNA sequence (e.g., an aldehyde or alkane biosynthetic polynucleotide sequence) in one of these strains will eventually generate random mutations within the DNA. Mutator strains suitable for use for in vivo mutagenesis are described in, for example, PCT Publication No. WO 91/16427.

Variants can also be generated using cassette mutagenesis. In cassette mutagenesis, a small region of a double stranded DNA molecule is replaced with a synthetic oligonucleotide “cassette” that differs from the native sequence. The oligonucleotide often contains a completely and/or partially randomized native sequence.

Recursive ensemble mutagenesis can also be used to generate variants. Recursive ensemble mutagenesis is an algorithm for protein engineering (i.e., protein mutagenesis) developed to produce diverse populations of phenotypically related mutants whose members differ in amino acid sequence. This method uses a feedback mechanism to control successive rounds of combinatorial cassette mutagenesis. Recursive ensemble mutagenesis is described in, for example, Arkin et al., PNAS, USA 89:7811-7815, 1992.

In some embodiments, variants are created using exponential ensemble mutagenesis. Exponential ensemble mutagenesis is a process for generating combinatorial libraries with a high percentage of unique and functional mutants, wherein small groups of residues are randomized in parallel to identify, at each altered position, amino acids which lead to functional proteins. Exponential ensemble mutagenesis is described in, for example, Delegrave et al., Biotech. Res. 11:1548-1552, 1993. Random and site-directed mutagenesis are described in, for example, Arnold, Curr. Opin. Biotech. 4:450-455, 1993.

In some embodiments, variants are created using shuffling procedures wherein portions of a plurality of nucleic acids that encode distinct polypeptides are fused together to create chimeric nucleic acid sequences that encode chimeric polypeptides as described in, for example, U.S. Pat. Nos. 5,965,408 and 5,939,250.

Polynucleotide variants also include nucleic acid analogs. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine and 5-methyl-2′-deoxycytidine or 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered, morpholino ring, or peptide nucleic acids, in which the deoxyphosphate backbone is replaced by a pseudopeptide backbone and the four bases are retained. (See, e.g., Summerton et al., Antisense Nucleic Acid Drug Dev. (1997) 7:187-195; and Hyrup et al., Bioorgan. Med. Chem. (1996) 4:5-23.) In addition, the deoxyphosphate backbone can be replaced with, for example, a phosphorothioate or phosphorodithioate backbone, a phosphoroamidite, or an alkyl phosphotriester backbone.

The aldehyde and alkane biosynthetic polypeptides Synpcc7942_{—}1594 (SEQ ID NO:66) and Synpcc7942_{—}1593 (SEQ ID NO:2) have homologs in other cyanobacteria (nonlimiting examples are depicted in Table 1). Thus, any polynucleotide sequence encoding a homolog listed in Table 1, or a variant thereof, can be used as an aldehyde or alkane biosynthetic polynucleotide in the methods described herein. Each cyanobacterium listed in Table 1 has copies of both genes. The level of sequence identity of the gene products ranges from 61% to 73% for Synpcc7942_{—}1594 (SEQ ID NO:66) and from 43% to 78% for Synpcc7942_{—}1593 (SEQ ID NO:2).

Further homologs of the aldehyde biosynthetic polypeptide Synpcc7942_{—}1594 (SEQ ID NO:66) are listed in FIG. 39 of W02009/140646, expressly incorporated by reference herein, and any polynucleotide sequence encoding a homolog listed in FIG. 39 of W02009/140646, expressly incorporated by reference herein, or a variant thereof, can be used as an aldehyde biosynthetic polynucleotide in the methods described herein. Further homologs of the alkane biosynthetic polypeptide Synpcc7942_{—}1593 (SEQ ID NO:2) are listed in FIG. 38 of W02009/140646, expressly incorporated by reference herein, and any polynucleotide sequence encoding a homolog listed in FIG. 38 of W02009/140646, expressly incorporated by reference herein, or a variant thereof, can be used as an alkane biosynthetic polynucleotide in the methods described herein.

In certain instances, an aldehyde, alkane, and/or alkene biosynthetic gene is codon optimized for expression in a particular host cell. For example, for expression in E. coli, one or more codons can be optimized as described in, e.g., Grosjean et al., Gene 18:199-209 (1982).

### Aldehyde, Alkane, and Alkene Biosynthetic Polypeptides and Variants

The methods and compositions described herein also include alkane or alkene biosynthetic polypeptides having the amino acid sequence of SEQ ID NO:2, 4, 6, 8, 10, 12, 14, 16, 18, 20, 22, 24, 26, 28, 30, 32, 34, or 36, as well as polypeptide variants thereof. In some instances, an alkane or alkene biosynthetic polypeptide is one that includes one or more of the amino acid motifs described herein. For example, the alkane or alkene biosynthetic polypeptide can include the amino acid sequence of SEQ ID NO: 37, 38, 39, 41, 42, 43, or 44. The alkane or alkene biosynthetic polypeptide can also include the amino acid sequence of SEQ ID NO:40 and also any one of SEQ ID NO:37, 38, or 39.

The methods and compositions described herein also include aldehyde biosynthetic polypeptides having the amino acid sequence of SEQ ID NO:66, 68, 70, 72, 74, 76, 78, 80, or 82, as well as polypeptide variants thereof. In some instances, an aldehyde biosynthetic polypeptide is one that includes one or more of the amino acid motifs described herein. For example, the aldehyde biosynthetic polypeptide can include the amino acid sequence of SEQ ID NO:54, 55, 56, 57, 58, 59, 60, 61, 62, 63, or 64.

Aldehyde, alkane, and alkene biosynthetic polypeptide variants can be variants in which one or more amino acid residues are substituted with a conserved or non-conserved amino acid residue (preferably a conserved amino acid residue). Such substituted amino acid residue may or may not be one encoded by the genetic code.

Conservative substitutions are those that substitute a given amino acid in a polypeptide by another amino acid of similar characteristics. Typical conservative substitutions are the following replacements: replacement of an aliphatic amino acid, such as alanine, valine, leucine, and isoleucine, with another aliphatic amino acid; replacement of a serine with a threonine or vice versa; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue.

Other polypeptide variants are those in which one or more amino acid residues include a substituent group. Still other polypeptide variants are those in which the polypeptide is associated with another compound, such as a compound to increase the half-life of the polypeptide (e.g., polyethylene glycol).

Additional polypeptide variants are those in which additional amino acids are fused to the polypeptide, such as a leader sequence, a secretory sequence, a proprotein sequence, or a sequence which facilitates purification, enrichment, or stabilization of the polypeptide.

In some instances, an alkane or alkene biosynthetic polypeptide variant retains the same biological function as a polypeptide having the amino acid sequence of SEQ ID NO:2, 4, 6, 8, 10, 12, 14, 16, 18, 20, 22, 24, 26, 28, 30, 32, 34, or 36 (e.g., retains alkane or alkene biosynthetic activity) and has an amino acid sequence substantially identical thereto.

In other instances, the alkane or alkene biosynthetic polypeptide variants have at least about 50%, at least about 55%, at least about 60%, at least about 65%, at least about 70%, at least about 75%, at least about 80%, at least about 85%, at least about 90%, at least about 95%, or more than about 95% homology to the amino acid sequence of SEQ ID NO:2, 4, 6, 8, 10, 12, 14, 16, 18, 20, 22, 24, 26, 28, 30, 32, 34, or 36. In another embodiment, the polypeptide variants include a fragment comprising at least about 5, 10, 15, 20, 25, 30, 35, 40, 50, 75, 100, or 150 consecutive amino acids thereof.

In some instances, an aldehyde biosynthetic polypeptide variant retains the same biological function as a polypeptide having the amino acid sequence of SEQ ID NO:66, 68, 70, 72, 74, 76, 78, 80, or 82 (e.g., retains aldehyde biosynthetic activity) and has an amino acid sequence substantially identical thereto.

In yet other instances, the aldehyde biosynthetic polypeptide variants have at least about 50%, at least about 55%, at least about 60%, at least about 65%, at least about 70%, at least about 75%, at least about 80%, at least about 85%, at least about 90%, at least about 95%, or more than about 95% homology to the amino acid sequence of SEQ ID NO:66, 68, 70, 72, 74, 76, 78, 80, or 82. In another embodiment, the polypeptide variants include a fragment comprising at least about 5, 10, 15, 20, 25, 30, 35, 40, 50, 75, 100, or 150 consecutive amino acids thereof.

The polypeptide variants or fragments thereof can be obtained by isolating nucleic acids encoding them using techniques described herein or by expressing synthetic nucleic acids encoding them. Alternatively, polypeptide variants or fragments thereof can be obtained through biochemical enrichment or purification procedures. The sequence of polypeptide variants or fragments can be determined by proteolytic digestion, gel electrophoresis, and/or microsequencing. The sequence of the alkane or alkene biosynthetic polypeptide variants or fragments can then be compared to the amino acid sequence of SEQ ID NO:2, 4, 6, 8, 10, 12, 14, 16, 18, 20, 22, 24, 26, 28, 30, 32, 34, or 36 using any of the programs described herein. The sequence of the aldehyde biosynthetic polypeptide variants or fragments can be compared to the amino acid sequence of SEQ ID NO:66, 68, 70, 72, 74, 76, 78, 80, or 82 using any of the programs described herein.

The polypeptide variants and fragments thereof can be assayed for aldehyde-, fatty alcohol-, alkane-, and/or alkene-producing activity using routine methods. For example, the polypeptide variants or fragment can be contacted with a substrate (e.g., a fatty acid derivative substrate or other substrate described herein) under conditions that allow the polypeptide variant to function. A decrease in the level of the substrate or an increase in the level of an aldehyde, alkane, or alkene can be measured to determine aldehyde-, fatty alcohol-, alkane-, or alkene-producing activity, respectively.

### Anti-Aldehyde, Anti-Fatty Alcohol, Anti-Alkane, and Anti-Alkene Biosynthetic Polypeptide Antibodies

The aldehyde, fatty alcohol, alkane, and alkene biosynthetic polypeptides described herein can also be used to produce antibodies directed against aldehyde, fatty alcohol, alkane, and alkene biosynthetic polypeptides. Such antibodies can be used, for example, to detect the expression of an aldehyde, fatty alcohol, alkane, or alkene biosynthetic polypeptide using methods known in the art. The antibody can be, e.g., a polyclonal antibody; a monoclonal antibody or antigen binding fragment thereof; a modified antibody such as a chimeric antibody, reshaped antibody, humanized antibody, or fragment thereof (e.g., Fab′, Fab, F(ab′)₂); or a biosynthetic antibody, e.g., a single chain antibody, single domain antibody (DAB), Fv, single chain Fv (scFv), or the like.

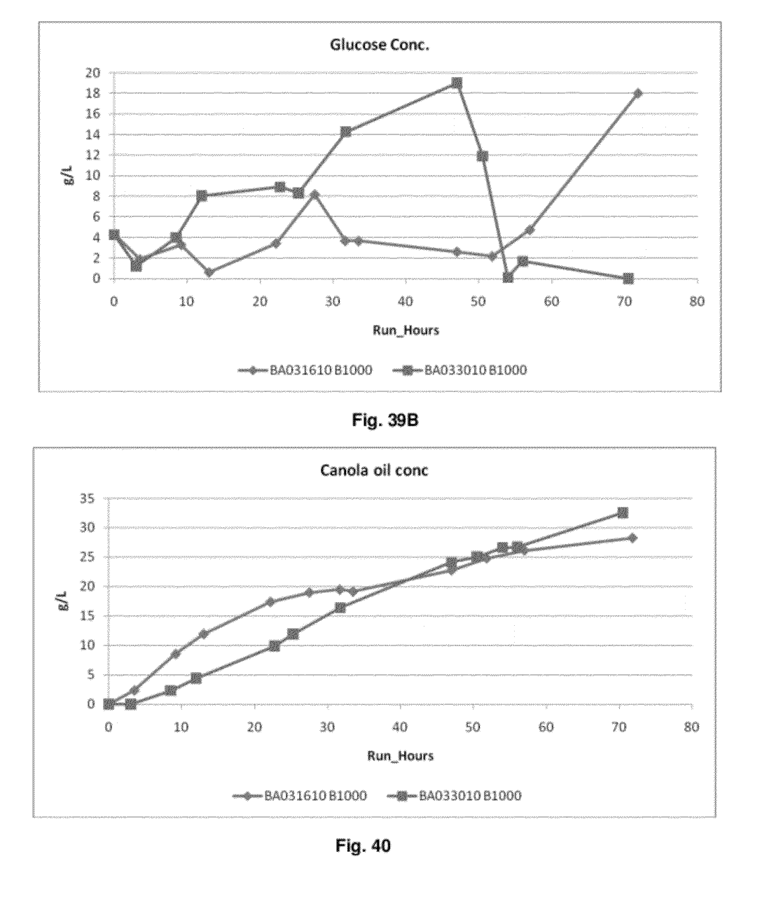

Accordingly, each step within a biosynthetic pathway that leads to the production of these substrates can be modified to produce or overproduce the substrate of interest. For example, known genes involved in the fatty acid biosynthetic pathway, the fatty aldehyde pathway, and the fatty alcohol pathway can be expressed, overexpressed, or attenuated in host cells to produce a desired substrate (see, e.g., PCT/US08/058,788, specifically incorporated by reference herein). Exemplary genes are provided in FIG. 40 of W02009/140646, expressly incorporated by reference herein.

Synthesis of Substrates

Fatty acid synthase (FAS) is a group of polypeptides that catalyze the initiation and elongation of acyl chains (Marrakchi et al., Biochemical Society, 30:1050-1055, 2002). The acyl carrier protein (ACP) along with the enzymes in the FAS pathway control the length, degree of saturation, and branching of the fatty acid derivatives produced. The fatty acid biosynthetic pathway involves the precursors acetyl-CoA and malonyl-CoA. The steps in this pathway are catalyzed by enzymes of the fatty acid biosynthesis (fab) and acetyl-CoA carboxylase (acc) gene families (see, e.g., Heath et al., Prog. Lipid Res. 40(6):467-97 (2001)).

Host cells can be engineered to express fatty acid derivative substrates by recombinantly expressing or overexpressing acetyl-CoA and/or malonyl-CoA synthase genes. For example, to increase acetyl-CoA production, one or more of the following genes can be expressed in a host cell: pdh, panK, aceEF (encoding the E1p dehydrogenase component and the E2p dihydrolipoamide acyltransferase component of the pyruvate and 2-oxoglutarate dehydrogenase complexes), fabH, fabD, fabG, acpP, and fabF. Exemplary GenBank accession numbers for these genes are: pdh (BAB34380, AAC73227, AAC73226), panK (also known as coaA, AAC76952), aceEF (AAC73227, AAC73226), fabH (AAC74175), fabD (AAC74176), fabG (AAC74177), acpP (AAC74178), fabF (AAC74179). Additionally, the expression levels of fadE, gpsA, ldhA, pflb, adhE, pta, poxB, ackA, and/or ackB can be attenuated or knocked-out in an engineered host cell by transformation with conditionally replicative or non-replicative plasmids containing null or deletion mutations of the corresponding genes or by substituting promoter or enhancer sequences. Exemplary GenBank accession numbers for these genes are: fadE (AAC73325), gspA (AAC76632), ldhA (AAC74462), pflb (AAC73989), adhE (AAC74323), pta (AAC75357), poxB (AAC73958), ackA (AAC75356), and ackB (BAB81430). The resulting host cells will have increased acetyl-CoA production levels when grown in an appropriate environment.

Malonyl-CoA overexpression can be effected by introducing accABCD (e.g., accession number AAC73296, EC 6.4.1.2) into a host cell. Fatty acids can be further overexpressed in host cells by introducing into the host cell a DNA sequence encoding a lipase (e.g., accession numbers CAA89087, CAA98876).

In addition, inhibiting PlsB can lead to an increase in the levels of long chain acyl-ACP, which will inhibit early steps in the pathway (e.g., accABCD, fabH, and fabI). The plsB (e.g., accession number AAC77011) D311E mutation can be used to increase the amount of available acyl-CoA.

In addition, a host cell can be engineered to overexpress a sfa gene (suppressor of fabA, e.g., accession number AAN79592) to increase production of monounsaturated fatty acids (Rock et al., J. Bacteriology 178:5382-5387, 1996).

In some instances, host cells can be engineered to express, overexpress, or attenuate expression of a thioesterase to increase fatty acid substrate production. The chain length of a fatty acid substrate is controlled by thioesterase. In some instances, a tes or fat gene can be overexpressed. In other instances, C₁₀ fatty acids can be produced by attenuating thioesterase C₁₈ (e.g., accession numbers AAC73596 and POADA1), which uses C_{18:1}-ACP, and expressing thioesterase C₁₀ (e.g., accession number Q39513), which uses C₁₀-ACP. This results in a relatively homogeneous population of fatty acids that have a carbon chain length of 10. In yet other instances, C₁₄ fatty acids can be produced by attenuating endogenous thioesterases that produce non-C₁₄ fatty acids and expressing the thioesterases, that use C₁₄-ACP (for example, accession number Q39473). In some situations, C₁₂ fatty acids can be produced by expressing thioesterases that use C₁₂-ACP (for example, accession number Q41635) and attenuating thioesterases that produce non-C₁₂ fatty acids. Acetyl-CoA, malonyl-CoA, and fatty acid overproduction can be verified using methods known in the art, for example, by using radioactive precursors, HPLC, and GC-MS subsequent to cell lysis. Non-limiting examples of thioesterases that can be used in the methods described herein are listed in Table 2.

TABLE 2
Thioesterases

| | | | Preferential |
|---|---|---|---|
| Accession | | | product |
| Number | Source Organism | Gene | produced |
| AAC73596 | E. coli | tesA without leader | C_{18:1} |
| | | sequence | |
| AAC73555 | E. coli | tesB | |
| Q41635, | Umbellularia california | fatB | C_{12:0} |
| AAA34215 | | | |
| Q39513; | Cuphea hookeriana | fatB2 | C_{8:0}-C_{10:0} |
| AAC49269 | | | |
| AAC49269; | Cuphea hookeriana | fatB3 | C_{14:0}-C_{16:0} |
| AAC72881 | | | |
| Q39473, | Cinnamonum camphorum | fatB | C_{14:0} |
| AAC49151 | | | |
| CAA85388 | Arabidopsis thaliana | fatB [M141T]* | C_{16:1} |
| NP189147; | Arabidopsis thaliana | fatA | C_{18:1} |
| NP193041 | | | |
| CAC39106 | Bradyrhiizobium | fatA | C_{18:1} |
| | japonicum | | |
| AAC72883 | Cuphea hookeriana | fatA | C_{18:1} |
| AAL79361 | Helianthus annus | fatA1 | |

*Mayer et al., BMC Plant Biology 7:1-11, 2007

### Saturation Levels

The degree of saturation in fatty acid derivatives can be controlled by regulating the degree of saturation of fatty acid derivative intermediates. The sfa, gns, and fab families of genes can be expressed or overexpressed to control the saturation of fatty acids. FIG. 40 of W02009/140646, expressly incorporated by reference herein, lists non-limiting examples of genes in these gene families that may be used in the methods and host cells described herein.

Host cells can be engineered to produce unsaturated fatty acids by engineering the host cell to overexpress fabB or by growing the host cell at low temperatures (e.g., less than 37° C.). FabB has preference to cis-δ3decenoyl-ACP and results in unsaturated fatty acid production in E. coli. Overexpression of fabB results in the production of a significant percentage of unsaturated fatty acids (de Mendoza et al., J. Biol. Chem. 258:2098-2101, 1983). The gene fabB may be inserted into and expressed in host cells not naturally having the gene. These unsaturated fatty acid derivatives can then be used as intermediates in host cells that are engineered to produce fatty acid derivatives, such as fatty aldehydes, fatty alcohols, or alkenes.

Other Substrates

Other substrates that can be used to produce aldehydes, fatty alcohols, alkanes, and alkenes in the methods described herein are acyl-ACP, acyl-CoA, a fatty aldehyde, or a fatty alcohol, which are described in, for example, PCT/US08/058,788. Exemplary genes that can be altered to express or overexpress these substrates in host cells are listed in FIG. 40 of W02009/140646, expressly incorporated by reference herein. Other exemplary genes are described in PCT/US08/058,788.

### Genetic Engineering of Host Cells to Produce Aldehydes, Fatty Alcohols, Alkanes, and Alkenes

Various host cells can be used to produce aldehydes, fatty alcohols, alkanes, and/or alkenes, as described herein. A host cell can be any prokaryotic or eukaryotic cell. For example, a polypeptide described herein can be expressed in bacterial cells (such as E. coli), insect cells, yeast or mammalian cells.

Other exemplary host cells include cells from the members of the genus Escherichia, Bacillus, Lactobacillus, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Schizosaccharomyces, Yarrowia, or Streptomyces. Yet other exemplary host cells can be a Bacillus lentus cell, a Bacillus brevis cell, a Bacillus stearothermophilus cell, a Bacillus licheniformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, a Bacillus amyloliquefaciens cell, a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhizomucor miehei cell, a Mucor michei cell, a Streptomyces lividans cell, a Streptomyces murinus cell, or an Actinomycetes cell.

Other nonlimiting examples of host cells are those listed in Table 1.

In a preferred embodiment, the host cell is an E. coli cell. In a more preferred embodiment, the host cell is from E. coli strains B, C, K, or W. Other suitable host cells are known to those skilled in the art.

Various methods well known in the art can be used to genetically engineer host cells to produce aldehydes, fatty alcohols, alkanes and/or alkenes. The methods include the use of vectors, preferably expression vectors, containing a nucleic acid encoding an aldehyde, fatty alcohol, alkane, and/or alkene biosynthetic polypeptide described herein, or a polypeptide variant or fragment thereof. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell and are thereby replicated along with the host genome. Moreover, certain vectors, such as expression vectors, are capable of directing the expression of genes to which they are operatively linked.

The recombinant expression vectors described herein include a nucleic acid described herein in a form suitable for expression of the nucleic acid in a host cell. The recombinant expression vectors can include one or more control sequences, selected on the basis of the host cell to be used for expression. The control sequence is operably linked to the nucleic acid sequence to be expressed. Recombinant expression vectors can be designed for expression of an aldehyde, fatty alcohol, alkane, and/or alkene biosynthetic polypeptide or variant in prokaryotic or eukaryotic cells, e.g., bacterial cells, such as E. coli, insect cells (using baculovirus expression vectors), yeast cells, or mammalian cells. Alternatively, the recombinant expression vector can be transcribed and translated in vitro, for example, by using T7 promoter regulatory sequences and T7 polymerase.

Expression of polypeptides in prokaryotes, for example, E. coli, is most often carried out with vectors containing constitutive or inducible promoters directing the expression of either fusion or non-fusion polypeptides.

In another embodiment, the host cell is a yeast cell. In this embodiment, the expression vector is a yeast expression vector. Examples of vectors for expression in yeast S. cerevisiae include pYepSec1 (Baldari et al., EMBO J. (1987) 6:229-234), pMFa (Kurjan et al., Cell (1982) 30:933-943), pJRY88 (Schultz et al., Gene (1987) 54:113-123), pYES2 (Invitrogen Corporation, San Diego, Calif.), and picZ (Invitrogen Corp, San Diego, Calif.).

Alternatively, a polypeptide described herein can be expressed in insect cells using baculovirus expression vectors. Baculovirus vectors available for expression of proteins in cultured insect cells (e.g., Sf 9 cells) include, for example, the pAc series (Smith et al., Mol. Cell Biol. (1983) 3:2156-2165) and the pVL series (Lucklow et al., Virology (1989) 170:31-39).

Vectors can be introduced into prokaryotic or eukaryotic cells via conventional transformation or transfection techniques. As used herein, the terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in, for example, Sambrook et al. (supra).

For stable transformation of bacterial cells, a gene that encodes a selectable marker (e.g., resistance to antibiotics) can be introduced into the host cells along with the gene of interest. Selectable markers include those that confer resistance to drugs, such as ampicillin, kanamycin, chloramphenicol, or tetracycline. Nucleic acids encoding a selectable marker can be introduced into a host cell on the same vector as that encoding a polypeptide described herein or can be introduced on a separate vector. Cells stably transfected with the introduced nucleic acid can be identified by drug selection (e.g., cells that have incorporated the selectable marker gene will survive, while the other cells die).

In certain methods, an aldehyde biosynthetic polypeptide and an alkane or alkene biosynthetic polypeptide are co-expressed in a single host cell. In alternate methods, an aldehyde biosynthetic polypeptide and an alcohol dehydrogenase polypeptide are co-expressed in a single host cell.

### Transport Proteins

Transport proteins can export polypeptides and hydrocarbons (e.g., aldehydes, alkanes, and/or alkenes) out of a host cell. Many transport and efflux proteins serve to excrete a wide variety of compounds and can be naturally modified to be selective for particular types of hydrocarbons.

Non-limiting examples of suitable transport proteins are ATP-Binding Cassette (ABC) transport proteins, efflux proteins, and fatty acid transporter proteins (FATP). Additional non-limiting examples of suitable transport proteins include the ABC transport proteins from organisms such as Caenorhabditis elegans, Arabidopsis thalania, Alkaligenes eutrophus, and Rhodococcus erythropolis. Exemplary ABC transport proteins that can be used are listed in FIG. 40 of W02009/140646, expressly incorporated by reference herein (e.g., CER5, AtMRP5, AmiS2, and AtPGP1). Host cells can also be chosen for their endogenous ability to secrete hydrocarbons. The efficiency of hydrocarbon production and secretion into the host cell environment (e.g., culture medium, fermentation broth) can be expressed as a ratio of intracellular product to extracellular product. In some examples, the ratio can be about 5:1, 4:1, 3:1, 2:1, 1:1, 1:2, 1:3, 1:4, or 1:5.

### Fermentation

The production and isolation of aldehydes, fatty alcohols, alkanes and/or alkenes can be enhanced by employing beneficial fermentation techniques. One method for maximizing production while reducing costs is increasing the percentage of the carbon source that is converted to hydrocarbon products.

The percentage of input carbons converted to aldehydes, fatty alcohols, alkanes and/or alkenes can be a cost driver. The more efficient the process is (i.e., the higher the percentage of input carbons converted to aldehydes, fatty alcohols, alkanes and/or alkenes), the less expensive the process will be. Host cells engineered to produce aldehydes, alkanes and/or alkenes can have greater than about 1, 3, 5, 10, 15, 20, 25, and 30% efficiency.

The host cell can be additionally engineered to express recombinant cellulases, such as those described in WO 2010127318, expressly incorporated by reference herein. These cellulases allow the host cell to use cellulosic material as a carbon source. For example, the host cell can be additionally engineered to express invertases (EC 3.2.1.26) so that sucrose can be used as a carbon source. Similarly, the host cell can be engineered using the teachings described in U.S. Pat. Nos. 5,000,000; 5,028,539; 5,424,202; 5,482,846; and 5,602,030; so that the host cell can assimilate carbon efficiently and use cellulosic materials as carbon sources.

For small scale production, the engineered host cells can be grown in batches of, for example, around 100 mL, 500 mL, 1 L, 2 L, 5 L, or 10 L; fermented; and induced to express desired aldehydes, fatty alcohols, alkanes and/or alkenes. For large scale production, the engineered host cells can be grown in batches of 10 L, 100 L, 1000 L, or larger; fermented; and induced to express desired aldehydes, fatty alcohols, alkanes and/or alkenes. For example, E. coli BL21(DE3) cells harboring pBAD24 (with ampicillin resistance and the aldehyde and/or alkane synthesis pathway) as well as pUMVC1 (with kanamycin resistance and the acetyl-CoA/malonyl-CoA overexpression system) can be incubated from a 500 mL seed culture for 10 L fermentations (5 L for 100 L fermentations, etc.) in LB media (glycerol free) with 50 μg/mL kanamycin and 75 μg/mL ampicillin at 37° C., and shaken at >200 rpm until cultures reach an OD₆₀₀ of >0.8 (typically 16 hrs). Media can be continuously supplemented to maintain 25 mM sodium proprionate (pH 8.0) to activate the engineered gene systems for production and to stop cellular proliferation by activating umuC and umuD proteins. Media can be continuously supplemented with glucose to maintain for example, a concentration 25 g/100 mL.

After induction, aliquots can be removed from the cell culture and allowed to sit without agitation to allow the aldehydes, alkanes and/or alkenes to rise to the surface and undergo a spontaneous phase separation. The aldehyde, fatty alcohols, alkane and/or alkene component can then be collected, and the aqueous phase returned to the reaction chamber. The reaction chamber can be operated continuously.

### Producing Aldehydes, Fatty Alcohols, Alkanes and Alkenes Using Cell-Free Methods

In some methods described herein, an aldehyde, fatty alcohols, alkane and/or alkene can be produced using a purified polypeptide described herein and a substrate described herein. For example, a host cell can be engineered to express aldehyde, fatty alcohols, alkane and/or alkene biosynthetic polypeptide or variant as described herein. The host cell can be cultured under conditions suitable to allow expression of the polypeptide. Cell free extracts can then be generated using known methods. For example, the host cells can be lysed using detergents or by sonication. The expressed polypeptides can be purified using known methods. After obtaining the cell free extracts, substrates described herein can be added to the cell free extracts and maintained under conditions to allow conversion of the substrates to aldehydes, fatty alcohols, alkanes and/or alkenes. The aldehydes, fatty alcohols, alkanes and/or alkenes can then be separated and purified using known techniques.

### Post-Production Processing

The aldehydes, fatty alcohols, alkanes and/or alkenes produced during fermentation can be separated from the fermentation media. Any known technique for separating aldehydes, fatty alcohols, alkanes and/or alkenes from aqueous media can be used. One exemplary separation process is a two phase (bi-phasic) separation process. This process involves fermenting the genetically engineered host cells under conditions sufficient to produce an aldehyde, fatty alcohols, alkane and/or alkene, allowing the aldehyde, fatty alcohols, alkane and/or alkene to collect in an organic phase, and separating the organic phase from the aqueous fermentation broth. This method can be practiced in both a batch and continuous fermentation setting.

Bi-phasic separation uses the relative immiscibility of aldehydes, fatty alcohols, alkanes and/or alkenes to facilitate separation. Immiscible refers to the relative inability of a compound to dissolve in water and is defined by the compound's partition coefficient. One of ordinary skill in the art will appreciate that by choosing a fermentation broth and organic phase, such that the aldehyde, alkane and/or alkene being produced has a high log P value, the aldehyde, alkane and/or alkene can separate into the organic phase, even at very low concentrations, in the fermentation vessel.

The aldehydes, fatty alcohols, alkanes and/or alkenes produced by the methods described herein can be relatively immiscible in the fermentation broth, as well as in the cytoplasm. Therefore, the aldehyde, fatty alcohols, alkane and/or alkene can collect in an organic phase either intracellularly or extracellularly. The collection of the products in the organic phase can lessen the impact of the aldehyde, fatty alcohols, alkane and/or alkene on cellular function and can allow the host cell to produce more product.

The methods described herein can result in the production of homogeneous compounds wherein at least about 60%, 70%, 80%, 90%, or 95% of the aldehydes, fatty alcohols, alkanes and/or alkenes produced will have carbon chain lengths that vary by less than about 6 carbons, less than about 4 carbons, or less than about 2 carbons. These compounds can also be produced with a relatively uniform degree of saturation. These compounds can be used directly as fuels, fuel additives, specialty chemicals, starting materials for production of other chemical compounds (e.g., polymers, surfactants, plastics, textiles, solvents, adhesives, etc.), or personal care product additives. These compounds can also be used as feedstock for subsequent reactions, for example, hydrogenation, catalytic cracking (via hydrogenation, pyrolisis, or both), to make other products.

In some embodiments, the aldehydes, fatty alcohols, alkanes and/or alkenes produced using methods described herein can contain between about 50% and about 90% carbon; or between about 5% and about 25% hydrogen. In other embodiments, the aldehydes, fatty alcohols, alkanes and/or alkenes produced using methods described herein can contain between about 65% and about 85% carbon; or between about 10% and about 15% hydrogen.

### Production of Linear Alkyl Benzene (LAB)

The alkylation of aromatic hydrocarbons such as benzene is practiced commercially using solid catalysts in large scale industrial units. The alkylation of benzene with olefins having from 8 to 28 carbons produces alkylbenzenes that have various commercial uses. One use is to sulfonate the alkylbenzenes to produced sulfonated alkylbenzenes for use as detergents. The alkylation process can occur by reacting benzene with an olefin in the presence of a catalyst at an elevated temperature and pressure.

The alkylation may rely on a process that uses two feedstocks, a substantially linear (non-branched) olefin and an aryl compound. The linear olefin can be a mixture of linear olefins with double bonds at terminal and internal positions or a linear alpha olefin with double bonds located at terminal positions. For example, the olefin can be an olefin produced by a method described herein or produced by a method described in, e.g., WO 2008/147781 or WO 2009/085278 (both of which are specifically incorporated by reference herein). Preferably the aryl compound is benzene.

The linear olefin can comprise a molecule having from 8 to 28 carbon atoms, such as from 8 to 15 carbon atoms or from 10 to 14 carbon atoms. The olefin and aryl compounds are reacted in the presence of a catalyst under reaction conditions. The catalyst can comprise a layered composition having an inner core and an outer layer bonded to the inner core. The outer layer can include a molecular sieve and a binder.

The reaction conditions for alkylation can be selected to minimize isomerization of the alkyl group and minimize polyalkylation of the benzene, while trying to maximize the consumption of the olefins to maximize product. Alkylation conditions can include a reaction temperature from about 50° C. to about 200° C., such as from about 80° C. to about 175° C. The pressures in the reactor can be from about 1.4 MPa (203 psia) to about 7 MPa (1015 psia), such as from 2 MPa (290 psia) to 3.5 MPa (507 psia). To minimize polyalkylation of the benzene, the aryl to monoolefin molar ratio can be from about 2.5:1 to about 50:1, such as from about 5:1 to about 35:1. The average residence time in the reactor can contribute to product quality, and the process can be operated at a liquid hourly space velocity (LHSV) from about 0.1 to about 30 hr⁻¹, such as from 0.3 to 6 hr⁻¹.

The olefins can be produced from the dehydrogenation of paraffins, cracking of paraffins and subsequent oligomerization of smaller olefinic molecules, or other known processes for the production of linear monoolefins. The separation of linear paraffins from a mixture comprising normal paraffins, isoparaffins and cycloparaffins for dehydrogenation can include the use of known separation processes, such as the use of UOP Sorbex separation technology. UOP Sorbex technology can also be used to separate linear olefins from a mixture of linear and branched olefins.

One method for the production of a paraffinic feedstock is the separation of linear (nonbranched) hydrocarbons or lightly branched hydrocarbons from a kerosene boiling range petroleum fraction. Several known processes that accomplish such a separation are known. One process, the UOP MoleX™ process, is an established, commercially proven method for the liquid-phase adsorption separation of normal paraffins from isoparaffins and cycloparaffins using the UOP Sorbex separation technology.

Paraffins can also be produced in a gas to liquids (GTL) process, where synthesis gas made up of CO and H₂ at a controlled stoichiometry are reacted to form larger paraffinic molecules. The resulting paraffinic mixture can then be separated into normal paraffins and non-normal paraffins, with the normal paraffins dehydrogenated to produce substantially linear olefins.

In the process of producing olefins from paraffins, by products include diolefins and alkynes, or acetylenes. The streams comprising diolefins and acetylenes can be passed to a selective hydrogenation reactor, where the diolefins and alkynes can be converted to olefins.

Alkylbenzenes can be used as a base chemical for surfactant based detergents. The alkylbenzenes can be typically sulfonated to produce the surfactants. However, branched alkylbenzenes have poor biodegradability and create foam in rivers and lakes where the detergents wash into. Having a biodegradable detergent has a less adverse affect on the environment, and linear alkylbenzenes are much more biodegradable and consequently have a lower environmental impact. Reducing the amount of branching produces a higher quality base product for use in detergents.

In detergent alkylation, skeletal isomerization of the olefin is kinetically controlled and not desirable. As a result, skeletal isomerization can be sensitive to operating conditions such as temperature and relative amounts of catalyst in the reactor. In contrast, alkylation is predominantly diffusion controlled and thus not as sensitive to the relative amounts of catalyst as the isomerization reaction in the reactor. By layering the catalyst, the isomerization can be suppressed without sacrificing the alkylation performance. This improves the linearity of the alkylbenzene, which is one measure of LAB product quality, (greater linearity is perceived as higher quality). In addition, the operating temperatures can be increased to improve catalyst reactivity, and stability, while maintaining product linearity.

By “skeletal isomerization” of an alkyl group is meant isomerization that increases the number of primary carbon atoms of the alkyl group. The skeletal isomerization of the alkyl group increases the number of methyl group branches of the aliphatic alkyl chain. Because the total number of carbon atoms of the alkyl group remains the same, each additional methyl group branch causes a corresponding reduction by one of the number of carbon atoms in the aliphatic alkyl chain.

A catalyst can comprise an inner core composed of a material that has substantially lower isomerization reactivity relative to the outer layer. Some of the inner core materials are also not substantially penetrated by liquids. Examples of the inner core material include, but are not limited to, refractory inorganic oxides, silicon carbide, and metals. Examples of refractory inorganic oxides include, without limitation, alpha alumina, cordierite, magnesia, metals, silicon carbide, theta alumina, titania, zirconia, and mixtures thereof. Inorganic oxides can be alumina of various crystalline phases and cordierite.

The materials that form the inner core can be formed into a variety of shapes such as pellets, extrudates, spheres, or irregularly shaped particles, although not all materials can be formed into each shape. The inner core can be prepared by any means known in the art such as oil dropping, pressure molding, metal forming, pelletizing, granulation, extrusion, rolling methods, and marumerizing. In certain embodiments, the inner core is spherical.

The inner core can have an effective diameter of about 0.05 mm (0.0020 in) to about 5 mm (0.2 in), such as from about 0.8 mm (0.031 in) to about 3 mm (0.12 in). For a non-spherical inner core, effective diameter is defined as the diameter the shaped article would have if it were molded into a sphere. Once the inner core is prepared, it can be calcined at a temperature of from about 400° C. (752° F.) to about 1800° C. (3272° F.). When the inner core comprises cordierite, it can be calcined at a temperature of from about 1000° C. (1832° F.) to about 1800° C. (3272° F.).

The outer layer of the catalyst can be applied by forming a slurry of the molecular sieve material and then coating the inner core with the slurry by any means known in the art. The slurry can include an organic bonding agent that aids in the adhesion of the molecular sieve material to the inner core. Examples of the organic bonding agent include, but are not limited to, polyvinyl alcohol (PVA), hydroxy propyl cellulose, methyl cellulose, and carboxy methyl cellulose. The bonding agent can be present in the slurry in an amount of between about 0.1 wt % and about 3 wt %, which can be consumed during the calcination of the catalyst. The outer layer can further include a binder that is resistant to temperature and reaction conditions while providing hardness and attrition resistance.

Molecular sieves that can be used include, but are not limited to, zeolites such as UZM-8, Faujasite, beta, MTW, MOR, LTL, MWW, EMT, UZM-4 and mixtures thereof. UZM-4 is a silica alumina version of the BPH structure and has the substantial acidity needed for the alkylation reaction. The binders used are inorganic metal oxides and examples include, but are not limited to, alumina, silica, magnesia, titania, zirconia, and mixtures thereof.

The inner core can be coated with the slurry by any means known in the art, such as rolling, dipping, spraying, etc. One technique includes spraying the slurry into a fluidized bed of inner core particles. This procedure coats the particles in a fairly uniform manner and provides for a thickness of the layer from between about 10 and about 300 micrometers. The thickness can be controlled by time and other operating parameters. The coated particles can then be dried at a temperature from about 100° C. (212° F.) to about 300° C. (572° F.) for a time from about 1 to about 24 hours and then calcined at a temperature from about 400° C. (752° F.) to about 900° C. (1652° F.) for a time from about 0.5 to about 10 hours to effectively bond the outer layer to the inner core and provide a layered catalyst. For operating efficiency, the drying and calcining steps can be combined into one step.

### Surfactants or Detersive Surfactants

An alkylbenzene, such as a sulfonated alkylbenzene, produced as described herein can be used in surfactant compositions, which can comprise about 0.001 wt. % to about 100 wt. % of an alkylbenzene described herein. Preferably, a surfactant composition is a blend of an alkylbenzene in combination with one or more other surfactants and/or surfactant systems that have been derived from similar (e.g., microbially derived) or different sources (e.g., synthetic, petroleum-derived). Those other surfactants and/or surfactant systems can confer additional desirable properties. In some embodiments, the one or more other surfactants and/or surfactant systems that are blended with the alkylbenzene can comprise linear or branched fatty alcohol derivatives, or they can be other types of surfactants such as, cationic surfactants, anionic surfactants and/or amphoteric/zwitterionic surfactants. These other surfactants and/or surfactants systems are collectively referred to as “co-surfactants” herein. For example, a surfactant composition of the invention can be a blend of an alkylbenzene prepared in accordance with the disclosure herein, and a cationic surfactant derived from a petrochemical source, and the resulting surfactant composition only has good cleaning properties but also contributes certain disinfecting and/sanitizing benefits.

The cleaning composition of the invention can comprise, in addition to an alkylbenzene described herein, co-surfactants selected from nonionic surfactants, anionic surfactants, cationic surfactants, ampholytic surfactants, squitterionic surfactants, semi-polar nonionic surfactants, and mixtures thereof. When present, the total amount of surfactants, including the alkylbenzene and the co-surfactants, is typically present at a level of about 0.1 wt. % or higher (e.g., about 1.0 wt. % or higher, about 10 wt. % or higher, about 25 wt. % or higher, about 50 wt. % or higher, about 70 wt. % or higher). For example, the total amount of surfactant in a cleaning composition can vary from about 0.1 wt. % to about 80 wt. % (e.g., from about 0.1 wt. % to about 40 wt. %, from about 0.1 wt % to about 12 wt. %, from about 1.0 wt. % to about 50 wt. %, or from about 5 wt. % to about 40 wt. %).

Various known surfactants can be suitable co-surfactants. In some embodiments, the co-surfactant can comprise an anionic surfactant. In certain embodiments, the amount of one or more anionic surfactants in the cleaning composition can be, for example, about 1 wt. % or more (e.g., about 5 wt. % or more, about 10 wt. % or more, about 20 wt. % or more, about 30 wt. % or more, about 40 wt. % or more). For example, the amount of one or more anionic surfactants in the cleaning composition can vary from about 1 wt. % to about 40 wt. %. Suitable anionic surfactants include, for example, linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl esters, alkyl- or alkenylsuccinic acid or soap. In some embodiments, an anionic surfactant can be selected from, for example, a C₁₀-C₁₈ alkyl akoxy es (AEₓS) wherein x is from 1-30. Other suitable anionic surfactants can be found in WO98/39403, Surface Active Agents and Detergents (Vol. 1, & II, by Schwartz, Perry and Berch), and U.S. Pat. Nos. 3,929,678, 6,020,303, 6,060,443, 6,008,181, International Publications WO 99/05243, WO 99/05242 and WO 99/05244, which are incorporated herein by reference.

In another embodiment, the co-surfactant can comprise a cationic surfactant. Suitable cationic surfactants include, for example, those having long-chain hydrocarbyl groups. Examples include the ammonium surfactants such as alkyltrimethylammonium halogenides, and those surfactants having the formula [R²(OR³)y][R⁴(OR³)y]₂R⁵N+X⁻, wherein R² is an alkyl or alkyl benzyl group having from about 8 to about 18 carbon atoms in the alkyl chain, each R³ is selected from the group consisting of —CH₂CH₂—, CH₂CH(CH₃)—, CH₂(CH(CH₂OH)—, CH₂CH₂CH₂—, and mixtures thereof; each R⁴ is selected from the group consisting of C₁-C₄ alkyl, C₁-C₄ hydroxyalkyl, benzyl ring structures formed by joining the two R⁴ groups, —CH₂CHOH—CHOHCOR⁶CHOHCH₂OH wherein R⁶ is any hexose or hexose polymer having a molecular weight less than about 1000, and hydrogen when y is not 0; R⁵ is the same as R₄ or is an alkyl chain wherein the total number of carbon atoms of R² plus R⁵ is not more than about 18; each y is from 0 to about 10 and the sum of the y values is from 0 to about 15; and X is any compatible anion.

Certain quaternary ammonium surfactants may also be suitable as cationic co-surfactants, and examples of those are described in WO 98/39403. Examples of suitable quaternary ammonium compounds include coconut trimethyl ammonium chloride or bromide; coconut methyl dihydroxyethyl ammonium chloride or bromide; decyl triethyl ammonium chloride; decyl di methyl hydroxyethyl ammonium chloride or bromide; C₁₂₋₁₅ dimethyl hydroxyethyl ammonium chloride or bromide; coconut dimethyl hydroxyethyl ammonium chloride or bromide; myristyl trimethyl ammonium methyl sulphate; lauryl dimethyl benzyl ammonium chloride or bromide; lauryl di methyl(ethenoxy) 4 ammonium chloride or bromide. Other cationic surfactants have been described in U.S. Pat. Nos. 4,228,044, 4,228,042, 4,239,660 4,260,529 6,136,769, 6,004,922, 6,022,844, and 6,221,825, International Publications WO 98/35002, WO 98/35003, WO 98/35004, WO 98/35005, WO 98/35006, and WO 00/47708, as well as European Patent Application EP 000,224. When included herein, the cleaning compositions of the present invention can comprise, for example, from about 0.2 wt. % to about 25 wt. %, preferably from about 1 wt. % to about 8 wt. % by weight of cationic surfactants.

In certain embodiments, suitable co-surfactants can comprise nonionic surfactants. Polyethylene, polypropylene, and polybutylene oxide condensates of alkyl phenols are suitable, with the polyethylene oxide condensates being preferred. These compounds include the condensation products of alkyl phenols having an alkyl group containing from about 6 to about 14 carbon atoms, preferably from about 8 to about 14 carbon atoms, in either a straight-chain or branched-chain configuration with the alkylene oxide. In a preferred embodiment, the ethylene oxide is present in an amount of from about 2 to about 25 moles (e.g., from about 3 to about 15 moles) of ethylene oxide per mole of alkyl phenol. Commercially available nonionic surfactants of this type include Igepal™ C0-630 (The GAF Corporation), Triton™ X-45, X-114, X-100 and X-102 (Dow Chemicals). These surfactants are commonly referred to as alkylphenol alkoxylates (e.g., alkyl phenol ethoxylates).

Moreover, condensation products of primary and secondary aliphatic alcohols with from about 1 to about 25 moles of ethylene oxide are suitable nonionic co-surfactants. The alkyl chain of the aliphatic alcohol can either be straight or branched, primary or secondary, and generally contains from about 8 to about 22 carbon atoms (e.g., about 8 to about 20 carbon atoms, from about 10 to about 18 carbon atoms) with about 2 to about 10 moles (e.g., about 2 to about 5 moles) of ethylene oxide per mole of alcohol present in the condensation products. Examples of commercially available nonionic surfactants of this type include Tergitol™ 15-S-9, Tergitol™ 24-L-6 NMW (Union Carbide); Neodol™ 45-9, Neodol™ 23-3, Neodol™ 45-7, Neodol™ 45-5 (Shell Chemical), Kyro™ EOB (Procter & Gamble), and Genapol LA 030 or 050 (Hoechst).

Further examples of nonionic co-surfactants can be C₁₂-C₁₈ alkyl ethoxylates (e.g., NEODOL® nonionic surfactants (shell)), C₆-C₁₂ alkyl phenol alkoxylates wherein the alkoxylate units are a mixture of ethyleneoxy and propyleneoxy units, C₁₂-C₁₈ alcohol and C₆-C₁₂ alkyl phenol condensates with ethylene oxide/propylene oxide block alkyl polyamine ethoxylates (e.g., PLURONIC® (BASF)), C₁₄-C₂₂ mid-chain branched alcohols as described in U.S. Pat. No. 6,150,322, C₁₄-C₂₂ mid-chain branched alkyl alkoxylates, BAEₓ, wherein x is from 1-30, as described in U.S. Pat. Nos. 6,153,577, 6,020,303 and 6,093,856, alkylpolysaccharides as described in U.S. Pat. No. 4,565,647, alkylpolyglycosides as described in U.S. Pat. No. 4,483,780 and U.S. Pat. No. 4,483,779, polyhydroxy detergent acid amides as described in U.S. Pat. No. 5,332,528, or ether capped poly(oxyalkylated) alcohol surfactants as described in U.S. Pat. No. 6,482,994 and International Patent WO 01/42408.

Semi-polar nonionic surfactants can also be suitable as co-surfactants, including, without limitation, water-soluble amine oxides containing 1 alkyl moiety of from about 10 to about 18 carbon atoms and 2 moieties selected from alkyl or hydroxyalkyl moieties containing about 1 to about 3 carbon atoms, water-soluble phosphine oxides containing 1 alkyl moiety of about 10 to about 18 carbon atoms and 2 moieties selected from alkyl or hydroxyalkyl moieties containing about 1 to about 3 carbon atoms; and water-soluble sulfoxides containing 1 alkyl moiety of about 10 to about 18 carbon atoms and a moiety selected from alkyl or hydroxyalkyl moieties of about 1 to about 3 carbon atoms. These semi-polar nonionic surfactants have been described in, for example, International Publication WO 01/32816, and U.S. Pat. Nos. 4,681,704 and 4,133,779.

Moreover, alkylpolysaccharides, such as those described in U.S. Pat. No. 4,565,647, having a hydrophobic group containing about 6 to about 30 carbon atoms (e.g., from about 10 to about 16 carbon atoms) and a polysaccharide can also be suitable semi-polar nonionic co-surfactants. Others have been described in, for example, International Publication WO 98/39403. When included herein, the cleaning compositions of the present invention can comprise, for example, about 0.2 wt. % or more (e.g., about 1 wt. % or more, about 5 wt. % or more, or about 8 wt. % or more) of such semi-polar nonionic surfactants. For example, the cleaning compositions of the invention can comprise about 0.2 wt. % to about 15 wt. % (e.g., about 1 wt. % to about 10 wt. %) of semi-polar nonionic surfactants.

In certain embodiments, the co-surfactants comprises ampholytic surfactants. Ampholytic surfactants can be broadly described as aliphatic derivatives of secondary or tertiary amines, or aliphatic derivatives of heterocyclic secondary and tertiary amines in which the aliphatic radical can be straight- or branched-chain. One of the aliphatic substituents contains at least about 8 carbon atoms (e.g., from about 8 to about 18 carbon atoms), and at least one contains an anionic water-solubilizing group, e.g. carboxy, sulfonate, sulfate. Ampholytica surfactants have been described in, for example, U.S. Pat. No. 3,929,678. When included therein, a cleaning composition of the invention can comprise, for example, about 0.2 wt. % to about 15 wt. % (e.g., about 1 wt. % to about 10 wt. %) of ampholytic surfactants. I

In certain other embodiments, especially in personal care cleaning compositions, zwitterionic surfactants are included as co-surfactants. These surfactants can be broadly described as derivatives of secondary and tertiary amines, derivatives of heterocyclic secondary and tertiary amines, or derivatives of quaternary ammonium, quaternary phosphonium or tertiary sulfonium compounds. Zwitterionic surfactants have been described in, for example, U.S. Pat. No. 3,929,678. When included therein, a cleaning composition of the invention can comprise, for example, about 0.2 wt. % to about 15 wt. % (e.g., about 1 wt. % to about 10 wt. %) of zwitterionic surfactants.

In further embodiments, primary or tertiary amines can be included as co-surfactants. Suitable primary amines include amines according to the formula R¹NH₂ wherein R¹ is a C₆-C₁₂, preferably C₆-C₁₀, alkyl chain, or R₄X(CH₂)n, wherein X is —O—, —C(O)NH— or —NH—, R⁴ is a C₆-C₁₂ alkyl chain, n is between 1 to 5 (e.g., 3). The alkyl chain of R¹ can be straight or branched, and can be interrupted with up to 12, but preferably less than 5 ethylene oxide moieties. Preferred amines include n-alkyl amines, selected from, for example, 1-hexylamine, 1-octylamine, 1-decylamine and laurylamine, C₈-C₁₀ oxypropylamine, octyloxypropylamine, 2-ethylhexyl-oxypropylamine, lauryl amido propylamine or amido propylamine. Suitable tertiary amines include those having the formula R¹R²R³N wherein R¹ and R² are C₁-C₈ alkyl chains, R³ is either a C₆-C₁₂, preferably C₆-C₁₀, alkyl chain, or R³ is R⁴X(CH₂)n, whereby X is —O—, —C(O)NH— or —NH—, R⁴ is a C₄-C₁₂, n is between 1 to 5 (e.g., 2-3), R⁵ is H or C₁-C₂ alkyl, and x is between 1 to 6. R³ and R⁴ may be linear or branched. The alkyl chain of R³ can be interrupted with up to 12, but preferably less than 5, ethylene oxide moieties. Preferred tertiary amines include, for example, 1-hexylamine, 1-octylamine, 1-decylamine, 1-dodecylamine, n-dodecyldimethylamine, bishydroxyethylcoconutalkylamine, oleylamine(7)ethoxylated, lauryl amido propylamine, and cocoamido propylamine.

In some embodiments, the cleaning composition of the invention comprises greater than about 5 wt. % anionic surfactant and/or less than about 25 wt. % nonionic surfactant. More preferably the composition comprises greater than about 10 wt. % anionic surfactant. More preferably the composition comprise less than 15%, more preferably less than 12% nonionic surfactants.

Other useful detersive surfactants have been described in the prior art, for example, in U.S. Pat. Nos. 3,664,961, 3,919,678, 4,222,905, and 4,239,659.

The total amount of surfactants included in a cleaning composition of the invention is typically about 0.1 wt. % or more (e.g., about 1 wt. % or more, about 10 wt. % or more, about 25 wt. % or more, about 50 wt. % or more, about 60 wt. % or more, about 70 wt. % or more). An exemplary cleaning composition of the invention comprises about 0.1 wt. % to about 80 wt. % total surfactants (e.g., about 1 wt. % to about 50 wt. %, about 10 wt. % to about 40 wt. %, about 20 wt. % to about 35 wt. %) of total surfactants, including the alkylbenzene and co-surfactants.

One criteria based on which to the type(s) and amount(s) of surfactants to be included in cleaning compositions can be determined is compatibility with the enzyme components present in the cleaning compositions. For example, in liquid or gel compositions, the cleaning composition (including all the surfactants, which are, for example, pre-formulated into a surfactant package) is prepared such that it promotes, or at least does not degrade, the stability of any enzyme in the cleaning composition.

A surfactant composition of the present invention, or a surfactant package which can be formulated and subsequently included in a cleaning composition, can be in any form, for example, a liquid; a solid such as a powder, granules, agglomerate, paste, tablet, pouches, bar; a gel; an emulsion; or in a suitable form to be delivered in dual-compartment containers. The composition can also be formulated into a spray or foam detergent, premoistened wipes (e.g., the cleaning composition in combination with a nonwoven material as described, for example, in U.S. Pat. No. 6,121,165), dry wipes (e.g., the cleaning composition in combination with a nonwoven material, activated with water by a consumer, as described, for example, in U.S. Pat. No. 5,980,931), and other homogeneous or multiphase consumer cleaning product forms.

### Cleaning Compositions

The surfactant compositions comprising an alkylbenzene, such as a sulfonated alkylbenzene, are particularly suitable as soil detachment-promoting ingredients of laundry detergents, dishwashing liquids and powders, and various other cleaning compositions. They exhibit high dissolving power especially when faced with greasy soils, and it is particular advantageous that they display the outstanding soil-detaching power even at low washing temperatures.

The alkylbenzene compositions according to the present invention can be included or blended into a surfactant package as described above, which comprises about 0.0001 wt. % to about 100 wt. % of one or more alkylbenzenes. That surfactant package can then be blended into a cleaning composition to impart detergency and cleaning power to the cleaning composition. In alternative embodiments, the alkylbenzene can be blended into a cleaning composition directly, in an amount of about 0.001 wt. % or more (e.g., about 0.001 wt. % or more, about 0.1 wt. % or more, about 1 wt. % or more, about 10 wt. % or more, about 20 wt. % or more, or about 40 wt. % or more) based on the total weight of the cleaning composition. For example, the alkylbenzene can be blended into a composition in an amount of about 0.001 wt. % to about 50 wt. % (e.g., about 0.01 wt. % to about 45 wt. %, about 0.1 wt. % to about 40 wt. %, about 1 wt. % to about 35 wt. %). Accordingly, a cleaning composition of the present invention, in either a solid form (e.g., a tablet, granule, powder, or compact), or a liquid form (e.g., a fluid, gel, paste, emulsion, or concentrate) can comprise about 0.001 wt. % to about 50 wt. % of an alkylbenzene. For example, a cleaning composition of the invention can comprise about 0.5 wt. % to about 44 wt. % of alkylbenzene. Preferably, the cleaning composition comprises about 1 wt. % to about 30 wt. % of alkylbenzene.

Alternatively, a cleaning composition of the present invention can comprise about 0.001 wt. % to about 80 wt. % of a surfactant package formulated to comprise about 0.001 wt. % to about 100 wt. % of alkylbenzene. For example, a cleaning composition of the present invention can comprise about 0.1 wt. % to about 50 wt. % of such a surfactant package. As described herein, the surfactant package can comprise other surfactants (i.e., co-surfactants), which can include surfactants derived from similar (e.g., alkylbenzene) or different sources (e.g., petroleum-derived surfactants). In a particular embodiment, however, the surfactant package can be entirely comprised of an alkylbenzene described herein.

### Industrial Cleaning Compositions, Household Cleaning Compositions & Personal Care Cleaning Compositions

In certain embodiments, the cleaning composition of the present invention is a liquid or solid laundry detergent composition. In certain alternative embodiments, the cleaning composition of the invention is a hard surface cleaning composition, wherein the hard surface cleaning composition preferably impregnates a nonwoven substrate. As used herein, “impregnate” means that the hard surface cleaning composition is placed in contact with a nonwoven substrate such that at least a portion of the nonwoven substrate is penetrated by the hard surface cleaning composition. Furthermore, the hard surface cleaning composition preferably saturates the nonwoven substrate. In other embodiments, the cleaning composition of the present invention is a car care composition, which is useful for cleaning various surfaces such as hard wood, tile, ceramic, plastic, leather, metal, or glass. In further embodiments, the cleaning composition is a dish-washing composition, such as, for example, a liquid hand dishwashing composition, a solid automatic dishwashing composition, a liquid automatic dishwashing composition, and a tab/unit dose form automatic dishwashing composition.

In further embodiments, the cleaning composition can be used in industrial environments for cleaning of various equipment, machinery, and for use in oil drilling operations. For example, the cleaning composition of the present invention can be particularly suited in environments wherein the surfactants come into contact with free hardness and in all compositions that require hardness tolerant surfactant systems, such as in compositions used to aid oil drilling.

In some embodiments, the cleaning composition of the invention can be designed or formulated into personal or pet care compositions such as shampoo compositions, body washes, or liquid or solid soaps.

Common cleaning adjuncts applicable to most cleaning compositions, including, household cleaning compositions, and personal care compositions and the like, include builders, enzymes, polymers, suds boosters, suds suppressors (antifoam), dyes, fillers, germicides, hydrotropes, anti-oxidants, perfumes, pro-perfumes, enzyme stabilizing agents, pigments, and the like. In some embodiments, the cleaning composition is a liquid cleaning composition, wherein the composition comprises one or more selected from solvents, chelating agents, dispersants, and water. In other embodiments, the cleaning composition is a solid, wherein the composition further comprises, for example, an inorganic filler salt. Inorganic filler salts are conventional ingredients of solid cleaning compositions, present in substantial amounts, varying from, for example, about 10 wt. % to about 35 wt. %. Suitable filler salts include, for example, alkali and alkaline-earth metal salts of sulfates and chlorides. An exemplary filler salt is sodium sulfate.

Household cleaning compositions, including, for example, laundry detergents and household surface cleaners typically comprise certain additional, in some embodiments, more specialized, ingredients or cleaning adjuncts selected from one or more of: bleaches, bleach activators, catalytic materials, suds boosters, suds suppressors (antifoams), diverse active ingredients or specialized materials such as dispersant polymers (e.g., various dispersant polymers made by BASF or Dow Chemicals), silver care, anti-tarnish and/or anti-corrosion agents, dyes, germicides, alkalinity sources, hydrotropes, anti-oxidants, enzyme stabilizing agents, pro-perfumes, perfumes, solubilizing agents, carriers, processing aids, pigments, and, for liquid formulations, solvents, chelating agents, dye transfer inhibiting agents, dispersants, brighteners, dyes, structure elasticizing agents, fabric softeners, anti-abrasion agents, hydrotropes, processing aids, and other fabric care agents. These more specialized cleaning adjuncts for household cleaning compositions, and the levels of use have been described in, for example, U.S. Pat. Nos. 5,576,282, 6,306,812 and 6,326,348. A comprehensive list of suitable laundry or other household cleaning adjuncts can be found, for example, in WO 99/05245.

Personal/pet or beauty care cleaning compositions including, for example, shampoos, facial cleansers, hand sanitizers, body wash, and the like, can also comprise, in some embodiments, other more specialized adjuncts, including, for example, conditioning agents such as vitamins, silicone, silicone emulsion stabilizing components, cationic cellulose or polymers such as Guar polymers, anti-dandruff agents, antibacterial agents, dispersed gel network phase, suspending agents, viscosity modifiers, dyes, non-volatile solvents or diluents (water soluble or insoluble), foam boosters, pediculocides, pH adjusting agents, perfumes, preservatives, chelates, proteins, skin active agents, sunscreens, UV absorbers, and minerals, herbal/fruit/food extracts, sphingolipids derivatives or synthetic derivatives and clay.

### Common Adjuncts

(1) Enzymes

Various known detersive enzymes can be blended into a cleaning composition of the present invention. Suitable enzymes include, for example, proteases, amylases, lipases, cellulases, pectinases, mannases, arabinases, galactanases, xylanases, oxidases (e.g., laccases), peroxidases, and/or mixtures thereof. These enzymes can provide enhanced cleaning performance and/or fabric care benefits. In general, just as the selection of the type and amount of surfactants to be formulated into a cleaning composition should take account of the enzymes therein, the types of enzyme chosen to be included in the composition should take account of the other components in the composition (including the various surfactants). Considerations may include, for example, the pH-optimum of the overall composition, the presence of absence of enzyme stabilization agents, etc. The enzymes should be present in the cleaning compositions in effective amounts.

Suitable proteases include those of animal, vegetable or microbial origin. Microbial origin is preferred. Chemically modified or engineered mutants (e.g., those described in International Publications WO 92/19729, 98/20115, 98/20116, 98/34946, etc.) can also be included. Suitable proteases can be a serine protease or a metallo protease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (as described in International Publications WO 89/06279 and WO 05/103244). Other suitable serine proteases include those from Micrococcineae sp. especially those from Cellulonas sp. and variants thereof as, e.g., described in International Publication WO05/052146. Examples of trypsin-like proteases including trypsin (e.g. of porcine or bovine origin) and the Fusarium proteases such as those described in International Publications WO 89/06270 and WO 94/25583. Many proteases are commercially available from Novozymes A/S and Genencor International Inc.

Suitable lipases also include those of bacterial or fungal origin. For example, suitable lipases can be selected from those derived from yeast, from genera such as a Candida, Kluyvermyces, pichia, Saccharomyces, Schizosaccharomyces, or Yarrowia, or derived from a filamentous fungi, such as an Acremonium, Aspergillus, Aureobasidum, Cryptococcus, Filobasidium, Fusarium, Humicolar, Magnaporthe, Mucor, Myceliophthora, Neocallimasix, Neurospora, Paecilomyces, Penicillium, Piromyces, Schizophyllum, Talaromyces, thermoascus, Thielavia, Tolypocladium, Thermomyces or Trichoderma. Many chemically modified lipases can also be suitable, including, for example, those from Humicola, those from Pseudomonas, a modified lipase from P. cepacia, a modified lipase from P. stutzeri, a modified lipase from P. fluoresces or Pseudomonas sp. strain SD 705, a modified lipase from P. wisconsinensis, those from Bacillus, a modified lipase from B. stearothermophilus and a modified lipase. A number of lipase enzymes, which can be included in a cleaning composition of the invention, are commercially available (Novozymes A/S). Suitable amylases (α and/or β) include those of bacterial or fungal origin. Chemically modified or engineered mutant amylases can also be suitably included in a cleaning composition of the invention. Amylases include, for example, α-amylases obtained from Bacillus. Various mutant amylases, which can be suitably included in a cleaning composition, have been described. A number of amylases, which can be included in a cleaning composition of the present invention, are commercially available from Novozymes A/S and Genencor International Inc. Suitable cellulases include those of bacterial or fungal origin. Chemically modified or engineered mutant cellulases can also be suitably included in a cleaning composition of the invention. A number of cellulases, especially those that provide added color care benefits, are commercially available, which can be included in a cleaning composition of the invention, especially in, for example, a laundry detergent composition. Commercially available cellulases are available from Genencor International Inc. and Kao Corporation.

Suitable peroxidases/oxidases include those of plant, bacterial or fungal origin. Chemically modified or engineered mutant peroxidases/oxidases can also be suitably included in a cleaning composition of the invention. Useful peroxidases include, for example, those obtained from the genera Coprinus. Commercially available peroxidases include, for example, Guardzyme™ (Novozymes A/S).

Suitable enzymes described above can be present in a cleaning composition of the present invention at levels of about 0.00001 wt. % or higher (e.g., about 0.01 wt % or higher, about 0.1 wt. % or higher, about 0.5 wt. % or higher, or about 1 wt. % or higher). For example, one or more such enzymes can be present in a cleaning composition of the invention in an amount of about 0.00001 wt. % to about 2 wt. % (e.g., about 0.0001 wt. % to about 1 wt. %, about 0.001 wt. % to about 0.5 wt. %) based on the total weight of the cleaning composition. In certain embodiments, the enzyme(s) can be present or used at very low levels, for example, at about 0.001 wt. % or lower. In alternative embodiments, enzyme(s) can be formulated, for example, into a heavier duty laundry detergent composition, at about 0.1 wt. % and higher, for example, at about 0.5 wt. % or higher.

(2) Enzyme Stabilizers

In certain embodiments, the cleaning composition of the present invention, which comprises one or more enzymes, for example, those described herein, further comprises one or more enzyme stabilizers. For example, the enzymes employed in the cleaning composition can be stabilized by the presence of water-soluble sources of calcium and/or magnesium ions in the finished compositions that provide such ions to the enzymes. Known stabilizing agents include, for example, a polyol such as propylene glycol or a glycerol, a sugar or a sugar alcohol, a lactic acid, a boric acid, a boric acid derivative such as an aromatic borate ester, a phenyl boronic acid derivative such as a 4-formylphenyl boronic acid. These enzyme stabilizers can be incorporated into the cleaning composition in accordance with known methods, such as, for example, those described in International Publications WO 92/19709 and WO 92/19708.

(3) Builders

Cleaning compositions of the present invention can optionally comprise one or more detergent builders or builder systems. When a builder is used, the subject composition can comprise, for example, at least about 1 wt. % (e.g., at least about 1 wt. %, at least about 5 wt. %, at least about 10 wt. %, at least about 20 wt. %, at least about 30 wt. %, at least about 40 wt. %, at least about 50 wt. %, or more) of one or more builders. For example, a solid cleaning composition of the present invention can comprise, for example, about 1 wt. % to about 60 wt. % (e.g., about 5 wt. % to about 50 wt. %, about 10 wt. % to about 40 wt. %, about 15 wt. % to about 30 wt. %) of one or more builders or a builder system. For example, a liquid cleaning composition of the present invention can comprise about 0 wt. % to about 10 wt. % of one or more detergency builders.

Various known builder materials can be used, including, e.g., aluminosilicate materials, silicates, polycarboxylates, alkyl- or alkenyl-succinic acid, and fatty acids, materials such as ethylenediamine tetraacetate, diethylene triamine pentamethyleneacetate, metal ion sequestrants such as aminopolyphosphonates, particularly ethylenediamine tetramethylene phosphonic acid and diethylene triamine pentamethylene phosphonic acid. Particularly, builder materials such as calcium sequestrant materials, precipitating materials, calcium ion-exchange materials, polycarboxylate materials, citrate builder, succinic acid builders, aminocarboxylates, and mixtures thereof are preferred.

Examples of calcium sequestrant builder materials include alkali metal polyphosphates, such as sodium tripolyphosphate and organic sequestrants, such as ethylene diamine tetra-acetic acid. Examples of precipitating builder materials include sodium orthophosphate and sodium carbonate. Examples of calcium ion-exchange builder materials include the various types of water-insoluble crystalline or amorphous aluminosilicates, of which zeolites are the best known representatives, for example, zeolite A, zeolite B (also known as zeolite P), zeolite C, zeolite X, zeolite Y, and also the zeolite P-type as described in, for example, EP Patent 0 384 070.

Of particular importance are citrate builders, including, for example, citric acid and soluble salts thereof (particularly sodium salt), are polycarboxylate builders of particular importance for heavy duty liquid detergent formulations due to their availability from renewable resources and their biodegradability. Oxydisuccinates are also especially useful in such compositions and combinations. Useful succinic acid builders can also be C₅-C₂₀ alkyl and alkenyl succinic acids and salts thereof, including laurylsuccinate, myristylsuccinate, palmitylsuccinate, 2-dodecenylsuccinate, 2-pentadecenylsuccinate. with dodecenylsuccinic acid being particularly preferred.

A number of suitable polycarboxylate builders include cyclic compounds, particularly alicyclic compounds, such as those described in U.S. Pat. Nos. 3,308,067, 3,723,322, 3,835,163; 3,923,679; 4,102,903, 4,120,874, 4,144,226, and 4,158,635.

Ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, various alkali metal, ammonium, and substituted ammonium salts of poly acetic acids such as ethylenediamine tetraacetic acid and nitrilotriacetic acid, and polycarboxylates such as mellitic acid, succinic acid, oxy-disuccinic acid, polymaleic acid, benzene 1,3,5-tricarboxylic acid, carboxymethyloxy-succinic acid, and soluble salts thereof can be used as builders. Other nitrogen-containing, phosphate-free aminocarboxylates are sometimes used. Specific examples include ethylene diamine disuccinic acid and salts thereof (ethylene diamine disuccinates, EDDS), ethylene diamine tetraacetic acid and salts thereof (ethylene diamine tetraacetates, EDTA), and diethylene triamine penta acetic acid and salts thereof (diethylene triamine penta acetates, DTPA). In particular embodiments of a liquid composition, 3,3-dicarboxy-4-oxa-1,6-hexanedioates and related compounds as described in U.S. Pat. No. 4,566,984 can be suitable.

(4) Chelating Agents

Cleaning compositions of the present invention can optionally comprise one or a mixture of more than one copper, iron and/or manganese chelating agents. When such an agent is used, the subject cleaning composition can comprise, for example, about 0.005 wt. % or more (e.g., about 0.01 wt. % or more, about 1 wt. % or more, about 5 wt. % or more, about 10 wt. % or more) chelating agents. For example, a cleaning composition of the invention comprises about 0.005 wt. % to about 15 wt. % (e.g., about 0.01 wt. % to about 12 wt. %, about 0.1 wt. % to about 10 wt. %, about 1 wt. % to about 8 wt. %, about 2 wt. % to about 6 wt. %) chelating agents.

Suitable chelating agents can be selected from amino carboxylates, amino phosphonates, polyfunctionally-substituted aromatic chelating agents, or mixtures thereof, which are capable of removing copper, iron or manganese ions from washing mixtures by formation of soluble chelates.

Amino carboxylates include, for example, ethylenediaminetetracetates, N-hydroxyethylethylenediaminetriacetates, nitrilotriacetates, ethylenediamine tetraproprionates, triethylenetetraamine-hexacetates, diethylenetriamine penta-acetates, and ethanol diglycines, alkali metal, ammonium, and substituted ammonium salts thereof.

Amino phosphonates are selectively used in cleaning compositions because they inevitably increase the amount of total phosphorus. For certain applications, the amount of total phosphorus in a cleaning composition may need to be limited. Under such circumstances, amino phosphonates may not be a suitable chelating agent or should be used in low amounts. Amino phosphonates include, without limitation, ethylenediamine tetrakis(methylenephosphonates). Preferably, the amino phosphonates do not contain alkyl or alkenyl groups with more than about 6 carbon atoms.

Suitable polyfunctionally-substituted aromatic chelating agents have been described in, for example, U.S. Pat. No. 3,812,044. Exemplary polyfunctionally-substituted aromatic chelating agents include a dihydroxydisulfobenzene, such as a 1,2-dihydroxy-3,5-disulfobenzene.

In some embodiments, biodegradable chelators can be included in a cleaning composition of the invention. An exemplary biodegradable chelator is ethylenediamine disuccinate (“EDDS”), especially the [S,S] isomer as described in U.S. Pat. No. 4,704,233.

The compositions herein may also contain water-soluble methyl glycine diacetic acid (MGDA) salts (or acid form) as a chelate or co-builder useful with, for example, insoluble builders such as zeolites, layered silicates and the like.

(5) Hydrotropes

Hydrotropes can be optionally included in cleaning compositions of the present invention to improve the physical and chemical stability of the compositions. Suitable hydrotropes include sulfonated hydrotropes, which include, for example, alkyl aryl sulfonates, or alkyl aryl sulfonic acids. Alkyl aryl sulfonates can be sodium, potassium, calcium, or ammonium xylene sulfonates; sodium, potassium, calcium, or ammonium toluene sulfonates; sodium, potassium, calcium, or ammonium euraene sulfonates; sodium, potassium, calcium, or ammonium substituted or unsubstituted naphthalene sulfonates, and mixtures thereof. Preferred among these are the sodium salts. Alkyl aryl sulfonic acids can be xylenesulfonic acid, toluenesulfonic acid, cumenesulfonic acid, substituted or unsubstituted naphthalenesulfonic acid, or salts thereof. In certain embodiments, a mixture of xylenesulfonic acid and p-toluene sulfonate can be used.

If present, a cleaning composition of the present invention comprises hydrotropes in an amount of about 0.01 wt. % or more (e.g., about 0.02 wt. % or more, about 0.05 wt. % or more, about 0.1 wt. % or more, about 1 wt. % or more, about 5 wt. % or more, about 10 wt. % or more, or about 15 wt. % or more). On the other hand, a cleaning composition of the present invention comprises hydrotropes in an amount of no more bout 20 wt. % (e.g., no more than about 20 wt. %, no more than about 15 wt. %, no more than about 10 wt. %, no more than about 5 wt. %, no more than about 1 wt. %). In certain embodiments, the cleaning composition can comprise hydrotropes in an amount of about 0.01 wt. % to about 20 wt. % (e.g., about 0.02 wt. % to about 18 wt. %, about 0.05 wt. % to about 15 wt. %, about 0.1 wt. % to about 10 wt. %, about 1 wt. % to about 5 wt. %), based on the total weight of the cleaning composition.

(6) Rheology Modifier

A cleaning composition, when in the form of a liquid, of the present invention can suitably comprise a rheology modifier, which provides a matrix that is “shear-thinning”. A shear-thinning fluid, as it is understood by those skilled in the art, is a fluid the viscosity of which decreases as shear is applied to the fluid. Thus, at rest, for example, during storage or shipping of a liquid cleaning composition, the liquid matrix of the composition preferably has a relatively high viscosity. When shear is applied to the composition, however, such as in the act of pouring or squeezing the composition from its container, the viscosity of the matrix should be lowered to the extent that dispensing of the fluid product is easily and readily accomplished.

Various materials that are capable of forming shear-thinning fluids when combined with water or other aqueous liquids are known in the art. One type of structuring agent that is especially useful for this purpose comprises non-polymeric (except for conventional alkoxylation) crystalline hydroxy-functional materials that can form thread-like structuring systems throughout the liquid matrix when crystallized within the matrix in situ. Such materials include, for example, crystalline hydroxyl-containing fatty acids, fatty esters, or fatty waxes. Specific examples of preferred crystalline hydroxyl-containing rheology modifiers include castor oil and its derivatives. Especially preferred are hydrogenated castor oil derivatives such as hydrogenated castor oil and hydrogenated castor wax. A number of these materials are commercially available.

Suitable polymeric rheology modifiers include those of the polyacrylate, polysaccharide or polysaccharide derivative type. Polysaccharide derivatives typically used as rheology modifiers comprise polymeric gum materials. Such gums include pectine, alginate, arabinogalactan (gum Arabic), carrageenan, gellan gum, xanthan gum and guar gum. A further alternative and suitable rheology modifier is a combination of a solvent and a polycarboxylate polymer. The solvent can be, for example, an alkylene glycol, more preferably dipropy glycol. For example, the solvent can comprise a mixture of dipropyleneglycol and 1,2-propanediol, with a ratio of dipropyleneglycol to 1,2-propanediol being about 3:1 to about 1:3 (e.g., about 1:1). The polycarboxylate polymer can be, for example, a polyacrylate, polymethacrylate, or mixtures thereof. For example, the polyacrylate can be a copolymer of unsaturated mono- or di-carbonic acid and 1-30 C alkyl ester of the (meth) acrylic acid, or a polyacrylate of unsaturated mono- or di-carbonic acid and 1-30 C alkyl ester of the (meth) acrylic acid. Some of these polymers are commercially available, e.g., from Lubrizol (Wickliffe, Ohio).

The solvent can be present at a level of about 0.5 wt. % to about 15 wt. % (e.g., about 1 wt. % to about 12 wt. %, about 2 wt. % to about 9 wt. %), based on the total weight of the cleaning composition. The polycarboxylate polymer is suitably present at a level of about 0.1 wt. % to about 10 wt. % (e.g., about 1 wt. % to about 8 wt. %, about 1.5% to about 6 wt. %, about 2 wt. % to about 5 wt. %) in the cleaning composition.

(6) Solvents or Solvent Systems

A cleaning composition of the invention can be in a liquid form, wherein one or more suitable solvents or solvent systems are included. Suitable solvents include water and other solvents such as lipophilic fluids or organic solvents. Examples of suitable lipophilic fluids include siloxanes, other types of silicones, hydrocarbons, glycol ethers, glycerine derivatives such as glycerine ethers, perfluorinated amines, perfluorinated and hydrofluoroether solvents, low-volatility nonfluorinated organic solvents, diol solvents, other environmentally-friendly solvents and mixtures thereof. Particularly suitable solvents include low molecular weight primary and secondary alcohols, such as methanol, ethanol, propanol, and isopropanol. Monohydric alcohols, such as polyols containing from about 2 to about 6 carbon atoms, and/or about 2 to about 6 hydroxy groups (e.g., propylene glycol, ethylene glycol, glycerin, and 1,2-propanediol) are also suitable.

Solvents can be absent, for example, from anhydrous solid embodiments of the cleaning compositions of the invention. But in a liquid cleaning composition, they are typically present at levels of about 0.1 wt. % to about 98 wt. % (e.g., about 1 wt. % to about 90 wt. %, about 10 wt. % to about 80 wt. %, about 20 wt. % to about 75 wt. %).

(7) Organic Sequestering Agent

A cleaning composition of the invention can optionally comprise about 0.01 wt. % to about 1.0 wt. % of an organic sequestering agent. Non-limiting example of organic sequestering agent include nitriloacetic acid, EDTA, organic phosphonates, sodium citrate, sodium tartrate monosuccinate, sodium tartrate disuccinate, and mixture thereof.

### Adjuncts Particularly Suitable for Laundry/Household Applications

(1) Bleach System

A bleach system suitable for use herein typically contains one or more bleaching agents. Suitable bleaching agents include, for example, catalytic metal complexes, activated peroxygen sources, bleach activators, bleach boosters, photobleaches, bleaching enzymes, free radical initiators, and hyohalite bleaches.

Suitable activated peroxygen sources include, without limitation, preformed peracids, a hydrogen peroxide source in combination with a bleach activator, or a mixture thereof. Suitable preformed peracids include, without limitation, percarboxylic acids and salts, percarbonic acids and salts, perimidic acids and salts, peroxymonosulfuric acids and salts, and mixtures thereof. Suitable sources of hydrogen peroxide include, without limitation, perborate compounds, percarbonate compounds, perphosphate compounds and mixtures thereof. Suitable types and levels of activated peroxygen sources have been described in, for example, U.S. Pat. Nos. 5,576,282, 6,306,812, and 6,326,348.

A household cleaning composition of the invention can optionally comprise photobleach, which can be, for example, a xanthene dye photobleach, a photo-initiator, or mixtures thereof. Suitable photobleaches can also catalytic photobleaches and photo-initiators. In certain embodiments, catalytic photobleaches are selected from the group consisting of water soluble phthalocyanines of the formula:

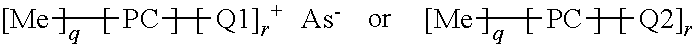

wherein: PC is the phthalocyanine ring system; Me is Zn; Fe(II); Ca; Mg; Na; K; Al—Z₁; Si(IV); P(V); Ti(IV); Ge(IV); Cr(VI); Ga(III); Zr(IV); In(III); Sn(IV) or Hf(VI); Z₁ is a halide; sulfate; nitrate; carboxylate; alkanoate; or hydroxyl ion; q is 0; 1 or 2; r is 1 to 4; Q1 is a sulfo or carboxyl group; or a radical of the formula: —SO₂X₂—R₁—X₃⁺; —O—R₁—X₃⁺; or —(CH₂), —Y₁⁺; in which R₁ is a branched or unbranched C₁-C₈ alkylene; or 1,3- or 1,4-phenylene; X₂ is —NH—; or —N—C₁-C₅ alkyl; X₃⁺ is a group of the formula:

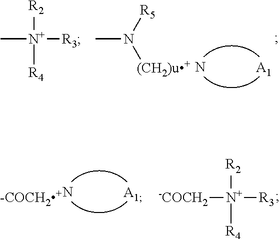

or, in the case where R₁═C₁-C₅ alkylene, also a group of the formula:

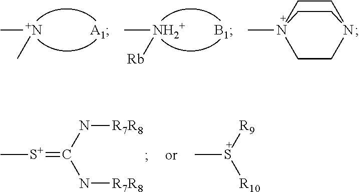

Y₁⁺ is a group of the formula:

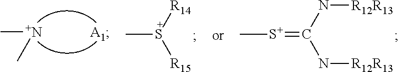

wherein t is 0 or 1; R₂ and R₃ independently of one another are C₁-C₆ alkyl; R₄ is C₁-C₅ alkyl; C₅-C₇ cycloalkyl or NR₇R₈; R₅ and R₆ independently of one another are C₁-C₅ alkyl; R₇ and R₈ independently of one another are hydrogen or C₁-C₅ alkyl; R₉ and R₁₀ independently of one another are unsubstituted C₁-C₆ alkyl or C₁-C₆ alkyl substituted by hydroxyl, cyano, carboxyl, carb-C₁-C₆ alkoxy, C₁-C₆ alkoxy, phenyl, naphthyl or pyridyl; u is from 1 to 6; A₁ is a unit which completes an aromatic 5- to 7-membered nitrogen heterocycle, which may where appropriate also contain one or two further nitrogen atoms as ring members, and B₁ is a unit which completes a saturated 5- to 7-membered nitrogen heterocycle, which may where appropriate also contain 1 to 2 nitrogen, oxygen and/or sulfur atoms as ring members; Q2 is hydroxyl; C₁-C₂₂ alkyl; branched C₃-C₂₂ alkyl; C₂-C₂₂ alkenyl; branched C₃-C₂₂ alkenyl and mixtures thereof; C₁-C₂₂ alkoxy; a sulfo or carboxyl radical; a radical of the formula:

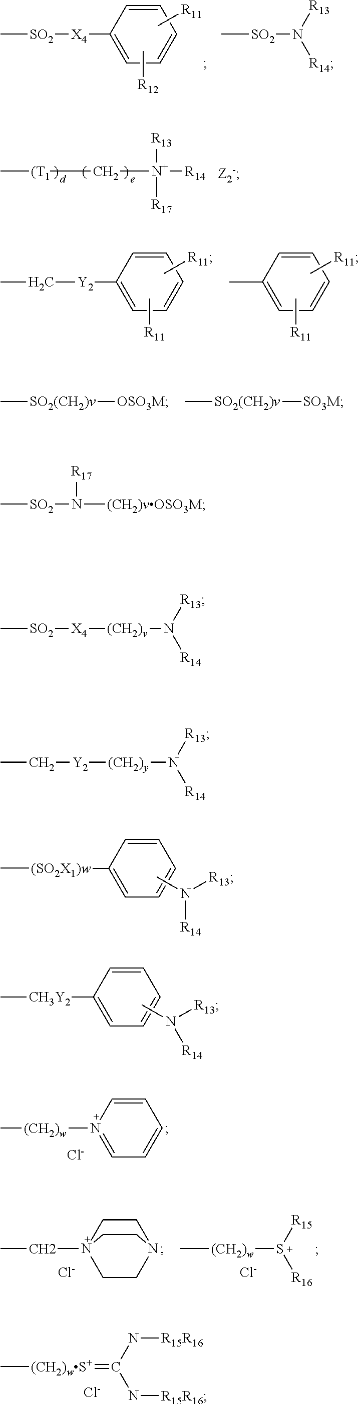

a branched alkoxy radical of the formula:

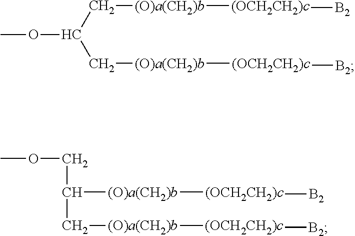

an alkylethyleneoxy unit of the formula:

-(T₁)d-(CH₂)_{b}(OCH₂CH₂)e-B₃

or an ester of the formula: COOR₁₈
wherein B₂ is hydrogen; hydroxyl; C₁-C₃₀ alkyl; C₁-C₃₀ alkoxy; —CO₂H; —CH₂COOH; —SO₃-M₁; —OSO₃-M₁; —PO₃²⁻M₁; —OPO₃²⁻M₁; and mixtures thereof; B₃ is hydrogen; hydroxyl; —COOH; —SO₃-M₁; —OSO₃-M₁ or C₁-C₆ alkoxy; M₁ is a water-soluble cation;
T₁ is —O—; or —NH—; X₁ and X₄ independently of one another are —O—; —NH— or —N—C₁-C₅alkyl;
R₁₁ and R₁₂ independently of one another are hydrogen; a sulfo group and salts thereof; a carboxyl group and salts thereof or a hydroxyl group; at least one of the radicals R₁₁ and R₁₂ being a sulfo or carboxyl group or salts thereof, Y₂ is —O—; —S—; —NH— or —N—C₁-C₅alkyl;
R₁₃ and R₁₄ independently of one another are hydrogen; C₁-C₆ alkyl; hydroxy-C₁-C₆ alkyl; cyano-C₁-C₆ alkyl; sulfo-C₁-C₆ alkyl; carboxy or halogen-C₁-C₆ alkyl; unsubstituted phenyl or phenyl substituted by halogen, C₁-C₄ alkyl or C₁-C₄ alkoxy; sulfo or carboxyl or R₁₃ and R₁₄ together with the nitrogen atom to which they are bonded form a saturated 5- or 6-membered heterocyclic ring which may additionally also contain a nitrogen or oxygen atom as a ring member; R₁₅ and R₁₆ independently of one another are C₁-C₆ alkyl or aryl-C₁-C₆ alkyl radicals; R₁₇ is hydrogen; an unsubstituted C₁-C₆ alkyl or C₁-C₆ alkyl substituted by halogen, hydroxyl, cyano, phenyl, carboxyl, carb-C₁-C₆ alkoxy or C₁-C₆ alkoxy; R₁₈ is C₁-C₂₂ alkyl; branched C₃-C₂₂ alkyl; C₁-C₂₂ alkenyl or branched C₃-C₂₂ alkenyl; C₃-C₂₂ glycol; C₁-C₂₂ alkoxy; branched C₃-C₂₂ alkoxy; and mixtures thereof;
M is hydrogen; or an alkali metal ion or ammonium ion, Z₂⁻ is a chlorine; bromine; alkylsulfate or arylsulfate ion; a is 0 or 1; b is from 0 to 6; c is from 0 to 100; d is 0; or 1; e is from 0 to 22; v is an integer from 2 to 12; w is 0 or 1; and A⁻ is an organic or inorganic anion, and s is equal to r in cases of monovalent anions A⁻ and less than or equal to r in cases of polyvalent anions, it being necessary for Aₛ⁻ to compensate the positive charge; where, when r is not equal to 1, the radicals Q₁ can be identical or different, and where the phthalocyanine ring system may also comprise further solubilizing groups.

Other suitable catalytic photobleaches include xanthene dyes, sulfonated zinc phthalocyanine, sulfonated aluminum phthalocyanine, Eosin Y, Phoxine B, Rose Bengal, C.I. Food Red 14, and mixtures. In some embodiment, a photobleach can be a mixture of sulfonated zinc phthalocyanine and sulfonated aluminum phthalocyanine, wherein the weight ratio of sulfonated zinc phthalocyanine to sulfonated aluminum phthalocyanine is greater than 1, greater than 1 but less than about 100, or from 1 to about 4.

Suitable photo-initiators include, for example, aromatic 1,4-quinones such as anthraquinones and naphthaquinones; alpha amino ketones, particularly those containing a benzoyl moiety; alphahydroxy ketones, particularly alpha-hydroxy acetophenones; phosphorus-containing photoinitiators, including monoacyl, bisacyl and trisacyl phosphine oxide and sulphides; dialkoxy acetophenones; alpha-haloacetophenones; trisacyl phosphine oxides; benzoin and benzoin based photoinitiators; and mixtures thereof. In some embodiments, photo-initiators can be 2-ethyl anthraquinone; Vitamin K3; 2-sulphate-anthraquinone; 2-methyl 1-[4-phenyl]-2-morpholinopropan-1-one (Irgacure® 907); (2-benzyl-2-dimethyl amino-1-(4-morpholinophenyl)-butan-1-one (Irgacure® 369); (1-[4-(2-hydroxyethoxy)-phenyl]-2 hydroxy-2-methyl-1-propan-1-one) (Irgacure® 2959); 1-hydroxy-cyclohexyl-phenyl-ketone (Irgacure® 184) (Ciba); oligo[2-hydroxy 2-methyl-1-[4(1-methyl)-phenyl]propanone (Esacure® KIP 150) (Lamberti); 2-4-6-(trimethyl-benzoyl)diphenyl-phosphine oxide, bis(2,4,6-trimethylbenzoyl)-phenyl-phosphine oxide (Irgacure® 819); (2,4,6 trimethylbenzoyl)phenyl phosphinic acid ethyl ester (Lucirin® TPO-L(BASF)); and mixtures thereof.

A number of photobleaches are commercially available, including those described above, from, e.g., Aldrich (Milwaukee, Wis.); Frontier Scientific (Logan, Utah); Ciba (Basel, Switzerland); BASF (Ludwigshafen, Germany); Lamberti S.p.A (Gallarate, Italy); Dayglo Color Corporation (Mumbai, India); Organic Dyestuffs Corp., (East Providence, R.I.).

(2) Pearlescent Agents

Pearlescent agents are optional but commonly included ingredients of a number of various household cleaners, especially, for example, in hard surface cleaners. They are typically crystalline or glassy solids, transparent or translucent compounds capable of reflecting and/or refracting light to produce a pearlescent effects. For example, they are crystalline particles insoluble in the composition in which they are incorporated. Preferably the pearlescent agents have the shape of thin plates or spheres (which are generally spherical). As commonly practiced in the art, particle sizes are measured across the largest diameter of spheres. Plate-like particles are defined as those wherein the two dimensions of the particle (length and width) are at least 5 times the third dimension (depth or thickness). Other crystal shapes like cubes or needles typically do not display pearlescent effect and thus are not used as pearlescent agents.

Suitable pearlescent agents preferably have D0.99 (sometimes referred to as D99) volume particle size of less than 50 μm. More preferably the pearlescent agents have D0.99 of less than 40 μm, most preferably less than 30 μm. Most preferably the particles have volume particle size greater than 1 μm. The D0.99 is a measure of particle size relating to particle size distribution and meaning in this instance that 99% of the particles have volume particle size of less than 50 μm. Volume particle size and particle size distribution can be measured using conventional methods and equipment, such as, for example, a Hydro 2000G (Malvern Instruments Ltd.). The choice of a particle size needs to balance the ease of distribution vs. the efficacy of the pearlescent agent, as it is known in the art that the smaller the particle size, the easier they are suspended, but the less the efficacy.

Liquid compositions containing less water and more organic solvents will typically have a refractive index that is higher in comparison to the more aqueous compositions. In these compositions, pearlescent agents with high refractive index are preferably included because otherwise the pearlescent agents do not impart sufficient visual pearlescence even when introduced at high levels (e.g., more than about 3 wt. %). In liquid compositions containing less water and more organic solvents, the pearlescent agent is preferably one having a refractive index of more than 1.41 (e.g., more than 1.8, more than 2.0. In some embodiments, the difference in refractive index between the pearlescent agent and the cleaning composition or medium, to which pearlescent agent is added, is at least 0.02, or at least 0.2, or at least 0.6.

A liquid cleaning composition of the present invention may comprise about 0.01 wt. % or more (e.g., about 0.02 wt. % or more, about 0.05 wt. % or more, about 0.1 wt. % or more, about 0.5 wt. % or more, about 1.0 wt. % or more, about 1.5 wt. % or more) of one or more pearlescent agents. Typically, however, the liquid composition comprises no more than about 2 wt. % (e.g., no more than about 1.5 wt. %, no more than about 1.0 wt. %, no more than about 0.5 wt. %) of one or more pearlescent agents. For example, a liquid cleaning composition of the invention comprises about 0.01 wt. % to about 2.0 wt. % (e.g., about 0.1 wt. % to about 1.5 wt. %) of one or more pearlescent agents.

Suitable pearlescent agents may be organic or inorganic. Organic pearlescent agents include, for example, monoester and/or diester of alkylene glycols, propylene glycol, diethylene glycol, dipropylene glycol, methylene glycol or tetraethylene glycol with fatty acids containing from about 6 to about 22, preferably from about 12 to about 18 carbon atoms, such as caproic acid, caprylic acid, 2-ethyhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, arachic acid, gadoleic acid, behenic acid, erucic acid, and mixtures thereof.

Inorganic pearlescent agents include mica, metal oxide coated mica, silica coated mica, bismuth oxychloride coated mica, bismuth oxychloride, myristyl myristate, glass, metal oxide coated glass, guanine, glitter, and mixtures thereof.

Organic pearlescent agent such as ethylene glycol mono stearate and ethylene glycol distearate provide pearlescence, but typically only when the composition is in motion. Hence only when the composition is poured will the composition exhibit pearlescence. Inorganic pearlescent materials are preferred as the provide both dynamic and static pearlescence. By dynamic pearlescence it is meant that the composition exhibits a pearlescent effect when the composition is in motion. By static pearlescence it is meant that the composition exhibits pearlescence when the composition is static.

Inorganic pearlescent agents are available as a powder, or as a slurry of the powder in an appropriate suspending agent. Suitable suspending agents include ethylhexyl hydroxystearate, hydrogenated castor oil. The powder or slurry of the powder can be added to the composition without the need for any additional process steps.

Optionally, co-crystallizing agents can be used to enhance the crystallization of the organic pearlescent agents. Suitable co-crystallizing agents include but are not limited to fatty acids and/or fatty alcohols having a linear or branched, optionally hydroxyl substituted, alkyl group containing from about 12 to about 22, preferably from about 16 to about 22, and more preferably from about 18 to 20 carbon atoms, such as palmitic acid, linoleic acid, stearic acid, oleic acid, ricinoleic acid, behenyl acid, cetearyl alcohol, hydroxystearyl alcohol, behenyl alcohol, linolyl alcohol, linolenyl alcohol, and mixtures thereof.

(3) Perfumes/Fragrances

The term “perfume” as used herein encompasses individual perfume ingredients as well as perfume accords. The perfume ingredients are often premixed to form a perfume accord prior to adding to a cleaning composition. As used herein, the term “perfume” can also include perfume microencapsulates. Perfume microcapsules comprise perfume raw materials encapsulated within a capsule made with materials selected from urea and formaldehyde; melamine and formaldehyde; phenol and formaldehyde; gelatine; polyurethane; polyamides; cellulose ethers; cellulose esters; polymethacrylate; and mixtures thereof. Encapsulation techniques are known and described in, for example, “Microencapsulation”: methods and industrial applications, Benita & Simon, eds. (Marcel Dekker, Inc., 1996).

The perfume ingredients that can be included in a cleaning composition can include various natural and synthetic chemicals. Exemplary perfume ingredients include aldehydes, ketones, esters, natural extracts, natural essences and the like.

Industrial cleaning compositions often do not comprise perfume ingredients. However, perfume ingredients are commonly found in household and personal care cleaning compositions. When present, the level of perfume or perfume accord is typically present in an amount of about 0.0001 wt. % or more (e.g., about 0.01 wt. % or more, about 0.1 wt. % or more, about 0.5 wt. % or more, about 2 wt. % or more), based on the total weight of the cleaning composition. For example, the level of perfume or perfume accord can be present in an amount of about 0.0001 wt. % to about 10 wt. % (e.g., about 0.01 wt. % to about 5 wt. %, about 0.1 wt. % to about 2 wt. %, preferably about 0.02 wt. % to about 0.8 wt. %, more preferably from about 0.003 wt. % to about 0.6 wt. %) by weight of the detergent composition. The level of perfume ingredients in a perfume accord, if one exists, is typically from about 0.0001 wt. % to about 99 wt. % by weight of the perfume accord. Exemplary perfume ingredients and perfume accords are disclosed in, for example, U.S. Pat. Nos. 5,445,747, 5,500,138, 5,531,910, 6,491,840, and 6,903,061.

(4) Dyes, Colorants, and Preservatives

The cleaning compositions herein can optionally contain dyes, colorants, and/or preservatives, or contain one or more, or none of these components. The dyes, colorants and/or preservatives can be naturally occurring or slightly processed from natural materials, or they can be synthetic. For example, natural-occurring preservatives include benzyl alcohol, potassium sorbate and bisabalol, sodium benzoate, and 2-phenoxyethanol. Synthetic preservatives can be selected from, for example, mildewstate or bacteriostate, methyl, ethyl, and propyl parabens, bisguamidine components (e.g., Dantagard™ and/or Glydant™ (Lonza Group)). Midewstate or bacteriostate compounds include, without limitation, KATHON® GC, a 5-chloro-3-methyl-4-isothiazolin-3-one, KATHON® ICP, a 2-methyl-4-isothiazolin-4-one, and a blend thereof, and KATHON® 886, a 5-chloro-2-methyl-4-isothazolin-3-one (Dow Chemicals); BRONOPOL, a 2-bromo-2-nitropropane 1,3 diol (Boots, Co. Ltd.); DOWICIDE™ A, a 1,2-benzoisothiazolin-3-one (Dow Chemicals); and IRGASAN® DP 200, a 2,4,4′-trichloro-2-hydroxydiphenylether (Ciba-Geigy, AG).

Dyes and colorants include synthetic dyes such as Liquitint® Yellow or Blue or natural plant yes or pigments, such as natural yellow, orange, red, and/or brown pigment, such as carotenoids, including, for example, beta-carotene and lycopene. The composition can additionally contain fluorescent whitening agents or bluing agents.

Certain dyes can also be light sensitive, including for example Acid Blue 145 (Crompton), Hidacid® blue (Hilton, Davis, Knowles & Triconh); Pigment Green No. 7, FD&C Green No. 7, Acid Blue 1, Acid Blue 80, Acid Violet 48, and Acid Yellow 17 (Sandoz Corp.); D&C Yellow No. 10 (Warner Jenkinson Corp.).

If present, dyes or colorants are present in an amount of about 0.001 wt. % or more (e.g., about 0.002 wt. % or more, 0.01 wt. % or more, 0.05 wt. % or more, 0.1 wt. % or more; 0.5 wt. % or more). Dyes and colorants are typically present, if at all, in an amount of no more than about 1 wt. % (e.g., no more than about 0.8 wt. %, no more than about 0.5 wt. %, no more than about 0.2 wt. %, no more than about 0.1 wt. %, no more than about 0.01 wt. %). For example, dyes and colorants can be present in a cleaning composition of the invention in an amount of about 0.001 wt. % to about 1 wt. % (e.g., about 0.01 wt. % to about 0.4 wt. %), based on the total weight of the composition.

(5) Fabric Care Benefit Agents

A household cleaning composition can be a laundry detergent, wherein a preferred optional ingredient can be a fabric care benefit agent. As used herein, “fabric care benefit agent” refers to any material that can provide fabric care benefits such as fabric softening, color protection, pill/fuzz reduction, anti-abrasion, anti-wrinkle, and the like to garments and fabrics, particularly on cotton and cotton-rich garments and fabrics, when an adequate amount of the material is present on the garment/fabric. Non-limiting examples of fabric care benefit agents include cationic surfactants, silicones, poly olefin waxes, latexes, oily sugar derivatives, cationic polysaccharides, polyurethanes and mixtures thereof. Suitable silicones include, for example, silicone fluids such as poly(di)alkyl siloxanes, especially polydimethyl siloxanes and cyclic silicones.

Polydimethyl siloxane derivatives include, for example, organofunctional silicones. One embodiment of functional silicone are the ABn type silicones, as described in U.S. Pat. Nos. 6,903,061, 6,833,344, and International Publication WO-02/018528. A number of silicones are commercially available, including, for example, Waro™ and Silsoft™ 843 (GE Silicones, Wilton, Conn.). Functionalized silicones or copolymers with one or more different types of functional groups such as amino, alkoxy, alkyl, phenyl, polyether, acrylate, silicon hydride, mercaptoproyl, carboxylic acid, quaternized nitrogen are also suitable as fabric care benefit agents. A number of these are commercially available including, for example, SM2125, Silwet 7622 (GE Silicones), DC8822, PP-5495, DC-5562 (Dow Chemicals), KF-888, KF-889 (Shin Etsu Silicones, Akron, Ohio); Ultrasil® SW-12, Ultrasil® DW-18, Ultrasil® DW-AV, Ultrasil® Q-Plus, Ultrasil® Ca-I, Ultrasil® CA-2, Ultrasil® SA-I, Ultrasil® PE-100 (Noveon Inc., Cleveland, Ohio), Pecosil® CA-20, Pecosil® SM-40, Pecosil® PAN-150 (Phoenix Chemical, Somerville, N.J.).

The oily sugar derivatives suitable as fabric care benefit agents have been described in International Publication WO 98/16538. Olean® is a commercial brand for certain oily sugar derivatives marketed by The Procter and Gamble Co., in Cincinnati Ohio.

Many dispersible polyolefins can be used to provide fabric care benefits. The polyolefins can be in the form of waxes, emulsions, dispersions, or suspensions. Preferably, the polyolefin is a polyethylene, polypropylene, or a mixture thereof. The polyolefin may be at least partially modified to contain various functional groups, such as carboxyl, alkylamide, sulfonic acid or amide groups. More preferably, the polyolefin is at least partially carboxyl modified or, in other words, oxidized.

Polymer latex can also be used to provide fabric care benefits in a water based cleaning composition. Non-limiting examples of polymer latexes include those described in, for example, International Publication WO 02/018451. Additional non-limiting examples include the monomers used in producing polymer latexes, such as 100% or pure butylacrylate, butylacrylate and butadiene mixtures with at least 20 wt. % of butylacrylate, butylacrylate and less than 20 wt. % of other monomers excluding butadiene, alkylacrylate with an alkyl carbon chain at or greater than C₆, alkylacrylate with an alkyl carbon chain at or greater than C₆ and less than 50 wt. % of other monomers, or a third monomer added into monomer systems above.

Cationic surfactants are another class of care actives useful in this invention. Examples of cationic surfactants have been described in, for example, US Patent Publication US2005/0164905.

Fatty acids can also be used as fabric care benefit agents. When deposited on fabrics, fatty acids or soaps thereof, provide fabric care benefits (e.g., softness, shape retention) to laundry fabrics. Useful fatty acids (or soaps, such as alkali metal soaps) are the higher fatty acids containing from about 8 to about 24 carbon atoms, more preferably from about 12 to about 18 carbon atoms. Soaps can be made by direct saponification of fats and oils or by the neutralization of free fatty acids. Particularly useful are the sodium and potassium salts of the mixtures of fatty acids derived from coconut oil and tallow. Fatty acids can be from natural or synthetic origin, both saturated and unsaturated with linear or branched chains.

Color care agents are another type of fabric care benefit agent that can be suitably included in a cleaning composition. Examples include metallo catalysts for color maintenance, such as those described in International Publication WO 98/39403.

Fabric care benefit agents, when present in a household cleaning composition such as a laundry detergent composition, can suitably be present at a level of up to about 30 wt. % (e.g., up to about 20 wt. %, up to about 15 wt. %, up to about 10 wt. %, up to about 5 wt. %, up to about 2 wt. %), based on the total weight of the cleaning composition. For example, a cleaning composition of the invention comprises about 1 wt. % to about 20 wt. % (e.g., about 2 wt. % to about 15 wt. %, about 5 wt. % to about 10 wt. %) of one or more fabric care benefit agents.

(6) Deposition Aid

As used herein, “deposition aid” refers to any cationic polymer or combination of cationic polymers that significantly enhance the deposition of the fabric care benefit agent onto the fabric during laundering. An effective deposition aid typically has a strong binding capability with the water insoluble fabric care benefit agents via physical forces such as van der Waals forces or non-covalent chemical bonds such as hydrogen bonding and/or ionic bonding.

An exemplary deposition aid is a cationic or amphoteric polymer. Amphoteric polymers have a net cationic charge. The cationic charge density of the polymer can range from about 0.05 milliequivalents/g to about 6 milliequivalents/g. The charge density is calculated by dividing the number of net charge per repeating unit by the molecular weight of the repeating unit. Nonlimiting examples of deposition aids include cationic polysaccharides, chitosan and its derivatives, and cationic synthetic polymers. Specific deposition aids include, for example, cationic hydroxy ethyl cellulose, cationic starch, cationic guar derivatives, and mixtures thereof. Certain deposition aids are commercially available, including, for example, the JR 30M, JR 400, JR 125, LR 400 and LK 400 polymers (Amerchol Corporation, Edgewater N.J.), Celquat® H200, Celquat® L-200, and the Cato® starch (National Starch and Chemical Co., Bridgewater, N.J.), and Jaguar Cl 3 and Jaguar Excel (Rhodia, Inc., Cranburry N.J.).

(7) Fabric Substantive and Hueing Dye

Dyes can be included in a cleaning composition of the invention, for example, a laundry detergent. Conventionally, dyes include certain types of acid, basic, reactive, disperse, direct, vat, sulphur or solvent dyes. For inclusion in cleaning compositions, direct dyes, acid dyes, and reactive dyes are preferred. Direct dye is a group of water-soluble dye taken up directly by fibers from an aqueous solution containing an electrolyte, presumably due to selective adsorption. In the Color Index system, directive dye refers to various planar, highly conjugated molecular structures that contain one or more anionic sulfonate group. Acid dye is a group of water soluble anionic dyes that is applied from an acidic solution. Reactive dye is a group of dyes containing reactive groups capable of forming covalent linkages with certain portions of the molecules of natural or synthetic fibers. Suitable fabric substantive dyes that can be included in a cleaning composition of the invention include, for example, an azo compound, stilbenes, oxazines and phthalocyanines.

Hueing dyes are another type of dyes that may be present in a household cleaning composition of the invention. Such dyes have been found to exhibit good tinting efficiency during a laundry wash cycle without exhibiting excessive undesirable build up during laundering. Typically, a hueing dye is included in the laundry detergent composition in an amount sufficient to provide a tinting effect to fabric washed in a solution containing the detergent. In one embodiment, the detergent composition comprises, for example, from about 0.0001 wt. % to about 0.05 wt. % (e.g., about 0.001 wt. % to about 0.01 wt. %) of a hueing dye.

(8) Dye Transfer Inhibitors

A household cleaning composition of the invention, for example, a laundry detergent composition, can comprise one or more compounds for inhibiting dye transfer from one fabric to another of solubilized and suspended dyes encountered during fabric laundering operations involving colored fabrics. Exemplary dye transfer inhibitors include polymedc dye transfer inhibiting agents, which are capable of complexing or absorbing the fugitive dyes washed out of dyed fabrics before the dyes have an opportunity to become attached to other articles in the wash. Polymedc dye transfer agents are described in, for example, International Publication WO 98/39403. Modified polyethyleneimine polymers, such as those described in International Publication WO 00/05334, which are water-soluble or dispersible, modified polyamines can also be used. Other exemplary dye transfer inhibiting agents include, without limitation, polyvinylpyrridine N-oxide (PVNO), polyvinyl pyrrolidone (PVP), polyvinyl imidazole, N-vinyl-pyrrolidone and N-vinylimidazole copolymers (PVPVI), copolymers thereof, and mixtures thereof.

The amount of dye transfer inhibiting agents in the cleaning composition can be, for example, about 0.01 wt. % to about 10 wt. % (e.g., about 0.02 wt. % to about 5 wt. %, about 0.03 wt. % to about 2 wt. %).

(9) Optional Ingredients

Unless specified herein below, an “effective amount” of a particular adjunct or ingredient is preferably present in an amount of about 0.01 wt. % or more (e.g., about 0.1 wt. % or more, about 0.5 wt. % or more, about 1.0 wt. % or more, about 2.0 wt. % or more), based on the total weight of the detergent composition. Optional adjuncts however are usually presented in an amount of no more than about 20 wt. % (e.g., no more than about 15 wt. %, no more than about 10 wt. %, no more than about 5 wt. %, no more than about 2.5 wt. %, or no more than about 1 wt. %).

Examples of other suitable cleaning adjunct materials, one or more of which may be included in a cleaning composition, include, without limitation, effervescent systems comprising hydrogen peroxide and catalase; optical brighteners or fluorescers; soil release polymers; dispersants; suds suppressors; photoactivators; hydrolysable surfactants; preservatives; anti-oxidants; anti-shrinkage agents; gelling agents (e.g., amidoamines, amidoamine oxides, gellan gums); anti-wrinkle agents; germicides; fungicides; color speckles; antideposition agents such as celluose derivatives, colored beads, spheres or extrudates; sunscreens; fluorinated compounds; clays; luminescent agents or chemiluminescent agents; anti-corrosion and/or appliance protectant agents; alkalinity sources or other pH adjusting agents; solubilizing agents; processing aids; pigments; free radical scavengers, and mixtures thereof. Suitable materials and effective amounts have been described in, e.g., U.S. Pat. Nos. 5,705,464, 5,710,115, 5,698,504, 5,695,679, 5,686,014 and 5,646,101. Mixtures of the above components can be made in any proportion.

(10) Encapsulated Composition

A cleaning composition, such as a household cleaning composition including a laundry detergent, a dishwashing liquid, or a surface cleaning composition, of the present invention can optionally be encapsulated within a water soluble film. The water-soluble film can be made from polyvinyl alcohol or other suitable variations, carboxy methyl cellulose, cellulose derivatives, starch, modified starch, sugars, PEG, waxes, or combinations thereof.

In certain embodiment the water-soluble film may comprise other adjuncts such as copolymer of vinyl alcohol and a carboxylic acid, the advantages of which have been described in, for example, U.S. Pat. No. 7,022,656. An exemplary benefit of such encapsulation practice is the improvement of the shelf-life of the pouched composition. Another exemplary advantage is that this practice provides improved cold water (e.g., less than 10° C.) solubility to the cleaning composition. The level of the co-polymer in the film material is at least about 60 wt. % (e.g., about 65 wt. %, about 70 wt. %, about 80 wt. %) by weight. The polymer can have any average molecular weight, preferably about 1,000 daltons to 1,000,000 daltons (e.g., about 10,000 daltons to about 300,000 daltons, about 15,000 daltons to 200,000 daltons, about 20,000 daltons to 150,000 daltons). In certain embodiments, the copolymer present in the film is about 60% to about 98% hydrolyzed (e.g., about 80% to 95% hydrolyzed), to improve the dissolution of the material. In certain embodiments, the copolymer comprises about 0.1 mol % to about 30 mol % (e.g., about 1 mol % to about 6 mol %) of carboxylic acid. In certain embodiments, the water-soluble film comprises additional co-monomers, including, for example, sulfonates and ethoxylates such as 2-acrylamido-2-methyl-1-propane sulphonic acid. In further embodiments, the film can also comprise other ingredients, including, for example, plasticizers, for example, glycerol, ethylene glycol, diethyleneglycol, propane diol, 2-methyl-1,3-propane diol, sorbitol, and mixtures thereof, additional water, disintegrating aids, fillers, anti-foaming agents, emulsifying/dispersing agents, and/or antiblocking agents. It may be useful that the pouch or water-soluble film itself comprises a detergent additive to be delivered to the wash water, for example organic polymeric soil release agents, dispersants, dye transfer inhibitors. Optionally the surface of the film of the pouch may be dusted with fine powder to reduce the coefficient of friction. Sodium aluminosilicate, silica, talc and amylose are examples of suitable fine powders.

Certain water-soluble films are commercially available, for example, those marketed under the tradename M8630™ (Mono-Sol, Merriville, Ind.).

### Adjuncts Particularly Suitable for Personal Care Applications

(1) Hair Conditioning Agents

Cleaning compositions of the invention may comprise, in some embodiments such as, for example, used in personal or beauty care applications, various known conditioning agents. An exemplary conditioning agent especially suitable for personal care compositions such as shampoos, is a silicone or a silicone-containing material. Such materials can be selected from, for example, non-volatile silicones, siloxane gums and resins, aminofunctional silicones, quaternary silicones, and mixtures thereof with each other and with volatile silicones. Examples of these silicone polymers have been disclosed, for example, in U.S. Pat. No. 6,316,541.

Silicone oils are flowable silicone materials having a viscosity, as measured at 25° C., of less than about 50,000 centistokes (e.g., less than about 30,000 centistokes). For example, silicone oils typically have a viscosity of about 5 centistokes to about 50,000 centistokes (e.g., about 10 centistokes to about 30,000 centistokes). Suitable silicone oils include polyalkyl siloxanes, polyaryl siloxanes, polyalkylaryl siloxanes, polyether siloxane copolymers, and mixtures thereof. Other insoluble, non-volatile silicone fluids having hair conditioning properties can also be used.

Methods of making microemulsions of silicone particles have been described in the art, including, for example, the technique described in U.S. Pat. No. 6,316,541.

The silicone may, for example, be a liquid at ambient temperatures, so as to be of a suitable viscosity to enable the material itself to be readily emulsified to the required particle size of about 0.15 microns or less.

The amount of silicone incorporated into a cleaning composition of the invention may depend on the type of composition and the particular silicone materials used. A preferred amount is from about 0.01 wt. % to about 10 wt. %, although these limits are not absolute. The lower limit is determined by the minimum level to achieve acceptable conditioning for a target consumer group and the upper limit by the maximum level to avoid making the hair and/or skin unacceptably greasy. The activity of the microemulsion can be adjusted accordingly to achieve the desired amount of silicone or a lower level of the preformed microemulsion may be added to the composition.

The microemulsion of silicone oil may be further stabilized by sodium lauryl sulfate or sodium lauryl ether sulfate with 1-10 moles of ethoxylation. Additional emulsifier, preferably chosen from anionic, cationic, nonionic, amphoteric and zwitterionic surfactants, and mixtures thereof may be present. The amount of emulsifier will typically be in the ratio of about 1:1 to about 1:7 parts by weight of the silicone, although larger amounts of emulsifier can be used, for example, in about 5:1 parts by weight of the silicone or more. Use of these emulsifiers may be necessary to maintain clarity of the microemulsion if the microemulsion is diluted prior to addition to the personal care cleaning composition. The same detersive surfactants in the cleaning composition can also serve as the emulsifier in the preformed microemulsion.

The silicone microemulsion may be further stabilized using an emulsion polymerization process. A suitable emulsion polymerization process has been described by, for example, U.S. Pat. No. 6,316,541. A typical emulsifier is TEA dodecyl benzene sulfonate which is formed in the process when triethanolamine (TEA) is used to neutralize the dodecyl benzene sulfonic acid used as the emulsion polymerization catalyst. It has been found that selection of the anionic counterion, typically an amine, and/or selection of the alkyl or alkenyl group in the sulfonic acid catalyst can further improve the stability of the microemulsion in the shampoo composition. Examples of preferred amines include, without limitation, triisopropanol amine, diisopropanol amine, and aminomethyl propanol.

(2) Pearlescent Agents

Pearlescent agents, such as those described herein (e.g., supra) can be suitably included in a personal care cleaning composition such as a shampoo. They are defined, for the purpose of the present disclosure, as materials which impart, to a composition, the appearance of mother of pearl. It is believed that pearlescence is produced by specular reflection of light. Light reflected from pearl platelets or spheres as they lie essentially parallel to each other at different levels in the composition creates a sense of depth and luster. Some light is reflected off the pearlescent agent, and the remainder will pass through the agent. Light passing through the pearlescent agent, may pass directly through or be refracted. Reflected, refracted light produces a different color, brightness and luster.

(3) Cationic Cellulose or Guar Polymer

Cleaning compositions of the present invention can further contain a cationic polymer to aid the deposition of the silicone oil component and enhance conditioning performance. Non limiting examples of such polymers are described in the CTFA Cosmetic Ingredient Dictionary, 3rd ed, Estrin, Crosley, & Haynes eds., (The Cosmetic, Toiletry, and Fragrance Association, Inc., Washington, D.C. (1982)). Suitable cationic polymers include polysaccharide polymers, such as cationic cellulose derivatives, for example, salts of hydroxyethyl cellulose reacted with trimethyl ammonium substituted epoxide, referred to in the industry (CTFA) as Polyquaternium 10, as well as Polymer LR, JR, JP and KG series polymers (Amerchol Corporation, Edison, N.J.). Other suitable cationic cellulose polymers includes the polymeric quaternary ammonium salts of hydroxyethyl cellulose reacted with lauryl dimethyl ammonium-substituted epoxide referred to in the industry (CTFA) as Polyquaternium 24, available under the tradename Polymer LM-200 (Amerchol Corp., Edison N.J.).

Suitable cationic guar polymers include cationic guar gum derivatives, such as guar hydroxypropyltrimonium chloride, and those described in, for example, U.S. Pat. No. 5,756,720. Certain of these polymers are commercially available, including, for example, Jaguar® Excel (Rhodia Corporation, Cranbury, N.J.).

When used, the cationic polymers herein are either soluble in the cleaning composition or are soluble in a complex coacervate phase in the cleaning composition formed by the cationic polymer and the anionic, amphoteric and/or zwitterionic detersive surfactant component described hereinbefore. Complex coacervates of the cationic polymer can also be formed with other charged materials in the composition.

Concentrations of the cationic polymer in the composition can range from about 0.01 wt. % to about 3 wt. % (e.g., about 0.05 wt. % to about 2 wt. %, about 0.1 wt. % to about 1 wt. %. Suitable cationic polymers have cationic charge densities of at least about 0.4 meq/gm (e.g., at least about 0.6 meq/gm). Suitable cationic polymers have cationic charge densities of no more than about 5 meq/gm, at the pH of intended use of the cleaning composition. In an exemplary personal care cleaning composition, such as, for example, a shampoo, which generally has a pH range of about 3 to about 9 (e.g., about 4 to about 8). As used herein, “cationic charge density” of a polymer refers to the ratio of the number of positive charges on the polymer to the molecular weight of the polymer.

For example, suitable cationic polymers, which can be included in a cleaning composition of the present invention, is one of sufficiently high cationic charge density to effectively enhance deposition efficiency of the solid particle components in the cleaning composition. Cationic polymers comprising cationic cellulose polymers and cationic guar derivatives with cationic charge densities of at least about 0.5 meq/gm and preferably less than about 7 meq/gm are suitable for this purpose.

Preferably, the deposition polymers give good clarity and adequate flocculation on dilution with water during use, provided sufficient electrolyte is added to the formulation. Suitable electrolytes include, without limitation, sodium chloride, sodium benzoate, magnesium chloride, and magnesium sulfate.

(4) Perfumes/Fragrances

Just as perfumes or perfume accords are typically included in a household cleaning composition of the invention, perfumes or perfume accords as described herein (e.g., supra) are often included in a personal care cleaning composition, such as a shampoo or a body wash composition. The perfume ingredients, which optionally can be formulated into a perfume accord prior to blending or formulating the cleaning composition, can be obtained from a wide variety of natural or synthetic sources. They include, without limitation, aldehydes, ketones, esters, and the like. They also include, for example, natural extracts and essences, which can include complex mixtures of ingredients, such as orange oils, lemon oils, rose extracts, lavender, musk, patchouli, balsamic essence, sandalwood oil, pine oil, cedar, and the like. The amount of perfume to be included in a cleaning composition of the invention can vary, for example, from about 0.0001 wt. % to about 2 wt. % (e.g., about 0.01 wt. % to about 1.0 wt. %, about 0.1 wt. % to about 0.5 wt. %), based on the total weight of the cleaning composition.

(5) Sensory Indicators—Silica Particles

Optionally, in a personal care cleaning composition of the invention, various sensory indicators can be included. These agents provide a change in sensory feel after an appropriate usage time, allowing for easy and precise recognition for the appropriate time of washing. For example, these agents are particularly suitable for cleaning compositions such as hand cleansers. An exemplary type of sensory indicators are silica particles. The properties of the silica particle may be adjusted to provide the desired end point in time.

Various silica particles are commercially available, including, for example, those made and distributed by INEOS Silicas Ltd (Joliet, Ill.). These particles have also been described in, for example, U.S. Pat. No. 6,165,510, US Patent Publication 2003/0044442.

Silica particles can be present in an amount that can initially be felt by hands when starting washing with the cleaning composition. In one embodiment, the amount of silica particles is about 0.05 wt. % to about 8 wt. %. In some embodiments, suitable silica particles can have an initial average diameter of about 50 μm to about 600 μm (e.g., about 180 to about 420 μm). In alternative embodiments, suitable silica particles can further comprise color or pigment on the surface of the silica particles. In other embodiments, suitable silica particles diminish in size and cannot be felt by users during washing before about 5 minutes, about 2 minutes, about 30 seconds, about 25 seconds, about 20 seconds, about 15 seconds, about 10 seconds, about 5 seconds, about 5 to about 30 seconds, or about 10 to about 30 seconds.

Silica particles can also, in addition to providing sensory indications, improve the dispensing of the cleaning composition. For example, by including these particles, the cleaning composition, such as a liquid hand cleaner or a shampoo, may achieve a desirable thickness such that it is easier to be dispensed with a pump.

It is often desirable to regulate the viscosity of a composition comprising silica particles, however. Addition of glycerin has been found to be an effective approach to achieve this regulation. Glycerin is typically added to a composition comprising silica particles in an amount of at least about 1 wt. % (e.g., about 2 wt. %, about 2.5 wt. %, about 3 wt. %, about 4 wt. %, about 5 wt. %, or about 6 wt. %), based on the total weight of the cleaning composition. In some embodiments, glycerin is added in an amount of less than about 10 wt. % (e.g., less than about 8 wt. %, less than about 6 wt. %, less than about 4 wt. %, less than about 2 wt. %). The addition of glycerin may, in certain embodiments, help prevent clogging of pumps.

(6) Suspension Agents—Viscosity Control

Cleaning compositions of the invention can further include a suspending agent that allows the particulate matters therein, including, for example, the silica particles, to remain suspended. Suspending agents refer to materials that are capable of increasing the ability of the composition to suspend material. Examples of suspending agents include, but are not limited to, synthetic structuring agents, polymeric gums, polysaccharides, pectin, alginate, arabinogalactan, carrageen, gellan gum, xanthum gum, guar gum, rhamsan gum, furcellaran gum, and other natural gum. An exemplary synthetic structuring agent is a polyacrylate. An exemplary acrylate aqueous solution used to form a stable suspension of the solid particles is manufactured by Lubrizol as CARBOPOL™ resins, also known as CARBOMER™, which are hydrophilic high molecular weight, crosslinked acrylic acid polymers. Other polymers, which can be used as suspension agents, include, without limitation, CARBOPOL™ Aqua 30, CARBOPOL™ 940 and CARBOPOL™ 934.

The suspending agents can be used alone or in combination. The amount of suspending agent can be any amount that provides for a desired level of suspending ability. In certain embodiment, the suspending agent is present in an amount of about 0.01 wt. % to about 15 wt. % (e.g., about 0.1 wt. % to about 12 wt. %, about 1 wt. % to about 10 wt. %, about 2 wt % to about 5 wt. %) by weight of the cleaning composition.

(7) Other Suitable Adjuncts

A number of other adjuncts can be suitable for inclusion in a personal care cleaning composition. Those include, for example, thickeners, such as hydroxylethyl cellulose derivatives (e.g., Methocel™ products, Dow Chemicals, Inc., Philadelphia, Pa.; Natrosol® products, Aqualon Ashland, Wilmington, Del.; Carbopol™ products, Lubrizol; and Gellan Gum, Atlanta, Ga.).

Stability enhancers can also be included as suitable adjuncts. They are typically nonionic surfactants, including those having an hydrophilic-lipophilic balance range of about 9-18. These surfactants can be straight chained or branched chained, and they typically containing various levels of ethoxylation/propoxylation. The nonionic surfactants useful in the present invention are preferably formed from a fatty alcohol, a fatty acid, or a glyceride with a Cs to C24 carbon chain, preferably a C12 to C18 carbon chain derivatized to yield a Hydrophilic-Lipophilic Balance (HLB) of at least 9. HLB is understood to mean the balance between the size and strength of the hydrophilic group and the size and strength of the lipophilic group of the surfactant. Suitable adjuncts for personal care cleaning compositions can also include various vitamins, including, for example, vitamin B complex; including thiamine, nicotinic acid, biotin, pantothenic acid, choline, riboflavin, vitamin B6, vitamin B12, pyridoxine, inositol, carnitine, vitamins A, C, D, E, K, and their derivatives.

Further suitable adjuncts may include one or more materials selected from antimicrobial agents, antifungal agents, antidandruff agents, dyes, foam boosters, pediculocides, pH adjusting agents, preservatives, proteins, skin active agents, sunscreens, UV absorbers, minerals, herbal/fruit/food extracts, sphingolipid derivatives or synthetic derivatives, and clay.

### EXAMPLES

The invention is further described in the following examples, which do not limit the scope of the invention described in the claims.

### Example 1

### Detection and Verification of Alkane Biosynthesis in Selected Cyanobacteria

Seven cyanobacteria, whose complete genome sequences are publicly available, were selected for verification and/or detection of alkane biosynthesis: Synechococcus elongatus PCC7942, Synechococcus elongatus PCC6301, Anabaena variabilis ATCC29413, Synechocystis sp. PCC6803, Nostoc punctiforme PCC73102, Gloeobacter violaceus ATCC 29082, and Prochlorococcus marinus CCMP1986. Only the first three cyanobacterial strains from this list had previously been reported to contain alkanes (Han et al., J. Am. Chem. Soc. 91:5156-5159 (1969); Fehler et al., Biochem. 9:418-422 (1970)). The strains were grown photoautotrophically in shake flasks in 100 mL of the appropriate media (listed in Table 8) for 3-7 days at 30° C. at a light intensity of approximately 3,500 lux. Cells were extracted for alkane detection as follows: cells from 1 mL culture volume were centrifuged for 1 min at 13,000 rpm, the cell pellets were resuspended in methanol, vortexed for 1 min and then sonicated for 30 min. After centrifugation for 3 min at 13,000 rpm, the supernatants were transferred to fresh vials and analyzed by GC-MS. The samples were analyzed on either 30 m DP-5 capillary column (0.25 mm internal diameter) or a 30 m high temperature DP-5 capillary column (0.25 mm internal diameter) using the following method.

After a 1 μL splitless injection (inlet temperature held at 300° C.) onto the GC/MS column, the oven was held at 100° C. for 3 mins. The temperature was ramped up to 320° C. at a rate of 20° C./min. The oven was held at 320° C. for an additional 5 min. The flow rate of the carrier gas helium was 1.3 mL/min. The MS quadrupole scanned from 50 to 550 m/z. Retention times and fragmentation patterns of product peaks were compared with authentic references to confirm peak identity.

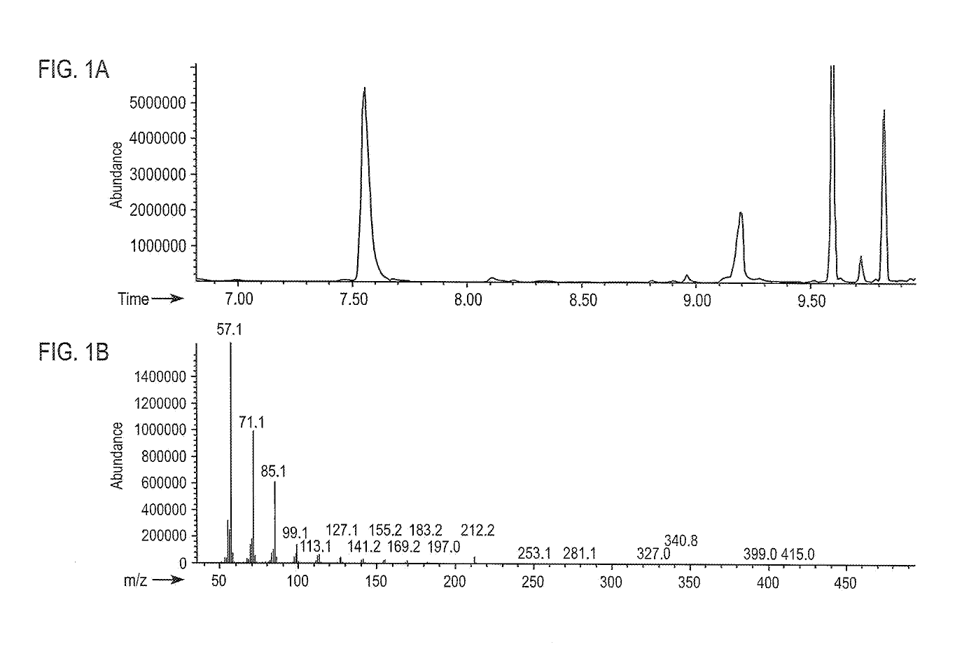

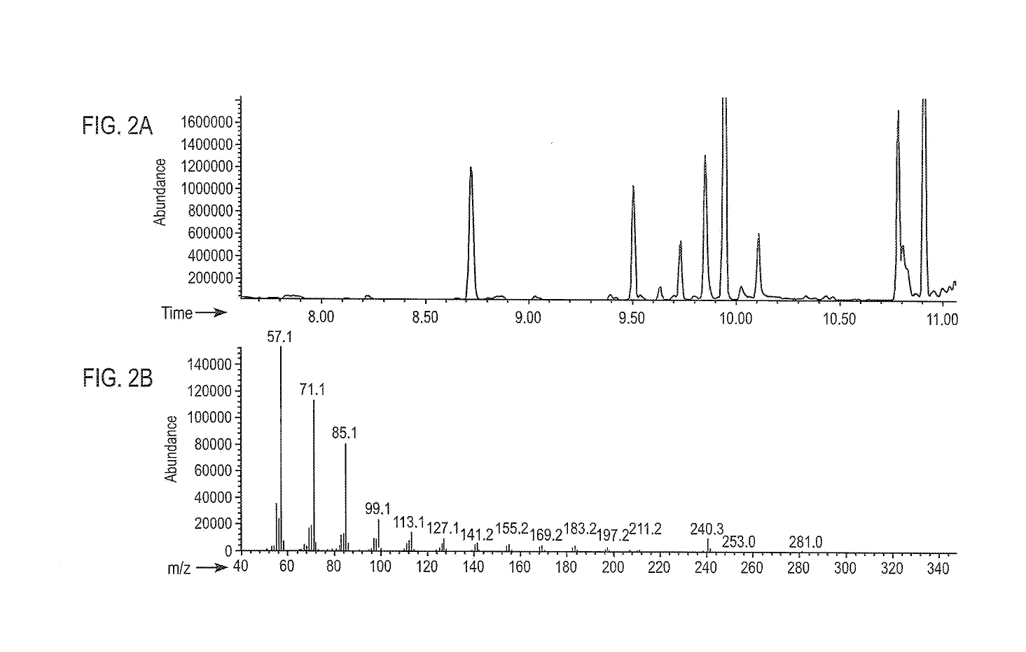

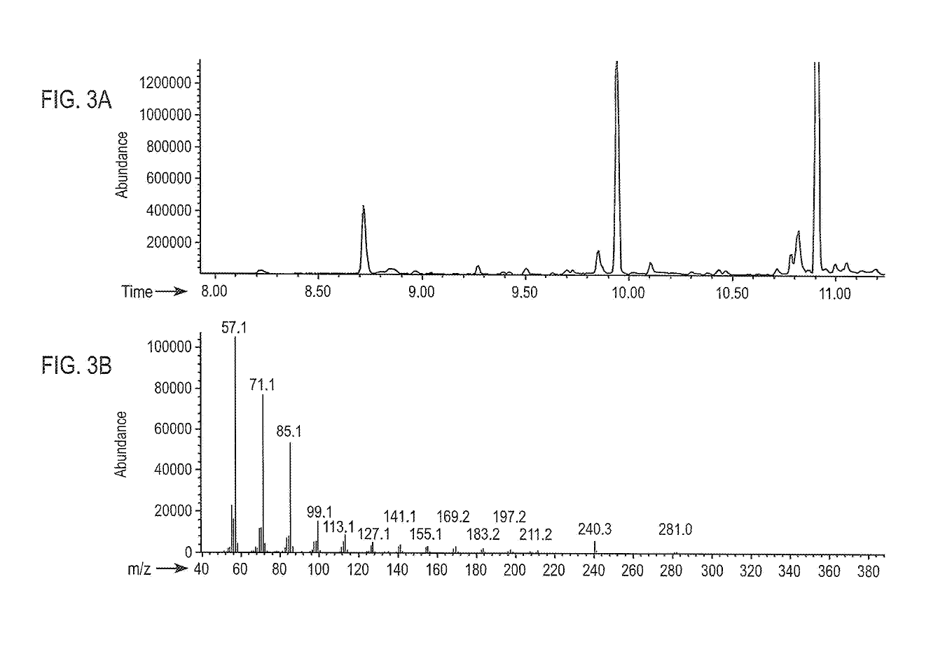

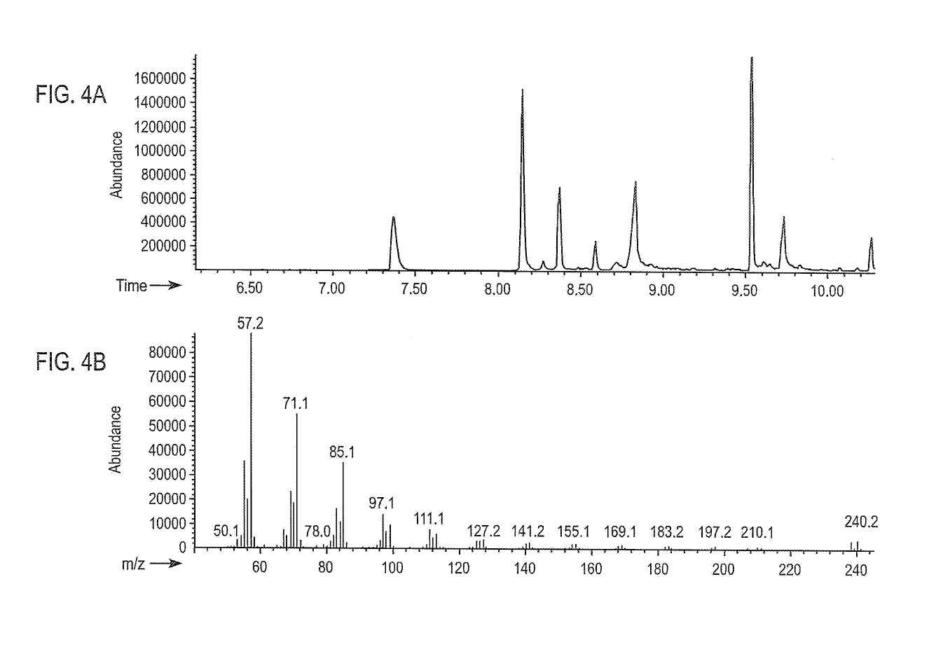

Out of the seven strains, six produced mainly heptadecane and one produced pentadecane (P. marinus CCMP1986); one of these strains produced methyl-heptadecane in addition to heptadecane (A. variabilis ATCC29413) (see Table 8). Therefore, alkane biosynthesis in three previously reported cyanobacteria was verified, and alkane biosynthesis was detected in four cyanobacteria that were not previously known to produce alkanes: P. marinus CCMP1986 (see FIG. 1), N. punctiforme PCC73102 (see FIG. 2), G. violaceus ATCC 29082 (see FIG. 3) and Synechocystis sp. PCC6803 (see FIG. 4).

FIG. 1A depicts the GC/MS trace of Prochlorococcus marinus CCMP1986 cells extracted with methanol. The peak at 7.55 min had the same retention time as pentadecane (Sigma). In FIG. 1B, the mass fragmentation pattern of the pentadecane peak is shown. The 212 peak corresponds to the molecular weight of pentadecane.

FIG. 2A depicts the GC/MS trace of Nostoc punctiforme PCC73102 cells extracted with methanol. The peak at 8.73 min has the same retention time as heptadecane (Sigma). In FIG. 2B, the mass fragmentation pattern of the heptadecane peak is shown. The 240 peak corresponds to the molecular weight of heptadecane.

FIG. 3A depicts the GC/MS trace of Gloeobaceter violaceus ATCC29082 cells extracted with methanol. The peak at 8.72 min has the same retention time as heptadecane (Sigma). In FIG. 3B, the mass fragmentation pattern of the heptadecane peak is shown. The 240 peak corresponds to the molecular weight of heptadecane.

FIG. 4A depicts the GC/MS trace of Synechocystic sp. PCC6803 cells extracted with methanol. The peak at 7.36 min has the same retention time as heptadecane (Sigma). In FIG. 4B, the mass fragmentation pattern of the heptadecane peak is shown. The 240 peak corresponds to the molecular weight of heptadecane.

TABLE 8
Hydrocarbons detected in selected cyanobacteria

| | | | | Alkanes | |
|---|---|---|---|---|---|
| Cyanobacterium | ATCC# | Genome | Medium | reported | verified ² |
| Synechococcus elongatus | 27144 | 2.7 Mb | BG-11 | C17:0 | C17:0, C15:0 |
| PCC7942 | | | | | |
| Synechococcus elongatus | 33912 | 2.7 Mb | BG-11 | C17:0 | C17:0, C15:0 |
| PCC6301 | | | | | |
| Anabaena variabilis | 29413 | 6.4 Mb | BG-11 | C17:0, 7- or | C17:0, |
| | | | | 8-Me-C17:0 | Me-C17:0 |
| Synechocystis sp. PCC6803 | 27184 | 3.5 Mb | BG-11 | — | C17:0, C15:0 |
| Prochlorococcus marinus | — | 1.7 Mb | — | — | C15:0 |
| CCMP1986 ¹ | | | | | |
| Nostoc punctiforme | 29133 | 9.0 Mb | ATCC819 | — | C17:0 |
| PCC73102 | | | | | |
| Gloeobacter violaceus | 29082 | 4.6 Mb | BG11 | — | C17:0 |

¹ cells for extraction were a gift from Jacob Waldbauer (MIT)
² major hydrocarbon is in bold

Genomic analysis yielded two genes that were present in the alkane-producing strains. The Synechococcus elongatus PCC7942 homologs of these genes are depicted in Table 9 and are Synpcc7942_{—}1593 (SEQ ID NO:1) and Synpcc7942_{—}1594 (SEQ ID NO:65).

TABLE 9
Alkane-producing cyanobacterial genes

| Gene Object | | Genbank | | | | | | |
|---|---|---|---|---|---|---|---|---|
| ID | Locus Tag | accession | Gene Name | Length | COG | Pfam | InterPro | Notes |
| 637800026 | Synpcc7942_1593 | YP_400610 | hypothetical | 231 aa | — | pfam02915 | IPR009078 | ferritin/ribonucleotide |
| | | | protein | | | | | reductase-like |
| | | | | | | | IPR003251 | rubreryhtrin |
| 637800027 | Synpcc7942_1594 | YP_400611 | hypothetical | 341 aa | COG5322 | pfam00106 | IPR000408 | predicted dehydrogenase |
| | | | protein | | | | IPR016040 | NAD(P)-binding |
| | | | | | | | IPR002198 | short chain dehydrogenase |

### Example 2

### Deletion of the sll0208 and sll0209 Genes in Synechocystis sp. PCC6803 Leads to Loss of Alkane Biosynthesis

The genes encoding the putative decarbonylase (sll0208; NP_{—}442147) (SEQ ID NO:3) and aldehyde-generating enzyme (sll0209; NP_{—}442146) (SEQ ID NO:67) of Synechocystis sp. PCC6803 were deleted as follows. Approximately 1 kb of upstream and downstream flanking DNA were amplified using primer sll0208/9-KO1 (CGCGGATCCCTTGATTCTACTGCGGCGAGT) with primer sll0208/9-KO2 (CACGCACCTAGGTTCACACTCCCATGGTATAACAGGGGCGTTGGACTCCTGTG) and primer sll0208/9-KO3 (GTTATACCATGGGAGTGTGAACCTAGGTGCGTGGCCGACAGGATAGGGCGTGT) with primer sll0208/9-KO4 (CGCGGATCCAACGCATCCTCACTAGTCGGG), respectively. The PCR products were used in a cross-over PCR with primers sll0208/9-KO1 and sll0208/9-KO4 to amplify the approximately 2 kb sll0208/sll0209 deletion cassette, which was cloned into the BamHI site of the cloning vector pUC19. A kanamycin resistance cassette (aph, KanR) was then amplified from plasmid pRL27 (Larsen et al., Arch. Microbiol. 178:193 (2002)) using primers Kan-aph-F (CATGCCATGGAAAGCCACGTTGTGTCTCAAAATCTCTG) and Kan-aph-R (CTAGTCTAGAGCGCTGAGGTCTGCCTCGTGAA), which was then cut with NcoI and XbaI and cloned into the NcoI and AvrII sites of the sll0208/sll0209 deletion cassette, creating a sll0208/sll0209-deletion KanR-insertion cassette in pUC19. The cassette-containing vector, which does not replicate in cyanobacteria, was transformed into Synechocystis sp. PCC6803 (Zang et al., 2007, J. Microbiol., vol. 45, pp. 241) and transformants (e.g., chromosomal integrants by double-homologous recombination) were selected on BG-11 agar plates containing 100 μg/mL Kanamycin in a light-equipped incubator at 30° C. Kanamycin resistant colonies were restreaked once and then subjected to genotypic analysis using PCR with diagnostic primers.

Confirmed deletion-insertion mutants were cultivated in 12 mL of BG11 medium with 50 μg/mL Kanamycin for 4 days at 30° C. in a light-equipped shaker-incubator. 1 mL of broth was then centrifuged (1 min at 13,000 g) and the cell pellets were extracted with 0.1 mL methanol. After extraction, the samples were again centrifuged and the supernatants were subjected to GC-MS analysis as described in Example 1.

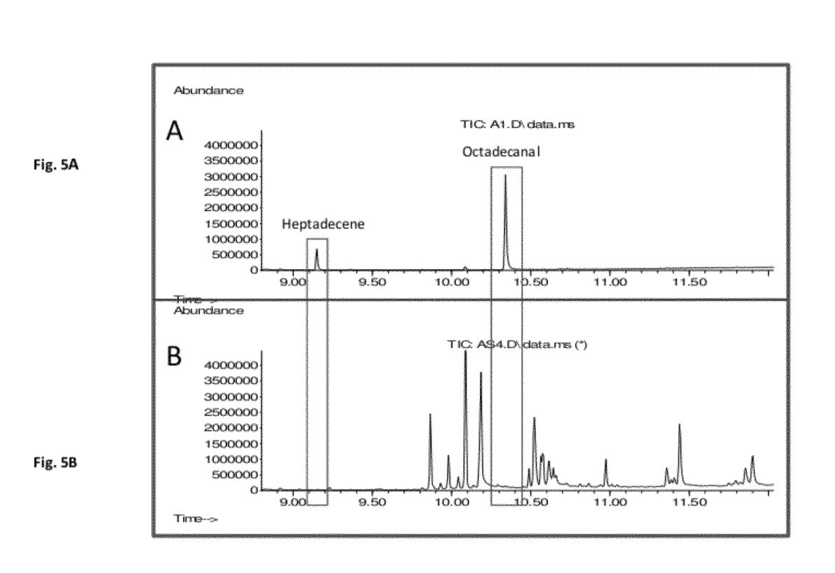

As shown in FIG. 5, the Synechocystis sp. PCC6803 strains in which the sll0208 and sll0209 genes were deleted lost their ability to produce heptadecene and octadecenal. This result demonstrates that the sll0208 and sll0209 genes in Synechocystis sp. PCC6803 and the orthologous genes in other cyanobacteria (see Table 1) are responsible for alkane and fatty aldehyde biosynthesis in these organisms.

### Example 3

### Production of Fatty Aldehydes and Fatty Alcohols in E. coli Through Heterologous Expression of Synechococcus elongatus PCC7942 orf1594

The genomic DNA encoding Synechococcus elongatus PCC7942 orf1594 (YP_{—}400611; putative aldehyde-generating enzyme) (SEQ ID NO:65) was amplified and cloned into the NcoI and EcoRI sites of vector OP-80 (pCL1920 derivative) under the control of the P_{trc} promoter. The resulting construct (“OP80-PCC7942_{—}1594”) was transformed into E. coli MG1655 and the cells were grown at 37° C. in M9 minimal media with 1% (w/v) glucose as carbon source and supplemented with 100 μg/mL spectinomycin. When the culture reached OD₆₀₀ of 0.8-1.0, it was induced with 1 mM IPTG and cells were grown for an additional 18-20 h at 37° C. Cells from 0.5 mL of culture were extracted with 0.5 mL of ethyl acetate. After sonication for 60 min, the sample was centrifuged at 15,000 rpm for 5 min. The solvent layer was analyzed by GC-MS as described in Example 1.

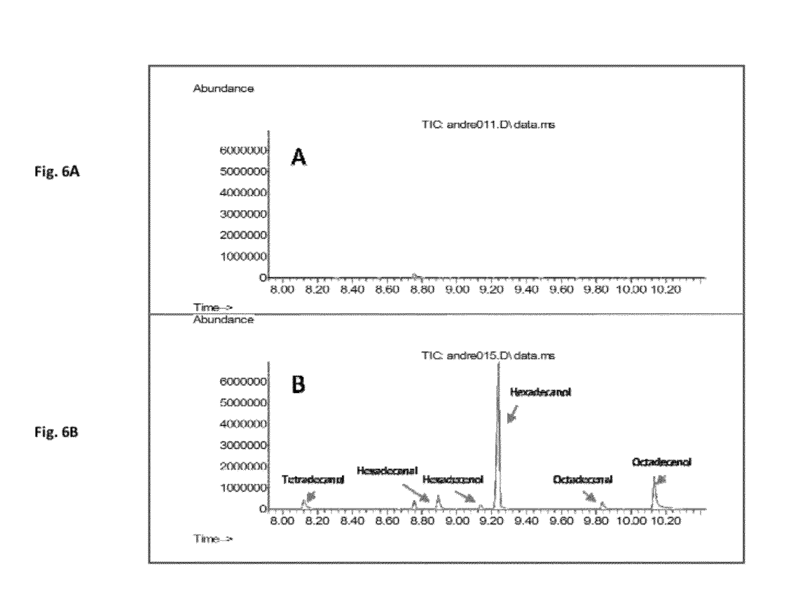

As shown in FIG. 6, E. coli cells transformed with the Synechococcus elongatus PCC7942 orf1594-bearing vector produced the following fatty aldehydes and fatty alcohols: hexadecanal, octadecenal, tetradecenol, hexadecenol, hexadecanol and octadecenol. This result indicates that PCC7942 orf1594 (i) generates aldehydes in-vivo as possible substrates for decarbonylation and (ii) may reduce acyl-ACPs as substrates, which are the most abundant form of activated fatty acids in wild type E. coli cells. Therefore, the enzyme was named Acyl-ACP reductase. In-vivo, the fatty aldehydes apparently are further reduced to the corresponding fatty alcohols by an endogenous E. coli aldehyde reductase activity.

### Example 4

### Production of Fatty Aldehydes and Fatty Alcohols in E. coli Through Heterologous Expression of Cyanothece sp. ATCC51142 cce_{—}1430

The genomic DNA encoding Cyanothece sp. ATCC51142 cce_{—}1430 (YP_{—}001802846; putative aldehyde-generating enzyme) (SEQ ID NO:69) was amplified and cloned into the NcoI and EcoRI sites of vector OP-80 (pCL1920 derivative) under the control of the P_{trc} promoter. The resulting construct was transformed into E. coli MG1655 and the cells were grown at 37° C. in M9 minimal media with 1% (w/v) glucose as carbon source and supplemented with 100 μg/mL spectinomycin. The cells were cultured and extracted as in Example 3 and analyzed by GC-MS as described in Example 26.

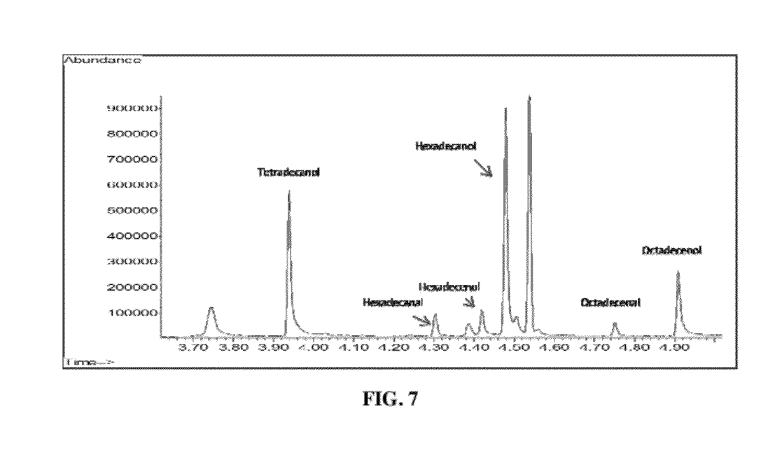

As shown in FIG. 7, E. coli cells transformed with the Cyanothece sp. ATCC51142 cce_{—}1430-bearing vector produced the following fatty aldehydes and fatty alcohols: hexadecanal, octadecenal, tetradecenol, hexadecenol, hexadecanol and octadecenol. This result indicates that ATCC51142 cce_{—}1430 (i) generates aldehydes in-vivo as possible substrates for decarbonylation and (ii) may reduce acyl-ACPs as substrates, which are the most abundant form of activated fatty acids in wild type E. coli cells. Therefore, this enzyme is also an Acyl-ACP reductase.

### Example 5

### Production of Alkanes and Alkenes in E. coli Through Heterologous Expression of Synechococcus elongatus PCC7942 orf1594 and Synechococcus elongatus PCC7942 orf1593

The genomic DNA encoding Synechococcus elongatus PCC7942 orf1593 (YP_{—}400610; putative decarbonylase) (SEQ ID NO:1) was amplified and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/mL spectinomycin and 100 μg/mL carbenicillin. The cells were cultured and extracted as in Example 3 and analyzed by GC-MS as described in Example 1.

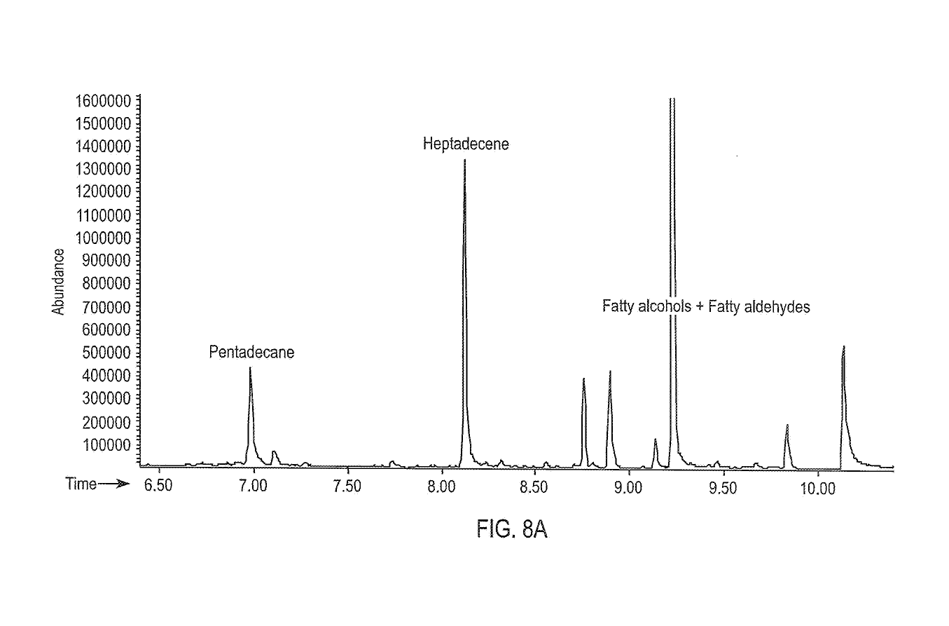

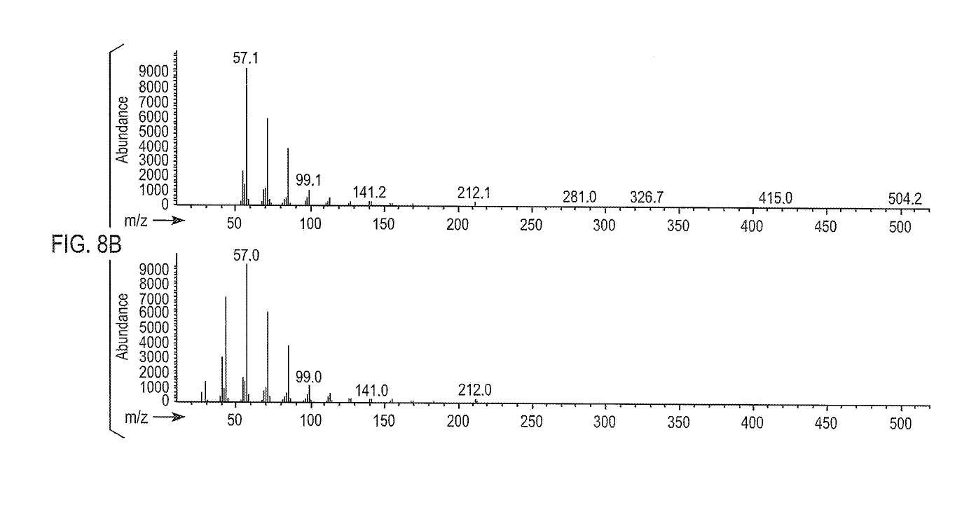

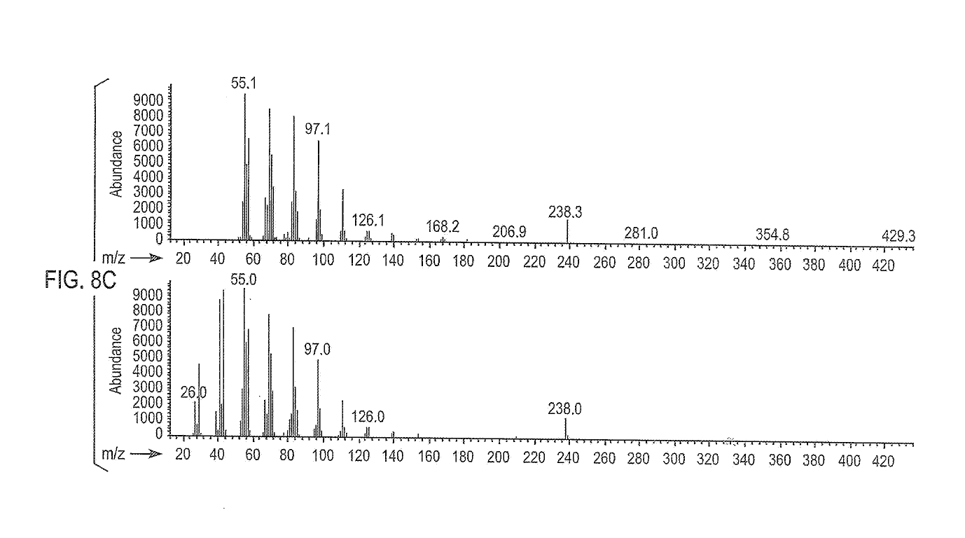

As shown in FIG. 8, E. coli cells cotransformed with the S. elongatus PCC7942_{—}1594 and S. elongatus PCC7942_{—}1593-bearing vectors produced the same fatty aldehydes and fatty alcohols as in Example 3, but also pentadecane and heptadecene. This result indicates that PCC7942_{—}1593 in E. coli converts hexadecanal and octadecenal to pentadecane and heptadecene, respectively, and therefore is an active fatty aldehyde decarbonylase.

### Example 6

### Production of Alkanes and Alkenes in E. coli Through Heterologous Expression of Synechococcus elongatus PCC7942 orf1594 and Nostoc punctiforme PCC73102 Npun02004178

The genomic DNA encoding Nostoc punctiforme PCC73102 Npun02004178 (ZP_{—}00108838; putative decarbonylase) (SEQ ID NO:5) was amplified and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/mL spectinomycin and 100 μg/mL carbenicillin. The cells were cultured and extracted as in Example 3 and analyzed by GC-MS as described in Example 1.

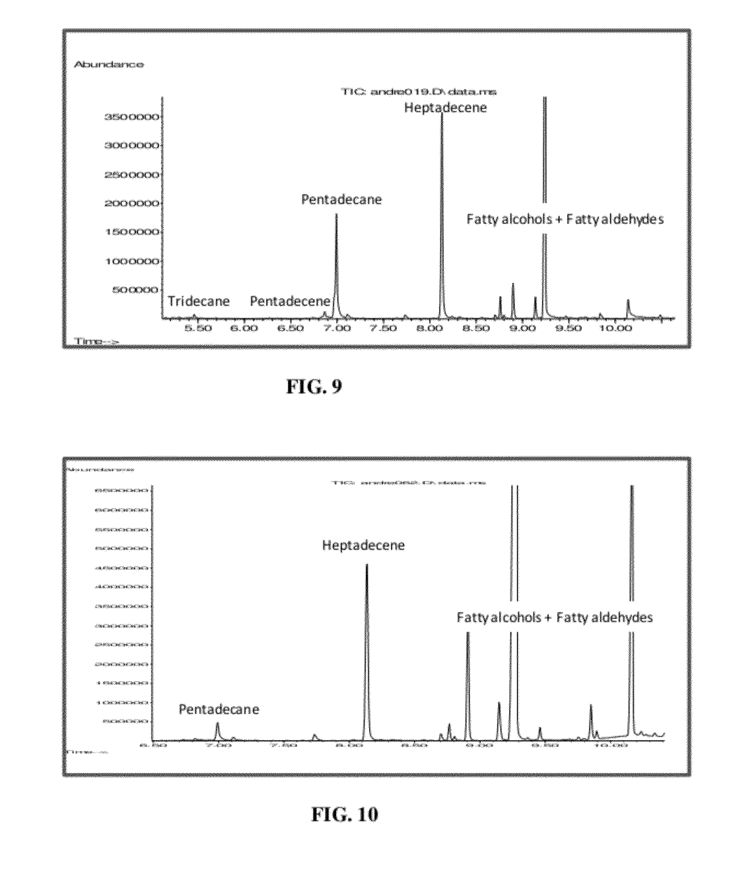

As shown in FIG. 9, E. coli cells cotransformed with the S. elongatus PCC7942_{—}1594 and N. punctiforme PCC73102 Npun02004178-bearing vectors produced the same fatty aldehydes and fatty alcohols as in Example 3, but also tridecane, pentadecene, pentadecane and heptadecene. This result indicates that Npun02004178 in E. coli converts tetradecanal, hexadecenal, hexadecanal and octadecenal to tridecane, pentadecene, pentadecane and heptadecene, respectively, and therefore is an active fatty aldehyde decarbonylase.

### Example 7

### Production of Alkanes and Alkenes in E. coli Through Heterologous Expression of Synechococcus elongatus PCC7942 orf1594 and Synechocystis sp. PCC6803 sll0208

The genomic DNA encoding Synechocystis sp. PCC6803 sll0208 (NP_{—}442147; putative decarbonylase) (SEQ ID NO:3) was amplified and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/mL spectinomycin and 100 μg/mL carbenicillin. The cells were cultured and extracted as in Example 3 and analyzed by GC-MS as described in Example 1.

As shown in FIG. 10, E. coli cells cotransformed with the S. elongatus PCC7942_{—}1594 and Synechocystis sp. PCC6803 sll0208-bearing vectors produced the same fatty aldehydes and fatty alcohols as in Example 3, but also pentadecane and heptadecene. This result indicates that Npun02004178 in E. coli converts hexadecanal and octadecenal to pentadecane and heptadecene, respectively, and therefore is an active fatty aldehyde decarbonylase.

### Example 8

### Production of Alkanes and Alkenes in E. coli Through Heterologous Expression of Synechococcus elongatus PCC7942 orf1594 and Nostoc sp. PCC7210 alr5283

The genomic DNA encoding Nostoc sp. PCC7210 alr5283 (NP_{—}489323; putative decarbonylase) (SEQ ID NO:7) was amplified and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/mL spectinomycin and 100 μg/mL carbenicillin. The cells were cultured and extracted as in Example 3 and analyzed by GC-MS as described in Example 1.

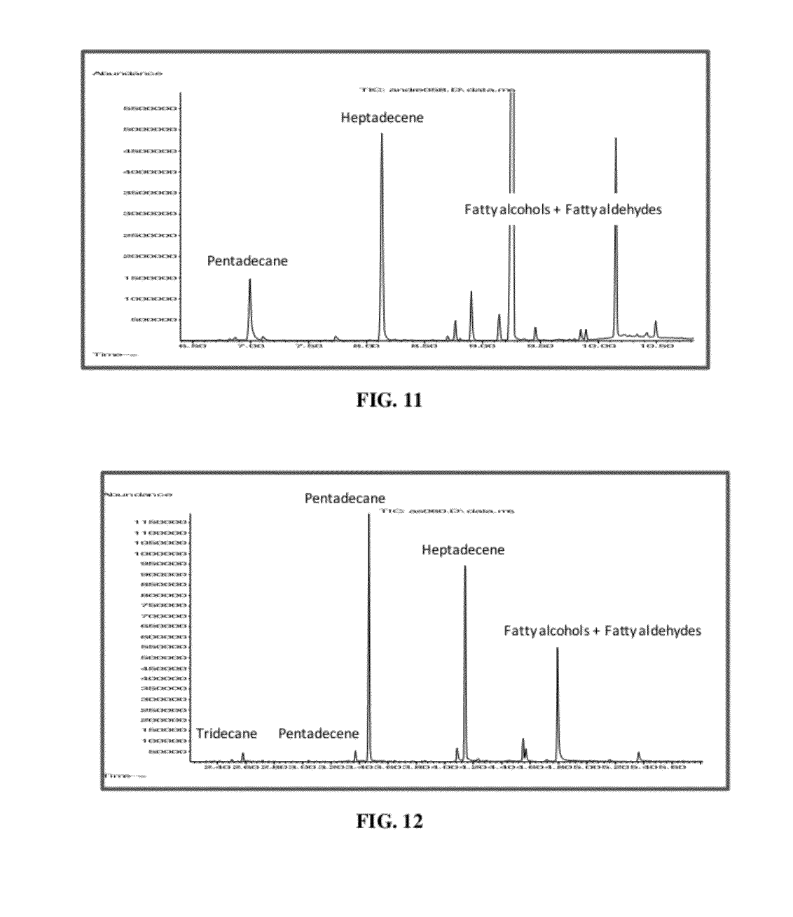

As shown in FIG. 11, E. coli cells cotransformed with the S. elongatus PCC7942_{—}1594 and Nostoc sp. PCC7210 alr5283-bearing vectors produced the same fatty aldehydes and fatty alcohols as in Example 3, but also pentadecane and heptadecene. This result indicates that alr5283 in E. coli converts hexadecanal and octadecenal to pentadecane and heptadecene, respectively, and therefore is an active fatty aldehyde decarbonylase.

### Example 9

### Production of Alkanes and Alkenes in E. coli Through Heterologous Expression of Synechococcus elongatus PCC7942 orf1594 and Acaryochloris marina MBIC11017 AM1_{—}4041

The genomic DNA encoding Acaryochloris marina MBIC11017 AM1_{—}4041 (YP_{—}001518340; putative decarbonylase) (SEQ ID NO:9) was codon optimized for expression in E. coli (SEQ ID NO:46), synthesized, and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/mL spectinomycin and 100 μg/mL carbenicillin. The cells were cultured and extracted as in Example 3 and analyzed by GC-MS as described in Example 26.

As shown in FIG. 12, E. coli cells cotransformed with the S. elongatus PCC7942_{—}1594 and A. marina MBIC11017 AM1_{—}4041-bearing vectors produced the same fatty aldehydes and fatty alcohols as in Example 3, but also tridecane, pentadecene, pentadecane and heptadecene. This result indicates that AM1_{—}4041 in E. coli converts tetradecanal, hexadecenal, hexadecanal and octadecenal to tridecane, pentadecene, pentadecane and heptadecene, respectively, and therefore is an active fatty aldehyde decarbonylase.

### Example 10

### Production of Alkanes and Alkenes in E. coli Through Heterologous Expression of Synechococcus elongatus PCC7942 orf1594 and Thermosynechococcus elongatus BP-1 tll1313

The genomic DNA encoding Thermosynechococcus elongatus BP-1 tll1313 (NP_{—}682103; putative decarbonylase) (SEQ ID NO:11) was codon optimized for expression in E. coli (SEQ ID NO:47), synthesized, and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/mL spectinomycin and 100 μg/mL carbenicillin. The cells were cultured and extracted as in Example 3 and analyzed by GC-MS as described in Example 26.

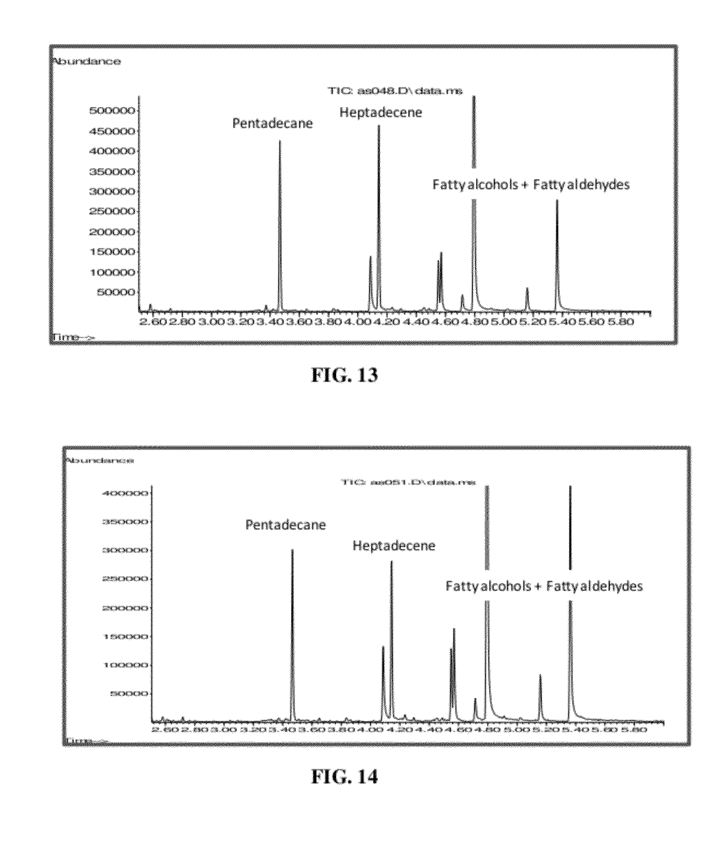

As shown in FIG. 13, E. coli cells cotransformed with the S. elongatus PCC7942_{—}1594 and T. elongatus BP-1 tll1313-bearing vectors produced the same fatty aldehydes and fatty alcohols as in Example 3, but also pentadecane and heptadecene. This result indicates that tll1313 in E. coli converts hexadecanal and octadecenal to pentadecane and heptadecene, respectively, and therefore is an active fatty aldehyde decarbonylase.

### Example 11

### Production of Alkanes and Alkenes in E. coli Through Heterologous Expression of Synechococcus elongatus PCC7942 orf1594 and Synechococcus sp. JA-3-3Ab CYA_{—}0415

The genomic DNA encoding Synechococcus sp. JA-3-3Ab CYA_{—}0415 (YP_{—}473897; putative decarbonylase) (SEQ ID NO:13) was codon optimized for expression in E. coli (SEQ ID NO:48), synthesized, and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/mL spectinomycin and 100 μg/mL carbenicillin. The cells were cultured and extracted as in Example 3 and analyzed by GC-MS as described in Example 26.

As shown in FIG. 14, E. coli cells cotransformed with the S. elongatus PCC7942_{—}1594 and Synechococcus sp. JA-3-3Ab CYA_{—}0415-bearing vectors produced the same fatty aldehydes and fatty alcohols as in Example 3, but also pentadecane and heptadecene. This result indicates that Npun02004178 in E. coli converts hexadecanal and octadecenal to pentadecane and heptadecene, respectively, and therefore is an active fatty aldehyde decarbonylase.

### Example 12

### Production of Alkanes and Alkenes in E. coli Through Heterologous Expression of Synechococcus elongatus PCC7942 orf1594 and Gloeobacter violaceus PCC7421 gll3146

The genomic DNA encoding Gloeobacter violaceus PCC7421 gll3146 (NP_{—}926092; putative decarbonylase) (SEQ ID NO:15) was amplified and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/mL spectinomycin and 100 μg/mL carbenicillin. The cells were cultured and extracted as in Example 3 and analyzed by GC-MS as described in Example 1.

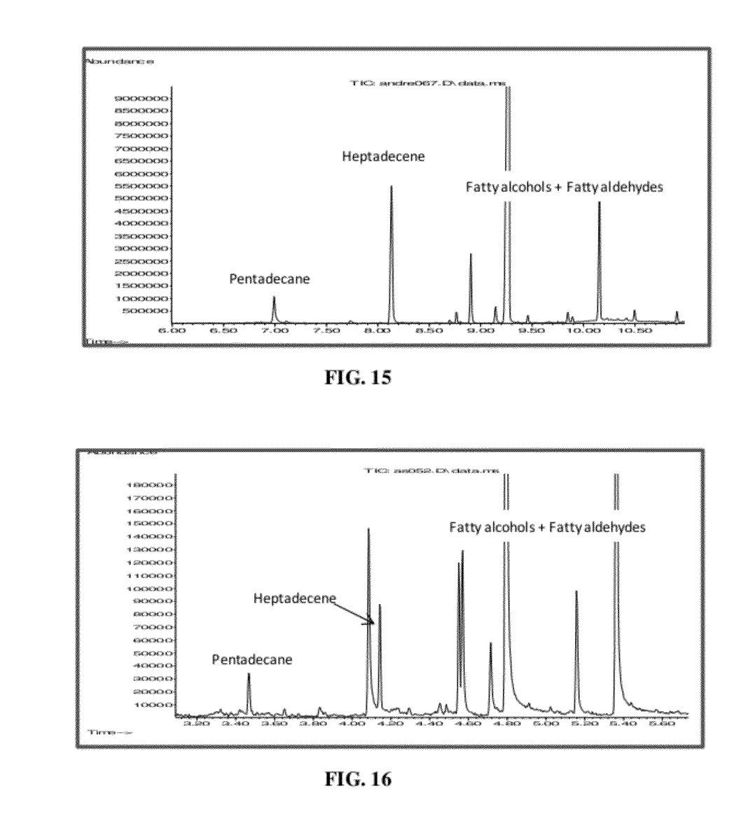

As shown in FIG. 15, E. coli cells cotransformed with the S. elongatus PCC7942_{—}1594 and G. violaceus PCC7421 gll3146-bearing vectors produced the same fatty aldehydes and fatty alcohols as in Example 3, but also pentadecane and heptadecene. This result indicates that gll3146 in E. coli converts hexadecanal and octadecenal to pentadecane and heptadecene, respectively, and therefore is an active fatty aldehyde decarbonylase.

### Example 13

### Production of Alkanes and Alkenes in E. coli Through Heterologous Expression of Synechococcus elongatus PCC7942 orf1594 and Prochlorococcus marinus MIT9313 PMT1231

The genomic DNA encoding Prochlorococcus marinus MIT9313 PMT1231 (NP_{—}895059; putative decarbonylase) (SEQ ID NO:17) was codon optimized for expression in E. coli (SEQ ID NO:49), synthesized, and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/mL spectinomycin and 100 μg/mL carbenicillin. The cells were cultured and extracted as in Example 3 and analyzed by GC-MS as described in Example 26.

As shown in FIG. 16, E. coli cells cotransformed with the S. elongatus PCC7942_{—}1594 and P. marinus MIT9313 PMT1231-bearing vectors produced the same fatty aldehydes and fatty alcohols as in Example 3, but also pentadecane and heptadecene. This result indicates that PMT1231 in E. coli converts hexadecanal and octadecenal to pentadecane and heptadecene, respectively, and therefore is an active fatty aldehyde decarbonylase.

### Example 14

### Production of Alkanes and Alkenes in E. coli Through Heterologous Expression of Synechococcus elongatus PCC7942 orf1594 and Prochlorococcus marinus CCMP1986 PMM0532

The genomic DNA encoding Prochlorococcus marinus CCMP1986 PMM0532 (NP_{—}892650; putative decarbonylase) (SEQ ID NO:19) was amplified and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/mL spectinomycin and 100 μg/mL carbenicillin. The cells were cultured and extracted as in Example 3 and analyzed by GC-MS as described in Example 1.

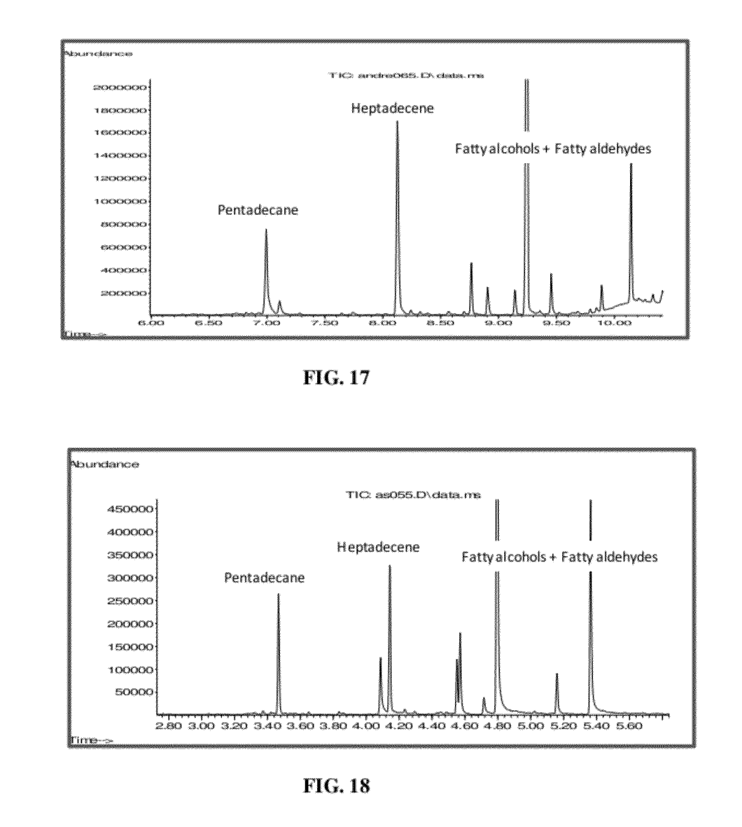

As shown in FIG. 17, E. coli cells cotransformed with the S. elongatus PCC7942_{—}1594 and P. marinus CCMP1986 PMM0532-bearing vectors produced the same fatty aldehydes and fatty alcohols as in Example 3, but also pentadecane and heptadecene. This result indicates that PMM0532 in E. coli converts hexadecanal and octadecenal to pentadecane and heptadecene, respectively, and therefore is an active fatty aldehyde decarbonylase.

### Example 15

### Production of Alkanes and Alkenes in E. coli Through Heterologous Expression of Synechococcus elongatus PCC7942 orf1594 and Prochlorococcus marinus NATL2A PMN2A_{—}1863

The genomic DNA encoding Prochlorococcus marinus NATL2A PMN2A_{—}1863 (YP_{—}293054; putative decarbonylase) (SEQ ID NO:21) was codon optimized for expression in E. coli (SEQ ID NO:51), synthesized, and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/mL spectinomycin and 100 μg/mL carbenicillin. The cells were cultured and extracted as in Example 3 and analyzed by GC-MS as described in Example 26.

As shown in FIG. 18, E. coli cells cotransformed with the S. elongatus PCC7942_{—}1594 and P. marinus NATL2A PMN2A_{—}1863-bearing vectors produced the same fatty aldehydes and fatty alcohols as in Example 3, but also pentadecane and heptadecene. This result indicates that PMN2A_{—}1863 in E. coli converts hexadecanal and octadecenal to pentadecane and heptadecene, respectively, and therefore is an active fatty aldehyde decarbonylase.

### Example 16

### Production of Alkanes and Alkenes in E. coli Through Heterologous Expression of Synechococcus elongatus PCC7942 orf1594 and Synechococcus sp. RS9917 RS9917_{—}09941

The genomic DNA encoding Synechococcus sp. RS9917 RS9917_{—}09941 (ZP_{—}01079772; putative decarbonylase) (SEQ ID NO:23) was codon optimized for expression in E. coli (SEQ ID NO:52), synthesized, and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/mL spectinomycin and 100 μg/mL carbenicillin. The cells were cultured and extracted as in Example 3 and analyzed by GC-MS as described in Example 26.

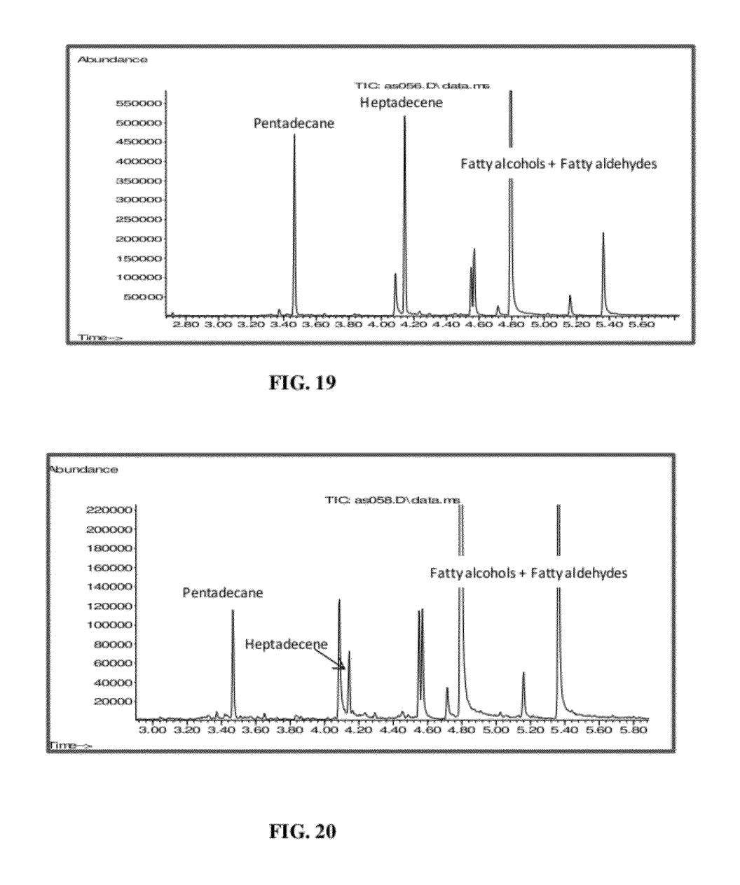

As shown in FIG. 19, E. coli cells cotransformed with the S. elongatus PCC7942_{—}1594 and Synechococcus sp. RS9917 RS9917_{—}09941-bearing vectors produced the same fatty aldehydes and fatty alcohols as in Example 3, but also pentadecane and heptadecene. This result indicates that RS9917_{—}09941 in E. coli converts hexadecanal and octadecenal to pentadecane and heptadecene, respectively, and therefore is an active fatty aldehyde decarbonylase.

### Example 17

### Production of Alkanes and Alkenes in E. coli Through Heterologous Expression of Synechococcus elongatus PCC7942 orf1594 and Synechococcus sp. RS9917 RS9917_{—}12945

The genomic DNA encoding Synechococcus sp. RS9917 RS9917_{—}12945 (ZP_{—}01080370; putative decarbonylase) (SEQ ID NO:25) was codon optimized for expression in E. coli (SEQ ID NO:53), synthesized, and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/mL spectinomycin and 100 μg/mL carbenicillin. The cells were cultured and extracted as in Example 3 and analyzed by GC-MS as described in Example 26.

As shown in FIG. 20, E. coli cells cotransformed with the S. elongatus PCC7942_{—}1594 and Synechococcus sp. RS9917 RS9917_{—}12945-bearing vectors produced the same fatty aldehydes and fatty alcohols as in Example 3, but also pentadecane and heptadecene. This result indicates that RS9917_{—}12945 in E. coli converts hexadecanal and octadecenal to pentadecane and heptadecene, respectively, and therefore is an active fatty aldehyde decarbonylase.

### Example 18

### Production of Alkanes and Alkenes in E. coli Through Heterologous Expression of Synechococcus elongatus PCC7942 orf1594 and Cyanothece sp. ATCC51142 cce_{—}0778

The genomic DNA encoding Cyanothece sp. ATCC51142 cce_{—}0778 (YP_{—}001802195; putative decarbonylase) (SEQ ID NO:27) was synthesized and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/mL spectinomycin and 100 μg/mL carbenicillin. The cells were cultured and extracted as in Example 3 and analyzed by GC-MS as described in Example 26.

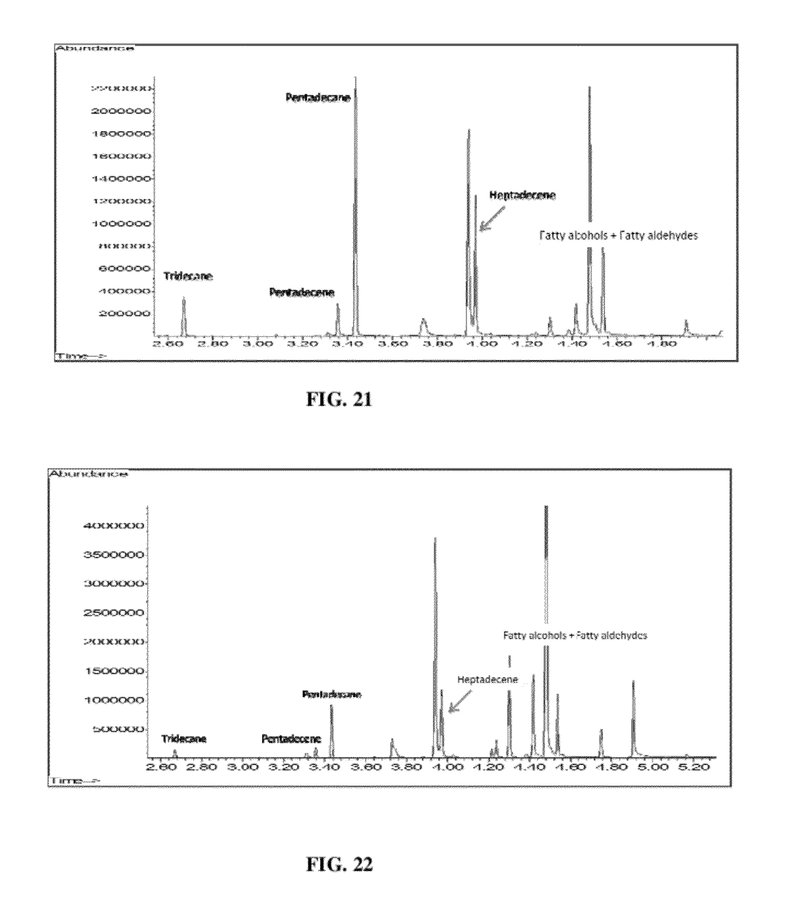

As shown in FIG. 21, E. coli cells cotransformed with the S. elongatus PCC7942_{—}1594 and Cyanothece sp. ATCC51142 cce_{—}0778-bearing vectors produced the same fatty aldehydes and fatty alcohols as in Example 3, but also tridecane, pentadecene, pentadecane and heptadecene. This result indicates that ATCC51142 cce_{—}0778 in E. coli converts tetradecanal, hexadecenal, hexadecanal and octadecenal to tridecane, pentadecene, pentadecane and heptadecene, respectively, and therefore is an active fatty aldehyde decarbonylase.

### Example 19

### Production of Alkanes and Alkenes in E. coli Through Heterologous Expression of Synechococcus elongatus PCC7942 orf1594 and Cyanothece sp. PCC7425 Cyan7425_{—}0398

The genomic DNA encoding Cyanothece sp. PCC7425 Cyan7425_{—}0398 (YP_{—}002481151; putative decarbonylase) (SEQ ID NO:29) was synthesized and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/mL spectinomycin and 100 μg/mL carbenicillin. The cells were cultured and extracted as in Example 3 and analyzed by GC-MS as described in Example 26.

As shown in FIG. 22, E. coli cells cotransformed with the S. elongatus PCC7942_{—}1594 and Cyanothece sp. PCC7425 Cyan7425_{—}0398-bearing vectors produced the same fatty aldehydes and fatty alcohols as in Example 3, but also tridecane, pentadecene, pentadecane and heptadecene. This result indicates that Cyan7425_{—}0398 in E. coli converts tetradecanal, hexadecenal, hexadecanal and octadecenal to tridecane, pentadecene, pentadecane and heptadecene, respectively, and therefore is an active fatty aldehyde decarbonylase.

### Example 20

### Production of Alkanes and Alkenes in E. coli Through Heterologous Expression of Synechococcus elongatus PCC7942 orf1594 and Cyanothece sp. PCC7425 Cyan7425_{—}2986

The genomic DNA encoding Cyanothece sp. PCC7425 Cyan7425_{—}2986 (YP_{—}002483683; putative decarbonylase) (SEQ ID NO:31) was synthesized and cloned into the NdeI and XhoI sites of vector OP-183 (pACYC derivative) under the control of the P_{trc} promoter. The resulting construct was cotransformed with OP80-PCC7942_{—}1594 into E. coli MG1655 and the cells were grown at 37° C. in M9 minimal media supplemented with 100 μg/mL spectinomycin and 100 μg/mL carbenicillin. The cells were cultured and extracted as in Example 3 and analyzed by GC-MS as described in Example 26.

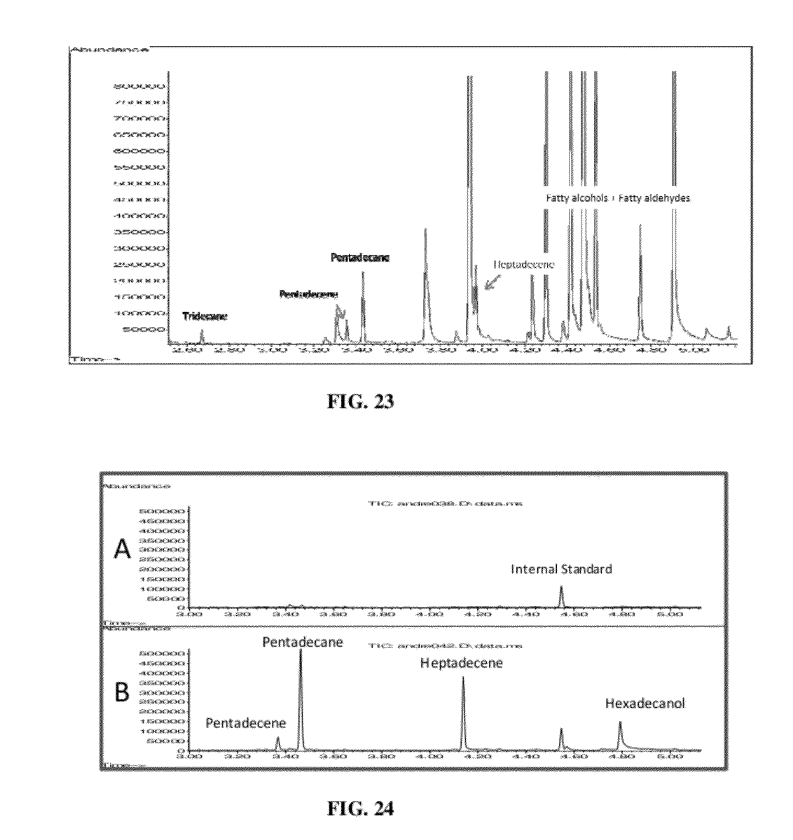

As shown in FIG. 23, E. coli cells cotransformed with the S. elongatus PCC7942_{—}1594 and Cyanothece sp. PCC7425 Cyan7425_{—}2986-bearing vectors produced the same fatty aldehydes and fatty alcohols as in Example 3, but also tridecane, pentadecene, pentadecane and heptadecene. This result indicates that Cyan7425_{—}2986 in E. coli converts tetradecanal, hexadecenal, hexadecanal and octadecenal to tridecane, pentadecene, pentadecane and heptadecene, respectively, and therefore is an active fatty aldehyde decarbonylase.

### Example 21

### Production of Alkanes and Alkenes in E. coli Through Heterologous Expression of Prochlorococcus marinus CCMP1986 PMM0533 and Prochlorococcus marinus CCMP1986 PMM0532

The genomic DNA encoding P. marinus CCMP1986 PMM0533 (NP_{—}892651; putative aldehyde-generating enzyme) (SEQ ID NO:71) and Prochlorococcus marinus CCMP1986 PMM0532 (NP_{—}892650; putative decarbonylase) (SEQ ID NO:19) were amplified and cloned into the NcoI and EcoRI sites of vector OP-80 and the NdeI and XhoI sites of vector OP-183, respectively. The resulting constructs were separately transformed and cotransformed into E. coli MG1655 and the cells were grown at 37° C. in M9 minimal media supplemented with 100 μg/mL spectinomycin and 100 μg/mL carbenicillin. The cells were cultured and extracted as in Example 3 and analyzed by GC-MS as described in Example 26.

As shown in FIG. 24A, E. coli cells transformed with only the P. marinus CCMP1986 PMM0533-bearing vector did not produce any fatty aldehydes or fatty alcohols. However, E. coli cells cotransformed with PMM0533 and PMM0532-bearing vectors produced hexadecanol, pentadecane and heptadecene (FIG. 24B). This result indicates that PMM0533 only provides fatty aldehyde substrates for the decarbonylation reaction when it interacts with a decarbonylase, such as PMM0532.

### Example 22

### Production of Alkanes and Alkenes in a Fatty Acyl-CoA-Producing E. coli Strain Through Heterologous Expression of Synechococcus elongatus PCC7942 orf1594 and Acaryochloris marina MBIC11017 AM1_{—}4041

The genomic DNA encoding Acaryochloris marina MBIC11017 AM1_{—}4041 (YP_{—}001518340; putative fatty aldehyde decarbonylase) (SEQ ID NO:9) was synthesized and cloned into the NdeI and XhoI sites of vector OP-183 (pACYC derivative) under the control of the P_{trc} promoter. The resulting construct was cotransformed with OP80-PCC7942_{—}1594 into E. coli MG1655 ΔfadE lacZ::P_{trc} ′tesA-fadD. This strain expresses a cytoplasmic version of the E. coli thioesterase, ′TesA, and the E. coli acyl-CoA synthetase, FadD, under the control of the P_{trc} promoter, and therefore produces fatty acyl-CoAs. The cells were grown at 37° C. in M9 minimal media supplemented with 100 μg/mL spectinomycin and 100 μg/mL carbenicillin. The cells were cultured and extracted as in Example 3 and analyzed by GC-MS as described in Example 1.

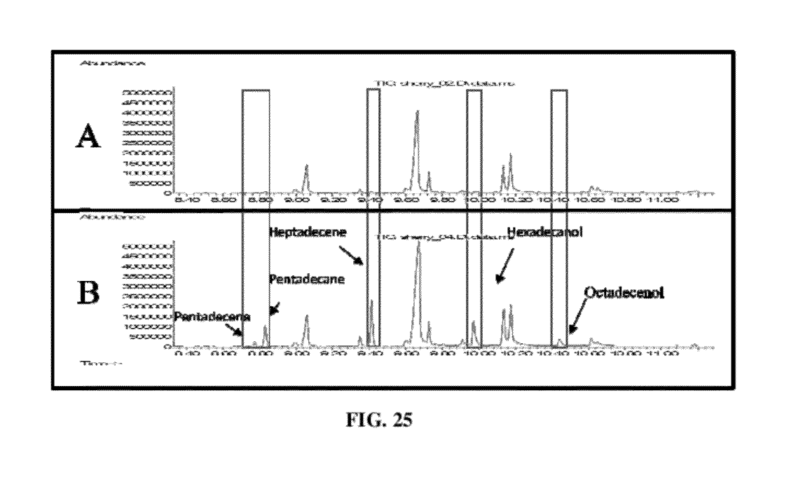

As shown in FIG. 25, these E. coli cells cotransformed with S. elongatus PCC7942_{—}1594 and A. marina MBIC11017 AM1_{—}4041 also produced alkanes and fatty alcohols. This result indicates that S. elongatus PCC7942_{—}1594 is able to use acyl-CoA as a substrate to produce hexadecenal, hexadecanal and octadecenal, which is then converted into pentadecene, pentadecane and heptadecene, respectively, by A. marina MBIC11017 AM1_{—}4041.

### Example 23

### Production of Alkanes and Alkenes in a Fatty Acyl-CoA-Producing E. coli Strain Through Heterologous Expression of Synechocystis sp. PCC6803 sll0209 and Synechocystis sp. PCC6803 sll0208

The genomic DNA encoding Synechocystis sp. PCC6803 sll0208 (NP_{—}442147; putative fatty aldehyde decarbonylase) (SEQ ID NO:3) was synthesized and cloned into the NdeI and XhoI sites of vector OP-183 (pACYC derivative) under the control of the P_{trc} promoter. The genomic DNA encoding Synechocystis sp. PCC6803 sll0209 (NP_{—}442146; acyl-ACP reductase) (SEQ ID NO:67) was synthesized and cloned into the NcoI and EcoRI sites of vector OP-183 (pACYC derivative) under the control of the P_{trc} promoter. The resulting constructs were cotransformed with into E. coli MG1655 ΔfadE lacZ::P_{trc} ′tesA-fadD. This strain expresses a cytoplasmic version of the E. coli thioesterase, ′TesA, and the E. coli acyl-CoA synthetase, FadD, under the control of the P_{trc} promoter, and therefore produces fatty acyl-CoAs. The cells were grown at 37° C. in M9 minimal media supplemented with 100 μg/mL spectinomycin and 100 μg/mL carbenicillin. The cells were cultured and extracted as in Example 3 and analyzed by GC-MS as described in Example 26.

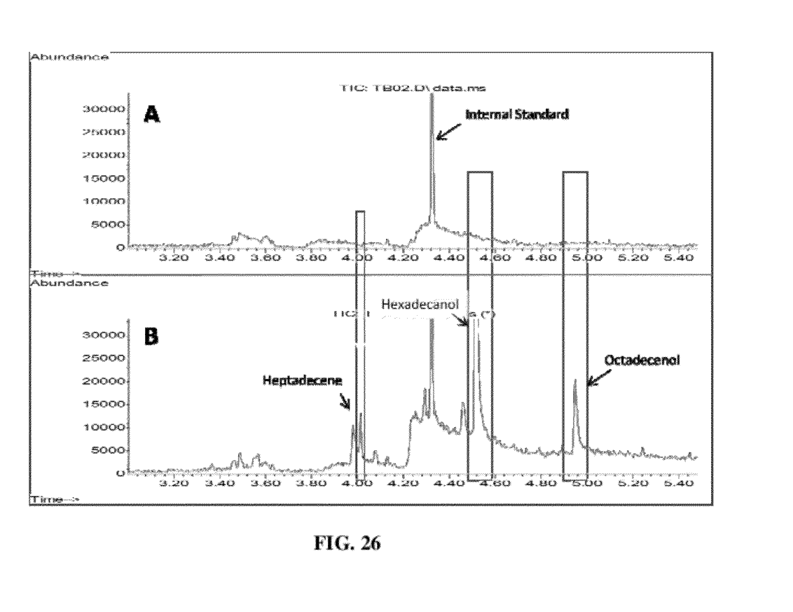

As shown in FIG. 26, these E. coli cells transformed with Synechocystis sp. PCC6803 sll0209 did not produce any fatty aldehydes or fatty alcohols. However, when cotransformed with Synechocystis sp. PCC6803 sll0208 and sll0209, they produced alkanes, fatty aldehydes and fatty alcohols. This result indicates that Synechocystis sp. PCC6803 sll0209 is able to use acyl-CoA as a substrate to produce fatty aldehydes such as tetradecanal, hexadecanal and octadecenal, but only when coexpressed with a fatty aldehyde decarbonylase. The fatty aldehydes apparently are further reduced to the corresponding fatty alcohols, tetradecanol, hexadecanol and octadecenol, by an endogenous E. coli aldehyde reductase activity. In this experiment, octadecenal was converted into heptadecene by Synechocystis sp. PCC6803 sll0208.

### Example 24

### Production of Alkanes and Alkenes in a Fatty Aldehyde-Producing E. coli Strain Through Heterologous Expression of Nostoc punctiforme PCC73102 Npun02004178 and Several of its Homologs

The genomic DNA encoding Nostoc punctiforme PCC73102 Npun02004178 (ZP_{—}00108838; putative fatty aldehyde decarbonylase) (SEQ ID NO:5) was amplified and cloned into the NdeI and XhoI sites of vector OP-183 (pACYC derivative) under the control of the P_{trc} promoter. The genomic DNA encoding Mycobacterium smegmatis strain MC2 155 orf MSMEG_{—}5739 (YP_{—}889972, putative carboxylic acid reductase) (SEQ ID NO:85) was amplified and cloned into the NcoI and EcoRI sites of vector OP-180 (pCL1920 derivative) under the control of the P_{trc} promoter. The two resulting constructs were cotransformed into E. coli MG1655 fadD lacZ::P_{trc}-′tesA. In this strain, fatty aldehydes were provided by MSMEG_{—}5739, which reduces free fatty acids (formed by the action of ′TesA) to fatty aldehydes. The cells were grown at 37° C. in M9 minimal media supplemented with 100 μg/mL spectinomycin and 100 μg/mL carbenicillin. The cells were cultured and extracted as in Example 3 and analyzed by GC-MS as described in Example 1.

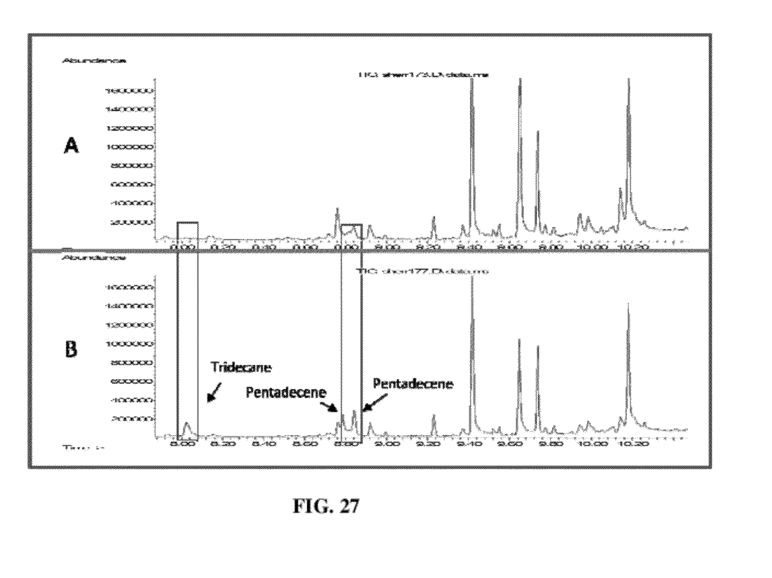

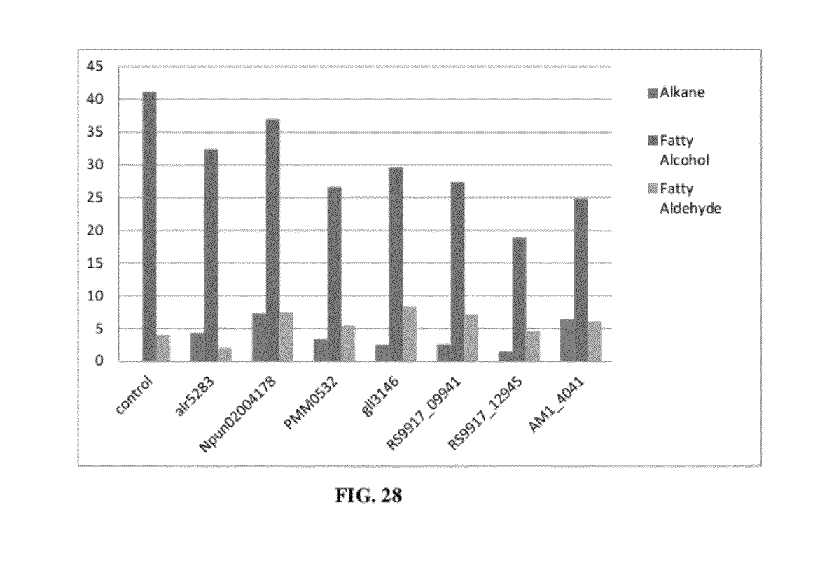

As shown in FIG. 27, these E. coli cells cotransformed with the N. punctiforme PCC73102 Npun02004178 and M. smegmatis strain MC2 155 MSMEG_{—}5739-bearing vectors produced tridecane, pentadecene and pentadecane. This result indicates that Npun02004178 in E. coli converts tetradecanal, hexadecenal and hexadecanal provided by the carboxylic acid reductase MSMEG_{—}5739 to tridecane, pentadecene and pentadecane. As shown in FIG. 28, in the same experimental set-up, the following fatty aldehyde decarbonylases also converted fatty aldehydes provided by MSMEG_{—}5739 to the corresponding alkanes when expressed in E. coli MG1655 fadD lacZ::P_{trc}-′tesA: Nostoc sp. PCC7210 alr5283 (SEQ ID NO:7), P. marinus CCMP1986 PMM0532 (SEQ ID NO:19), G. violaceus PCC7421 gll3146 (SEQ ID NO:15), Synechococcus sp. RS9917_{—}09941 (SEQ ID NO:23), Synechococcus sp. RS9917_{—}12945 (SEQ ID NO:25), and A. marina MBIC11017 AM1_{—}4041 (SEQ ID NO:9).

### Example 25

### Cyanobacterial Fatty Aldehyde Decarbonylases Belong to the Class of Non-Heme Diiron Proteins. Site-Directed Mutagenesis of Conserved Histidines to Phenylalanines in Nostoc punctiforme PCC73102 Npun02004178 does not Abolish its Catalytic Function

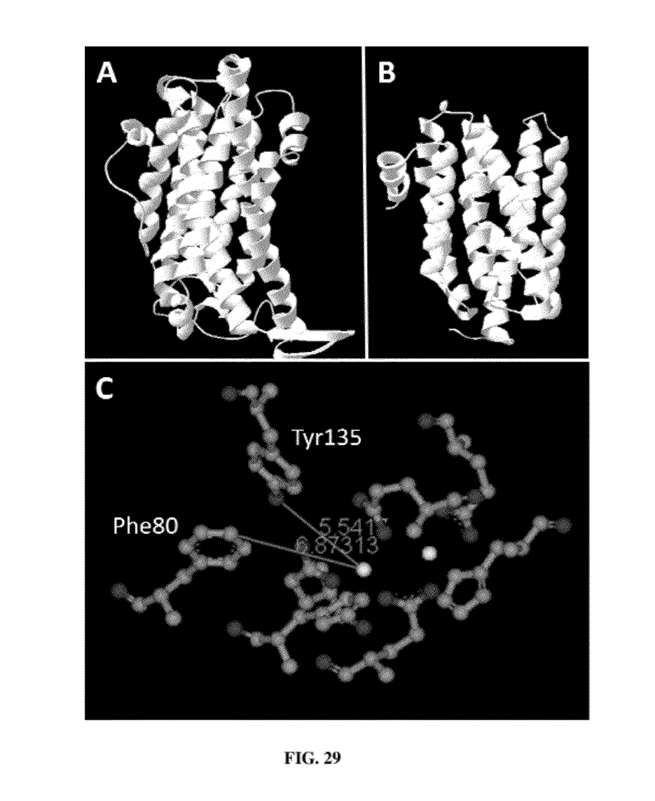

As discussed in Example 13, the hypothetical protein PMT1231 from Prochlorococcus marinus MIT9313 (SEQ ID NO:18) is an active fatty aldehyde decarbonylase. Based on the three-dimensional structure of PMT1231, which is available at 1.8 Å resolution (pdb2OC5A) (see FIG. 29B), cyanobacterial fatty aldehyde decarbonylases have structural similarity with non-heme diiron proteins, in particular with class I ribonuclease reductase subunit β proteins, RNRβ (Stubbe and Riggs-Gelasco, TIBS 1998, vol. 23., pp. 438) (see FIG. 29A). Class Ia and Ib RNRβ contains a diferric tyrosyl radical that mediates the catalytic activity of RNRβ (reduction of ribonucleotides to deoxyribonucleotides). In E. coli RNRβ, this tyrosine is in position 122 and is in close proximity to one of the active site's iron molecules. Structural alignment showed that PMT1231 contained a phenylalanine in the same position as RNRb tyr122, suggesting a different catalytic mechanism for cyanobacterial fatty aldehyde decarbonylases. However, an alignment of all decarbonylases showed that two tyrosine residues were completely conserved in all sequences, tyr135 and tyr138 with respect to PMT1231, with tyr135 being in close proximity (5.5 Å) to one of the active site iron molecules (see FIG. 29C). To examine whether either of the two conserved tyrosine residues is involved in the catalytic mechanism of cyanobacterial fatty aldehyde decarbonylases, these residues were replaced with phenylalanine in Npun02004178 (tyr 123 and tyr126) as follows.

The genomic DNA encoding S. elongatus PCC7942 ORF1594 (SEQ ID NO:65) was cloned into the NcoI and EcoRI sites of vector OP-80 (pCL1920 derivative) under the control of the P_{trc} promoter. The genomic DNA encoding N. punctiforme PCC73102 Npun02004178 (SEQ ID NO:5) was also cloned into the NdeI and XhoI sites of vector OP-183 (pACYC177 derivative) under the control of the P_{trc} promoter. The latter construct was used as a template to introduce a mutation at positions 123 and 126 of the decarbonylase protein, changing the tyrosines to phenylalanines using the primers gttttgcgatcgcagcatttaacatttacatccccgttgccgacg and gttttgcgatcgcagcatataacattttcatccccgttgccgacg, respectively. The resulting constructs were then transformed into E. coli MG1655. The cells were grown at 37° C. in M9 minimal media supplemented with 1% glucose (w/v), and 100 μg/mL carbenicillin and spectinomycin. The cells were cultured and extracted as in Example 3.

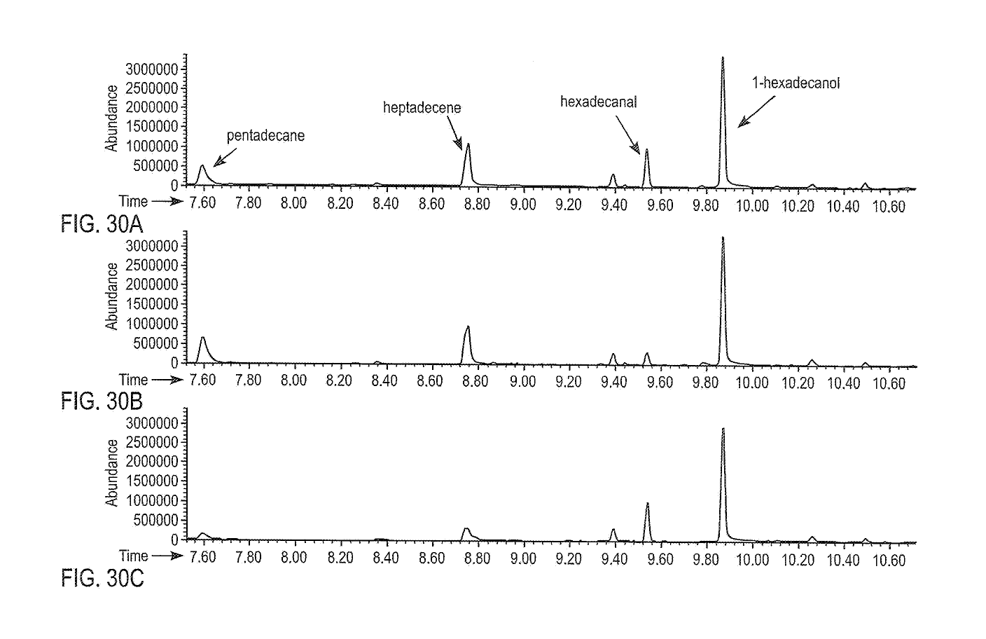

As shown in FIG. 30, the two Npun02004178 Tyr to Phe protein variants were active and produced alkanes when coexpressed with S. elongatus PCC7942 ORF1594. This result indicates that in contrast to class Ia and Ib RNRβ proteins, the catalytic mechanism of fatty aldehyde decarbonylases does not involve a tyrosyl radical.

### Example 26

### Biochemical Characterization of Nostoc punctiforme PCC73102 Npun02004178

The genomic DNA encoding N. punctiforme PCC73102 Npun02004178 (SEQ ID NO:5) was cloned into the NdeI and XhoI sites of vector pET-15b under the control of the T7 promoter. The resulting Npun02004178 protein contained an N-terminal His-tag. An E. coli BL21 strain (DE3) (Invitrogen) was transformed with the plasmid by routine chemical transformation techniques. Protein expression was carried out by first inoculating a colony of the E. coli strain in 5 mL of LB media supplemented with 100 mg/L of carbenicillin and shaken overnight at 37° C. to produce a starter culture. This starter cultures was used to inoculate 0.5 L of LB media supplemented with 100 mg/L of carbenicillin. The culture was shaken at 37° C. until an OD₆₀₀ value of 0.8 was reached, and then IPTG was added to a final concentration of 1 mM. The culture was then shaken at 37° C. for approximately 3 additional h. The culture was then centrifuged at 3,700 rpm for 20 min at 4° C. The pellet was then resuspended in 10 mL of buffer containing 100 mM sodium phosphate buffer at pH 7.2 supplemented with Bacterial ProteaseArrest (GBiosciences). The cells were then sonicated at 12 W on ice for 9 s with 1.5 s of sonication followed by 1.5 s of rest. This procedure was repeated 5 times with one min intervals between each sonication cycle. The cell free extract was centrifuged at 10,000 rpm for 30 min at 4° C. 5 mL of Ni-NTA (Qiagen) was added to the supernatant and the mixture was gently stirred at 4° C. The slurry was passed over a column removing the resin from the lysate. The resin was then washed with 30 mL of buffer containing 100 mM sodium phosphate buffer at pH 7.2 plus 30 mM imidazole. Finally, the protein was eluted with 10 mL of 100 mM sodium phosphate buffer at pH 7.2 plus 250 mM imidazole. The protein solution was dialyzed with 200 volumes of 100 mM sodium phosphate buffer at pH 7.2 with 20% glycerol. Protein concentration was determined using the Bradford assay (Biorad). 5.6 mg/mL of Npun02004178 protein was obtained.

To synthesize octadecanal for the decarbonylase reaction, 500 mg of octadecanol (Sigma) was dissolved in 25 mL of dichloromethane. Next, 200 mg of pyridinium chlorochromate (TCI America) was added to the solution and stirred overnight. The reaction mixture was dried under vacuum to remove the dichloromethane. The remaining products were resuspended in hexane and filtered through Whatman filter paper. The filtrate was then dried under vacuum and resuspended in 5 mL of hexane and purified by silica flash chromatography. The mixture was loaded onto the gravity fed column in hexane and then washed with two column volumes of hexane. The octadecanal was then eluted with an 8:1 mixture of hexane and ethyl acetate. Fractions containing octadecanal were pooled and analyzed using the GC/MS methods described below. The final product was 95% pure as determined by this method.

To test Npun02004178 protein for decarbonylation activity, the following enzyme assays were set-up. 200 μL reactions were set up in 100 mM sodium phosphate buffer at pH 7.2 with the following components at their respective final concentrations: 30 μM of purified Npun02004178 protein, 200 μM octadecanal, 0.11 μg/mL spinach ferredoxin (Sigma), 0.05 units/mL spinach ferredoxin reductase (Sigma), and 1 mM NADPH (Sigma). Negative controls included the above reaction without Npun02004178, the above reaction without octadecanal, and the above reaction without spinach ferredoxin, ferredoxin reductase and NADPH. Each reaction was incubated at 37° C. for 2 h before being extracted with 100 μL ethyl acetate. Samples were analyzed by GC/MS using the following parameters: run time: 13.13 min; column: HP-5-MS Part No. 190915-433E (length of 30 meters; I.D.: 0.25 mm narrowbore; film: 0.25 iM); inject: 1 il Agilent 6850 inlet; inlet: 300 C splitless; carrier gas: helium; flow: 1.3 mL/min; oven temp: 75° C. hold 5 min, 320 at 40° C./min, 320 hold 2 min; det: Agilent 5975B VL MSD; det. temp: 330° C.; scan: 50-550 M/Z. Heptadecane from Sigma was used as an authentic reference for determining compound retention time and fragmentation pattern.

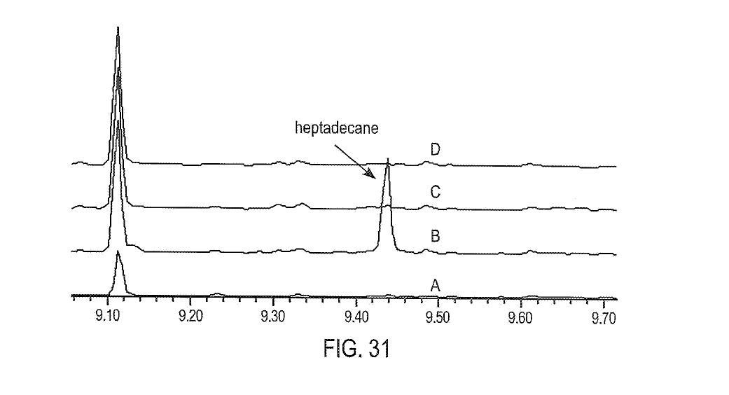

As shown in FIG. 31, in-vitro conversion of octadecanal to heptadecane was observed in the presence of Npun02004178. The enzymatic decarbonylation of octadecanal by Npun02004178 was dependent on the addition of spinach ferredoxin reductase, ferredoxin and NADPH.

Next, it was determined whether cyanobacterial ferredoxins and ferredoxin reductases can replace the spinach proteins in the in-vitro fatty aldehyde decarbonylase assay. The following four genes were cloned separately into the NdeI and XhoI sites of pET-15b: N. punctiforme PCC73102 Npun02003626 (ZP_{—}00109192, ferredoxin oxidoreductase petH without the n-terminal allophycocyanin linker domain) (SEQ ID NO:87), N. punctiforme PCC73102 Npun02001001 (ZP_{—}00111633, ferredoxin 1) (SEQ ID NO:89), N. punctiforme PCC73102 Npun02003530 (ZP_{—}00109422, ferredoxin 2) (SEQ ID NO:91) and N. punctiforme PCC73102 Npun02003123 (ZP_{—}00109501, ferredoxin 3) (SEQ ID NO:93). The four proteins were expressed and purified as described above. 1 mg/mL of each ferredoxin and 4 mg/mL of the ferredoxin oxidoreductase was obtained. The three cyanobacterial ferredoxins were tested with the cyanobacterial ferredoxin oxidoreductase using the enzymatic set-up described earlier with the following changes. The final concentration of the ferredoxin reductase was 60 μg/mL and the ferredoxins were at 50 μg/mL. The extracted enzymatic reactions were by GC/MS using the following parameters: run time: 6.33 min; column: J&W 122-5711 DB-5ht (length of 15 meters; I.D.: 0.25 mm narrowbore; film: 0.10 μM); inject: 1 μL Agilent 6850 inlet; inlet: 300° C. splitless; carrier gas: helium; flow: 1.3 mL/min; oven temp: 100° C. hold 0.5 min, 260 at 30° C./min, 260 hold 0.5 min; det: Agilent 5975B VL MSD; det. temp: 230° C.; scan: 50-550 M/Z.

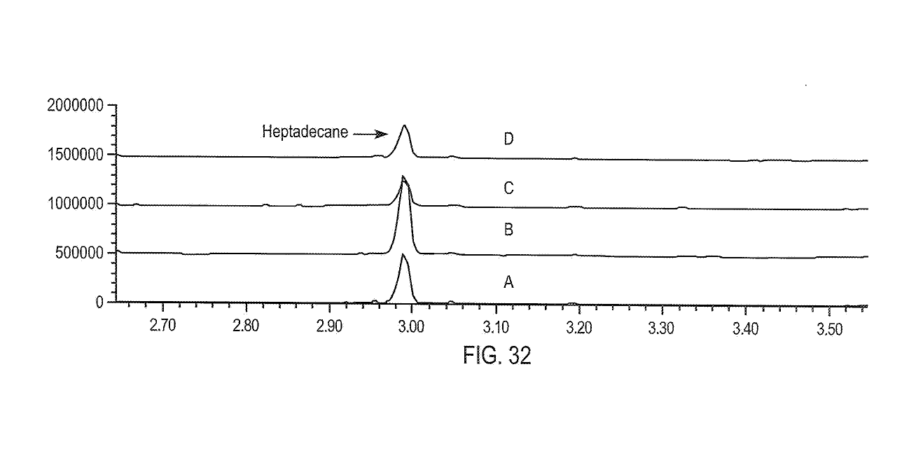

As shown in FIG. 32, Npun02004178-dependent in-vitro conversion of octadecanal to heptadecane was observed in the presence of NADPH and the cyanobacterial ferredoxin oxidoreductase and any of the three cyanobacterial ferredoxins.

### Example 27

### Biochemical Characterization of Synechococcus elongatus PCC7942 orf1594

The genomic DNA encoding S. elongatus PCC7492 orf1594 (SEQ ID NO:65) was cloned into the NcoI and XhoI sites of vector pET-28b under the control of the T7 promoter. The resulting PCC7942_orf1594 protein contained a C-terminal His-tag. An E. coli BL21 strain (DE3) (Invitrogen) was transformed with the plasmid and PCC7942_orf1594 protein was expressed and purified as described in Example 22. The protein solution was stored in the following buffer: 50 mM sodium phosphate, pH 7.5, 100 mM NaCl, 1 mM THP, 10% glycerol. Protein concentration was determined using the Bradford assay (Biorad). 2 mg/mL of PCC7942_orf1594 protein was obtained.

To test PCC7942_orf1594 protein for acyl-ACP or acyl-CoA reductase activity, the following enzyme assays were set-up. 100 μL reactions were set-up in 50 mM Tris-HCl buffer at pH 7.5 with the following components at their respective final concentrations: 10 μM of purified PCC7942_orf1594 protein, 0.01-1 mM acyl-CoA or acyl-ACP, 2 mM MgCl₂, 0.2-2 mM NADPH. The reactions were incubated for 1 h at 37° C. and where stopped by adding 100 μL ethyl acetate (containing 5 mg/l 1-octadecene as internal standard). Samples were vortexed for 15 min and centrifuged at max speed for 3 min for phase separation. 80 μL of the top layer were transferred into GC glass vials and analyzed by GC/MS as described in Example 26. The amount of aldehyde formed was calculated based on the internal standard.

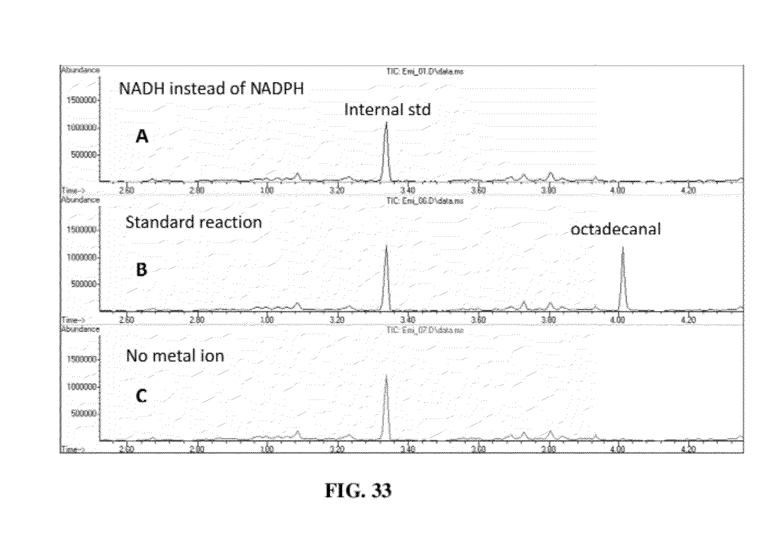

As shown in FIG. 33, PCC7942_orf1594 was able to reduce octadecanoyl-CoA to octadecanal. Reductase activity required divalent cations such as Mg²⁺, Mn²⁺ or Fe²⁺ and NADPH as electron donor. NADH did not support reductase activity. PCC7942_orf1594 was also able to reduce octadecenoyl-CoA and octadecenoyl-ACP to octadecenal. The Kₘ values for the reduction of octadecanoyl-CoA, octadecenoyl-CoA and octadecenoyl-ACP in the presence of 2 mM NADPH were determined as 45±20 μM, 82±22 μM and 7.8±2 μM, respectively. These results demonstrate that PCC7942_orf1594, in vitro, reduces both acyl-CoAs and acyl-ACPs and that the enzyme apparently has a higher affinity for acyl-ACPs as compared to acyl-CoAs. The Kₘ value for NADPH in the presence of 0.5 mM octadecanoyl-CoA for PCC7942_orf1594 was determined as 400±80 μM.

Next, the stereospecific hydride transfer from NADPH to a fatty aldehyde catalyzed by PCC7942_orf1594 was examined. Deutero-NADPH was prepared according to the following protocol. 5 mg of NADP⁺ and 3.6 mg of D-glucose-1-d was added to 2.5 mL of 50 mM sodium phosphate buffer (pH 7.0). Enzymatic production of labeled NADPH was initiated by the addition of 5 units of glucose dehydrogenase from either Bacillus megaterium (USB Corporation) for the production of R-(4-²H)NADPH or Thermoplasma acidophilum (Sigma) for the production of S-(4-²H)NADPH. The reaction was incubated for 15 min at 37° C., centrifuge-filtered using a 10 KDa MWCO Amicon Ultra centrifuge filter (Millipore), flash frozen on dry ice, and stored at −80° C.

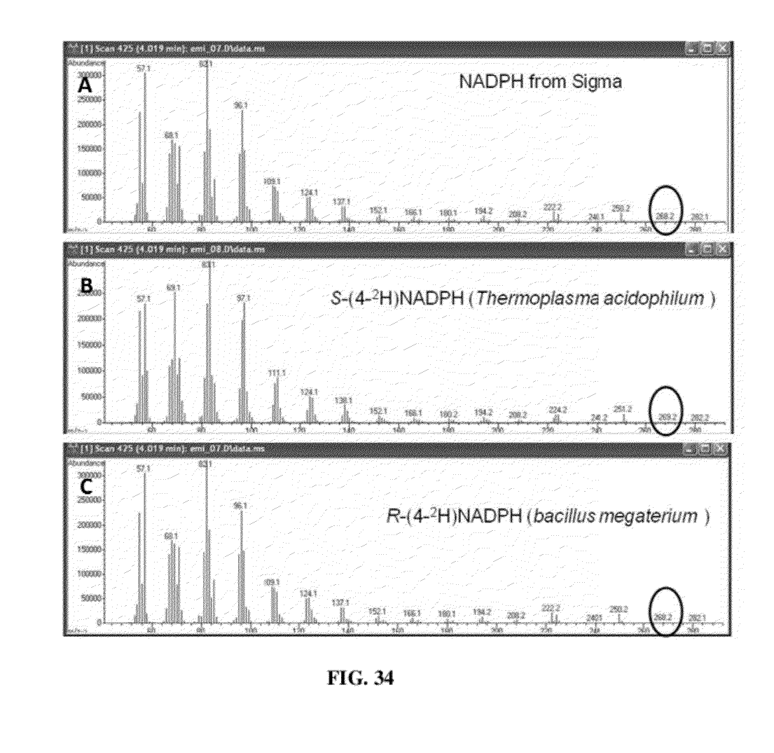

The in vitro assay reaction contained 50 mM Tris-HCl (pH 7.5), 10 μM of purified PCC7942_orf1594 protein, 1 mM octadecanoyl-CoA, 2 mM MgCl₂, and 50 μL deutero-NADPH (prepared as described above) in a total volume of 100 μL. After a 1 h incubation, the product of the enzymatic reaction was extracted and analyzed as described above. The resulting fatty aldehyde detected by GC/MS was octadecanal (see FIG. 34). Because hydride transfer from NADPH is stereospecific, both R-(4-²H)NADPH and S-(4-²H)NADPH were synthesized. Octadecanal with a plus one unit mass was observed using only the S-(4-²H)NADPH. The fact that the fatty aldehyde was labeled indicates that the deuterated hydrogen has been transferred from the labeled NADPH to the labeled fatty aldehyde. This demonstrates that NADPH is used in this enzymatic reaction and that the hydride transfer catalyzed by PCC7942_orf1594 is stereospecific.

### Example 28

### Intracellular and Extracellular Production of Fatty Aldehydes and Fatty Alcohols in E. coli Through Heterologous Expression of Synechococcus elongatus PCC7942 orf1594

The genomic DNA encoding Synechococcus elongatus PCC7942 orf1594 (YP_{—}400611; acyl-ACP reductase) (SEQ ID NO:65) was amplified and cloned into the NcoI and EcoRI sites of vector OP-80 (pCL1920 derivative) under the control of the P_{trc} promoter. The resulting construct was cotransformed into E. coli MG1655 fadE and the cells were grown at 37° C. in 15 mL Che-9 minimal media with 3% (w/v) glucose as carbon source and supplemented with 100 μg/mL spectinomycin and carbenicillin, respectively. When the culture reached OD₆₀₀ of 0.8-1.0, it was induced with 1 mM IPTG and cells were grown for an additional 24-48 h at 37° C. Che-9 minimal medium is defined as: 6 g/L Na₂HPO₄, 3 g/L KH₂PO₄, 0.5 g/L NaCl, 2 g/L NH₄C1, 0.25 g/L MgSO₄×7 H₂O, 11 mg/L CaCl₂, 27 mg/L Fe₃Cl×6H₂O, 2 mg/L ZnCl×4H₂O, 2 mg/L Na₂MoO₄×2H₂O, 1.9 mg/L CuSO₄×5 H₂O, 0.5 mg/L H₃BO₃, 1 mg/L thiamine, 200 mM Bis-Tris (pH 7.25) and 0.1% (v/v) Triton-X100. When the culture reached OD₆₀₀ of 1.0-1.2, it was induced with 1 mM IPTG and cells were allowed to grow for an additional 40 hrs at 37° C. Cells from 0.5 mL of culture were extracted with 0.5 mL of ethyl acetate for total hydrocarbon production as described in Example 26. Additionally, cells and supernatant were separated by centrifugation (4,000 g at RT for 10 min) and extracted separately.

The culture produced 620 mg/L fatty aldehydes (tetradecanal, heptadecenal, heptadecanal and octadecenal) and 1670 mg/L fatty alcohols (dodecanol, tetradecenol, tetradecanol, heptadecenol, heptadecanol and octadecenol). FIG. 35 shows the chromatogram of the extracted supernatant. It was determined that 73% of the fatty aldehydes and fatty alcohols were in the cell-free supernatant.

### Example 29

### Intracellular and Extracellular Production of Alkanes and Alkenes in E. coli Through Heterologous Expression of Synechococcus elongatus PCC7942 orf1594 and Nostoc punctiforme PCC73102 Npun02004178

The genomic DNA encoding Synechococcus elongatus PCC7942 orf1594 (YP_{—}400611; acyl-ACP reductase) (SEQ ID NO:65) was amplified and cloned into the NcoI and EcoRI sites of vector OP-80 (pCL1920 derivative) under the control of the P_{trc} promoter. The genomic DNA encoding Nostoc punctiforme PCC73102 Npun02004178 (ZP_{—}00108838; fatty aldehyde decarbonylase) (SEQ ID NO:5) was amplified and cloned into the NdeI and XhoI sites of vector OP-183 (pACYC derivative) under the control of the P_{trc} promoter. The resulting constructs were cotransformed into E. coli MG1655 fadE and the cells were grown at 37° C. in 15 mL Che9 minimal media with 3% (w/v) glucose as carbon source and supplemented with 100 μg/mL spectinomycin and carbenicillin, respectively. The cells were grown, separated from the broth, extracted, and analyzed as described in Example 28.

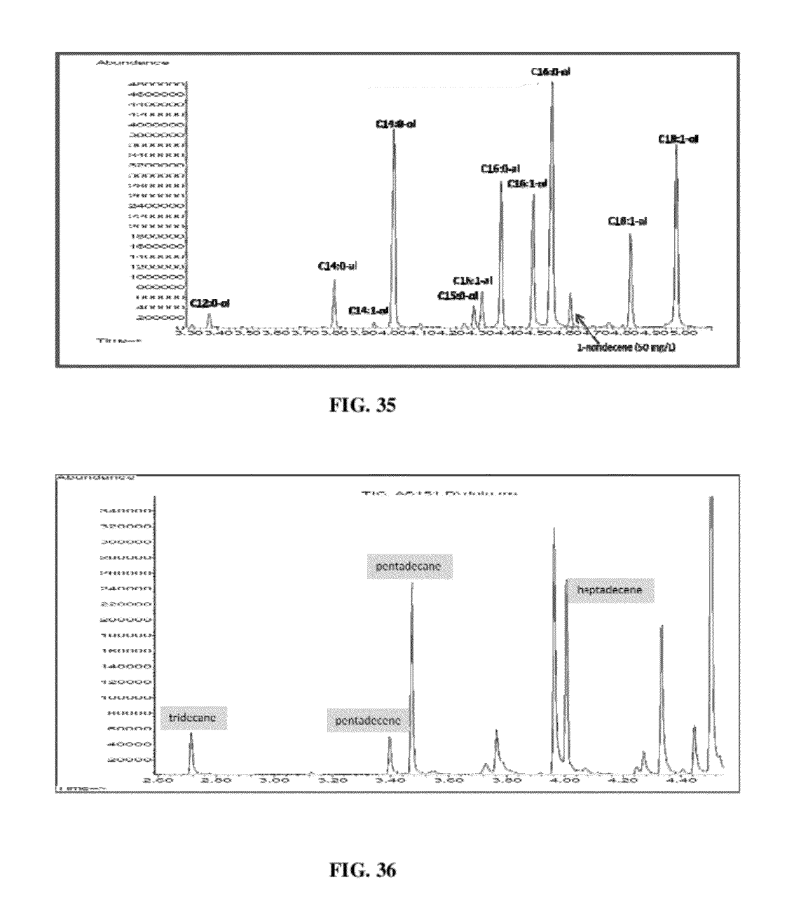

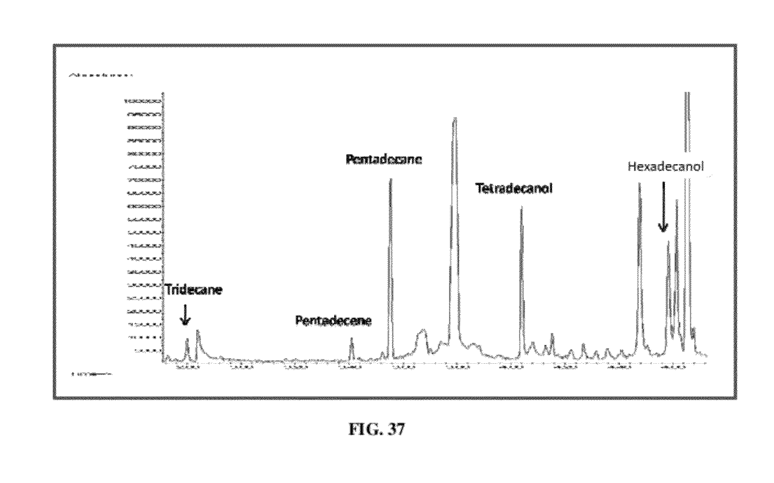

The culture produced 323 mg/L alkanes and alkenes (tridecane, pentadecene, pentadecane and heptadecene), 367 mg/L fatty aldehydes (tetradecanal, heptadecenal, heptadecanal and octadecenal) and 819 mg/L fatty alcohols (tetradecanol, heptadecenol, heptadecanol and octadecenol). FIG. 36 shows the chromatogram of the extracted supernatant. It was determined that 86% of the alkanes, alkenes, fatty aldehydes and fatty alcohols were in the cell-free supernatant.

### Example 30

### Production of Alkanes and Alkenes in E. coli Through Heterologous Expression of Nostoc sp. PCC7210 alr5284 and Nostoc sp. PCC7210 alr5283

The genomic DNA encoding Nostoc sp. PCC7210 alr5284 (NP_{—}489324; putative aldehyde-generating enzyme) (SEQ ID NO:81) was amplified and cloned into the NcoI and EcoRI sites of vector OP-80 (pCL1920 derivative) under the control of the P_{trc} promoter. The genomic DNA encoding Nostoc sp. PCC7210 alr5283 (NP_{—}489323; putative decarbonylase) (SEQ ID NO:7) was amplified and cloned into the NdeI and XhoI sites of vector OP-183 (pACYC derivative) under the control of the P_{trc} promoter. The resulting constructs were cotransformed into E. coli MG1655 and the cells were grown at 37° C. in 15 mL Che9 minimal media with 3% (w/v) glucose as carbon source and supplemented with 100 μg/mL spectinomycin and carbenicillin, respectively (as described in Example 28). Cells from 0.5 mL of culture were extracted and analyzed as described in Example 3 and analyzed by GC-MS as described in Example 26.

As shown in FIG. 37, E. coli cells cotransformed with the Nostoc sp. PCC7210 alr5284 and Nostoc sp. PCC7210 alr5283-bearing vectors produced tridecane, pentadecene, pentadecane, tetradecanol and hexadecanol. This result indicates that coexpression of Nostoc sp. PCC7210 alr5284 and alr5283 is sufficient for E. coli to produce fatty alcohols, alkanes and alkenes.

### Example 31

This example demonstrates the construction of a genetically engineered microorganism wherein the cyanobacterial genes Nostoc punctiforme PCC73102 ferrodoxin Npun_R1710 (petF) (SEQ ID NO:95) and ferrodoxin oxidoreductase Npun02003623 petH (ZP_{—}00109192) (SEQ ID NO:96) were integrated into the chromosome under the control of a Ptrc promoter.

The fadE gene of E. coli MG1655 (an E. coli K strain) was deleted using the procedure described by Datsenko et al., Proc. Natl. Acad. Sci. USA 97: 6640-6645 (2000), with the following modifications described herein.

The two primers used to create the deletion were:

Del-fadE-F
(SEQ ID NO: 97)
5′-AAAAACAGCAACAATGTGAGCTTTGTTGTAATTATATTGTAAACATATTGATTCCG
GGGATCCGTCGACC;
and
Del-fadE-R
(SEQ ID NO: 98)
5′-AAACGGAGCCTTTCGGCTCCGTTATTCATTTACGCGGCTTCAACTTTCCTGTAGGC
TGGAGCTGCTTC.

The Del-fadE-F and Del-fadE-R primers each contained 50 bases of homology to the E. coli fadE gene, and were used to amplify the Kanamycin resistance cassette from plasmid pKD13 by PCR, as described by Datsenko et al., supra. The resulting PCR product was used to transform electrocompetent E. coli MG1655 cells containing pKD46, which cells were previously induced with arabinose for 3-4 h as described by Datsenko et al., supra. Following a 3 h outgrowth in a super optimal broth with catabolite repression (SOC) medium at 37° C., the cells were plated on Luria agar plates containing 50 μg/mL of Kanamycin. Resistant colonies were isolated after an overnight incubation at 37° C. Disruption of the fadE gene was confirmed in some of the colonies by PCR amplification using primers fadE-L2 and fadE-R1, which were designed to flank the fadE gene. The fadE deletion confirmation primers used were:

(SEQ ID NO: 99)
fadE-L2 5′-CGGGCAGGTGCTATGACCAGGAC;
and
(SEQ ID NO: 100)
fadE-R1 5′-CGCGGCGTTGACCGGCAGCCTGG

After the proper fadE deletion was confirmed, one colony was used to remove the Km^{R} marker using the pCP20 plasmid as described by Datsenko et al., supra. The resulting MG1655 E. coli strain with the fadE gene deleted and the Km^{R} marker removed was named E. coli MG1655 D1.

The fhuA gene (also known as the tonA gene) of E. coli MG1655, which encodes a ferrichrome outer membrane transporter (GenBank Accession No. NP_{—}414692), was then deleted from strain MG1655 D1 using the procedure described by Datsenko et al., supra, but with the following modifications described herein. The two primers used to create the deletion were:

Del-fhuA-F
(SEQ ID NO: 101)
5′-ATCATTCTCGTTTACGTTATCATTCACTTTACATCAGAGATATACC
AATGATTCCGGGGATCCGTCGACC;
and
Del-fhuA-R
(SEQ ID NO: 102)
5′-GCACGGAAATCCGTGCCCCAAAAGAGAAATTAGAAACGGAAGGTTG
CGGTTGTAGGCTGGAGCTGCTTC

The Del-fhuA-F and Del-fhuA-R primers each contained 50 bases of homology to the E. coli fhuA gene, and were used to amplify the Kanamycin resistance cassette from plasmid pKD13 by PCR as described by Datsenko et al., supra. The PCR product obtained in this way was used to transform electrocompetent E. coli MG1655 D1 cells containing pKD46, which cells were previously induced with arabinose for 3-4 h as described by Datsenko et al., supra. Following a 3 h outgrowth in SOC medium at 37° C., cells were plated on Luria agar plates containing 50 μg/mL of Kanamycin. Resistant colonies were isolated after an overnight incubation at 37° C. Disruption of the fhuA gene was confirmed using primers fhuA-verF and fhuA-verR, which were designed to flank the fhuA gene.

(SEQ ID NO: 103)
fhuA-verF 5′-CAACAGCAACCTGCTCAGCAA;
and
(SEQ ID NO: 104)
fhuA-verR 5′-AAGCTGGAGCAGCAAAGCGTT

After the proper fhuA deletion was confirmed, one colony was used to remove the Km^{R} marker using the pCP20 plasmid as described by Datsenko et al., supra. The resulting MG1655 E. coli strain having fadE and fhuA gene deletions was named E. coli MG1655 DV2.

An expression cassette derived from pACYC177 (Chang et al., J. Bacteriol. 134:1141-1156 (1978)) called OP-183 (SEQ ID NO:105), which comprised a lacI sequence, was subject to restriction digestions by ZraI and NheI. Another expression cassette pCOLA-Duet1 (EMD Chemicals, Inc., Gibbstown, N.J.), which comprised a Kanamycin marker and a COLA replicon, was also digested with ZraI and XbaI. A1960-bp fragment from the digestion of OP-183 and a 2150-bp fragment from the digestion of pCOLA-Duet1 were ligated to form plasmid pAS52-123.

The following primers were used to amplify the ferrodoxin gene petF from the genomic DNA of Nostoc punctiforme PCC73102 (ZP_{—}00108837):

petF-forward:
(SEQ ID NO: 106)
5′-GCAATTCATATGCCAACTTATAAAGTGACACTAATTAACG-3′;
and
petF-reverse:
(SEQ ID NO: 107)
3′-TGAGTCATTTTGTTTTCCTCCTTATTAATAGAGTTCTTCTTCTTTG
TG AG-5′.

The following primers were used to amplify the ferrodoxin reductase gene petH from the genomic DNA of Nostoc punctiforme PCC73102 (ZP_{—}00108837):

petH-forward:
(SEQ ID NO: 108)
5′-TCTATTAATAAGGAGGAAAACAAAATGACTCAAGCGAAAGCCAA
AAAAGA-3′;
and
petH-reverse:
(SEQ ID NO: 109)
3′-AGCTTCGAATTCTTAGTAAGTTTCTACGTGCCAGC-5′.

Using SOEing PCR techniques (see, Horton et al., Biotechniques, 8(5):528-535 (1990)), a petF-petH operon was cloned into the NdeI and EcoRI sites of the plasmid pAS52-123 (described above). This plasmid was then used as a template from which the petF-petH operon piece was obtained for integration into the genomic DNA of E. coli MG1655 DV2.

Plasmid pDS57 (SEQ ID NO:110) was used as a template from which the Ptrc linked with an optimized ribosomal binding sequence were obtained. A “½ Kan” (SEQ ID NO:111) was obtained from the plasmid pEG63, which plasmid was constructed as follows.

A low copy plasmid pCL1920 (see, Lerner, et al., Nucleic Acid Res., 18(15):4631 (1990)), which contains a wild type E. coli tesA flanked by NdeI and EcoRI restriction sites, was used as a starting template. One of the three NdeI sites in this plasmid was then removed using the QuickChange™ Site-Directed Mutagenesis kit (Stratagene, La Jolla, Calif.). Following removal of the NdeI site, the plasmid was subjected to restriction digestions by NdeI and TatI. The digestion product was ligated with wild type E. coli tesA and a “½ Kan” sequence (SEQ ID NO:111) obtained from pKD13 (see, Datsensko, et al., supra). These pieces were gel-purified and connected using SOEing PCR techniques to form the following construct: E. coli lacI-Ptrc-optimized ribosomal binding sequence-petF-petH-½ Kan-homology E. coli lacZ. Specifically, the following primer was used for SOEing the 5′ end of the petF-petH piece to the 3′-end of the Ptrc-ribosomal binding sequence piece:

Primer 1:
(SEQ ID NO: 112)
AAAGAGGTATATATTAATGTATCGATTAAATAAGGAGGAATAACATATG
CCA ACTTATAAAGTGACACTAAT.

The following primer was used for SOEing the 3′-end of the petF-petH piece to the 5′-end of the Kanamycin marker in pEG63 (described above) to compliment the “½ Kan” in the E. coli genome:

Primer 2:
(SEQ ID NO: 113)
GCCTTCTTGACGAGTTCTTCTAAGATGAGTTTTTGTTCGGGCCCAAGC.

The SOEing PCR product was then electroporated into E. coli MG1655 DV2 (as described above), resulting in E. coli MG1655 DV2-petF-petH (integrated) cells.

### Example 32

This example describes the construction of a plasmid comprising a Synechococcus elongatus PCC7942 fatty aldehyde biosynthetic gene orf1594. The genomic DNA encoding Synechococcus elongatus PCC7942 orf1594 (YP_{—}400611) (SEQ ID NO:65) was amplified and cloned into the NcoI and EcoRI sites of plasmid OP-80 (pCL1920 derivative) (SEQ ID NO: 114) under the control of a Ptrc promoter. The OP-80 vector was constructed as follows.

A commercial vector pCL1920 (see, Lerner, et al., Nucleic Acids Res. 18:4631 (1990)), carrying a strong transcriptional promoter, was used as the starting point. The pCL1920 was digested with AflII and sfoI (New England Biolabs, Ipswich, Mass.). Three DNA fragments were produced as a result. The 3737-bp fragment was gel-purified using a gel-purification kit (Qiagen, Inc., Valencia, Calif.).

In parallel, a DNA sequence fragment comprising the Ptrc promoter and lacI sequence was obtained from a plasmid pTrcHis2 (Invitrogen, Carlsbad, Calif.) using the following primers:

(SEQ ID NO: 115)
LF302: 5′-ATATGACGTCGGCATCCGCTTACAGACA-3′;
and
(SEQ ID NO: 116)
LF303: 5′-AATTCTTAAGTCAGGAGAGCGTTCACCGACAA-3′.

These primers also introduced the restriction sites for ZraI and AflII. The PCR product was purified using a PCR-purification kit (Qiagen, Inc., Valencia, Calif.) and digested with ZraI and AflII. The PCR product was then gel-purified and ligated with the 3737-bp fragment (described above). The ligation mixture was transformed into TOP10® chemically competent cells (Invitrogen, Carlsbad, Calif.). The transformants were selected on Luria agar plates containing 100 μg/mL spectinomycin during overnight incubation. Plasmids within the resistant colonies were purified, verified with restriction digestion and confirmed with sequencing. One plasmid produced this way was retained, given the name of OP-80 (SEQ ID NO:114).

The resulting construct “OP80-PCC7942_{—}1594” was then used to transform the E. coli MG1655 DV2-petF-petH (integrated) cells, as described in Example 31.

### Example 33

This example describes the construction of a plasmid comprising a Nostoc punctiforme PCC73102 Npun02004178 decarbonylase. (ZP_{—}00108838) (SEQ ID NO:5) was amplified and cloned into the NdeI and XholI sites of vector OP-183 (pACYC derivative) (SEQ ID NO:105) under the control of a Ptrc promoter. The resulting construct was used, together with the OP80-PCC7942_{—}1594 construct above, to transform the E. coli MG1655 DV2-petF-petH (integrated) cells, as described in Example 31, resulting in a hydrocarbon production cell.

### Example 34

This example demonstrates fermentation and recovery processes to produce an alkane mixture of commercial grade quality for LAB synthesis, by fermentation of carbohydrates. A fermentation process was developed to produce a mix of hydrocarbons for use as LAB feedstock using the hydrocarbon production cell constructed as described in Examples 31-33 above. Two fermentation runs were performed with somewhat differing feed rates at stage 3, as described below. The two runs were named 031610 and 033010.

### Fermentation

The hydrocarbon production cell of Example 33 was maintained at −80° C. as a 20% (v/v) glycerol stock frozen after growth in an LB medium. The seed strain was cultivated as follows. A 1-mL vial of frozen cells was thawed and transferred into a 50-mL stage 1 medium (including LB broth supplemented with 100 mg/L carbenicillin and 100 mg/L spectinomycin), and incubated at 32° C. with shaking for 3-5 h, to an optical density at 600 nm (OD₆₀₀) of between 1 and 2. Next, 20-25 mL of the seed culture was transferred into 225 mL of a stage 2 medium (including 1.5 g/L KH₂PO₄, 3.3 g/L K₂HPO₄, 2.0 g/L (NH₄)₂SO₄, 40 mL/L 2M bis-tris buffer pH 7, 20 g/L glucose, 5 g/L casaminoacids, 0.12 g/L MgSO₄-7H₂O, 1 mL/L TM1 solution, 1 mL/L TV1 solution, 100 mg/L carbenicillin, and 100 mg/L spectinomycin) and incubated with shaking at 32° C. for 3-6 h, to reach an OD₆₀₀ of between 2 and 6. Then about 100 to about 250 mL of the seed culture was transferred into 3 L of a stage 3 medium (including 0.5 g/L (NH₄)₂SO₄, 2.0 g/L KH₂PO₄, 10 mL/L TM2 Solution, 0.034 g/L Iron Citrate, 5.0 g/L casaminoacids, 0.15 g/L MgSO₄-7H₂O, 20.0 g/L Feed Solution, 1.25 mL/L TV1 solution, 100 mg/L carbenicillin, and 100 mg/L spectinomycin, adjusted to pH 6.8) in a 5-L bioreactor to achieve an OD₆₀₀ of between 0.1 and 0.4 at inoculation.

The TV1 solution comprised 0.42 g/L riboflavin, 5.4 g/L pantothenic acid, 6 g/L niacin, 1.4 g/L pyridoxine, 0.06 g/L biotin, and 0.04 g/L folic acid. The TM1 solution comprised 27 g/L FeCl₃-6H₂O, 2 g/L ZnCl₂-4H₂O, 2 g/L CaCl₂-6H₂O, 2 g/L Na₂MoO₄-2H₂O, 1.9 g/L CuSO₄-5H₂O, 0.5 g/L H₃BO₃, and 100 mL/L concentrated HCl. The TM2 solution comprised 2 g/L ZnCl₂-4H₂O, 2 g/L CaCl₂-6H₂O, 2 g/L Na₂MoO₄-2H₂O, 1.9 g/L CuSO₄-5H₂O, 0.5 g/L H₃BO₃, and 40 mL/L concentrated HCl. The Feed Solution comprised 600 g/L glucose, 0.075 mL/L concentrated sulfuric acid, 3.9 g/L MgSO₄-7H₂O, 0.175 g/L Iron Citrate, 2.0 mL/L TV1 solution, and 1.6 g/L KH₂PO₄.

The bioreactor was operated at 1 LPM (liter per minute) airflow, pH 6.8 (which was controlled using ammonium hydroxide) and a temperature of 32° C. The agitation rate was automatically controlled to be between 300 and 1365 rpm, in coordination with the oxygen supplementation rate of 0 to 10%, in order to maintain a dissolved oxygen level (DO) of equal to or above 30% air saturation. The bioreactor was operated in a fed-batch mode with a ramp feed profile described in Table 11 below:

TABLE 11
stage 3 seed culture feed profile.

| | Run# 031610 Seed | Run# 033010 Seed |
|---|---|---|
| Time | Target Feed Rate | Target Feed Rate |
| (h) | (mL/h) | (mL/h) |
| 0 | 0 | 0 |
| 9 | 0 | 0 |
| 11 | 14 | 14 |
| 13 | 42 | 28 |
| 14 | 49 | 42 |
| 15 | 49 | 49 |
| 16 | 44 | 49 |
| 17 | 38.5 | 44 |
| 18 | 38.5 | 38.5 |

The feed rate was linearly ramped to meet target feed rate at the appropriate time points. The stage 3 seed cultures were transferred to the production bioreactor at 13-16 h after inoculation and/or at an OD₆₀₀ of between 25 and 60.

A 500-L production bioreactor containing about 250 L of a Production Culture Medium (containing 0.5 g/L (NH₄)₂SO₄, 3.5 g/L KH₂PO₄, 10 mL/L TM2 Solution, 0.034 g/L Iron Citrate, 5.0 g/L casaminoacids, 0.5 g/L MgSO₄-7H₂O, 10.0 g/L Feed Solution, 1.25 mL/L TV1 solution, 100 mg/L carbenicillin, and 100 mg/L spectinomycin, adjusted to pH 6.8) was inoculated with sufficient stage 3 seed culture to achieve an OD₆₀₀ of between 0.75 and 1.5. The culture was operated at 32° C. and 60-120 SLPM airflow, 0.3 bar headspace pressure and pH 6.8 (which was controlled using ammonium hydroxide). The agitation rate (150-314 rpm) and oxygen supplementation (0-40 SLPM) were automatically controlled to maintain a dissolved oxygen level of equal to or above 10% of air saturation. The headspace pressure was also adjusted (0.3-0.6 bar) as necessary. After inoculation, canola oil was fed to the bioreactor at 2-4 mL/min to target a total of about 15 kg of canola oil added over the process run time.

After an initial growth period that resulted in an OD₆₀₀ of 5 to 10, IPTG was added to a 1 mM final concentration to induce protein production. After induction, the cells were allowed to recover from induction. The Feed Solution was then fed to the bioreactor using the ramped profile as described in Table 12 below:

| | Target feed rate |
|---|---|
| Feed run | (g glucose/L |
| time (h) | initial volume/h) |
| 0 | 1.6 |
| 2 | 3.2 |
| 4 | 6.3 |
| 5 | 9.0 |
| 6 | 12.0 |
| 16 | 12.0 |
| 16-harvest | ≦12.0 |

After the initial growth period, the feed rate was manually adjusted as necessary to provide sufficient glucose for growth and production, and to maintain glucose at a level below 20 g/L, preferably below 5 g/L, and to meet the target feed rate at the designated time points as indicated in Table 12.

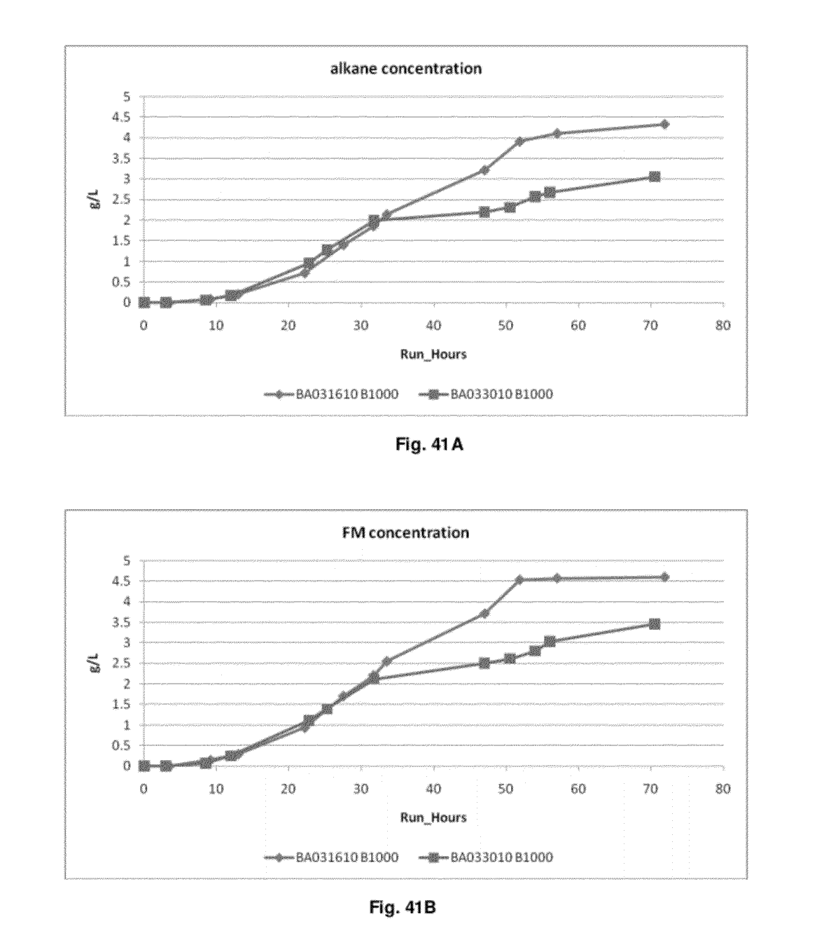

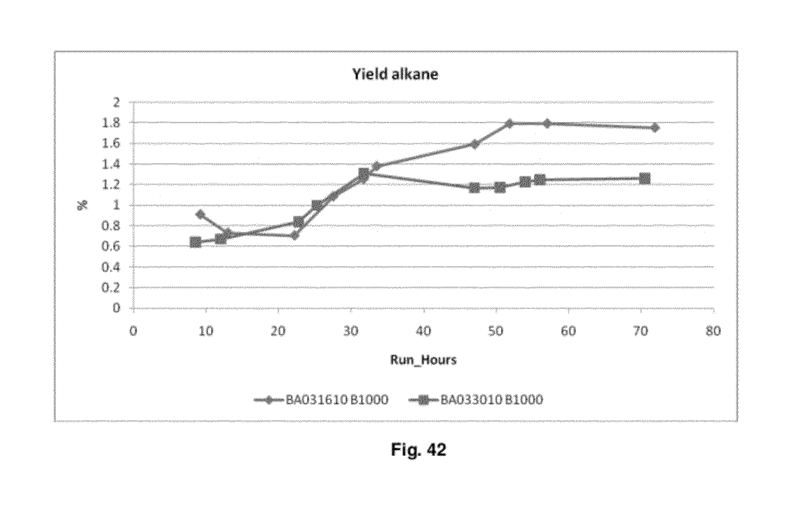

The cultures were harvested at about 72 h after inoculation for recovery of the hydrocarbon products. Throughout the bioreactor run, cell growth was monitored using OD₆₀₀, as depicted in FIG. 38. Glucose consumption or usage rate was also monitored at various time intervals as depicted in FIG. 39A. Glucose concentration in the medium was monitored by sampling at various time points as depicted in FIG. 39B. The concentration of canola oil in the culture medium was monitored throughout the run and depicted in FIG. 40. The amounts of alkane and fatty matters produced by the hydrocarbon production cells were monitored and depicted in FIG. 41A and FIG. 41B, respectively. The percentage yield of alkane vs. glucose feed was also monitored and depicted in FIG. 42.

Glucose consumption throughout the fermentation was analyzed by High Pressure Liquid Chromatography (HPLC). The HPLC analysis was performed according to methods commonly used for some sugars and organic acids in the art, which included the following conditions: Agilent HPLC 1200 Series with Refractive Index detector; Column: Aminex HPX-87H, 300 mm×7.8 mm; column temperature: 350° C.; mobile phase: 0.01M H₂SO₄ (aqueous); flow rate: 0.6 mL/m; injection volume: 20 μL.

The production of hydrocarbons and/or fatty matters was analyzed by gas chromatography with flame ionization detector (GC-FID). Hydrocarbon titers were determined by first taking 200 μL of broth and adding 200-800 μL of butyl acetate with 500 mg/L of n-tetracosane as an internal standard. The sample was then vortexed vigorously for 15 m, followed by centrifugation at 15,000×g for 5 m. The organic phase was derivatized with and equal volume of N,O-Bis(trimethylsilyl)trifluoroacetamide with 1% trimethylchlorosilane. The sample was then analyzed using a Thermo GC Ultra Fast equipped with an FID detector and a 5 m, 0.1 μm film thickness, 0.1 mm inner diameter DB1 Ultra Fast column. Briefly, the GC method used started with an oven temperature at 140° C. The oven was held at this temperature for 0.3 m after a 1-μL sample injection. The oven temperature was then ramped up to 300° C. at a rate of 300° C./m. The helium flow rate was set to a constant column flow rate of 0.5 mL/m. A split ratio of 1/100 was used. To quantify the hydrocarbon products, authentic references obtained from Sigma-Aldrich (St. Louis, Mo.) were used to make standard curves.

### Recovery

Two different processes were used to recover the alkane and canola products. The first recovery process was used to recover products from the fermentation run 031610, whereas the second recovery process was improved upon from the recovery process used for the 031610 run, and was used to recover products from the fermentation run 033010.

In the first recovery process, the whole broth was passed through an Alfa-Laval LAPX-404 Lab Separation Module (Alpha Laval, Lund, Sweden) at a normal feed rate of 2 Lpm to achieve an 85:15 heavy phase:light phase split. Back pressure of nearly 60 psig was placed on the heavy phase pump. About 90 kg of a light phase (containing about 27.5 g/L of alkane) and about 382 g/L of a heavy phase (containing about 0.45 g/L alkane) were recovered. The heavy phase was discarded.

The light phase was re-introduced through the Alfa-Laval LAPX-404 Lab Separation Module to obtain a second light phase. The feed rate was maintained at about 1 LPM with a heavy phase back pressure of about 8 to 10 psig. About 17.5 L of a second light phase was obtained. An about 77-L heavy phase containing about 0.64 g/L of alkane was discarded.

The light phase contained solids and water in addition to the desired alkane-canola oil product. It was subject to batch centrifugation in bottles at about 5,000×g to reduce impurities. The resulting centrate weighed about 12.5 kg, having a concentration of alkane of about 110 g/L. The material was odorous but was subject to subsequent distillation.

In the second recovery process, about 456 kg of whole broth was centrifuged directly to yield a light phase of about 20 L. A starting flow rate of about 3 LPM was applied to the first one third of the broth from the 033010 run. The heavy phase back pressure was regulated to be between about 15 and 35 psi and achieve about 150 to about 175 mL/min. The second one third of the broth from the 033010 run was used to ascertain whether a lower feed rate would reduce heavy phase alkane loss. This portion of the broth was subject to a starting flow rate of about 2 Lpm. Little if any difference in heavy phase alkane loss was found. In the last one third of the broth from the 033010 run, 3 Lpm was used as a starting flow rate. From these, a final light phase of about 22.4 kg was obtained.

The light phase was then centrifuged in bottles for about 15 m at 5,000×g. The top fraction of about 10 L was aspirated. The remaining volume (about 12 L) appeared to be a gelatinous gel phase. That remaining volume was filtered through a nominal 1.6 micron glass fiber filter (Whatman, Inc., Piscataway, N.J.) using a Buchner funnel (Sigma-Aldrich, St. Louis, Mo.). Post filtration, the alkane product in the filtrate took on a sparkling clear appearance, and was in a volume of about 8.8 liters, which contained about 55 g/L alkanes.

### Polishing

Two different polishing methods were used to further purify the alkane products. The first polishing method was used to purify the alkane product recovered from the 031610 fermentation run, whereas the second polishing method was used to purify the alkane product recovered from the 033010 fermentation run.

In the first polishing method, a distillation unit was established using a 2 L bottom flask, a column, a condenser, and four 50-mL product receiving flasks. An initial distillation was performed by keeping the vacuum level in the distillation unit at about 1 torr, the bottom flask at a temperature of lower than 200° C., and the column vapor temperature of below about 105° C. About 1,800 mL of composite distillate was collected after a few successive distillation runs.

Analysis of the distillate found that there was a substantial amount of higher molecular weight alkanes (e.g., C₁₆ or higher) remaining in the composite bottoms. The distillate at this stage was faintly yellow but had considerable odor. The composite bottom was re-distilled using a bottom temperature of about 260° C. and a column temperature of lower than about 150° C. An orange distillate of about 500 mL or more was recovered from this distillation run. This distillate, however, was found to contain oils and insoluble components. It was re-distilled at a bottom temperature of about 160° C. and a column temperature of about 105 to about 110° C. The resulting distillate was about 350 mL and was yellow.

In the second polishing method, a single distillation step was carried out at a bottom temperature of about 160° C. and a column temperature of about 105 to about 110° C. This resulted in about 500 mL of a yellow distillate. This distillate from the 033010 fermentation run/second recovery method/second polishing method was passed through a hexane-washed silica gel to remove a large portion of fatty materials. The material was then treated with bicarbonate, followed by treatment with anhydrous sodium sulfate, to remove residual water. Bleaching clay was used to further clean up the product in a final step, giving a clear, colorless, odorless alkane sample of high purity. This product was sent to Intertek, Inc. (Benica, Calif.) for testing.

### Example 35

This example describes a method for increasing the olefin content of hydrocarbons produced from Example 31-34.

The preferred precursors used to alkylate benzene are linear olefins with C₁₀ to C₁₆ chain lengths. The linear paraffin feedstock used to generate this molecule must first go through a dehydrogenation step to form mono-olefins. To prevent the formation of di-olefinic compounds, the percent conversion of paraffins to olefins must be minimized. As a result the feedstock for alkylation can consist of upwards of 90% unreactive paraffins. After alkylation, the paraffins are re-isolated and sent back for re-dehydrogenation. Creating a feedstock isolated enriched in mono-olefin compounds is desirable. The material isolated from Example 31 contains 20-30% olefinic compounds, higher than the typical alkylation feedstock, making it a more desirable feedstock then petroleum derived olefins. Increasing the olefinic content produced by the strain in Example 31 is desirable.

### Example 36

This example describes the production of linear alkyl benzyl sulfonates from hydrocarbons produced in Examples 31-35.

First, microbially-derived hydrocarbons from Examples 31-35 are used to form linear alkyl benzene using known methods. One exemplary method is described in WO2009/048761 (specifically incorporated by reference herein).

Next, the linear alkyl benzenes are sulfonated to produce molecules with detergent like properties. The linear alkyl benzene produced and described above are converted to linear alkyl benzyl sulfonates using well established manufacturing techniques. The linear alkyl benzene is sulfonated with SO₃ in air in a falling film reactor, as described in Synthetic Detergents, 7^{th} ed. A. S. Davidson & B. Milwidsky, John Wiley & Sons, Inc. 1987, pp. 151-186.

### Example 37

Anionic surfactant agglomerate

| Ingredient | Amount |
|---|---|
| C11-C13 linear alkyl benzene sulphonate (LAS) | 20 wt % |
| C12-C15 alkyl ethoxylated sulphate having an average | 2.4 wt % |
| degree of ethoxylation of 3 (AE₃S) | |
| Co-polymer of maleic acid and acrylic acid having a | 5.5 wt % |
| weight average molecular weight of from 50,000 Da to | |
| 90,000 Da, and a molar ratio of maleic acid to acrylic | |
| acid of from 0.25 to 0.35 (copolymer) | |
| Tallow alkyl ethoxylated alcohol having an average | 2.9 wt % |
| degree of ethoxylation of 80 (TAE₈₀) | |
| Polyethylene glycol | 0.1 wt % |
| Sodium sulphate | 40 wt % |
| Sodium carbonate | 20 wt % |
| Water and miscellaneous | 9.1 wt % |

### Agglomeration Process

The above-described anionic surfactant agglomerate is prepared by the following process. The TAE₈₀, polyethylene glycol, co-polymer and aqueous anionic surfactant paste comprising the LAS and AE₃S are introduced into a twin screw extruder and extruded into a Lodige CB mixer. Dry material comprising the sodium sulphate and sodium carbonate is introduced into the Lodige CB mixer and mixed with the TAE₈₀, polyethylene glycol, co-polymer and anionic surfactant paste to form a mixture. The mixture is then transferred into a Lodige KM mixer, water is sprayed into the KM and the mixture is agglomerated to form intermediate agglomerates. The intermediate agglomerates exiting the Lodige KM mixer are passed through a sieve and intermediate agglomerates having a particle size greater than 5 millimeters are removed from the remainder of the intermediate agglomerates and recycled back to the Lodige CB mixer. The remaining portion of the intermediate agglomerates is transferred into a fluid bed dryer and then a fluid bed cooler. Intermediate agglomerates having a very small particle size (i.e., the fines having a particle size of less than 250 micrometers) are elutriated by the fluid bed exhaust system where they are collected and recycled back to the CB mixer. The remaining portion of the intermediate agglomerates exiting the fluid bed cooler is passed through a sieve and intermediate agglomerates having a particle size greater than 850 micrometers are removed from the remainder of the intermediate agglomerates, passed through a grinder where they are ground into particles having a smaller particle size and are then recycled back to the fluid bed dryer. The remaining portion of the intermediate agglomerates is collected and is suitable for use in the present invention; this remaining portion is the anionic surfactant agglomerates having the above described formulation.

Solid Laundry Detergent Composition

| Ingredient | Amount |
|---|---|
| Anionic surfactant agglomerate | 78 wt % |
| Sodium bicarbonate | 19.3 wt % |
| Sodium sulphite | 0.5 wt % |
| Polyvinylpyrrolidone | 0.2 wt % |
| Hydrophobic silica | 0.5 wt % |
| Dry-add perfume | 0.5 wt % |
| Spray-on perfume | 0.2 wt % |
| Orange Dye | 0.8 wt % |

### Finished Product Process

The above described anionic surfactant agglomerate is mixed with solid material comprising sodium bicarbonate, sodium sulphite, polyvinylpyrrolidone, hydrophobic silica and dry-add perfume. The sprayed-on perfume and orange dye (in liquid form) are then sprayed on to this mixture to obtain a solid laundry detergent composition described in more detail above.

### Example 38

As in Example 37, except that some of the sodium sulphate is added into the Lodige KM mixer, in addition to the Lodige CB mixer.

### Example 39

As in Example 37, except that the agglomerate comprises 37 wt % sodium sulphate (instead of 40 wt %) and 3 wt % zeolite A. The zeolite A is added into the fluid bed dryer in fine particulate form having a weight average particle size of from 2 micrometers to 25 micrometers.

### Example 40

As in Example 37, except that the solid laundry detergent composition comprises 76 wt % anionic surfactant agglomerate (described in Example 37.1) and 2 wt % zeolite A. The zeolite A is in fine particulate form having an average particle size of from 2 micrometers to 25 micrometers and is added to the anionic surfactant agglomerate in the finished product process along with the other dry-added materials such as the sodium bicarbonate.

### Example 41

The following formulas are prepared at room temperature by simple liquid mixing procedures.

| | 1 | 2 | 3 | 4 | 5 | 6 | 7 |
|---|---|---|---|---|---|---|---|
| Mg Linear alkyl | 9.02 | 6.31 | 6.31 | 6.31 | 6.31 | 6.31 | 6.31 |
| Benzene sulfonate | | | | | | | |
| Na Linear alkyl | 3.00 | 2.10 | 2.10 | 2.10 | 2.10 | 2.10 | 2.10 |
| Benzene sulfonate | | | | | | | |
| Lauryl myristal amine | 5.00 | 3.50 | 3.50 | 3.50 | 3.50 | 3.50 | 3.50 |
| oxide | | | | | | | |
| SD No. 3 alcohol | 2.15 | 1.51 | 1.51 | 1.51 | 1.51 | 1.51 | 1.51 |
| NH4AEOS 1:3 OXO | 11.50 | 8.05 | 8.05 | 8.05 | 8.05 | 8.05 | 8.05 |
| APG625 | 9.50 | 6.65 | 6.65 | 6.65 | 6.65 | 6.65 | 6.65 |
| Dimethyol dimethyl | 0.11 | 0.08 | 0.08 | 0.08 | 0.08 | 0.08 | 0.08 |
| hydantoin | | | | | | | |
| 40% SXS solution | 1.25 | 0.88 | 0.88 | 0.88 | 0.88 | 0.88 | 0.88 |
| Dissolvine D-40 | 0.13 | 0.09 | 0.09 | 0.09 | 0.09 | 0.09 | 0.09 |
| Neodol 1-3 | 0.00 | 15.00 | 30.00 | 13.75 | 12.50 | 10.00 | 7.50 |
| Water | 58.26 | 55.78 | 40.78 | 57.03 | 58.28 | 60.78 | 63.28 |

| | 8 | 9 | 10 | 11 | 12 | 13 |
|---|---|---|---|---|---|---|
| Mg Linear alkyl | 6.31 | 5.05 | 5.05 | 5.37 | 4.74 | 6.00 |
| Benzene sulfonate | | | | | | |
| Na Linear alkyl | 2.10 | 1.68 | 1.68 | 1.79 | 1.58 | 2.00 |
| Benzene sulfonate | | | | | | |
| Lauryl myristal amine | 3.50 | 2.80 | 2.80 | 2.98 | 2.63 | 3.33 |
| oxide | | | | | | |
| SD No. 3 alcohol | 1.51 | 1.20 | 1.20 | 1.28 | 1.13 | 1.43 |
| NH4AEOS 1:3 OXO | 8.05 | 6.44 | 6.44 | 6.84 | 6.04 | 7.65 |
| APG625 | 6.65 | 5.32 | 5.32 | 5.65 | 4.99 | 6.32 |
| Dimethyol dimethyl | 0.08 | 0.06 | 0.06 | 0.07 | 0.06 | 0.07 |
| hydantoin | | | | | | |
| 40% SXS solution | 0.88 | 0.70 | 0.70 | 0.74 | 0.66 | 0.83 |
| Dissolvine D-40 | 0.09 | 0.07 | 0.07 | 0.08 | 0.07 | 0.09 |
| Neodol 1-3 | 5.00 | 10.00 | 15.00 | 15.00 | 15.00 | 5.00 |
| Water | 65.78 | 66.63 | 61.63 | 60.16 | 63.09 | 67.24 |

### Example 42

The following compositions in wt. % are prepared by a simple mixing procedure.

| | Standard | |
|---|---|---|
| Surfactant | Reference Formula | A |
| MgLAS | 9 | 9 |
| NaLAS | 3 | 3 |
| NH4AEOS 1.3 | 11.5 | 11.5 |
| mole EO | | |
| Amine Oxide | 5.417 | 5.417 |
| APG | 10 | — |
| NaAEOS 5EO | — | 10 |
| SXS hydrotrope | 1.5 | |
| Salt | — | 1 |
| DMDMH | .11 | .11 |
| Pentasodium | .125 | .125 |
| pentetate | | |
| Ethanol | 6.1 | 6.1 |
| pH | 7 | 7 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A recombinant host cell culture for production of a linear alkene or alkane, the host cell culture comprising a recombinant microorganism engineered to express a polynucleotide encoding a polypeptide having the amino acid sequence presented as SEQ ID NO:2, 4, 6, 8, 10, 12, 14, 16, 18, 20, 22, 24, 26, 28, 30, 32, 34, or 36, wherein a linear alkene or alkane is found in the cell-free culture supernatant.

2. A method of producing a linear alkyl benzene, the method comprising:
(i) fermenting the host cell culture of claim 1 in the presence of a carbon source, thereby producing a linear alkene;
(ii) isolating the linear alkene from the fermented host cell culture; and
(iii) reacting the linear alkene with benzene in the presence of a catalyst under reaction conditions sufficient for alkylation of the benzene, thereby producing a linear alkyl benzene.

3. A method of producing a linear alkyl benzene, the method comprising:
(i) expressing in a host cell a polynucleotide comprising the nucleotide sequence of SEQ ID NO:1, 3, 5, 7, 9, 11, 13, 15, 17, 19, 21, 23, 25, 27, 29, 31, 33, or 35,
(ii) culturing the host cell in the presence of a carbon source, thereby producing a linear alkene;
(iii) isolating the linear alkene from the host cell culture; and
(iv) reacting the linear alkene with benzene in the presence of a catalyst under reaction conditions sufficient for alkylation of the benzene, thereby producing a linear alkyl benzene.

4. A method of producing a linear alkyl benzene according to claim 3, further comprising:
(i) expressing a polynucleotide comprises the nucleotide sequence presented as SEQ ID NO: 117, 119, 120, 122, 124, 125, 127, or 129;
(ii) culturing the host cell in the presence of a carbon source, thereby producing a linear alkene;
(iii) isolating the linear alkene from the host cell culture; and
(iv) reacting the linear alkene with benzene in the presence of a catalyst under reaction conditions sufficient for alkylation of the benzene, thereby producing a linear alkyl benzene.

5. A method of producing a linear alkyl benzene according to claim 2, further comprising:
(i) expressing in the host cell a polynucleotide encoding a polypeptide comprising the amino acid sequence presented as SEQ ID NO: 118, 121, 123, 126, 128, or 130,
(ii) fermenting the host cell culture in the presence of a carbon source, thereby producing a linear alkene;
(iii) isolating the linear alkene from the host cell; and
(iv) reacting the linear alkene with benzene in the presence of a catalyst under reaction conditions sufficient for alkylation of the benzene, thereby producing a linear alkyl benzene.

6. The method of claim 2, wherein the host cell is a bacterial cell.

7. The method of claim 6, wherein the host cell is an E. coli cell.

8. The method of claim 3, wherein the host cell is a bacterial cell.

9. The method of claim 7, wherein the host cell is an E. coli cell.

10. The method of claim 2, wherein the alkene comprises a C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, or C₂₅ alkene.

11. The method of claim 3, wherein the alkene comprises a C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, or C₂₅ alkene.

12. The method of claim 2, further comprising culturing the host cell in the presence of an unsaturated aldehyde comprising a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or C₂₆ aldehyde.

13. The method of claim 3, further comprising culturing the host cell in the presence of an unsaturated aldehyde comprising a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or C₂₆ aldehyde.

14. The method of claim 2, further comprising culturing the host cell in the presence of an unsaturated fatty acid comprising a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or C₂₆ fatty acid.

15. The method of claim 3, further comprising culturing the host cell in the presence of an unsaturated fatty acid comprising a C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, or C₂₆ fatty acid.

16. The method of claim 2, further comprising sulfonating the linear alkyl benzene to produce a linear alkyl sulfonate.

17. The method of claim 3, further comprising sulfonating the linear alkyl benzene to produce a linear alkyl sulfonate.

18. A surfactant composition comprising the linear alkyl sulfonate of claim 16.

19. A surfactant composition comprising the linear alkyl sulfonate of claim 17.
